(19) United States
(12) Patent Application Publication
(10) Pub. No.: US 2024/0218386 A1
(43) Pub. Date: Jul. 4, 2024

(54) Compositions and Methods for Producing Tobacco Plants and Products Having Altered Alkaloid Levels

(71) Applicant: ALTRIA CLIENT SERVICES LLC, Richmod, VA (US)

(72) Inventors: Chengalrayan KUDITHIPUDI, Hennico, VA (US); Chengalrayan KUDITHIPUDI, Midiothian, VA (US); Yanxin SHEN, Hennico, VA (US); Dongmei XU, Glen Allen, VA (US)

(73) Assignee: ALTRIA CLIENT SERVICES LLC, Richmod, VA (US)

(21) Appl. No.: 17/781,797

(22) PCT Filed: Dec. 2, 2020
(86) PCT No.: PCT/US2020/062857

§ 371 Date: Dec. 12, 2022

Related U.S. Application Data
(60) Provisional application No. 62/942,957, filed on Dec. 3, 2019.

(51) Int. Cl. C12N 15/82; A24B 13/00; A24B 15/00

(52) U.S. Cl. CPC C12N 15/8243; A24B 13/00; A24B 15/00

(57) ABSTRACT

The present disclosure provides composition and methods for manipulating alkaloid or nicotine levels in tobacco plants. Also provided are the identification and genetic engineering of target genes (e.g., arginine decarboxylase (ADC), aspartate oxidase (AO), or ornithine decarboxylase (ODC)) for producing tobacco plants with altered total alkaloid and nicotine levels and commercially acceptable leaf grade, their development via breeding or transgenic approaches, and production of tobacco products from these tobacco plants.

### CROSS-REFERENCE TO RELATED APPLICATIONS

This application claims the benefit of priority of U.S. Provisional Application 62/942,957, filed Dec. 3, 2019, which is herein incorporated by reference in its entirety.

### INCORPORATION OF SEQUENCE LISTING

A sequence listing contained in the file named “P34775WO00_SL.txt” which is 283,115 bytes (measured in MS-Windows®) and created on Dec. 2, 2020, is filed electronically herewith and incorporated by reference in its entirety.

### FIELD

The present disclosure includes tobacco plants having altered total alkaloid and nicotine levels and commercially acceptable leaf grade, their development via breeding or transgenic approaches, and production of tobacco products from these tobacco plants.

### BACKGROUND

Nicotine is the main alkaloid accumulating in tobacco leaves. Nicotine and other minor alkaloids (e.g., nornicotine, anabasine, and anatabine) are also precursors to tobacco-specific nitrosamines (TSNA). Demands exist for development of tobacco cultivars with lower levels of nicotine.

In commercial tobacco cultivars, nicotine represents 90-95% of the total alkaloid pool or 2-5% of total leaf dry weight. Nicotine is synthesized in the roots, and translocated through the xylem to aerial parts of the plant where it accumulates in the leaves and is exuded by trichomes in response to insect herbivory.

There is a need to identify genes that can be engineered to reduce alkaloid, more specifically, nicotine without impacting leaf phenotypes, and to develop tobacco plants and products that contain altered nicotine levels (e.g., reduced nicotine) while maintaining (if not making superior) tobacco leaf quality.

### SUMMARY

In an aspect, the present disclosure provides a modified tobacco plant, or part thereof, comprising a genetic modification in a gene and downregulating the expression or activity of the gene, wherein the gene encodes a nucleic acid sequence having at least 80% identity to a polynucleotide sequence selected from the group consisting of SEQ ID NOs: 19-36.

In an aspect, the present disclosure provides a modified tobacco plant, or part thereof, comprising a genetic modification targeting a gene and downregulating the expression or activity of the gene, wherein the gene encodes a nucleic acid sequence having at least 80% identity to a polynucleotide sequence selected from the group consisting of SEQ ID NOs: 19-36.

In another aspect, the present disclosure provides a modified tobacco plant, or part thereof, comprising a non-natural mutation in a polynucleotide having at least 80% identity to a polynucleotide sequence selected from the group consisting of SEQ ID NOs: 1-36.

In an aspect, the present disclosure provides a modified tobacco plant, or part thereof, comprising a recombinant nucleic acid construct comprising a heterologous promoter operably linked to a polynucleotide that encodes a non-coding RNA molecule, where the non-coding RNA molecule is capable of binding to an mRNA having at least 80% identity to a polynucleotide sequence selected from the group consisting of SEQ ID NOs: 19-36.

In another aspect, the present disclosure provides a modified tobacco plant, or part thereof, comprising a genetic modification in a gene and downregulating the expression or activity of the gene, wherein the gene encodes a polypeptide having at least 80% identity or similarity to an amino acid sequence selected from the group consisting of SEQ ID NOs: 37-54.

In an aspect, the present disclosure provides a modified tobacco plant, or part thereof, comprising a genetic modification targeting a gene and downregulating the expression or activity of the gene, wherein the gene encodes a polypeptide having at least 80% identity or similarity to an amino acid sequence selected from the group consisting of SEQ ID NOs: 37-54.

In an aspect, the present disclosure provides a modified tobacco plant, or part thereof, comprising a non-natural mutation in a polynucleotide having a nucleic acid sequence encoding a polypeptide having at least 80% identity or similarity to an amino acid sequence selected from the group consisting of SEQ ID NOs: 37-54.

In another aspect, the present disclosure provides a modified tobacco plant, or part thereof, comprising a recombinant nucleic acid construct comprising a heterologous promoter operably linked to a polynucleotide that encodes a non-coding RNA molecule, where the non-coding RNA molecule is capable of binding to an RNA encoding a polypeptide having at least 80% identity or similarity to an amino acid sequence selected from the group consisting of SEQ ID NOs: 37-54, wherein the non-coding RNA molecule suppresses the expression of the polypeptide.

In an aspect, the present disclosure provides a population of the tobacco plants described here, cured tobacco material from the tobacco plant described here, and reconstituted tobacco, a tobacco blend and a tobacco product made from the cured tobacco material.

In another aspect, the present disclosure provides a method for producing a reduced-alkaloid tobacco plant, the method comprising: (a) downregulating the expression or activity of a gene encoding (i) a nucleic acid sequence having at least 90% identity to a polynucleotide sequence selected from the group consisting of SEQ ID NOs: 1-36, or (ii) an amino acid sequence having at least 90% identity or similarity to a polypeptide sequence selected from the group consisting of SEQ ID NOs: 37-54; and (b) harvesting leaves or seeds from the tobacco plant.

### BRIEF DESCRIPTION OF THE DRAWINGS

FIG. 1 a. In-vitro Nicotiana tabacum (tobacco) plantlets in Dixie cups are obtained after seed germination and initial growth on solid Murashige and Skoog agar media, supplemented with vitamins and 30 g L⁻¹ sucrose. Seedlings are maintained at 24° C. under a 16/8-h photoperiod. b. Rooted transformants after approximately 5-weeks of growth in Dixie cups are transferred to soil in the greenhouse and cultivated autotrophically under ambient sunlight conditions.

FIG. 2 Transient expression upon in vitro agroinfiltration of Nicotiana tabacum (tobacco) leaves with Agrobacterium transformants comprising the RNAi constructs used in this work. A minimum of three leaves per Agrobacterium transformant, including the wild type control, are infiltrated by pressing the tip of a 3 mL sterile syringe with the Agrobacterium mix into the abaxial side of the leaves. Samples are collected for analysis 48 hours after the agroinfiltration.

FIG. 3 Spectrophotometric determination of total alkaloid leaf extracts from Nicotiana tabacum (tobacco). (Upper) Absorbance spectrum of the alkaloid-containing solution measured in the 350-550 nm region defining a 415 nm absorbance peak. (Lower) Calibration curve of the absorbance maximum at 415 nm, as a function of the concentration of nicotine in the 400 μL assay solution.

FIG. 4 GC-FID analysis with a Shimadzu GC-2014 Gas Chromatography apparatus equipped with a flame-ionization detector (FID) showing the signal amplitude of the samples with different concentrations of nicotine. The latter has a retention time of 10.2 minutes under the experimental conditions employed. The analysis shows a linear response of the apparatus to nicotine concentration and a detection limit of 12.5 μg/mL nicotine.

FIG. 5 Transcript levels as a measure of gene expression in T1 RNAi transformant tobacco leaves. The results show a substantially lower transcript level for all independent event lines derived from the ADC, AIC, AO, and ODC RNAi transformations. The ARG and SAMS transformant lines show mixed and/or inconsistent results, with some lines having transcript levels comparable to the wild type, while others show lower levels. Each sample is run in triplicate. Levels of gene expression at the mRNA level are reported as percentage of each gene transcript in the presence of the RNAi construct, compared to those of the control.

FIG. 6 Total alkaloids content of leaves from 36 independent transformant lines with 6 different RNAi constructs of T1 plants plus the wild type control in the early growth phase while seedlings are still in Dixie cups in the lab. Note that at this early vegetative stage only the AO1 RNAi and some of the ODC RNAi lines show a significantly lower total alkaloids content than the wild type (WT). In contrast, all AO2 and SAMS RNAi lines show significantly greater alkaloid content values than the wild type (WT). The average dry weight of multiple samples is measured upon lyophilizing a known weight of fresh material (FW) and, subsequently, measuring the weight of the resulting dry biomass (DW). The latter is found to account for about 39.5% (±2.0%) of the fresh weight in seedling leaves.

FIG. 7 Total alkaloids content of leaves harvested at the greenhouse from 36 independent transformant lines with 6 different RNAi constructs of T1 plants plus the wild type control following the early budding stage. At this plant development stage, the content of total alkaloids in most of the transgenic lines is lower compared to the control (WT), while some of the AO2 and ADC lines continued to show significantly greater values. The average dry weight of multiple samples is measured upon lyophilizing a known weight of fresh material (FW) and, subsequently, measuring the weight of the resulting dry biomass (DW). The latter is found to be about 34.5% (±2.7%) of the fresh weight in greenhouse-developed leaves.

FIG. 8 Nicotine content of leaves from T1 plants grown at the greenhouse. The assay include 36 independent transformant lines with 6 different RNAi constructs of T1 plants plus the wild type control following the early budding stage. Transformants expressing the AO1-RNAi and AO2-RNAi have the lowest nicotine content compared to the control (WT). The next most significant suppression of nicotine content is observed in the ODC-RNAi lines 1, 13, 14 and 15. Also, ADC-RNAi has at least two lines with lower than wild type nicotine content. In contrast, lines AIC-RNAi, ARG-RNAi and SAMS2-RNAi do not display significant differences compared with the wild type control.

FIG. 9 Mean content of putrescine (a), spermidine (b), and cadaverine (c) in each type of RNAi lines examined in this work. Compared to wild type, ADC-RNAi and ODC-RNAi lines have significant lower content of putrescine, while AIC-RNAi, ARG-RNAi and SAMS-RNAi lines have levels of putrescine equivalent to that measured in the control (WT). The spermidine and cadaverine content of the RNAi transformants is statistically invariable from that of the control.

FIG. 10 Phenotypic variation noted for the older leaves in the AIC-RNAi (a) and AO1-RNAi (b) lines. In all AIC-RNAi lines (a), the fully expanded and oldest leaves show signs of early senescence-like discoloration. Similarly, in some of the AO1-RNAi lines (b), the fully expanded and older (lower) leaves also show symptoms of early senescence-like discoloration. Lines that show this phenotype (AO1 lines 7, 10, and 11) are the ones with the lowest level of nicotine content. It should be noted that these “early” coloration changes affect the oldest leaves of the transformants, as compared with the wild type control, whereas the 3^{rd} and 4^{th} leaves from the top that are harvested for alkaloids and nicotine analyses have a normally green pigmentation and otherwise healthy phenotype, very much like the wild type control. This phenomenology is limited to the lower leaves and does not seem to affect the upper leaves on these plants, including those that are samples for alkaloids, nicotine, and polyamines analysis.

FIG. 11 Putative alkaloid biosynthetic pathways schematic in Nicotiana tabacum. Shown are the arginine and proline metabolism, alkaloids biosynthesis, and nicotinate and nicotinamide metabolism pathways, as they may be involved in the analysis reported in this work.

### BRIEF DESCRIPTION OF THE SEQUENCES

SEQ ID NOs: 1 to 18 set forth genomic DNA sequence (including regions such as promoter, 5′ UTR, introns, 3′ UTR, and terminator) of various genes involved in alkaloid biosynthesis.

SEQ ID NOs: 19 to 36 set forth cDNA sequences of various genes involved in alkaloid biosynthesis.

SEQ ID NOs: 37 to 54 set forth polypeptide sequences of various genes involved in alkaloid biosynthesi.

SEQ ID NOs: 55 to 64 set forth exemplary RNAi sequences for targeting various genes involved in alkaloid biosynthesis.

SEQ ID NOs: 65 to 84 set forth exemplary primer sequences.

Various sequences may include “N” in nucleotide sequences or “X” in amino acid sequences. “N” can be any nucleotide, e.g., A, T, G, C, or a deletion or insertion of one or more nucleotides. In some instant, a string of “N” are shown. The number of “N” does not necessarily correlate with the actual number of undetermined nucleotides at that position. The actual nucleotide sequences can be longer or shorter than the shown segment of “N”. Similarly, “X” can be any amino acid residue or a deletion or insertion of one or more amino acids. Again, the number of “X” does not necessarily correlate with the actual number of undetermined amino acids at that position. The actual amino acid sequences can be longer or shorter than the shown segment of “X”. Notwithstanding the use of A, T, G, C (compared to A, U, G, C) in describing any SEQ ID in the sequence listing, that SEQ ID can also refer to a RNA sequence, depending on the context in which the SEQ ID is mentioned.

### DETAILED DESCRIPTION

Unless defined otherwise, technical and scientific terms used herein have the same meaning as commonly understood by one of ordinary skill in the art. One skilled in the art will recognize many methods can be used in the practice of the present disclosure. Indeed, the present disclosure is in no way limited to the methods and materials described. Where a term is provided in the singular, the inventors also contemplate aspects of the disclosure described by the plural of that term, and vice versa. Where there are discrepancies in terms and definitions used in references that are incorporated by reference, the terms used in this application shall have the definitions given herein. Other technical terms used have their ordinary meaning in the art in which they are used, as exemplified by various art-specific dictionaries, for example, “The American Heritage® Science Dictionary” (Editors of the American Heritage Dictionaries, 2011, Houghton Mifflin Harcourt, Boston and New York), the “McGraw-Hill Dictionary of Scientific and Technical Terms” (6th edition, 2002, McGraw-Hill, New York), or the “Oxford Dictionary of Biology” (6th edition, 2008, Oxford University Press, Oxford and New York).

Any references cited herein, including, e.g., all patents and publications are incorporated by reference in their entirety and to the same extent as if each individual publication, patent, or patent application is specifically and individually indicated to be incorporated by reference.

When a grouping of alternatives is presented, any and all combinations of the members that make up that grouping of alternatives is specifically envisioned. For example, if an item is selected from a group consisting of A, B, C, and D, the inventors specifically envision each alternative individually (e.g., A alone, B alone, etc.), as well as combinations such as A, B, and D; A and C; B and C; etc. The term “and/or” when used in a list of two or more items means any one of the listed items by itself or in combination with any one or more of the other listed items. For example, the expression “A and/or B” is intended to mean either or both of A and B—i.e., A alone, B alone, or A and B in combination. The expression “A, B and/or C” is intended to mean A alone, B alone, C alone, A and B in combination, A and C in combination, B and C in combination, or A, B, and C in combination.

When a range of numbers is provided herein, the range is understood to inclusive of the edges of the range as well as any number between the defined edges of the range. For example, “between 1 and 10” includes any number between 1 and 10, as well as the number 1 and the number 10.

As used herein, the singular form “a,” “an,” and “the” include plural references unless the context clearly dictates otherwise. For example, the term “a compound” or “at least one compound” may include a plurality of compounds, including mixtures thereof.

The term “about” is used herein to mean approximately, roughly, around, or in the region of. When the term “about” is used in conjunction with a numerical range, it modifies that range by extending the boundaries above and below the numerical values set forth, and is understood to mean plus or minus 10%. For example, “about 100” would include from 90 to 110.

To avoid any doubt, used herein, terms or phrases such as “about”, “at least”, “at least about”, “at most”, “less than”, “greater than”, “within” or alike, when followed by a series of list of numbers of percentages, such terms or phrases are deemed to modify each and every number of percentage in the series or list, regardless whether the adverb, preposition, or other modifier phrase is reproduced prior to each and every member.

As used herein, a “low alkaloid variety” (also referred to as “LA variety”) of tobacco refers to tobacco variety comprising one or more genetic modifications reducing the total alkaloids (measured via dry weight) to a level less than 25% of the total alkaloid level in a control tobacco variety of a substantially similar genetic background except for the one or more genetic modifications. As a non-limiting example, KY171 can serve as a control for a low-alkaloid variety LA KY171. Without being limiting, low-alkaloid tobacco varieties include LA Burley 21, LAFC53, LN B&W, and LN KY171. Similarly, a “low nicotine variety” (also referred to as “LN variety”) of tobacco refers to tobacco variety comprising one or more genetic modifications reducing nicotine (measured via dry weight) to a level less than 25% of the nicotine level in a control tobacco variety of a substantially similar genetic background except for the one or more genetic modifications.

As used herein, a “genetic modification” refers to a change in the genetic makeup of a plant or plant genome. A genetic modification can be introduced by methods including, but not limited to, mutagenesis, genome editing, genetic transformation, or a combination thereof. A genetic modification includes, for example, a mutation (e.g., a non-natural mutation) in a gene or a transgene targeting a gene (e.g., an arginine decarboxylase (ADC) transgene targets an ADC gene). As used here, “targeting” refers to either directly upregulating or directly downregulating the expression or activity of a gene. As used here, “directly”, in the context of a transgene impacting the expression or activity of a gene, refers to the impact being exerted over the gene via a physical contact or chemical interaction between the gene (e.g., a promoter region or a UTR region) or a product encoded therein (e.g., a mRNA molecule or a polypeptide) and a product encoded by the transgene (e.g., a small non-coding RNA molecule or a protein such as a transcription factor or a dominant negative polypeptide variant). In an aspect, a transgene impacts the expression or activity of a target gene without involving a transcription factor (e.g., the transgene does not encode a transcription factor and/or does not suppress the expression or activity of a transcription factor that in turn regulates the target gene).

As used herein, a “mutation” refers to an inheritable genetic modification introduced into a gene to alter the expression or activity of a product encoded by a reference sequence of the gene. A mutation in a certain gene, e.g., an arginine decarboxylase (ADC) is referred to as an ADC mutant. Such a modification can be in any sequence region of a gene, for example, in a promoter, 5′ UTR, exon, intron, 3′ UTR, or terminator region. In an aspect, a mutation reduces, inhibits, or eliminates the expression or activity of a gene product. In another aspect, a mutation increases, elevates, strengthens, or augments the expression or activity of a gene product. In an aspect, mutations are not natural polymorphisms that exist in a particular tobacco variety or cultivar. It will be appreciated that, when identifying a mutation, the reference sequence should be from the same tobacco variety or background. For example, if a modified tobacco plant comprising a mutation is from the variety TN90, then the corresponding reference sequence should be the endogenous TN90 sequence, not a homologous sequence from a different tobacco variety (e.g., K326). In an aspect, a mutation is a “non-natural” or “non-naturally occurring” mutation. As used herein, a “non-natural” or “non-naturally occurring” mutation refers to a mutation that is not, and does not correspond to, a spontaneous mutation generated without human intervention. Non-limiting examples of human intervention include mutagenesis (e.g., chemical mutagenesis, ionizing radiation mutagenesis) and targeted genetic modifications (e.g., CRISPR-based methods, TALEN-based methods, zinc finger-based methods). Non-natural mutations and non-naturally occurring mutations do not include spontaneous mutations that arise naturally (e.g., via aberrant DNA replication in a germ line of a plant.

As used herein, a tobacco plant can be from any plant from the Nicotiana genus including, but not limited to Nicotiana tabacum, Nicotiana amplexicaulis PI 271989; Nicotiana benthamiana PI 555478; Nicotiana bigelovii PI 555485; Nicotiana debneyi; Nicotiana excelsior PI 224063; Nicotiana glutinosa PI 555507; Nicotiana goodspeedii PI 241012; Nicotiana gossei PI 230953; Nicotiana hesperis PI 271991; Nicotiana knightiana PI 555527; Nicotiana maritima PI 555535; Nicotiana megalosiphon PI 555536; Nicotiana nudicaulis PI 555540; Nicotiana paniculata PI 555545; Nicotiana plumbaginifolia PI 555548; Nicotiana repanda PI 555552; Nicotiana rustica; Nicotiana suaveolens PI 230960; Nicotiana sylvestris PI 555569; Nicotiana tomentosa PI 266379; Nicotiana tomentosiformis; and Nicotiana trigonophylla PI 555572. In an aspect, a tobacco plant described here is a Nicotiana tabacum plant.

In an aspect, the present disclosure provides a tobacco plant, or part thereof, comprising a genetic modification upregulating or downregulating the expression or activity of one or more genes encoding arginine decarboxylase (ADC), aspartate oxidase (AO), or ornithine decarboxylase (ODC). As used herein, the upregulation or downregulation of a gene by a genetic modification is determined by comparing a plant having the genetic modification with a corresponding control plant not having the genetic modification.

### Plants

In an aspect, the present disclosure provides a modified tobacco plant, or part thereof, comprising a genetic modification in or targeting a gene, wherein the gene encodes a nucleic acid sequence having at least 70%, at least 80%, at least 90%, at least 95%, at least 97%, at least 99%, or 100% identity to a polynucleotide sequence selected from the group consisting of SEQ ID NOs: 19-36. In an aspect, the present disclosure provides a modified tobacco plant, or part thereof, comprising a genetic modification in or targeting a gene and downregulating the expression or activity of the gene, wherein the gene encodes a nucleic acid sequence having at least 70%, at least 80%, at least 90%, at least 95%, at least 97%, at least 99%, or 100% identity to a polynucleotide sequence selected from the group consisting of SEQ ID NOs: 19-36. In an aspect, a gene being downregulated encodes a nucleic acid sequence having at least 90% identity to a polynucleotide sequence selected from the group consisting of SEQ ID NOs: 19-36. In an aspect, a gene being downregulated encodes a nucleic acid sequence having at least 90% identity to a polynucleotide sequence selected from the group consisting of SEQ ID NOs: 19, 20, 23-26, 29, and 30. In an aspect, a gene being downregulated encodes a nucleic acid sequence having at least 90% identity to a polynucleotide sequence selected from the group consisting of SEQ ID NOs: 19 and 20. In an aspect, a gene being downregulated encodes a nucleic acid sequence having at least 90% identity to a polynucleotide sequence selected from the group consisting of SEQ ID NOs: 23-26. In an aspect, a gene being downregulated encodes a nucleic acid sequence having at least 90% identity to a polynucleotide sequence selected from the group consisting of SEQ ID NOs: 25 and 26. In an aspect, a gene being downregulated encodes a nucleic acid sequence having at least 90% identity to a polynucleotide sequence selected from the group consisting of SEQ ID NOs: 29 and 30. In an aspect, a gene being downregulated encodes a nucleic acid sequence having at least 95% identity to a polynucleotide sequence selected from the group consisting of SEQ ID NOs: 19-36. In an aspect, a gene being downregulated encodes a nucleic acid sequence having at least 95% identity to a polynucleotide sequence selected from the group consisting of SEQ ID NOs: 19, 20, 23-26, 29, and 30. In an aspect, a gene being downregulated encodes a nucleic acid sequence having at least 95% identity to a polynucleotide sequence selected from the group consisting of SEQ ID NOs: 19 and 20. In an aspect, a gene being downregulated encodes a nucleic acid sequence having at least 95% identity to a polynucleotide sequence selected from the group consisting of SEQ ID NOs: 23-26. In an aspect, a gene being downregulated encodes a nucleic acid sequence having at least 95% identity to a polynucleotide sequence selected from the group consisting of SEQ ID NOs: 25 and 26. In an aspect, a gene being downregulated encodes a nucleic acid sequence having at least 95% identity to a polynucleotide sequence selected from the group consisting of SEQ ID NOs: 29 and 30. In an aspect, a gene being downregulated encodes a nucleic acid sequence having 100% identity to a polynucleotide sequence selected from the group consisting of SEQ ID NOs: 19-36. In an aspect, a gene being downregulated encodes a nucleic acid sequence having 100% identity to a polynucleotide sequence selected from the group consisting of SEQ ID NOs: 19, 20, 23-26, 29, and 30. In an aspect, a gene being downregulated encodes a nucleic acid sequence having 100% identity to a polynucleotide sequence selected from the group consisting of SEQ ID NOs: 19 and 20. In an aspect, a gene being downregulated encodes a nucleic acid sequence having 100% identity to a polynucleotide sequence selected from the group consisting of SEQ ID NOs: 23-26. In an aspect, a gene being downregulated encodes a nucleic acid sequence having 100% identity to a polynucleotide sequence selected from the group consisting of SEQ ID NOs: 25 and 26. In an aspect, a gene being downregulated encodes a nucleic acid sequence having 100% identity to a polynucleotide sequence selected from the group consisting of SEQ ID NOs: 29 and 30.

In another aspect, the present disclosure provides a modified tobacco plant, or part thereof, comprising a non-natural mutation in a polynucleotide having at least 70%, at least 80%, at least 90%, at least 95%, at least 97%, at least 99%, or 100% identity to a polynucleotide sequence selected from the group consisting of SEQ ID NOs: 1-36. In an aspect, a non-natural mutation in a polynucleotide having at least 90% identity to a polynucleotide sequence selected from the group consisting of SEQ ID NOs: 1, 2, 5-8, 11, 12, 19, 20, 23-26, 29, and 30. In an aspect, a non-natural mutation in a polynucleotide having at least 90% identity to a polynucleotide sequence selected from the group consisting of SEQ ID NOs: 1, 2, 19, and 20. In an aspect, a non-natural mutation in a polynucleotide having at least 90% identity to a polynucleotide sequence selected from the group consisting of SEQ ID NOs: 1 and 2. In an aspect, a non-natural mutation in a polynucleotide having at least 90% identity to a polynucleotide sequence selected from the group consisting of SEQ ID NOs: 5-8 and 23-26. In an aspect, a non-natural mutation in a polynucleotide having at least 90% identity to a polynucleotide sequence selected from the group consisting of SEQ ID NOs: 7-8 and 25-26. In an aspect, a non-natural mutation in a polynucleotide having at least 90% identity to a polynucleotide sequence selected from the group consisting of SEQ ID NOs: 7-8. In an aspect, a non-natural mutation in a polynucleotide having at least 90% identity to a polynucleotide sequence selected from the group consisting of SEQ ID NOs: 11, 12, 29, and 30. In an aspect, a non-natural mutation in a polynucleotide having at least 90% identity to a polynucleotide sequence selected from the group consisting of SEQ ID NOs: 11 and 12. In an aspect, a non-natural mutation in a polynucleotide having at least 95% identity to a polynucleotide sequence selected from the group consisting of SEQ ID NOs: 1, 2, 5-8, 11, 12, 19, 20, 23-26, 29, and 30. In an aspect, a non-natural mutation in a polynucleotide having at least 95% identity to a polynucleotide sequence selected from the group consisting of SEQ ID NOs: 1, 2, 19, and 20. In an aspect, a non-natural mutation in a polynucleotide having at least 95% identity to a polynucleotide sequence selected from the group consisting of SEQ ID NOs: 1 and 2. In an aspect, a non-natural mutation in a polynucleotide having at least 95% identity to a polynucleotide sequence selected from the group consisting of SEQ ID NOs: 5-8 and 23-26. In an aspect, a non-natural mutation in a polynucleotide having at least 95% identity to a polynucleotide sequence selected from the group consisting of SEQ ID NOs: 7-8 and 25-26. In an aspect, a non-natural mutation in a polynucleotide having at least 95% identity to a polynucleotide sequence selected from the group consisting of SEQ ID NOs: 7-8. In an aspect, a non-natural mutation in a polynucleotide having at least 95% identity to a polynucleotide sequence selected from the group consisting of SEQ ID NOs: 11, 12, 29, and 30. In an aspect, a non-natural mutation in a polynucleotide having at least 95% identity to a polynucleotide sequence selected from the group consisting of SEQ ID NOs: 11 and 12. In an aspect, a non-natural mutation in a polynucleotide having 100% identity to a polynucleotide sequence selected from the group consisting of SEQ ID NOs: 1, 2, 5-8, 11, 12, 19, 20, 23-26, 29, and 30. In an aspect, a non-natural mutation in a polynucleotide having 100% identity to a polynucleotide sequence selected from the group consisting of SEQ ID NOs: 1, 2, 19, and 20. In an aspect, a non-natural mutation in a polynucleotide having 100% identity to a polynucleotide sequence selected from the group consisting of SEQ ID NOs: 1 and 2. In an aspect, a non-natural mutation in a polynucleotide having 100% identity to a polynucleotide sequence selected from the group consisting of SEQ ID NOs: 5-8 and 23-26. In an aspect, a non-natural mutation in a polynucleotide having 100% identity to a polynucleotide sequence selected from the group consisting of SEQ ID NOs: 7-8 and 25-26. In an aspect, a non-natural mutation in a polynucleotide having 100% identity to a polynucleotide sequence selected from the group consisting of SEQ ID NOs: 7-8. In an aspect, a non-natural mutation in a polynucleotide having 100% identity to a polynucleotide sequence selected from the group consisting of SEQ ID NOs: 11, 12, 29, and 30. In an aspect, a non-natural mutation in a polynucleotide having 100% identity to a polynucleotide sequence selected from the group consisting of SEQ ID NOs: 11 and 12.

In an aspect, the present disclosure provides a modified tobacco plant, or part thereof, comprising a recombinant nucleic acid construct comprising a heterologous promoter operably linked to a polynucleotide that encodes a non-coding RNA molecule, where the non-coding RNA molecule is capable of binding to an mRNA having at least 70%, at least 80%, at least 90%, at least 95%, at least 97%, at least 99%, or 100% identity to a polynucleotide sequence selected from the group consisting of SEQ ID NOs: 19-36, wherein the non-coding RNA molecule suppresses the level or translation of the mRNA. In an aspect, a non-coding RNA molecule suppresses the level or translation of an mRNA having at least 90% identity to a polynucleotide sequence selected from the group consisting of SEQ ID NOs: 19, 20, 23-26, 29, and 30. In an aspect, a non-coding RNA molecule suppresses the level or translation of an mRNA having at least 95% identity to a polynucleotide sequence selected from the group consisting of SEQ ID NOs: 19, 20, 23-26, 29, and 30. In an aspect, a non-coding RNA molecule suppresses the level or translation of an mRNA having 100% identity to a polynucleotide sequence selected from the group consisting of SEQ ID NOs: 19, 20, 23-26, 29, and 30.

In another aspect, the present disclosure provides a modified tobacco plant, or part thereof, comprising a genetic modification in or targeting a gene, wherein the gene encodes a polypeptide having at least 70%, at least 80%, at least 90%, at least 95%, at least 97%, at least 99%, or 100% identity or similarity to an amino acid sequence selected from the group consisting of SEQ ID NOs: 37-54. In another aspect, the present disclosure provides a modified tobacco plant, or part thereof, comprising a genetic modification in or targeting a gene and downregulating the expression or activity of the gene, wherein the gene encodes a polypeptide having at least 70%, at least 80%, at least 90%, at least 95%, at least 97%, at least 99%, or 100% identity or similarity to an amino acid sequence selected from the group consisting of SEQ ID NOs: 37-54. In an aspect, a gene being downregulated encodes a polypeptide having at least 90% identity to an amino acid sequence selected from the group consisting of SEQ ID NOs: 37, 38, 41-44, 47, and 48. In an aspect, a gene being downregulated encodes a polypeptide having at least 90% identity to an amino acid sequence selected from the group consisting of SEQ ID NOs: 37 and 38. In an aspect, a gene being downregulated encodes a polypeptide having at least 90% identity to an amino acid sequence selected from the group consisting of SEQ ID NOs: 41-44. In an aspect, a gene being downregulated encodes a polypeptide having at least 90% identity to an amino acid sequence selected from the group consisting of SEQ ID NOs: 43-44. In an aspect, a gene being downregulated encodes a polypeptide having at least 90% identity to an amino acid sequence selected from the group consisting of SEQ ID NOs: 47-48. In an aspect, a gene being downregulated encodes a polypeptide having at least 95% identity to an amino acid sequence selected from the group consisting of SEQ ID NOs: 37, 38, 41-44, 47, and 48. In an aspect, a gene being downregulated encodes a polypeptide having at least 95% identity to an amino acid sequence selected from the group consisting of SEQ ID NOs: 37 and 38. In an aspect, a gene being downregulated encodes a polypeptide having at least 95% identity to an amino acid sequence selected from the group consisting of SEQ ID NOs: 41-44. In an aspect, a gene being downregulated encodes a polypeptide having at least 95% identity to an amino acid sequence selected from the group consisting of SEQ ID NOs: 43-44. In an aspect, a gene being downregulated encodes a polypeptide having at least 95% identity to an amino acid sequence selected from the group consisting of SEQ ID NOs: 47-48. In an aspect, a gene being downregulated encodes a polypeptide having 100% identity to an amino acid sequence selected from the group consisting of SEQ ID NOs: 37, 38, 41-44, 47, and 48. In an aspect, a gene being downregulated encodes a polypeptide having 100% identity to an amino acid sequence selected from the group consisting of SEQ ID NOs: 37 and 38. In an aspect, a gene being downregulated encodes a polypeptide having 100% identity to an amino acid sequence selected from the group consisting of SEQ ID NOs: 41-44. In an aspect, a gene being downregulated encodes a polypeptide having 100% identity to an amino acid sequence selected from the group consisting of SEQ ID NOs: 43-44. In an aspect, a gene being downregulated encodes a polypeptide having 100% identity to an amino acid sequence selected from the group consisting of SEQ ID NOs: 47-48.

In an aspect, the present disclosure provides a modified tobacco plant, or part thereof, comprising a non-natural mutation in a polynucleotide having a nucleic acid sequence encoding a polypeptide having at least 70%, at least 80%, at least 90%, at least 95%, at least 97%, at least 99%, or 100% identity or similarity to an amino acid sequence selected from the group consisting of SEQ ID NOs: 37-54. In an aspect, a non-natural mutation in a polynucleotide having a nucleic acid sequence encoding a polypeptide having at least 90% identity to an amino acid sequence selected from the group consisting of SEQ ID NOs: 37, 38, 41-44, 47, and 48. In an aspect, a non-natural mutation in a polynucleotide having a nucleic acid sequence encoding a polypeptide having at least 90% identity to an amino acid sequence selected from the group consisting of SEQ ID NOs: 37 and 38. In an aspect, a non-natural mutation in a polynucleotide having a nucleic acid sequence encoding a polypeptide having at least 90% identity to an amino acid sequence selected from the group consisting of SEQ ID NOs: 41-44. In an aspect, a non-natural mutation in a polynucleotide having a nucleic acid sequence encoding a polypeptide having at least 90% identity to an amino acid sequence selected from the group consisting of SEQ ID NOs: 43-44. In an aspect, a non-natural mutation in a polynucleotide having a nucleic acid sequence encoding a polypeptide having at least 90% identity to an amino acid sequence selected from the group consisting of SEQ ID NOs: 47-48. In an aspect, a non-natural mutation in a polynucleotide having a nucleic acid sequence encoding a polypeptide having at least 95% identity to an amino acid sequence selected from the group consisting of SEQ ID NOs: 37, 38, 41-44, 47, and 48. In an aspect, a non-natural mutation in a polynucleotide having a nucleic acid sequence encoding a polypeptide having at least 95% identity to an amino acid sequence selected from the group consisting of SEQ ID NOs: 37 and 38. In an aspect, a non-natural mutation in a polynucleotide having a nucleic acid sequence encoding a polypeptide having at least 95% identity to an amino acid sequence selected from the group consisting of SEQ ID NOs: 41-44. In an aspect, a non-natural mutation in a polynucleotide having a nucleic acid sequence encoding a polypeptide having at least 95% identity to an amino acid sequence selected from the group consisting of SEQ ID NOs: 43-44. In an aspect, a non-natural mutation in a polynucleotide having a nucleic acid sequence encoding a polypeptide having at least 95% identity to an amino acid sequence selected from the group consisting of SEQ ID NOs: 47-48. In an aspect, a non-natural mutation in a polynucleotide having a nucleic acid sequence encoding a polypeptide having 100% identity to an amino acid sequence selected from the group consisting of SEQ ID NOs: 37, 38, 41-44, 47, and 48. In an aspect, a non-natural mutation in a polynucleotide having a nucleic acid sequence encoding a polypeptide having 100% identity to an amino acid sequence selected from the group consisting of SEQ ID NOs: 37 and 38. In an aspect, a non-natural mutation in a polynucleotide having a nucleic acid sequence encoding a polypeptide having 100% identity to an amino acid sequence selected from the group consisting of SEQ ID NOs: 41-44. In an aspect, a non-natural mutation in a polynucleotide having a nucleic acid sequence encoding a polypeptide having 100% identity to an amino acid sequence selected from the group consisting of SEQ ID NOs: 43-44. In an aspect, a non-natural mutation in a polynucleotide having a nucleic acid sequence encoding a polypeptide having 100% identity to an amino acid sequence selected from the group consisting of SEQ ID NOs: 47-48.

In another aspect, the present disclosure provides a modified tobacco plant, or part thereof, comprising a recombinant nucleic acid construct comprising a heterologous promoter operably linked to a polynucleotide that encodes a non-coding RNA molecule, where the non-coding RNA molecule is capable of binding to an RNA encoding a polypeptide having at least 70%, at least 80%, at least 90%, at least 95%, at least 97%, at least 99%, or 100% identity or similarity to an amino acid sequence selected from the group consisting of SEQ ID NOs: 37-54, wherein the non-coding RNA molecule suppresses the expression of the polypeptide. In an aspect, the suppressed polypeptide has at least 90% identity or similarity to an amino acid sequence selected from the group consisting of SEQ ID NOs: 37, 38, 41-44, 47, and 48. In an aspect, the suppressed polypeptide has at least 95% identity or similarity to an amino acid sequence selected from the group consisting of SEQ ID NOs: 37, 38, 41-44, 47, and 48. In an aspect, the suppressed polypeptide has 100% identity or similarity to an amino acid sequence selected from the group consisting of SEQ ID NOs: 37, 38, 41-44, 47, and 48.

### Low-Alkaloid Tobacco

In one aspect, a tobacco plant comprising a genetic modification conferring a reduced level of nicotine, e.g., a genetic modification in one or more genes encoding arginine decarboxylase (ADC), aspartate oxidase (AO), or ornithine decarboxylase (ODC). In an aspect, a tobacco plant comprising a genetic modification described herein is a low-alkaloid variety or plant. In one aspect, tobacco plants of the present disclosure comprise a nic1 mutation, a nic2 mutation, or both. In an aspect, tobacco plants comprise nicotine at a level below 1%, below 2%, below 5%, below 8%, below 10%, below 12%, below 15%, below 20%, below 25%, below 30%, below 40%, below 50%, below 60%, below 70%, or below 80% of the nicotine level of a control plant when grown in similar growth conditions, where the control plant shares an essentially identical genetic background with the tobacco plant except a low-nicotine conferring mutation or transgene. In another aspect, tobacco plants comprise nicotine or total alkaloids at a level below 1%, below 2%, below 5%, below 8%, below 10%, below 12%, below 15%, below 20%, below 25%, below 30%, below 40%, below 50%, below 60%, below 70%, or below 80% of the nicotine or total alkaloids level of the control plant when grown in similar growth conditions. In another aspect, tobacco plants comprise a total alkaloid level selected from the group consisting of less than 3%, less than 2.75%, less than 2.5%, less than 2.25%, less than 2.0%, less than 1.75%, less than 1.5%, less than 1.25%, less than 1%, less than 0.9%, less than 0.8%, less than 0.7%, less than 0.6%, less than 0.5%, less than 0.4%, less than 0.3%, less than 0.2%, less than 0.1%, and less than 0.05% of the nicotine level of a control plant when grown in similar growth conditions, where the control plant shares an essentially identical genetic background with the tobacco plant except a low-nicotine conferring mutation or transgene. In another aspect, tobacco plants comprise a nicotine or total alkaloid level selected from the group consisting of less than 3%, less than 2.75%, less than 2.5%, less than 2.25%, less than 2.0%, less than 1.75%, less than 1.5%, less than 1.25%, less than 1%, less than 0.9%, less than 0.8%, less than 0.7%, less than 0.6%, less than 0.5%, less than 0.4%, less than 0.3%, less than 0.2%, less than 0.1%, and less than 0.05% of the nicotine or total alkaloids level of the control plant when grown in similar growth conditions.

In an aspect, a tobacco plant comprising a genetic modification in or targeting one or more ADC, AO, or ODC further comprises a transgene or mutation directly suppressing the expression or activity of one or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, or all twenty-one genes or loci encoding a protein selected from the group consisting of agmatine deiminase (AIC), arginase, diamine oxidase, methylputrescine oxidase (MPO), NADH dehydrogenase, phosphoribosylanthranilate isomerase (PRAI), putrescine N-methyltransferase (PMT), quinolate phosphoribosyl transferase (QPT), S-adenosyl-methionine synthetase (SAMS), A622, NBB1, berbcrine bridge enzyme-like (BBL), MYC2, Nic1_ERF, Nic2_ERF, ethylene response factor (ERF) transcription factor, nicotine uptake permease (NUP), and MATE transporter. See Dewey and Xie, Molecular genetics of alkaloid biosynthesis in Nicotiana tabacum, Phytochemistry 94 (2013) 10-27.

In an aspect, a tobacco plant comprising a genetic modification in or targeting one or more ADC, AO, or ODC genes further comprises a mutation in an ERF gene of Nic2 locus (Nic2_ERF). In an aspect, a tobacco plant comprising a genetic modification in or targeting one or more ADC, AO, or ODC genes further comprises one or more mutations in one or more, two or more, three or more, four or more, five or more, six or more, seven or more, eight or more, nine or more, or all ten genes selected from the group consisting of ERF32, ERF34, ERF39, ERF189, ERF115, ERF221, ERF104, ERF179, ERF17, and ERF168. See Shoji et al., Plant Cell, (10):3390-409 (2010); and Kajikawa et al., Plant physiol. 2017, 174:999-1011. In one aspect, a tobacco plant further comprises one or more mutations in ERF189, ERF115, or both. In an aspect, a tobacco plant comprising a genetic modification in or targeting one or more ADC, AO, or ODC genes further comprises one or more transgenes targeting and suppressing a gene encoding one or more, two or more, three or more, four or more, five or more, six or more, seven or more, eight or more, nine or more, or all ten proteins selected from the group consisting of ERF32, ERF34, ERF39, ERF189, ERF115, ERF221, ERF104, ERF179, ERF17, and ERF168.

In an aspect, a tobacco plant comprising a genetic modification in or targeting one or more ADC, AO, or ODC genes further comprises a mutation in an ERF gene of Nic1 locus (Nic1_ERF) (or Nic1b locus as in WO/2019/140297). See also WO/2018/237107. In an aspect, a tobacco plant comprising a genetic modification in or targeting one or more ADC, AO, or ODC genes further comprises one or more mutations in two or more, three or more, four or more, five or more, six or more, or seven or more genes selected from the group consisting of ERF101, ERF110, ERFnew, ERF199, ERF19, ERF130, ERF16, ERF29, ERF210, and ERF91L.2. See WO/2019/140297 and Kajikawa et al., Plant physiol. 2017, 174:999-1011. In an aspect, a tobacco plant comprising a genetic modification in or targeting one or more ADC, AO, or ODC genes further comprises one or more mutations in one or more, two or more, three or more, four or more, five or more, or all six genes selected from the group consisting of ERFnew, ERF199, ERF19, ERF29, ERF210, and ERF91L2. In an aspect, a tobacco plant comprising a genetic modification in or targeting one or more ADC, AO, or ODC genes further comprises one or more transgenes targeting and suppressing a gene encoding one or more, two or more, three or more, four or more, five or more, six or more, or seven or more genes selected from the group consisting of ERF101, ERF110, ERFnew, ERF199, ERF19, ERF130, ERF16, ERF29, ERF210, and ERF91L.2.

In an aspect, tobacco plants provided herein comprise a first genome modification comprising a mutation in a gene or locus encoding a protein selected from the group consisting of aspartate oxidase, agmatine deiminase (AIC), arginase, diamine oxidase, arginine decarboxylase (ADC), methylputrescine oxidase (MPO), NADH dehydrogenase, ornithine decarboxylase (ODC), phosphoribosylanthranilate isomerase (PRAI), putrescine N-methyltransferase (PMT), quinolate phosphoribosyl transferase (QPT), and S-adenosyl-methionine synthetase (SAMS), A622, NBB1, BBL, MYC2, Nic1_ERF, Nic2_ERF, ethylene response factor (ERF) transcription factor, nicotine uptake permease (NUP), and MATE transporter, and further comprises a second genetic modification targeting one or more ADC, AO, or ODC genes. In one aspect, tobacco plants provided herein comprise a first genome modification comprises a transgene targeting and suppressing a gene or locus encoding a protein selected from the group consisting of aspartate oxidase, agmatine deiminase (AIC), arginase, diamine oxidase, arginine decarboxylase (ADC), methylputrescine oxidase (MPO), NADH dehydrogenase, ornithine decarboxylase (ODC), phosphoribosylanthranilate isomerase (PRAI), putrescine N-methyltransferase (PMT), quinolate phosphoribosyl transferase (QPT), and S-adenosyl-methionine synthetase (SAMS), A622, NBB1, BBL, MYC2, Nic1, Nic2, ethylene response factor (ERF) transcription factor, nicotine uptake permease (NUP), and MATE transporter, and further comprises a second genetic modification targeting one or more ADC, AO, or ODC genes.

### Leaf Quality/Grading

In an aspect, the present disclosure provides a tobacco plant, or part thereof, comprising a genetic modification in or targeting one or more ADC, AO, or ODC genes with commercially acceptable leaf quality. The present disclosure also provides ADC, AO, or ODC mutant or transgenic tobacco plants having altered nicotine levels without negative impacts over other tobacco traits, e.g., leaf grade index value. In an aspect, a low-nicotine ADC, AO, or ODC mutant or transgenic tobacco variety provides cured tobacco of commercially acceptable grade.

Tobacco grades are evaluated based on factors including, but not limited to, the leaf stalk position, leaf size, leaf color, leaf uniformity and integrity, ripeness, texture, elasticity, sheen (related with the intensity and the depth of coloration of the leaf as well as the shine), hygroscopicity (the faculty of the tobacco leaves to absorb and to retain the ambient moisture), and green nuance or cast. Leaf grade can be determined, for example, using an Official Standard Grade published by the Agricultural Marketing Service of the US Department of Agriculture (7 U.S.C. § 511). See, e.g., Official Standard Grades for Burley Tobacco (U.S. Type 31 and Foreign Type 93), effective Nov. 5, 1990 (55 F.R. 40645); Official Standard Grades for Flue-Cured Tobacco (U.S. Types 11, 12, 13, 14 and Foreign Type 92), effective Mar. 27, 1989 (54 F.R. 7925); Official Standard Grades for Pennsylvania Seedleaf Tobacco (U.S. Type 41), effective Jan. 8, 1965 (29 F.R. 16854); Official Standard Grades for Ohio Cigar-Leaf Tobacco (U.S. Types 42, 43, and 44), effective Dec. 8, 1963 (28 F.R. 11719 and 28 F.R. 11926); Official Standard Grades for Wisconsin Cigar-Binder Tobacco (U.S. Types 54 and 55), effective Nov. 20, 1969 (34 F.R. 17061); Official Standard Grades for Wisconsin Cigar-Binder Tobacco (U.S. Types 54 and 55), effective Nov. 20, 1969 (34 F.R. 17061); Official Standard Grades for Georgia and Florida Shade-Grown Cigar-Wrapper Tobacco (U.S. Type 62), Effective April 1971. A USDA grade index value can be determined according to an industry accepted grade index. See, e.g., Bowman et al, Tobacco Science, 32:39-40(1988); Legacy Tobacco Document Library (Bates Document #523267826-523267833, Jul. 1, 1988, Memorandum on the Proposed Burley Tobacco Grade Index); and Miller et al., 1990, Tobacco Intern., 192:55-57 (all foregoing references are incorporated by inference in their entirety). Unless specified otherwise, a USDA grade index is a 0-100 numerical representation of federal grade received and is a weighted average of all stalk positions. A higher grade index indicates higher quality. Alternatively, leaf grade can be determined via hyper-spectral imaging. See e.g., WO 2011/027315 (published on Mar. 10, 2011, and incorporated by inference in its entirety).

In an aspect, ADC, AO, or ODC mutant or transgenic tobacco plants described here are capable of producing leaves, when cured, having a USDA grade index value selected from the group consisting of 55 or more, 60 or more, 65 or more, 70 or more, 75 or more, 80 or more, 85 or more, 90 or more, and 95 or more. In another aspect, tobacco plants are capable of producing leaves, when cured, having a USDA grade index value comparable to that of a control plant when grown and cured in similar conditions, where the control plant shares an essentially identical genetic background with the tobacco plant except a low-nicotine conferring mutation or transgene targeting ADC, AO, or ODC. In a further aspect, tobacco plants are capable of producing leaves, when cured, having a USDA grade index value of at least about 65%, at least about 70%, at least about 75%, at least about 80%, at least about 85%, at least about 90%, at least about 95%, or at least about 98% of the USDA grade index value of a control plant when grown in similar conditions, where the control plant shares an essentially identical genetic background with the tobacco plant except a low-nicotine conferring mutation or transgene. In a further aspect, tobacco plants are capable of producing leaves, when cured, having a USDA grade index value of between 65% and 130%, between 70% and 130%, between 75% and 130%, between 80% and 130%, between 85% and 130%, between 90% and 130%, between 95% and 130%, between 100% and 130%, between 105% and 130%, between 110% and 130%, between 115% and 130%, or between 120% and 130% of the USDA grade index value of the control plant. In a further aspect, tobacco plants are capable of producing leaves, when cured, having a USDA grade index value of between 70% and 125%, between 75% and 120%, between 80% and 115%, between 85% and 110%, or between 90% and 100% of the USDA grade index value of the control plant.

In another aspect, ADC, AO, or ODC mutant or transgenic tobacco plants described here are capable of producing leaves, when cured, having a USDA grade index value selected from the group consisting of 55 or more, 60 or more, 65 or more, 70 or more, 75 or more, 80 or more, 85 or more, 90 or more, and 95 or more. In another aspect, tobacco plants are capable of producing leaves, when cured, having a USDA grade index value selected from the group consisting of between 50 and 95, between 55 and 95, between 60 and 95, between 65 and 95, between 70 and 95, between 75 and 95, between 80 and 95, between 85 and 95, between 90 and 95, between 55 and 90, between 60 and 85, between 65 and 80, between 70 and 75, between 50 and 55, between 55 and 60, between 60 and 65, between 65 and 70, between 70 and 75, between 75 and 80, between 80 and 85, between 85 and 90, and between 90 and 95. In a further aspect, tobacco plants are capable of producing leaves, when cured, having a USDA grade index value of at least about 65%, at least about 70%, at least about 75%, at least about 80%, at least about 85%, at least about 90%, at least about 95%, or at least about 98% of the USDA grade index value of a control plant. In a further aspect, tobacco plants are capable of producing leaves, when cured, having a USDA grade index value of between 65% and 130%, between 70% and 130%, between 75% and 130%, between 80% and 130%, between 85% and 130%, between 90% and 130%, between 95% and 130%, between 100% and 130%, between 105% and 130%, between 110% and 130%, between 115% and 130%, or between 120% and 130% of the USDA grade index value of a control plant. In a further aspect, tobacco plants are capable of producing leaves, when cured, having a USDA grade index value of between 70% and 125%, between 75% and 120%, between 80% and 115%, between 85% and 110%, or between 90% and 100% of the USDA grade index value of a control plant.

In an aspect, the present disclosure further provides an ADC, AO, or ODC mutant or transgenic tobacco plant, or part thereof, comprising a nicotine or total alkaloid level selected from the group consisting of less than 3%, less than 2.75%, less than 2.5%, less than 2.25%, less than 2.0%, less than 1.75%, less than 1.5%, less than 1.25%, less than 1%, less than 0.9%, less than 0.8%, less than 0.7%, less than 0.6%, less than 0.5%, less than 0.4%, less than 0.3%, less than 0.2%, less than 0.1%, and less than 0.05%, where the tobacco plants are capable of producing leaves, when cured, having a USDA grade index value of 50 or more 55 or more, 60 or more, 65 or more, 70 or more, 75 or more, 80 or more, 85 or more, 90 or more, and 95 or more. In another aspect, such ADC, AO, or ODC mutant or transgenic tobacco plants comprise a nicotine level of less than 2.0% and are capable of producing leaves, when cured, having a USDA grade index value of 70 or more. In a further aspect, such ADC, AO, or ODC mutant or transgenic tobacco plants comprise a nicotine level of less than 1.0% and are capable of producing leaves, when cured, having a USDA grade index value of 70 or more.

In an aspect, the present disclosure also provides an ADC, AO, or ODC mutant or transgenic tobacco plant, or part thereof, comprising an ADC, AO, or ODC mutation or transgene, where the ADC, AO, or ODC mutation or transgene reduces the nicotine or total alkaloid level of the tobacco plant to below 1%, below 2%, below 5%, below 8%, below 10%, below 12%, below 15%, below 20%, below 25%, below 30%, below 40%, below 50%, below 60%, below 70%, or below 80% of the nicotine level of a control plant when grown in similar growth conditions, where the tobacco plant is capable of producing leaves, when cured, having a USDA grade index value comparable to the USDA grade index value of the control plant, and where the control plant shares an essentially identical genetic background with the tobacco plant except the ADC, AO, or ODC mutation or transgene.

### LA Leaf Phenotype

LA Burley 21 (also referenced as LA BU21) is a low total alkaloid tobacco line produced by incorporation of a low alkaloid gene(s) from a Cuban cigar variety into Burley 21 through several backcrosses. It has approximately 0.2% total alkaloids (dry weight) compared to the about 3.5% (dry weight) of its parent, Burley 21. LA BU21 has a leaf grade well below commercially acceptable standards. LA BU21 also exhibits other unfavorable leaf phenotypes characterized by lower yields, delayed ripening and senescence, higher susceptibility to insect herbivory, and poor end-product quality after curing. LA BU21 leaves further exhibit traits such as higher polyamine content, higher chlorophyll content and more mesophyll cells per unit leaf area. See US2019/0271000 for more characterization of LA BU21 leaf phenotypes.

In an aspect, the present disclosure provides tobacco plants, or part thereof, comprising a low nicotine or low alkaloid-conferring mutation or transgene (e.g., a genetic modification in or targeting one or more ADC, AO, or ODC) and capable of producing a leaf comprising a comparable level of one or more polyamines relative to a comparable leaf of a control plant not comprising the same mutation or transgene. In one aspect, a comparable level of one or more polyamines is within 20%, 17.5%, 15%, 12.5%, 10%, 7.5%, 5%, 2.5%, or 1% of the level in a comparable leaf of a control plant not comprising the same mutation or transgene. In an aspect, a comparable level of one or more polyamines is between 0.5% and 1%, between 1% and 2%, between 2% and 3%, between 3% and 4%, between 4% and 5%, between 5% and 6%, between 6% and 7%, between 7% and 8%, between 8% and 9%, between 9% and 10%, between 11% and 12%, between 12% and 13%, between 13% and 14%, between 14% and 15%, between 15% and 16%, between 16% and 17%, between 17% and 18%, between 18% and 19%, or between 19% and 20% of the level in a comparable leaf of a control plant not comprising the same mutation or transgene. In a further aspect, a comparable level of one or more polyamines is between 0.5% and 5%, between 5% and 10%, or between 10% and 20% of the level in a comparable leaf of a control plant not comprising the same mutation or transgene.

In an aspect, the present disclosure provides ADC, AO, or ODC mutant or transgenic tobacco plants, or part thereof, capable of producing a leaf comprising a comparable chlorophyll level relative to a comparable leaf of a control plant not comprising the same mutation or transgene. In one aspect, a comparable chlorophyll level is within 20%, 17.5%, 15%, 12.5%, 10%, 7.5%, 5%, 2.5%, or 1% of the level in a comparable leaf of a control plant not comprising the same mutation or transgene. In an aspect, a comparable chlorophyll level is between 0.5% and 1%, between 1% and 2%, between 2% and 3%, between 3% and 4%, between 4% and 5%, between 5% and 6%, between 6% and 7%, between 7% and 8%, between 8% and 9%, between 9% and 10%, between 11% and 12%, between 12% and 13%, between 13% and 14%, between 14% and 15%, between 15% and 16%, between 16% and 17%, between 17% and 18%, between 18% and 19%, or between 19% and 20% of the level in a comparable leaf of a control plant not comprising the same mutation or transgene. In a further aspect, a comparable chlorophyll level is between 0.5% and 5%, between 5% and 10%, or between 10% and 20% of the level in a comparable leaf of a control plant not comprising the same mutation or transgene.

In an aspect, the present disclosure provides ADC, AO, or ODC mutant or transgenic tobacco plants, or part thereof, capable of producing a leaf comprising a comparable number of mesophyll cell per unit of leaf area relative to a comparable leaf of a control plant not comprising the same mutation or transgene. In one aspect, a comparable number of mesophyll cell per unit of leaf area is within 20%, 17.5%, 15%, 12.5%, 10%, 7.5%, 5%, 2.5%, or 1% of the level in a comparable leaf of a control plant not comprising the same mutation or transgene. In an aspect, a comparable number of mesophyll cell per unit of leaf area is between 0.5% and 1%, between 1% and 2%, between 2% and 3%, between 3% and 4%, between 4% and 5%, between 5% and 6%, between 6% and 7%, between 7% and 8%, between 8% and 9%, between 9% and 10%, between 11% and 12%, between 12% and 13%, between 13% and 14%, between 14% and 15%, between 15% and 16%, between 16% and 17%, between 17% and 18%, between 18% and 19%, or between 19% and 20% of the level in a comparable leaf of a control plant not comprising the same mutation or transgene. In a further aspect, a comparable number of mesophyll cell per unit of leaf area is between 0.5% and 5%, between 5% and 10%, or between 10% and 20% of the level in a comparable leaf of a control plant not comprising the same mutation or transgene.

In an aspect, the present disclosure provides ADC, AO, or ODC mutant or transgenic tobacco plants, or part thereof, capable of producing a leaf comprising a comparable epidermal cell size relative to a comparable leaf of a control plant not comprising the same mutation or transgene. In one aspect, a comparable epidermal cell size is within 20%, 17.5%, 15%, 12.5%, 10%, 7.5%, 5%, 2.5%, or 1% of the level in a comparable leaf of a control plant not comprising the same mutation or transgene. In an aspect, a comparable epidermal cell size is between 0.5% and 1%, between 1% and 2%, between 2% and 3%, between 3% and 4%, between 4% and 5%, between 5% and 6%, between 6% and 7%, between 7% and 8%, between 8% and 9%, between 9% and 10%, between 11% and 12%, between 12% and 13%, between 13% and 14%, between 14% and 15%, between 15% and 16%, between 16% and 17%, between 17% and 18%, between 18% and 19%, or between 19% and 20% of the level in a comparable leaf of a control plant not comprising the same mutation or transgene. In a further aspect, a comparable epidermal cell size is between 0.5% and 5%, between 5% and 10%, or between 10% and 20% of the level in a comparable leaf of a control plant not comprising the same mutation or transgene.

In an aspect, the present disclosure provides ADC, AO, or ODC mutant or transgenic tobacco plants, or part thereof, capable of producing a leaf comprising a comparable leaf yield relative to a comparable leaf of a control plant not comprising the same mutation or transgene. In one aspect, a comparable leaf yield is within 20%, 17.5%, 15%, 12.5%, 10%, 7.5%, 5%, 2.5%, or 1% of the level in a comparable leaf of a control plant not comprising the same mutation or transgene. In an aspect, a comparable leaf yield is between 0.5% and 1%, between 1% and 2%, between 2% and 3%, between 3% and 4%, between 4% and 5%, between 5% and 6%, between 6% and 7%, between 7% and 8%, between 8% and 9%, between 9% and 10%, between 11% and 12%, between 12% and 13%, between 13% and 14%, between 14% and 15%, between 15% and 16%, between 16% and 17%, between 17% and 18%, between 18% and 19%, or between 19% and 20% of the level in a comparable leaf of a control plant not comprising the same mutation or transgene. In a further aspect, a comparable leaf yield is between 0.5% and 5%, between 5% and 10%, or between 10% and 20% of the level in a comparable leaf of a control plant not comprising the same mutation or transgene.

In an aspect, the present disclosure provides ADC, AO, or ODC mutant or transgenic tobacco plants, or part thereof, exhibiting a comparable insect herbivory susceptibility relative to a comparable leaf of a control plant not comprising the same mutation or transgene. In one aspect, a comparable insect herbivory susceptibility is within 20%, 17.5%, 15%, 12.5%, 10%, 7.5%, 5%, 2.5%, or 1% of the level in a comparable leaf of a control plant not comprising the same mutation or transgene. In an aspect, a comparable insect herbivory susceptibility is between 0.5% and 1%, between 1% and 2%, between 2% and 3%, between 3% and 4%, between 4% and 5%, between 5% and 6%, between 6% and 7%, between 7% and 8%, between 8% and 9%, between 9% and 10%, between 11% and 12%, between 12% and 13%, between 13% and 14%, between 14% and 15%, between 15% and 16%, between 16% and 17%, between 17% and 18%, between 18% and 19%, or between 19% and 20% of the level in a comparable leaf of a control plant not comprising the same mutation or transgene. In a further aspect, a comparable insect herbivory susceptibility is between 0.5% and 5%, between 5% and 10%, or between 10% and 20% of the level in a comparable leaf of a control plant not comprising the same mutation or transgene.

Insect herbivory susceptibility level can be assayed by methods known in the art, for example, in an insect feeding assay. In short, a quarter inch layer of 0.7% agar in water is added to a 100 mm Petri dish and allowed to solidify. Leaf discs are cut from the petri dish lid, placed in the plates and pushed gently into the agar. Leaf discs are taken from plants at the 4-5 leaf stage. Discs were taken from lamina only to exclude major midribs. A single disc is taken from each of the four largest leaves of the plant generating 4 replicates per plant. Four plants are sampled for a total of 16 biological replicates test line. A single budworm (e.g., Heliothis sp., Helicoverpa sp.) at the second instar stage is added to the leaf and allowed to feed for 48 hours at ambient temperature. After 48 hours the budworm larvae are weighed and final larval weights are recorded.

In an aspect, a tobacco plant, or part thereof, comprises relative to a control tobacco plant: a first genome modification providing a lower level of nicotine or total alkaloid (e.g., in or targeting one or more ADC, AO, or ODC genes), and a second genome modification providing a comparable level of one or more traits selected from the group consisting of total leaf polyamine level, total root polyamine level, total leaf chlorophyll level, mesophyll cell number per leaf area unit, and leaf epidermal cell size; and where the control plant does not have both the first and the second genome modifications. In one aspect, a tobacco plant, or part thereof, comprises relative to a control tobacco plant: a first genome modification providing a lower level of nicotine or total alkaloid (e.g., in or targeting one or more ADC, AO, or ODC genes), and a second genome modification providing a comparable level of total leaf polyamine level, where the control plant does not have both the first and the second genome modifications. In an aspect, a tobacco plant, or part thereof, comprises relative to a control tobacco plant: a first genome modification providing a lower level of nicotine or total alkaloid (e.g., in or targeting one or more ADC, AO, or ODC genes), and a second genome modification providing a comparable level of total root polyamine level, where the control plant does not have both the first and the second genome modifications. In one aspect, a tobacco plant, or part thereof, comprises relative to a control tobacco plant: a first genome modification providing a lower level of nicotine or total alkaloid (e.g., in or targeting one or more ADC, AO, or ODC genes), and a second genome modification providing a comparable level of total leaf chlorophyll level, where the control plant does not have both the first and the second genome modifications. In an aspect, a tobacco plant, or part thereof, comprises relative to a control tobacco plant: a first genome modification providing a lower level of nicotine or total alkaloid (e.g., in or targeting one or more ADC, AO, or ODC genes), and a second genome modification providing a comparable level of mesophyll cell number per leaf area unit, where the control plant does not have both the first and the second genome modifications. In one aspect, a tobacco plant, or part thereof, comprises relative to a control tobacco plant: a first genome modification providing a lower level of nicotine or total alkaloid (e.g., in or targeting one or more ADC, AO, or ODC genes), and a second genome modification providing a comparable level of leaf epidermal cell size, where the control plant does not have both the first and the second genome modifications. In an aspect, a second genome modification is in or targeting an ADC, AO, or ODC gene.

In an aspect, a first genome modification, a second genome modification, or both comprise a transgene, a mutation, or both. In one aspect, a genome modification, a second genome modification, or both comprise a transgene. In an aspect, a first genome modification, a second genome modification, or both comprise a mutation. In one aspect, a first genome modification, a second genome modification, or both are not transgene-based. In an aspect, a first genome modification, a second genome modification, or both are not mutation-based.

In an aspect, tobacco plants provided herein comprise a reduced amount of total conjugated polyamines in leaves relative to the control tobacco plant. In one aspect, tobacco plants provided herein comprise a reduced amount of total conjugated polyamines in roots relative to the control tobacco plant. Used here, conjugated polyamines include, but are not limited to, soluble conjugated polyamines such as phenolamides containing a backbone consisting of a free polyamine (e.g., putrescine, spermine, and/or spermidine) conjugated with one or more phenylpropanoids such as ferulic, caffeic and courmaric acids. Conjugated polyamines also include, but are not limited to, insoluble conjugated polyamines incorporated into structural polymers such as lignin. In an aspect, tobacco plants provided herein comprise a reduced amount of total free polyamines (e.g., putrescine, spermine, and spermidine) in leaves relative to the control tobacco plant. In one aspect, tobacco plants provided herein comprise a reduced amount of total conjugated polyamines in roots relative to the control tobacco plant. In an aspect, tobacco plants provided herein comprise a reduced amount of total conjugated form of one or more polyamines selected from the group consisting of putrescine, spermidine and spermine in leaves relative to the control tobacco plant. In one aspect, tobacco plants provided herein comprise a reduced amount of total conjugated form of one or more polyamines selected from the group consisting of putrescine, spermidine and spermine in roots relative to the control tobacco plant. In an aspect, tobacco plants provided herein comprise a reduced amount of total free form of one or more polyamines selected from the group consisting of putrescine, spermidine and spermine in leaves relative to the control tobacco plant. In one aspect, tobacco plants provided herein comprise a reduced amount of total conjugated form of one or more polyamines selected from the group consisting of putrescine, spermidine and spermine in roots relative to the control tobacco plant.

In an aspect, a characteristic or a trait of a tobacco plant described here are measured at a time selected from the group consisting of immediately before flowering, at topping, 1 week-post-topping (WPT), 2 WPT, 3 WPT, 4 WPT, 5 WPT, 6 WPT, 7 WPT, 8 WPT, and at harvest. In one aspect, tobacco plants provided herein comprising a first and a second genome modification are capable of producing a leaf with a leaf grade comparable to that of a leaf from a control plant. In an aspect, tobacco plants provided herein comprising a first and a second genome modification have a total leaf yield comparable to a control plant.

### Chemical Measurements

“Alkaloids” are complex, nitrogen-containing compounds that naturally occur in plants, and have pharmacological effects in humans and animals. “Nicotine” is the primary natural alkaloid in commercialized cigarette tobacco and accounts for about 90 percent of the alkaloid content in Nicotiana tabacum. Other major alkaloids in tobacco include cotinine, nornicotine, myosmine, nicotyrine, anabasine and anatabine. Minor tobacco alkaloids include nicotine-n-oxide, N-methyl anatabine, N-methyl anabasine, pseudooxynicotine, 2,3 dipyridyl and others.

In an aspect, an ADC, AO, or ODC mutant or transgenic tobacco plant provided herein comprises a genetic modification providing a lower level of one or more alkaloids selected from the group consisting of cotinine, nornicotine, myosmine, nicotyrine, anabasine and anatabine, compared to a control tobacco plant without the genetic modification, when grown in similar growth conditions. In an aspect, a lower alkaloid or nicotine level refers to an alkaloid or nicotine level of below 1%, below 2%, below 5%, below 8%, below 10%, below 12%, below 15%, below 20%, below 25%, below 30%, below 40%, below 50%, below 60%, below 70%, or below 80% of the alkaloid or nicotine level of a control tobacco plant. In another aspect, a lower alkaloid or nicotine level refers to an alkaloid or nicotine level of about between 0.5% and 1%, between 1% and 2%, between 2% and 3%, between 3% and 4%, between 4% and 5%, between 5% and 6%, between 6% and 7%, between 7% and 8%, between 8% and 9%, between 9% and 10%, between 11% and 12%, between 12% and 13%, between 13% and 14%, between 14% and 15%, between 15% and 16%, between 16% and 17%, between 17% and 18%, between 18% and 19%, between 19% and 20%, between 21% and 22%, between 22% and 23%, between 23% and 24%, between 24% and 25%, between 25% and 26%, between 26% and 27%, between 27% and 28%, between 28% and 29%, or between 29% and 30% of the alkaloid or nicotine level of a control tobacco plant. In a further aspect, a lower alkaloid or nicotine level refers to an alkaloid or nicotine level of about between 0.5% and 5%, between 5% and 10%, between 10% and 20%, between 20% and 30% of the alkaloid or nicotine level of a control tobacco plant.

In an aspect, an ADC, AO, or ODC mutant or transgenic tobacco plant provided herein comprises an average nicotine or total alkaloid level selected from the group consisting of about 0.01%, 0.02%, 0.05%, 0.75%, 0.1%, 0.15%, 0.2%, 0.3%, 0.35%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 5%, 6%, 7%, 8%, and 9% on a dry weight basis. In another aspect, tobacco plants provided herein comprise an average nicotine or total alkaloid level selected from the group consisting of about between 0.01% and 0.02%, between 0.02% and 0.05%, between 0.05% and 0.75%, between 0.75% and 0.1%, between 0.1% and 0.15%, between 0.15% and 0.2%, between 0.2% and 0.3%, between 0.3% and 0.35%, between 0.35% and 0.4%, between 0.4% and 0.5%, between 0.5% and 0.6%, between 0.6% and 0.7%, between 0.7% and 0.8%, between 0.8% and 0.9%, between 0.9% and 1%, between 1% and 1.1%, between 1.1% and 1.2%, between 1.2% and 1.3%, between 1.3% and 1.4%, between 1.4% and 1.5%, between 1.5% and 1.6%, between 1.6% and 1.7%, between 1.7% and 1.8%, between 1.8% and 1.9%, between 1.9% and 2%, between 2% and 2.1%, between 2.1% and 2.2%, between 2.2% and 2.3%, between 2.3% and 2.4%, between 2.4% and 2.5%, between 2.5% and 2.6%, between 2.6% and 2.7%, between 2.7% and 2.8%, between 2.8% and 2.9%, between 2.9% and 3%, between 3% and 3.1%, between 3.1% and 3.2%, between 3.2% and 3.3%, between 3.3% and 3.4%, between 3.4% and 3.5%, and between 3.5% and 3.6% on a dry weight basis. In a further aspect, tobacco plants provided herein comprise an average nicotine or total alkaloid level selected from the group consisting of about between 0.01% and 0.1%, between 0.02% and 0.2%, between 0.03% and 0.3%, between 0.04% and 0.4%, between 0.05% and 0.5%, between 0.75% and 1%, between 0.1% and 1.5%, between 0.15% and 2%, between 0.2% and 3%, and between 0.3% and 3.5% on a dry weight basis.

Unless specified otherwise, measurements of alkaloid, polyamine, or nicotine levels (or another leaf chemistry or property characterization) or leaf grade index values mentioned herein for a tobacco plant, variety, cultivar, or line refer to average measurements, including, for example, an average of multiple leaves of a single plant or an average measurement from a population of tobacco plants from a single variety, cultivar, or line. Unless specified otherwise, the nicotine, alkaloid, or polyamine level (or another leaf chemistry or property characterization) of a tobacco plant described here is measured 2 weeks after topping in a pooled leaf sample collected from leaf number 3, 4, and 5 after topping. In another aspect, the nicotine, alkaloid, or polyamine level (or another leaf chemistry or property characterization) of a tobacco plant is measured after topping in a leaf having the highest level of nicotine, alkaloid, or polyamine (or another leaf chemistry or property characterization). In an aspect, the nicotine, alkaloid, or polyamine level of a tobacco plant is measured after topping in leaf number 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. In another aspect, the nicotine, alkaloid, or polyamine level (or another leaf chemistry or property characterization) of a tobacco plant is measured after topping in a pool of two or more leaves with consecutive leaf numbers selected from the group consisting of leaf number 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30. In another aspect, the nicotine, alkaloid, or polyamine level (or another leaf chemistry or property characterization) of a tobacco plant is measured after topping in a leaf with a leaf number selected from the group consisting of between 1 and 5, between 6 and 10, between 11 and 15, between 16 and 20, between 21 and 25, and between 26 and 30. In another aspect, the nicotine, alkaloid, or polyamine level (or another leaf chemistry or property characterization) of a tobacco plant is measured after topping in a pool of two or more leaves with leaf numbers selected from the group consisting of between 1 and 5, between 6 and 10, between 11 and 15, between 16 and 20, between 21 and 25, and between 26 and 30. In another aspect, the nicotine, alkaloid, or polyamine level (or another leaf chemistry or property characterization) of a tobacco plant is measured after topping in a pool of three or more leaves with leaf numbers selected from the group consisting of between 1 and 5, between 6 and 10, between 11 and 15, between 16 and 20, between 21 and 25, and between 26 and 30.

Alkaloid levels can be assayed by methods known in the art, for example by quantification based on gas-liquid chromatography, high performance liquid chromatography, radio-immunoassays, and enzyme-linked immunosorbcnt assays. For example, nicotinic alkaloid levels can be measured by a GC-FID method based on CORESTA Recommended Method No. 7, 1987 and ISO Standards (ISO TC 126N 394 E. See also Hibi et al., Plant Physiology 100: 826-35 (1992) for a method using gas-liquid chromatography equipped with a capillary column and an FID detector. Unless specified otherwise, all alkaloid levels described here are measured using a method in accordance with CORESTA Method No 62, Determination of Nicotine in Tobacco and Tobacco Products by Gas Chromatographic Analysis, February 2005, and those defined in the Centers for Disease Control and Prevention's Protocol for Analysis of Nicotine, Total Moisture and pH in Smokeless Tobacco Products, as published in the Federal Register Vol. 64, No. 55 Mar. 23, 1999 (and as amended in Vol. 74, No. 4, Jan. 7, 2009).

Alternatively, tobacco total alkaloids can be measured using a segmented-flow colorimetric method developed for analysis of tobacco samples as adapted by Skalar Instrument Co (West Chester, PA) and described by Collins et al., Tobacco Science 13:79-81 (1969). In short, samples of tobacco are dried, ground, and extracted prior to analysis of total alkaloids and reducing sugars. The method then employs an acetic acid/methanol/water extraction and charcoal for decolorization. Determination of total alkaloids was based on the reaction of cyanogen chloride with nicotine alkaloids in the presence of an aromatic amine to form a colored complex which is measured at 460 nm. Unless specified otherwise, total alkaloid levels or nicotine levels shown herein are on a dry weight basis (e.g., percent total alkaloid or percent nicotine).

As used herein, leaf numbering is based on the leaf position on a tobacco stalk with leaf number 1 being the oldest leaf (at the base) after topping and the highest leaf number assigned to the youngest leaf (at the tip).

A population of tobacco plants or a collection of tobacco leaves for determining an average measurement (e.g., alkaloid or nicotine level or leaf grading) can be of any size, for example, 5, 10, 15, 20, 25, 30, 35, 40, or 50. Industry-accepted standard protocols are followed for determining average measurements or grade index values.

As used herein, “topping” refers to the removal of the stalk apex, including the SAM, flowers, and up to several adjacent leaves, when a tobacco plant is near vegetative maturity and around the start of reproductive growth. Typically, tobacco plants are topped in the button stage (soon after the flower begins to appear). For example, greenhouse or field-grown tobacco plants can be topped when 50% of the plants have at least one open flower. Topping a tobacco plant results in the loss of apical dominance and also induce increased alkaloid production.

Typically, the nicotine, alkaloid, or polyamine level (or another leaf chemistry or property characterization) of a tobacco plant is measured about 2 weeks after topping. Other time points can also be used. In an aspect, the nicotine, alkaloid, or polyamine level (or another leaf chemistry or property characterization) of a tobacco plant is measured about 1, 2, 3, 4, or 5 weeks after topping. In another aspect, the nicotine, alkaloid, or polyamine level (or another leaf chemistry or property characterization) of a tobacco plant is measured about 3, 5, 7, 10, 12, 14, 17, 19, or 21 days after topping.

As used herein, “similar growth conditions” refer to similar environmental conditions and/or agronomic practices for growing and making meaningful comparisons between two or more plant genotypes so that neither environmental conditions nor agronomic practices would contribute to or explain any difference observed between the two or more plant genotypes. Environmental conditions include, for example, light, temperature, water (humidity), and nutrition (e.g., nitrogen and phosphorus). Agronomic practices include, for example, seeding, clipping, undercutting, transplanting, topping, and suckering. See Chapters 4B and 4C of Tobacco, Production, Chemistry and Technology, Davis & Nielsen, eds., Blackwell Publishing, Oxford (1999), pp 70-103.

As used herein, “comparable leaves” refer to leaves having similar size, shape, age, and/or stalk position.

### Aroma/Flavor

In an aspect, ADC, AO, or ODC mutant or transgenic tobacco plants provided herein comprise a similar level of one or more tobacco aroma compounds selected from the group consisting of 3-methylvaleric acid, valeric acid, isovaleric acid, a labdenoid, a cembrenoid, a sugar ester, and a reducing sugar, compared to control tobacco plants when grown in similar growth conditions.

As used herein, tobacco aroma compounds are compounds associated with the flavor and aroma of tobacco smoke. These compounds include, but are not limited to, 3-methylvaleric acid, valeric acid, isovaleric acid, cembrenoid and labdenoid diterpenes, and sugar esters. Concentrations of tobacco aroma compounds can be measured by any known metabolite profiling methods in the art including, without limitation, gas chromatography mass spectrometry (GC-MS), Nuclear Magnetic Resonance Spectroscopy, liquid chromatography-linked mass spectrometry. See The Handbook of Plant Metabolomics, edited by Weckwerth and Kahl, (Wiley-Blackwell) (May 28, 2013).

As used herein, “reducing sugar(s)” are any sugar (monosaccharide or polysaccharide) that has a free or potentially free aldehyde or ketone group. Glucose and fructose act as nicotine buffers in cigarette smoke by reducing smoke pH and effectively reducing the amount of “free” unprotonated nicotine. Reducing sugars balances smoke flavor, for example, by modifying the sensory impact of nicotine and other tobacco alkaloids. An inverse relationship between sugar content and alkaloid content has been reported across tobacco varieties, within the same variety, and within the same plant line caused by planting conditions. Reducing sugar levels can be measured using a segmented-flow colorimetric method developed for analysis of tobacco samples as adapted by Skalar Instrument Co (West Chester, PA) and described by Davis, Tobacco Science 20:139-144 (1976). For example, a sample is dialyzed against a sodium carbonate solution. Copper neocuproin is added to the sample and the solution is heated. The copper neocuproin chelate is reduced in the presence of sugars resulting in a colored complex which is measured at 460 nm.

### TSNA

In still another aspect, a tobacco plant provided further comprises one or more mutations in one or more loci encoding a nicotine demethylase (e.g., CYP82E4, CYP82F5, CYP82E10) that confer reduced amounts of nornicotine (See U.S. Pat. Nos. 8,319,011; 8,124,851; 9,187,759; 9,228,194; 9,228,195; 9,247,706) compared to control plant lacking one or more mutations in one or more loci encoding a nicotine demethylase. In an aspect, a modified tobacco plant described further comprises reduced nicotine demethylase activity compared to a control plant when grown and cured under comparable conditions. In a further aspect, a tobacco plant provided further comprises one or more mutations or transgenes providing an elevated level of one or more antioxidants (See U.S. Patent Application Publication No. 2018/0119163 and WO 2018/067985). In another aspect, a tobacco plant provided further comprises one or more mutations or transgenes providing a reduced level of one or more tobacco-specific nitrosamines (TSNAs) (such as N′-nitrosonornicotine (NNN), 4-methylnitrosoamino-1-(3-pyridyl)-1-butanone (NNK), N′-nitrosoanatabine (NAT) N′-nitrosoanabasine (NAB)).

### Mutation Types

In an aspect, the present disclosure provides a tobacco plant, or part thereof, comprising a non-natural mutation in an ADC, AO, or ODC gene (e.g., as in an “ADC mutant”, “AO mutant”, or “ODC mutant”). In an aspect, a non-natural mutation comprises one or more mutation types selected from the group consisting of a nonsense mutation, a missense mutation, a frameshift mutation, a splice-site mutation, and any combinations thereof. As used herein, a “nonsense mutation” refers to a mutation to a nucleic acid sequence that introduces a premature stop codon to an amino acid sequence by the nucleic acid sequence. As used herein, a “missense mutation” refers to a mutation to a nucleic acid sequence that causes a substitution within the amino acid sequence encoded by the nucleic acid sequence. As used herein, a “frameshift mutation” refers to an insertion or deletion to a nucleic acid sequence that shifts the frame for translating the nucleic acid sequence to an amino acid sequence. A “splice-site mutation” refers to a mutation in a nucleic acid sequence that causes an intron to be retained for protein translation, or, alternatively, for an exon to be excluded from protein translation. Splice-site mutations can cause nonsense, missense, or frameshift mutations.

Mutations in coding regions of genes (e.g., exonic mutations) can result in a truncated protein or polypeptide when a mutated messenger RNA (mRNA) is translated into a protein or polypeptide. In an aspect, this disclosure provides a mutation that results in the truncation of a protein or polypeptide. As used herein, a “truncated” protein or polypeptide comprises at least one fewer amino acid as compared to an endogenous control protein or polypeptide. For example, if endogenous Protein A comprises 100 amino acids, a truncated version of Protein A can comprise between 1 and 99 amino acids.

Without being limited by any scientific theory, one way to cause a protein or polypeptide truncation is by the introduction of a premature stop codon in an mRNA transcript of an endogenous gene. In an aspect, this disclosure provides a mutation that results in a premature stop codon in an mRNA transcript of an endogenous gene. As used herein, a “stop codon” refers to a nucleotide triplet within an mRNA transcript that signals a termination of protein translation. A “premature stop codon” refers to a stop codon positioned earlier (e.g., on the 5′-side) than the normal stop codon position in an endogenous mRNA transcript. Without being limiting, several stop codons are known in the art, including “UAG,” “UAA,” “UGA,” “TAG,” “TAA,” and “TGA.”

In an aspect, a mutation provided herein comprises a null mutation. As used herein, a “null mutation” refers to a mutation that confers a complete loss-of-function for a protein encoded by a gene comprising the mutation, or, alternatively, a mutation that confers a complete loss-of-function for a small RNA encoded by a genomic locus. A null mutation can cause lack of mRNA transcript production, a lack of small RNA transcript production, a lack of protein function, or a combination thereof.

A mutation provided herein can be positioned in any part of an endogenous gene. In an aspect, a mutation provided herein is positioned within an exon of an endogenous gene. In another aspect, a mutation provided herein is positioned within an intron of an endogenous gene. In a further aspect, a mutation provided herein is positioned within a 5′-untranslated region (UTR) of an endogenous gene. In still another aspect, a mutation provided herein is positioned within a 3′-UTR of an endogenous gene. In yet another aspect, a mutation provided herein is positioned within a promoter of an endogenous gene. In yet another aspect, a mutation provided herein is positioned within a terminator of an endogenous gene.

In an aspect, a mutation in an endogenous gene results in a reduced level of expression as compared to the endogenous gene lacking the mutation. In another aspect, a mutation in an endogenous gene results in an increased level of expression as compared to the endogenous gene lacking the mutation.

In an aspect, a non-natural mutation results in a reduced level of expression as compared to expression of the gene in a control tobacco plant. In an aspect, a non-natural mutation results in an increased level of expression as compared to expression of the gene in a control tobacco plant.

In a further aspect, a mutation in an endogenous gene results in a reduced level of activity by a protein or polypeptide encoded by the endogenous gene having the mutation as compared to a protein or polypeptide encoded by the endogenous gene lacking the mutation. In a further aspect, a mutation in an endogenous gene results in an increased level of activity by a protein or polypeptide encoded by the endogenous gene having the mutation as compared to a protein or polypeptide encoded by the endogenous gene lacking the mutation.

In an aspect, a non-natural mutation results in a reduced level of activity by a protein or polypeptide encoded by the polynucleotide comprising the non-natural mutation as compared to a protein or polypeptide encoded by the polynucleotide lacking the non-natural mutation. In another aspect, a non-natural mutation results in an increased level of activity by a protein or polypeptide encoded by the polynucleotide comprising the non-natural mutation as compared to a protein or polypeptide encoded by the polynucleotide lacking the non-natural mutation.

In an aspect, a mutation provided here provides a dominant mutant that activates the expression or elevates the activity of a gene of interest, e.g., one or more ADC, AO, or ODC genes.

Levels of gene expression are routinely investigated in the art. As non-limiting examples, gene expression can be measured using quantitative reverse transcriptase PCR (qRT-PCR), RNA sequencing, or Northern blots. In an aspect, gene expression is measured using qRT-PCR. In another aspect, gene expression is measured using a Northern blot. In another aspect, gene expression is measured using RNA sequencing.

ADC, AO, or ODC mutant tobacco plants can be made by any method known in the art including random or targeted mutagenesis approaches. Such mutagenesis methods include, without limitation, treatment of seeds with ethyl methylsulfate (EMS) (Hildering and Verkerk, In, The use of induced mutations in plant breeding. Pergamon press, pp 317-320, 1965) or UV-irradiation, X-rays, and fast neutron irradiation (see, for example, Verkerk, Neth. J. Agric. Sci. 19:197-203, 1971; and Poehlman, Breeding Field Crops, Van Nostrand Reinhold, New York (3.sup.rd ed), 1987), transposon tagging (Fedoroff et al., 1984; U.S. Pat. Nos. 4,732,856 and 5,013,658), as well as T-DNA insertion methodologies (Hoekema et al., 1983; U.S. Pat. No. 5,149,645). EMS-induced mutagenesis consists of chemically inducing random point mutations over the length of the genome. Fast neutron mutagenesis consists of exposing seeds to neutron bombardment which causes large deletions through double stranded DNA breakage. Transposon tagging comprises inserting a transposon within an endogenous gene to reduce or eliminate expression of the gene. The types of mutations that may be present in a tobacco gene include, for example, point mutations, deletions, insertions, duplications, and inversions. Such mutations desirably are present in the coding region of a tobacco gene; however mutations in the promoter region, and intron, or an untranslated region of a tobacco gene may also be desirable.

In addition, a fast and automatable method for screening for chemically induced mutations, TILLING (Targeting Induced Local Lesions In Genomes), using denaturing HPLC or selective endonuclease digestion of selected PCR products is also applicable to the present disclosure. See, McCallum et al. (2000) Nat. Biotechnol. 18:455-457. Mutations that impact gene expression or that interfere with the function of genes can be determined using methods that are well known in the art. Insertional mutations in gene exons usually result in null-mutants. Mutations in conserved residues can be particularly effective in inhibiting the function of a protein. In an aspect, tobacco plants comprise a nonsense (e.g., stop codon) mutation in one or more NCG genes described in U.S. Provisional Application Nos. 62/616,959 and 62/625,878, both of which are incorporated by reference in their entirety.

In an aspect, the present disclosure also provides tobacco lines with altered nicotine levels while maintaining commercially acceptable leaf quality. In an aspect, such a line can be produced by introducing mutations into one or more ADC, AO, or ODC genes via precise genome engineering technologies, for example, Transcription activator-like effector nucleases (TALENs), meganuclease, zinc finger nuclease, and a clustered regularly-interspaced short palindromic repeats (CRISPR)/Cas9 system, a CRISPR/Cpf1 system, a CRISPR/Csm1 system, and a combination thereof (see, for example, U.S. Patent Application publication 2017/0233756). See, e.g., Gaj et al., Trends in Biotechnology, 31(7):397-405 (2013). The prime editing methodology, which uses a reverse transcriptase fused to an RNA-programmable nickase (e.g., a modified Cas9), described by Anzalone et al. (“Search-and-replace genome editing without double-stranded breaks or donor DNA,” Nature, 21 Oct. 2019 (doi[dot]org/10.1038/s41586-019-1711-4)), can also be used to introduce mutations into one or more ADC, AO, or ODC genes.

The screening and selection of mutagenized tobacco plants can be through any methodologies known to those having ordinary skill in the art. Examples of screening and selection methodologies include, but are not limited to, Southern analysis, PCR amplification for detection of a polynucleotide, Northern blots, RNase protection, primer-extension, RT-PCR amplification for detecting RNA transcripts, Sanger sequencing, Next Generation sequencing technologies (e.g., Illumina, PacBio, Ion Torrent, 454), enzymatic assays for detecting enzyme or ribozyme activity of polypeptides and polynucleotides, and protein gel electrophoresis, Western blots, immunoprecipitation, and enzyme-linked immunoassays to detect polypeptides. Other techniques such as in situ hybridization, enzyme staining, and immunostaining also can be used to detect the presence or expression of polypeptides and/or polynucleotides. Methods for performing all of the referenced techniques are known.

In an aspect, a tobacco plant or plant genome provided herein is mutated or edited by a genome editing technique, e.g., by a nuclease selected from the group consisting of a meganuclease, a zinc-finger nuclease (ZFN), a transcription activator-like effector nuclease (TALEN), a CRISPR/Cas9 nuclease, a CRISPR/Cpf1 nuclease, or a CRISPR/Csm1 nuclease.

As used herein, “editing” or “genome editing” refers to targeted mutagenesis of at least 1, at least 2, at least 3, at least 4, at least 5, at least 6, at least 7, at least 8, at least 9, or at least 10 nucleotides of an endogenous plant genome nucleic acid sequence, or removal or replacement of an endogenous plant genome nucleic acid sequence. In an aspect, an edited nucleic acid sequence provided has at least 99.9%, at least 99.5%, at least 99%, at least 98%, at least 97%, at least 96%, at least 95%, at least 94%, at least 93%, at least 92%, at least 91%, at least 90%, at least 85%, at least 80%, or at least 75% sequence identity with an endogenous nucleic acid sequence. In an aspect, an edited nucleic acid sequence provided has at least 99.9%, at least 99.5%, at least 99%, at least 98%, at least 97%, at least 96%, at least 95%, at least 94%, at least 93%, at least 92%, at least 91%, at least 90%, at least 85%, at least 80%, or at least 75% sequence identity with SEQ ID NOs: 1-36, and fragments thereof.

Meganucleases, ZFNs, TALENS, CRISPR/Cas9, CRISPR/Csm1 and CRISPR/Cpf1 induce a double-strand DNA break at a target site of a genomic sequence that is then repaired by the natural processes of homologous recombination (HR) or non-homologous end-joining (NHEJ). Sequence modifications then occur at the cleaved sites, which can include deletions or insertions that result in gene disruption in the case of NHEJ, or integration of donor nucleic acid sequences by HR. In an aspect, a method provided comprises editing a plant genome with a nuclease provided to mutate at least 1, at least 2, at least 3, at least 4, at least 5, at least 6, at least 7, at least 8, at least 9, at least 10, or more than 10 nucleotides in the plant genome via HR with a donor polynucleotide. In an aspect, a mutation provided is caused by genome editing using a nuclease. In another aspect, a mutation provided is caused by non-homologous end-joining or homologous recombination.

Meganucleases, which are commonly identified in microbes, are unique enzymes with high activity and long recognition sequences (>14 bp) resulting in site-specific digestion of target DNA. Engineered versions of naturally occurring meganucleases typically have extended DNA recognition sequences (for example, 14 to 40 bp). The engineering of meganucleases can be more challenging than that of ZFNs and TALENs because the DNA recognition and cleavage functions of meganucleases are intertwined in a single domain. Specialized methods of mutagenesis and high-throughput screening have been used to create novel meganuclease variants that recognize unique sequences and possess improved nuclease activity.

ZFNs are synthetic proteins consisting of an engineered zinc finger DNA-binding domain fused to the cleavage domain of the FokI restriction endonuclease. ZFNs can be designed to cleave almost any long stretch of double-stranded DNA for modification of the zinc finger DNA-binding domain. ZFNs form dimers from monomers composed of a non-specific DNA cleavage domain of FokI endonuclease fused to a zinc finger array engineered to bind a target DNA sequence.

The DNA-binding domain of a ZFN is typically composed of 3-4 zinc-finger arrays. The amino acids at positions −1, +2, +3, and +6 relative to the start of the zinc finger ∞-helix, which contribute to site-specific binding to the target DNA, can be changed and customized to fit specific target sequences. The other amino acids form the consensus backbone to generate ZFNs with different sequence specificities. Rules for selecting target sequences for ZFNs are known in the art.

The FokI nuclease domain requires dimerization to cleave DNA and therefore two ZFNs with their C-terminal regions are needed to bind opposite DNA strands of the cleavage site (separated by 5-7 bp). The ZFN monomer can cute the target site if the two-ZF-binding sites are palindromic. The term ZFN, as used herein, is broad and includes a monomeric ZFN that can cleave double stranded DNA without assistance from another ZFN. The term ZFN is also used to refer to one or both members of a pair of ZFNs that are engineered to work together to cleave DNA at the same site.

Without being limited by any scientific theory, because the DNA-binding specificities of zinc finger domains can in principle be re-engineered using one of various methods, customized ZFNs can theoretically be constructed to target nearly any gene sequence. Publicly available methods for engineering zinc finger domains include Context-dependent Assembly (CoDA), Oligomerized Pool Engineering (OPEN), and Modular Assembly.

TALENs are artificial restriction enzymes generated by fusing the transcription activator-like effector (TALE) DNA binding domain to a FokI nuclease domain. When each member of a TALEN pair binds to the DNA sites flanking a target site, the FokI monomers dimerize and cause a double-stranded DNA break at the target site. The term TALEN, as used herein, is broad and includes a monomeric TALEN that can cleave double stranded DNA without assistance from another TALEN. The term TALEN is also used to refer to one or both members of a pair of TALENs that work together to cleave DNA at the same site.

Transcription activator-like effectors (TALEs) can be engineered to bind practically any DNA sequence. TALE proteins are DNA-binding domains derived from various plant bacterial pathogens of the genus Xanthomonas. The Xanthomonas pathogens secrete TALEs into the host plant cell during infection. The TALE moves to the nucleus, where it recognizes and binds to a specific DNA sequence in the promoter region of a specific DNA sequence in the promoter region of a specific gene in the host genome. TALE has a central DNA-binding domain composed of 13-28 repeat monomers of 33-34 amino acids. The amino acids of each monomer are highly conserved, except for hypervariable amino acid residues at positions 12 and 13. The two variable amino acids are called repeat-variable diresidues (RVDs). The amino acid pairs NI, NG, HD, and NN of RVDs preferentially recognize adenine, thymine, cytosine, and guanine/adenine, respectively, and modulation of RVDs can recognize consecutive DNA bases. This simple relationship between amino acid sequence and DNA recognition has allowed for the engineering of specific DNA binding domains by selecting a combination of repeat segments containing the appropriate RVDs.

Besides the wild-type FokI cleavage domain, variants of the FokI cleavage domain with mutations have been designed to improve cleavage specificity and cleavage activity. The FokI domain functions as a dimer, requiring two constructs with unique DNA binding domains for sites in the target genome with proper orientation and spacing. Both the number of amino acid residues between the TALEN DNA binding domain and the FokI cleavage domain and the number of bases between the two individual TALEN binding sites are parameters for achieving high levels of activity.

A relationship between amino acid sequence and DNA recognition of the TALE binding domain allows for designable proteins. Software programs such as DNA Works can be used to design TALE constructs. Other methods of designing TALE constructs are known to those of skill in the art. See Doyle et al., Nucleic Acids Research (2012) 40: W117-122.; Cermak et al., Nucleic Acids Research (2011). 39:e82; and tale-nt.cac.cornell.edu/about.

A CRISPR/Cas9 system, CRISPR/Csm1, CRISPR/Cpf1 system, or a prime editing system (see Anzalone et al.) are alternatives to the FokI-based methods ZFN and TALEN. The CRISPR systems are based on RNA-guided engineered nucleases that use complementary base pairing to recognize DNA sequences at target sites.

CRISPR/Cas9, CRISPR/Csm1, and a CRISPR/Cpf1 systems are part of the adaptive immune system of bacteria and archaea, protecting them against invading nucleic acids such as viruses by cleaving the foreign DNA in a sequence-dependent manner. The immunity is acquired by the integration of short fragments of the invading DNA known as spacers between two adjacent repeats at the proximal end of a CRISPR locus. The CRISPR arrays, including the spacers, are transcribed during subsequent encounters with invasive DNA and are processed into small interfering CRISPR RNAs (crRNAs) approximately 40 nt in length, which combine with the trans-activating CRISPR RNA (tracrRNA) to activate and guide the Cas9 nuclease. This cleaves homologous double-stranded DNA sequences known as protospacers in the invading DNA. A prerequisite for cleavage is the presence of a conserved protospacer-adjacent motif (PAM) downstream of the target DNA, which usually has the sequence 5-NGG-3 but less frequently NAG. Specificity is provided by the so-called “seed sequence” approximately 12 bases upstream of the PAM, which must match between the RNA and target DNA. Cpf1 and Csm1 act in a similar manner to Cas9, but Cpf1 and Csm1 do not require a tracrRNA.

The prime editing system described by Anzalone et al. uses a reverse transcriptase fused to an RNA-programmable nickase with a prime editing extended guide RNA (pegRNA) to directly copy genetic information from the pegRNA into the targeted genomic locus.

### Transgenes

The present disclosure also provides compositions and methods for activating or inhibiting the expression or function of one or more ADC, AO, or ODC genes in a plant, particularly plants of the Nicotiana genus, including tobacco plants of the various commercial varieties.

As used herein, the terms “inhibit,” “inhibition,” and “inhibiting” are defined as any method known in the art or described herein that decreases the expression or function of a gene product of interest (e.g., a target gene product). “Inhibition” can be in the context of a comparison between two plants, for example, a genetically altered plant versus a wild-type plant. Alternatively, inhibition of expression or function of a target gene product can be in the context of a comparison between plant cells, organelles, organs, tissues, or plant parts within the same plant or between different plants, and includes comparisons between developmental or temporal stages within the same plant or plant part or between plants or plant parts. “Inhibition” includes any relative decrement of function or production of a gene product of interest, up to and including complete elimination of function or production of that gene product. The term “inhibition” encompasses any method or composition that down-regulates translation and/or transcription of the target gene product or functional activity of the target gene product. In an aspect, the mRNA or protein level of one or more genes in a modified plant is less than 95%, less than 90%, less than 80%, less than 70%, less than 60%, less than 50%, less than 40%, less than 30%, less than 20%, less than 10%, less than 5%, less than 4%, less than 3%, less than 2%, or less than 1% of the mRNA or protein level of the same gene in a plant that is not a mutant or that has not been genetically modified to inhibit the expression of that gene.

The use of the term “polynucleotide” is not intended to limit the present disclosure to polynucleotides comprising DNA. Those of ordinary skill in the art will recognize that polynucleotides can comprise ribonucleotides and combinations of ribonucleotides and deoxyribonucleotides. Such deoxyribonucleotides and ribonucleotides include both naturally occurring molecules and synthetic analogues. The polynucleotides of the present disclosure also encompass all forms of sequences including, but not limited to, single-stranded forms, double-stranded forms, hairpins, stem-and-loop structures, and the like.

In an aspect, the present disclosure provides recombinant DNA constructs comprising a promoter that is functional in a tobacco cell and operably linked to a polynucleotide that encodes an RNA molecule capable of binding to an RNA encoding a polypeptide having an amino acid sequence at least 80%, at least 85%, at least 90%, at least 95%, at least 97%, at least 98%, at least 99%, or 100% identical to an amino acid sequence selected from the group consisting of SEQ ID NOs: 37-54, and fragments thereof, and where the RNA molecule suppresses the expression of the polypeptide. In an aspect, the RNA molecule is selected from the group consisting of a microRNA, an siRNA, and a trans-acting siRNA. In another aspect, the recombinant DNA construct encodes a double stranded RNA. Also provided are transgenic tobacco plants or part thereof, cured tobacco material, or tobacco products comprising these recombinant DNA constructs. In an aspect, these transgenic plants, cured tobacco material, or tobacco products comprise a lower level of nicotine compared to a control tobacco plant without the recombinant DNA construct. Further provided are methods of reducing the nicotine level of a tobacco plant, the method comprising transforming a tobacco plant with any of these recombinant DNA constructs.

As used herein, “operably linked” refers to a functional linkage between two or more elements. For example, an operable linkage between a polynucleotide of interest and a regulatory sequence (e.g., a promoter) is a functional link that allows for expression of the polynucleotide of interest. Operably linked elements may be contiguous or non-contiguous.

As used herein and when used in reference to a sequence, “heterologous” refers to a sequence that originates from a foreign species, or, if from the same species, is substantially modified from its native form in composition and/or genomic location by deliberate human intervention. The term also is applicable to nucleic acid constructs, also referred to herein as “polynucleotide constructs” or “nucleotide constructs.” In this manner, a “heterologous” nucleic acid construct is intended to mean a construct that originates from a foreign species, or, if from the same species, is substantially modified from its native form in composition and/or genomic location by deliberate human intervention. Heterologous nucleic acid constructs include, but are not limited to, recombinant nucleotide constructs that have been introduced into a plant or plant part thereof, for example, via transformation methods or subsequent breeding of a transgenic plant with another plant of interest. In an aspect, a promoter used is heterologous to the sequence driven by the promoter. In another aspect, a promoter used is heterologous to tobacco. In a further aspect, a promoter used is native to tobacco.

In an aspect, a modified tobacco plant described is a cisgenic plant. As used herein, “cisgenesis” or “cisgenic” refers to genetic modification of a plant, plant cell, or plant genome in which all components (e.g., promoter, donor nucleic acid, selection gene) have only plant origins (i.e., no non-plant origin components are used). In an aspect, a modified plant, plant cell, or plant genome provided is cisgenic. Cisgenic plants, plant cells, and plant genomes provided can lead to ready-to-use tobacco lines. In another aspect, a modified tobacco plant provided comprises no non-tobacco genetic material or sequences.

As used herein, “gene expression” refers to the biosynthesis or production of a gene product, including the transcription and/or translation of the gene product.

In an aspect, recombinant DNA constructs or expression cassettes can also comprise a selectable marker gene for the selection of transgenic cells. Selectable marker genes include, but are not limited to, genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). Additional selectable markers include phenotypic markers such as ß-galactosidase and fluorescent proteins such as green fluorescent protein (GFP).

In an aspect, a tobacco plant provided further comprises increased or reduced expression of activity of genes involved in nicotine biosynthesis or transport. Genes involved in nicotine biosynthesis include, but are not limited to, arginine decarboxylase (ADC), methylputrescine oxidase (MPO), NADH dehydrogenase, ornithine decarboxylase (ODC), phosphoribosylanthranilate isomerase (PRAI), putrescine N-methyltransferase (PMT), quinolate phosphoribosyl transferase (QPT), and S-adenosyl-methionine synthetase (SAMS). Nicotine Synthase, which catalyzes the condensation step between a nicotinic acid derivative and methylpyrrolinium cation, has not been elucidated although two candidate genes (A622 and NBB1) have been proposed. See US 2007/0240728 A1 and US 2008/0120737A1. A622 encodes an isoflavone reductase-like protein. In addition, several transporters may be involved in the translocation of nicotine. A transporter gene, named MATE, has been cloned and characterized (Morita et al., PNAS 106:2447-52 (2009)).

In an aspect, a tobacco plant provided further comprises an increased or reduced level of mRNA, protein, or both of one or more genes encoding a product selected from the group consisting of PMT, MPO, QPT, ADC, ODC, PRAI, SAMS, BBL, MATE, A622, and NBB1, compared to a control tobacco plant. In another aspect, a tobacco plants provided further comprises a transgene directly suppressing the expression of one or more genes encoding a product selected from the group consisting of PMT, MPO, QPT, ADC, ODC, PRAI, SAMS, BBL, MATE, A622, and NBB1. In another aspect, a tobacco plant provided further comprises a transgene or mutation suppressing the expression or activity of one or more genes encoding a product selected from the group consisting of PMT, MPO, QPT, ADC, ODC, PRAI, SAMS, BBL, MATE, A622, and NBB1. In another aspect, a tobacco plant provided further comprises a transgene overexpressing one or more genes encoding a product selected from the group consisting of PMT, MPO, QPT, ADC, ODC, PRAI, SAMS, BBL, MATE, A622, and NBB1.

Also disclosed are the transformation of tobacco plants with recombinant constructs or expression cassettes described using any suitable transformation methods known in the art. Methods for introducing polynucleotide sequences into tobacco plants are known in the art and include, but are not limited to, stable transformation methods, transient transformation methods, and virus-mediated methods. “Stable transformation” refers to transformation where the nucleotide construct of interest introduced into a plant integrates into the genome of the plant and is capable of being inherited by the progeny thereof. “Transient transformation” is intended to mean that a sequence is introduced into the plant and is only temporally expressed or is only transiently present in the plant.

Suitable methods of introducing polynucleotides into plant cells of the present disclosure include microinjection (Crossway et al. (1986) Biotechniques 4:320-334), electroporation (Shillito et al. (1987) Meth. Enzymol. 153:313-336; Riggs et al. (1986) Proc. Natl. Acad. Sci. USA 83:5602-5606), Agrobacterium-mediated transformation (U.S. Pat. Nos. 5,104,310, 5,149,645, 5,177,010, 5,231,019, 5,463,174, 5,464,763, 5,469,976, 4,762,785, 5,004,863, 5,159,135, 5,563,055, and 5,981,840), direct gene transfer (Paszkowski et al. (1984) EMBO J. 3:2717-2722), and ballistic particle acceleration (see, for example, U.S. Pat. Nos. 4,945,050, 5,141,131, 5,886,244, 5,879,918, and 5,932,782; Tomes et al. (1995) in Plant Cell, Tissue, and Organ Culture Fundamental Methods, ed. Gamborg and Phillips (Springer-Verlag, Berlin); McCabe et al. (1988) Biotechnology 6:923-926). Also see Weissinger et al. (1988) Ann. Rev. Genet. 22:421-477; Christou et al. (1988) Plant Physiol. 87:671-674 (soybean); McCabe et al. (1988) Bio Technology 6:923-926 (soybean); Finer and McMullen (1991) In Vitro Cell Dev. Biol. 27P: 175-182 (soybean); Singh et al. (1998) Theor. Appl. Genet. 96:319-324 (soybean); De Wet et al. (1985) in The Experimental Manipulation of Ovule Tissues, ed. Chapman et al. (Longman, N.Y.), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation).

In another aspect, recombinant constructs or expression cassettes may be introduced into plants by contacting plants with a virus or viral nucleic acids. Generally, such methods involve incorporating an expression cassette of the present disclosure within a viral DNA or RNA molecule. It is recognized that promoters for use in expression cassettes also encompass promoters utilized for transcription by viral RNA polymerases. Methods for introducing polynucleotides into plants and expressing a protein encoded therein, involving viral DNA or RNA molecules, are known in the art. See, for example, U.S. Pat. Nos. 5,889,191, 5,889,190, 5,866,785, 5,589,367, 5,316,931, and Porta et al. (1996) Molecular Biotechnology 5:209-221.

Any plant tissue that can be subsequently propagated using clonal methods, whether by organogenesis or embryogenesis, may be transformed with a recombinant construct or an expression cassette. By “organogenesis” in intended the process by which shoots and roots are developed sequentially from meristematic centers. By “embryogenesis” is intended the process by which shoots and roots develop together in a concerted fashion (not sequentially), whether from somatic cells or gametes. Exemplary tissues that are suitable for various transformation protocols described include, but are not limited to, callus tissue, existing meristematic tissue (e.g., apical meristems, axillary buds, and root meristems) and induced meristem tissue (e.g., cotyledon meristem and hypocotyl meristem), hypocotyls, cotyledons, leaf disks, pollen, embryos, and the like.

### Promoters

In an aspect, the present disclosure provides a tobacco plant, or part thereof, comprising a transgene targeting one or more ADC, AO, or ODC gene (e.g., as in an “ADC transgenic plant”, “AO transgenic plant”, or “ODC transgenic plant”). Various types of promoters can be used in an ADC, AO, or ODC transgene or recombinant nucleic acid described here, which are classified according to a variety of criteria relating to the pattern of expression of a coding sequence or gene (including a transgene) operably linked to the promoter, such as constitutive, developmental, tissue-specific, tissue-preferred, inducible, etc. Promoters that initiate transcription in all or most tissues of the plant are referred to as “constitutive” promoters. Promoters that initiate transcription during certain periods or stages of development are referred to as “developmental” promoters. Promoters whose expression is enhanced in certain tissues of the plant relative to other plant tissues are referred to as “tissue-enhanced” or “tissue-preferred” promoters. Thus, a “tissue-preferred” promoter causes relatively higher or preferential expression in a specific tissue(s) of the plant, but with lower levels of expression in other tissue(s) of the plant. Promoters that express within a specific tissue(s) of the plant, with little or no expression in other plant tissues, are referred to as “tissue-specific” promoters. A promoter that expresses in a certain cell type of the plant is referred to as a “cell type specific” promoter. An “inducible” promoter is a promoter that initiates transcription in response to an environmental stimulus such as cold, drought, heat or light, or other stimuli, such as wounding or chemical application. Also used here are promoters that are classified in terms of its origin, such as being heterologous, homologous, chimeric, synthetic, etc. A “heterologous” promoter is a promoter sequence having a different origin relative to its associated transcribable sequence, coding sequence, or gene (or transgene), and/or not naturally occurring in the plant species to be transformed. The term “heterologous” more broadly includes a combination of two or more DNA molecules or sequences when such a combination is not normally found in nature. For example, two or more DNA molecules or sequences would be heterologous with respect to each other if they are normally found in different genomes or at different loci in the same genome, or if they are not identically combined in nature.

In an aspect, recombinant DNA constructs or expression cassettes (or plants containing such constructs or cassettes) described here comprise a promoter selected from the group consisting of a constitutive promoter, an inducible promoter, and a tissue-preferred promoter (for example, a leaf-specific or root-specific promoter). Exemplary constitutive promoters include the core promoter of the Rsyn7 promoter and other constitutive promoters disclosed in U.S. Pat. No. 6,072,050; the core CaMV 35S promoter (Odell et al. (1985) Nature 313:810-812); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J 3:2723-2730); ALS promoter (U.S. Pat. No. 5,659,026), and the like. Exemplary chemical-inducible promoters include the tobacco PR-1a promoter, which is activated by salicylic acid. Other chemical-inducible promoters of interest include steroid-responsive promoters (see, for example, the glucocorticoid-inducible promoter in Schena et al. (1991) Proc. Natl. Acad. Sci. USA 88:10421-10425 and McNellis et al. (1998) Plant J. 14(2):247-257) and tetracycline-inducible promoters (see, for example, Gatz et al. (1991) Mol. Gen. Genet. 227:229-237, and U.S. Pat. Nos. 5,814,618 and 5,789,156). Additional exemplary promoters that can be used are those responsible for heat-regulated gene expression, light-regulated gene expression (for example, the pea rbcS-3A; the maize rbcS promoter; the chlorophyll alb-binding protein gene found in pea; or the Arabssu promoter), hormone-regulated gene expression (for example, the abscisic acid (ABA) responsive sequences from the Em gene of wheat; the ABA-inducible HVA1 and HVA22, and rd29A promoters of barley and Arabidopsis; and wound-induced gene expression (for example, of wun1), organ specific gene expression (for example, of the tuber-specific storage protein gene; the 23-kDa zein gene from maize described by; or the French bean (β-phaseolin gene), or pathogen-inducible promoters (for example, the PR-1, prp-1, or (β-1,3 glucanase promoters, the fungal-inducible wir1a promoter of wheat, and the nematode-inducible promoters, TobRB7-5A and Hmg-1, of tobacco arid parsley, respectively).

In an aspect, an ADC, AO, or ODC transgene comprising an inducible promoter. In one aspect, an inducible promoter is a topping-inducible promoter. In an aspect, an inducible promoter is also a tissue-specific or tissue-preferred promoter. In one aspect, a tissue-specific or tissue-preferred promoter is specific or preferred for one or more tissues or organs selected from the group consisting of shoot, root, leaf, stem, flower, sucker, root tip, mesophyll cells, epidermal cells, and vasculature. In a further aspect, a topping inducible promoter comprises a promoter sequence from a tobacco nicotine demethylase gene, for example, CYP82E4, CYP82E5, or CYP82E10. In an aspect, an inducible promoter provides root specific or preferred expression. Exemplary root specific or preferred inducible promoter can be found in U.S. Patent Application Publication No. 2019/0271000. In an aspect, an inducible promoter provides leaf specific or preferred expression. Exemplary leaf specific or preferred inducible promoter can be found in U.S. Patent Application Publication No. 2019/0271000, which is herein incorporated by reference in its entirety.

In an aspect, an inducible promoter is a heterologous to the operably linked transcribable DNA sequence. In one aspect, a transcribable DNA sequence encodes a non-coding RNA selected from the group consisting of microRNA (miRNA), anti-sense RNA, small interfering RNA (siRNA), a trans-acting siRNA (ta-siRNA), and hairpin RNA (hpRNA). In an aspect, a non-coding RNA comprises a nucleotide sequence having 100%, at least 99.9%, at least 99.5%, at least 99%, at least 98%, at least 97%, at least 96%, at least 95%, at least 94%, at least 93%, at least 92%, at least 91%, at least 90%, at least 85%, at least 80%, or at least 75% identity or complementarity to a sequence selected from the group consisting of SEQ ID NOs: 1-36, 55-64, and any portions thereof.

In an aspect, a non-coding RNA comprises at least 18, at least 19, at least 20, at least 21, at least 22, at least 23, at least 24, at least 25, at least 26, at least 27, at least 28, at least 29, at least 30, at least 31, at least 32, at least 33, at least 34, or at least 35 nucleotides. In an aspect, a non-coding RNA sequence comprises at least 80% complementarity to at least 18, at least 19, at least 20, at least 21, at least 22, at least 23, at least 24, at least 25, at least 26, at least 27, at least 28, at least 29, at least 30, at least 31, at least 32, at least 33, at least 34, or at least 35 consecutive nucleotides of a sequence selected from the group consisting of SEQ ID NOs: 1-36 and 55-64. In an aspect, a non-coding RNA sequence comprises at least 90% complementarity to at least 18, at least 19, at least 20, at least 21, at least 22, at least 23, at least 24, at least 25, at least 26, at least 27, at least 28, at least 29, at least 30, at least 31, at least 32, at least 33, at least 34, or at least 35 consecutive nucleotides of a sequence selected from the group consisting of SEQ ID NOs: 1-36 and 55-64. In an aspect, a non-coding RNA sequence comprises at least 95% complementarity to at least 18, at least 19, at least 20, at least 21, at least 22, at least 23, at least 24, at least 25, at least 26, at least 27, at least 28, at least 29, at least 30, at least 31, at least 32, at least 33, at least 34, or at least 35 consecutive nucleotides of a sequence selected from the group consisting of SEQ ID NOs: 1-36 and 55-64. In an aspect, a non-coding RNA sequence comprises 100% complementarity to at least 18, at least 19, at least 20, at least 21, at least 22, at least 23, at least 24, at least 25, at least 26, at least 27, at least 28, at least 29, at least 30, at least 31, at least 32, at least 33, at least 34, or at least 35 consecutive nucleotides of a sequence selected from the group consisting of SEQ ID NOs: 1-36 and 55-64.

### Tobacco Types

In an aspect, a tobacco plant provided is from a tobacco type selected from the group consisting of flue-cured tobacco, air-cured tobacco, dark air-cured tobacco, dark fire-cured tobacco, Galpão tobacco, and Oriental tobacco. In another aspect, a tobacco plant provided is from a tobacco type selected from the group consisting of Burley tobacco, Maryland tobacco, and dark tobacco.

In an aspect, a tobacco plant provided is in a flue-cured tobacco background or exhibits one or more flue-cured tobacco characteristic described here. Flue-cured tobaccos (also called “Virginia” or “bright” tobaccos) amount to approximately 40% of world tobacco production. Flue-cured tobaccos are often also referred to as “bright tobacco” because of the golden-yellow to deep-orange color it reaches during curing. Flue-cured tobaccos have a light, bright aroma and taste. Flue-cured tobaccos are generally high in sugar and low in oils. Major flue-cured tobacco growing countries are Argentina, Brazil, China, India, Tanzania and the United States of America. In one aspect, tobacco plants or seeds or modified tobacco plants or seeds provided herein are of a flue-cured tobacco variety selected from the group consisting of the varieties listed in Table 1, and any variety essentially derived from any one of the foregoing varieties. See WO 2004/041006 A1. In a further aspect, modified tobacco plants or seeds provided herein are in a flue-cured variety selected from the group consisting of K326, K346, and NC196.

TABLE 1
Flue-cured Tobacco Varieties

400 (TC 225)
401 (TC 226)
401 Cherry Red (TC 227)
401 Cherry Red Free (TC 228)
Cash (TC 250)
Cash (TI 278)
CC 101
CC 1063
CC 13
CC 143
CC 200
CC 27
CC 301
CC 33
CC 35
CC 37
CC 400
CC 500
CC 600
CC 65
CC 67
CC 700
CC 800
CC 900
Coker 139 (TC 259)
Coker 139 yb1, yb2
Coker 140 (TC 260)
Coker 176 (TC 262)
Coker 187 (TC 263)
Coker 187-Hicks (TC 265)
Coker 209 (TC 267)
Coker 258 (TC 270)
Coker 298 (TC 272)
Coker 316 (TC 273)
Coker 319 (TC 274)
Coker 347 (TC 275)
Coker 371-Gold (TC 276)
Coker 411 (TC 277)
Coker 48 (TC 253)
Coker 51 (TC 254)
Coker 86 (TC 256)
CU 263 (TC 619)
CU 561
DH95-1562-1
Dixie Bright 101 (TC 290)
Dixie Bright 102 (TC 291)
Dixie Bright 244 (TC 292)
Dixie Bright 27 (TC 288)
Dixie Bright 28 (TC 289)
GF 157
GF 318
GL 26H
GL 338
GL 350
GL 368
GL 395
GL 600
GL 737
GL 939
GL 939 (TC 628)
Hicks (TC 310)
Hicks Broadleaf (TC 311)
K 149 (TC 568)
K 317
K 326
K 326 (TC 319)
K 340 (TC 320)
K 346
K 346 (TC 569)
K 358
K 394 (TC 321)
K 399
K 399 (TC 322)
K 730
Lonibow (TI 1573)
Lonibow (TI 1613)
McNair 10 (TC 330)
McNair 135 (TC 337)
McNair 30 (TC 334)
McNair 373 (TC 338)
McNair 944 (TC 339)
MK94 (TI 1512)
MS K 326
MS NC 71
MS NC 72
NC 100
NC 102
NC 1071 (TC 364)
NC 1125-2
NC 12 (TC 346)
NC 1226
NC 196
NC 2326 (TC 365)
NC 27 NF (TC 349)
NC 291
NC 297
NC 299
NC 37 NF (TC 350)
NC 471
NC 55
NC 567 (TC 362)
NC 60 (TC 352)
NC 606
NC 6140
NC 71
NC 72
NC 729 (TC 557)
NC 810 (TC 659)
NC 82 (TC 356)
NC 8640
NC 89 (TC 359)
NC 92
NC 925
NC 95 (TC 360)
NC 98 (TC 361)
NC EX 24
NC PY 10 (TC 367)
NC TG 61
Oxford 1 (TC 369)
Oxford 1-181 (TC 370)
Oxford 2 (TC 371)
Oxford 207 (TC 632)
Oxford 26 (TC 373)
Oxford 3 (TC 372)
Oxford 414 NF
PD 611 (TC 387)
PVH 03
PVH 09
PVH 1118
PVH 1452
PVH 1600
PVH 2110
PVH 2275
R 83 (Line 256-1) (TI 1400)
Reams 134
Reams 158
Reams 713
Reams 744
Reams M1
RG 11 (TC 600)
RG 13 (TC 601)
RG 17 (TC 627)
RG 22 (TC 584)
RG 8 (TC 585)
RG 81 (TC 618)
RG H51
RG4H 217
RGH 12
RGH 4
RGH 51
RGH 61
SC 58 (TC 400)
SC 72 (TC 403)
Sp. G-168
SPEIGHT 168
Speight 168 (TC 633)
Speight 172 (TC 634)
Speight 178
Speight 179
Speight 190
Speight 196
SPEIGHT 220
SPEIGHT 225
SPEIGHT 227
SPEIGHT 236
Speight G-10 (TC 416)
Speight G-102
Speight G-108
Speight G-111
Speight G-117
Speight G-126
Speight G-15 (TC 418)
Speight G-23
Speight G-28 (TC 420)
Speight G-33
Speight G-41
Speight G-5
Speight G-52
Speight G-58
Speight G-70
Speight G-70 (TC 426)
Speight G-80 (TC 427)
Speight NF3 (TC 629)
STNCB
VA 182
VA 45 (TC 559)
Vesta 30 (TC 439)
Vesta 33 (TC 440)
Vesta 5 (TC 438)
Vesta 62 (TC 441)
Virginia (TI 220)
Virginia (TI 273)
Virginia (TI 877)
Virginia 115 (TC 444)
Virginia 21 (TC 443)
Virginia Bright (TI 964)
Virginia Bright Leaf (TC 446)
Virginia Gold (TC 447)
White Stem Orinoco (TC 451)

In an aspect, a tobacco plant provided is in an air-cured tobacco background or exhibits one or more air-cured tobacco characteristic described here. Air-cured tobaccos include “Burley,” “Maryland,” and “dark” tobaccos. The common factor linking air-cured tobaccos is that curing occurs primarily without artificial sources of heat and humidity. Burley tobaccos are light to dark brown in color, high in oil, and low in sugar. Burley tobaccos are typically air-cured in barns. Major Burley growing countries include Argentina, Brazil, Italy, Malawi, and the United States of America.

Maryland tobaccos are extremely fluffy, have good burning properties, low nicotine and a neutral aroma. Major Maryland growing countries include the United States of America and Italy.

In one aspect, tobacco plants or seeds or modified tobacco plants or seeds provided herein are of a Burley tobacco variety selected from the group consisting of the tobacco varieties listed in Table 2, and any variety essentially derived from any one of the foregoing varieties. In a further aspect, modified tobacco plants or seeds provided herein are in a Burley variety selected from the group consisting of TN 90, KT 209, KT 206, KT212, and HB 4488.

TABLE 2
Burley Tobacco Varieties

4407 LC
AA-37-1
Burley 21 (TC 7)
Burley 49 (TC 10)
Burley 64 (TC 11)
Burley Mammoth KY 16 (TC 12)
Clay 402
Clay 403
Clay 502
Clays 403
GR 10 (TC 19)
GR 10 (TC 19)
GR 10A (TC 20)
GR 13 (TC 21)
GR 14 (TC 22)
GR 149 LC
GR 153
GR 17 (TC 23)
GR 17B (TC 24)
GR 18 (TC 25)
GR 19 (TC 26)
GR 2 (TC 15)
GR 24 (TC 27)
GR 36 (TC 28)
GR 38 (TC 29)
GR 38A (TC 30)
GR 40 (TC 31)
GR 42 (TC 32)
GR 42C (TC 33)
GR 43 (TC 34)
GR 44 (TC 35)
GR 45 (TC 36)
GR 46 (TC 37)
GR 48 (TC 38)
GR 5 (TC 16)
GR 53 (TC 39)
GR 6 (TC 17)
GR 9 (TC 18)
GR139 NS
GR139 S
HB 04P
HB 04P LC
HB 3307P LC
HB 4108P
HB 4151P
HB 4192P
HB 4194P
HB 4196
HB 4488
HB 4488P
HB04P
HB 4488 LC
HIB 21
HPB 21
HY 403
Hybrid 403 LC
Hybrid 404 LC
Hybrid 501 LC
KDH-959 (TC 576)
KDH-960 (TC 577)
KT 200 LC
KT 204 LC
KT 206 LC
KT 209 LC
KT 210 LC
KT 212 LC
KT 215 LC
KY 1 (TC 52)
KY 10 (TC 55)
KY 12 (TC 56)
KY 14 (TC 57)
KY 14 × L8 LC
KY 15 (TC 58)
KY 16 (TC 59)
KY 17 (TC 60)
KY 19 (TC 61)
KY 21 (TC 62)
KY 22 (TC 63)
KY 24 (TC 64)
KY 26 (TC 65)
KY 33 (TC 66)
KY 34 (TC 67)
KY 35 (TC 68)
KY 41A (TC 69)
KY 5 (TC 53)
KY 52 (TC 70)
KY 54 (TC 71)
KY 56 (TC 72)
KY 56 (TC 72)
KY 57 (TC 73)
KY 58 (TC 74)
KY 8654 (TC 77)
KY 8959
KY 9 (TC 54)
KY 907 LC
KY 908 (TC 630)
NBH 98 (Screened)
NC 1206
NC 129
NC 2000 LC
NC 2002 LC
NC 3 LC
NC 5 LC
NC 6 LC
NC 7 LC
NC BH 129 LC
NC03-42-2
Newton 98
R 610 LC
R 630 LC
R 7-11
R 7-12 LC
RG 17
TKF 1801 LC
TKF 2002 LC
TKF 4024 LC
TKF 4028 LC
TKF 6400 LC
TKF 7002 LC
TKS 2002 LC
TN 86 (TC 82)
TN 90 LC
TN 97 Hybrid LC
TN 97 LC
VA 116
VA 119
Virgin A Mutante (TI 1406)
Virginia 509 (TC 84)

In another aspect, tobacco plants or seeds or modified tobacco plants or seeds provided herein are of a Maryland tobacco variety selected from the group consisting of the tobacco varieties listed in Table 3, and any variety essentially derived from any one of the foregoing varieties.

TABLE 3
Maryland Tobacco Varieties

Maryland 10 (TC 498)
Maryland 14 D2 (TC 499)
Maryland 201 (TC 503)
Maryland 21 (TC 500)
Maryland 341 (TC 504)
Maryland 40
Maryland 402
Maryland 59 (TC 501)
Maryland 601
Maryland 609 (TC 505)
Maryland 64 (TC 502)
Maryland 872 (TC 506)
Maryland Mammoth (TC 507)

In an aspect, a tobacco plant provided is in a dark air-cured tobacco background or exhibits one or more dark air-cured tobacco characteristic described here. Dark air-cured tobaccos are distinguished from other tobacco types primarily by its curing process, which gives dark air-cured tobacco its medium-brown to dark-brown color and a distinct aroma. Dark air-cured tobaccos are mainly used in the production of chewing tobacco and snuff. In one aspect, modified tobacco plants or seeds provided herein are of a dark air-cured tobacco variety selected from the group consisting of Sumatra, Jatim, Dominican Cubano, Besuki, One sucker, Green River, Virginia sun-cured, and Paraguan Passado, and any variety essentially derived from any one of the foregoing varieties.

In an aspect, a tobacco plant provided is in a dark fire-cured tobacco background or exhibits one or more dark fire-cured tobacco characteristic described here. Dark fire-cured tobaccos are generally cured with low-burning wood fires on the floors of closed curing barns. Dark fire-cured tobaccos are typically used for making pipe blends, cigarettes, chewing tobacco, snuff, and strong-tasting cigars. Major growing regions for dark fire-cured tobaccos are Tennessee, Kentucky, and Virginia in the United States of America. In one aspect, tobacco plants or seeds or modified tobacco plants or seeds provided herein are of a dark fire-cured tobacco variety selected from the group consisting of the tobacco varieties listed in Table 4, and any variety essentially derived from any one of the foregoing varieties.

TABLE 4
Dark Fire-Cured Tobacco Varieties

Black Mammoth (TC 461)
Black Mammoth Small Stalk (TC 641)
Certified Madole (TC 463)
D-534-A-1 (TC 464)
DAC ULT 302
DAC ULT 303
DAC ULT 306
DAC ULT 308
DAC ULT 312
DF 300 (TC 465)
DF 485 (TC 466)
DF 516 (TC 467)
DF 911 (TC 468)
DT 508
DT 518 (Screened)
DT 538 LC
DT 592
Improved Madole (TC 471)
Jernigan's Madole (TC 472)
KT 14LC
KT D17LC
KT D4 LC
KT D6 LC
KT D8 LC
KY 153 (TC 216)
KY 157 (TC 217)
KY 160
KY 160 (TC 218)
KY 163 (TC 219)
KY 165 (TC 220)
KY 170 (TC 474)
KY 171 (PhPh)
KY 171 (TC 475)
KY 171 LC
KY 171 NS
KY 180 (TC 573)
KY 190 (TC 574)
Little Crittenden
Little Crittenden (TC 476)
Little Crittenden LC (certified)
Little Crittenden PhPh
Lizard Tail Turtle Foot
Madole (TC 478)
Madole (TC 479)
MS KY 171
MS NL Madole LC
MS TN D950 LC
Nance (TC 616)
Narrow Leaf Madole LC (certified)
Neal Smith Madole (TC 646)
Newtons VH Madole
NL Madole
NL Madole (PhPh)
NL Madole (TC 484)
NL Madole LC
NL Madole LC (PhPh)
NL Madole NS
One Sucker (TC 224)
OS 400
PD 302H
PD 312H
PD 318H
PD 7302 LC
PD 7305
PD 7309 LC
PD 7312 LC
PD 7318 LC
PD 7319 LC
Petico M PG04
PY KY 160 (TC 612)
PY KY 171 (TC 613)
Shirey
TI 1372
TN D94
TN D94 (TC 621)
TN D950
TN D950 (PhPh)
TN D950
TN D950 (TC 622)
TR Madole (TC 486)
VA 309
VA 309 (TC 560)
VA 309 LC (certified)
VA 310 (TC 487)
VA 331 (TC 592)
VA 355 (TC 638)
VA 359
VA 359 (Screened)
VA 359 (TC 639)
VA 359 LC (certified)
VA 403 (TC 580)
VA 405 (TC 581)
VA 409 (TC 562)
VA 510 (TC 572)

In an aspect, a tobacco plant provided is in an Oriental tobacco background or exhibits one or more Oriental tobacco characteristic described here. Oriental tobaccos are also referred to as Greek, aroma and Turkish tobaccos due to the fact that they are typically grown in eastern Mediterranean regions such as Turkey, Greece, Bulgaria, Macedonia, Syria, Lebanon, Italy, and Romania. The small plant size, small leaf size, and unique aroma properties of Oriental tobacco varieties are a result of their adaptation to the poor soil and stressful climatic conditions in which they have been developed. In one aspect, tobacco plants or seeds or modified tobacco plants or seeds provided herein are of an Oriental tobacco variety selected from the group consisting of the tobacco varieties listed in Table 5, and any variety essentially derived from any one of the foregoing varieties.

TABLE 5
Oriental Tobacco Varieties.

Bafra (TI 1641)
Bahce (TI 1730)
Bahia (TI 1416)
Bahia (TI 1455)
Baiano (TI 128)
Basma
Basma (TI 1666)
Basma Drama
Basma Hybrid (PhPh)
Basma Zihna I
Bitlis (TI 1667)
Bitlis (TI 1725)
Bubalovac (TI 1282)
Bursa (TI 1650)
Bursa (TI 1668)
Canik (TI 1644)
Djebel 174 (TI 1492)
Djebel 359 (TI 1493)
Djebel 81
Dubec 566 (TI 1409)
Dubec 7 (TI 1410)
Dubek 566 (TI 1567)
Duzce (TI 1670)
Edirne (TI 1671)
Ege (TI 1642)
Ege-64 (TI 1672)
Izmir (Akhisar) (TI 1729)
Izmir (Gavurkoy) (TI 1727)
Izmir Ege 64
Izmir-Incekara (TI 1674)
Izmir-Ozbas (TI 1675)
Jaka Dzebel (TI 1326)
Kaba-Kulak
Kagoshima Maruba (TI 158)
Katerini
Katerini S53
Krumovgrad 58
MS Basma
MS Katerini S53
Nevrokop 1146
Ozbas (TI 1645)
Perustitza (TI 980)
Prilep (TI 1291)
Prilep (TI 1325)
Prilep 12-2/1
Prilep 23
Samsun (TC 536)
Samsun 959 (TI 1570)
Samsun Evkaf (TI 1723)
Samsun Holmes NN (TC 540)
Samsun Maden (TI 1647)
Samsun NO 15 (TC 541)
Samsun-BLK SHK Tol (TC 542)
Samsun-Canik (TI 1678)
Samsun-Maden (TI 1679)
Saribaptar 407 - Izmir Region
Smyrna (TC 543)
Smyrna No. 23 (TC 545)
Smyrna No. 9 (TC 544)
Smyrna-Blk Shk Tol (TC 546)
Trabzon (TI 1649)
Trabzon (TI 1682)
Trapezund 161 (TI 1407)
Turkish (TC 548)
Turkish Angshit (TI 90)
Turkish Samsum (TI 92)
Turkish Tropizoid (TI 93)
Turkish Varotic (TI 89)
Xanthi (TI 1662)

In an aspect, tobacco plants or seeds or modified tobacco plants or seeds provided herein are of an cigar tobacco variety selected from the group consisting of the tobacco varieties listed in Table 6, and any variety essentially derived from any one of the foregoing varieties.

TABLE 6
Cigar Tobacco Varieties

Bahai (TI 62)
Beinhart 1000
Beinhart 1000 (TI 1562)
Beinhart 1000-1 (TI 1561)
Bergerac C
Bergerac C (TI 1529)
Big Cuban (TI 1565)
Castillo Negro, Blanco, Pina (TI 448)
Castillo Negro, Blanco, Pina (TI 448A)
Castillo Negro, Blanco, Pina (TI 449)
Caujaro (TI 893)
Chocoa (TI 289)
Chocoa (TI 313)
Connecticut 15 (TC 183)
Connecticut Broadleaf
Connecticut Broadleaf (TC 186)
Connecticut Shade (TC 188)
Criollo, Colorado (TI 1093)
Enshu (TI 1586)
Florida 301
Florida 301 (TC 195)
PA Broadleaf (TC 119)
Pennsylvania Broadleaf
Pennsylvania Broadleaf (TC 119)
Petite Havana SR1
Petite Havana SR1 (TC 105)

In an aspect, tobacco plants or seeds or modified tobacco plants or seeds provided herein are of a tobacco variety selected from the group consisting of the tobacco varieties listed in Table 7, and any variety essentially derived from any one of the foregoing varieties.

TABLE 7
Other Tobacco Varieties

Chocoa (TI 319)
Hoja Parada (TI 1089)
Hoja Parado (Galpoa) (TI 1068)
Perique (St. James Parrish)
Perique (TC 556)
Perique (TI 1374)
Sylvestris (TI 984)
TI 179

In an aspect, a modified tobacco plant, seed, or cell described here is from a variety selected from the group consisting of the tobacco varieties listed in Table 1, Table 2, Table 3, Table 4, Table 5, Table 6, and Table 7.

In an aspect, low-alkaloid or low-nicotine tobacco plants, seeds, hybrids, varieties, or lines are essentially derived from or in the genetic background of BU 64, CC 101, CC 200, CC 27, CC 301, CC 400, CC 500, CC 600, CC 700, CC 800, CC 900, Coker 176, Coker 319, Coker 371 Gold, Coker 48, CU 263, DF911, Galpao tobacco, GL 26H, GL 350, GL 600, GL 737, GL 939, GL 973, HB 04P, K 149, K 326, K 346, K 358, K394, K 399, K 730, KDH 959, KT 200, KT204LC, KY 10, KY 14, KY 160, KY 17, KY 171, KY 907, KY907LC, KTY14×L8 LC, Little Crittenden, McNair 373, McNair 944, msKY 14×L8, Narrow Leaf Madole, NC 100, NC 102, NC 2000, NC 291, NC 297, NC 299, NC 3, NC 4, NC 5, NC 6, NC7, NC 606, NC 71, NC 72, NC 810, NC BH 129, NC 2002, Neal Smith Madole, OXFORD 207, ‘Perique’ tobacco, PVH03, PVH09, PVH19, PVH50, PVH51, R 610, R 630, R 7-11, R 7-12, RG 17, RG 81, RG H51, RGH 4, RGH 51, RS 1410, Speight 168, Speight 172, Speight 179, Speight 210, Speight 220, Speight 225, Speight 227, Speight 234, Speight G-28, Speight G-70, Speight H-6, Speight H20, Speight NF3, T1 1406, T1 1269, TN 86, TN86LC, TN 90, TN 97, TN97LC, TN D94, TN D950, TR (Tom Rosson) Madole, VA 309, or VA359, Maryland 609, HB3307PLC, HB4488PLC, KT206LC, KT209LC, KT210LC, KT212LC, R610LC, PVH2310, NC196, KTD14LC, KTD6LC, KTD8LC, PD7302LC, PD7305LC, PD7309LC, PD7318LC, PD7319LC, PD7312LC, ShireyLC, or any commercial tobacco variety according to standard tobacco breeding techniques known in the art.

All foregoing mentioned specific varieties of dark air-cured, Burley, Maryland, dark fire-cured, or Oriental type are listed only for exemplary purposes. Any additional dark air-cured, Burley, Maryland, dark fire-cured, Oriental varieties are also contemplated in the present application.

Also provided are populations of tobacco plants described. In an aspect, a population of tobacco plants has a planting density of between about 5,000 and about 8,000, between about 5,000 and about 7,600, between about 5,000 and about 7,200, between about 5,000 and about 6,800, between about 5,000 and about 6,400, between about 5,000 and about 6,000, between about 5,000 and about 5,600, between about 5,000 and about 5,200, between about 5,200 and about 8,000, between about 5,600 and about 8,000, between about 6,000 and about 8,000, between about 6,400 and about 8,000, between about 6,800 and about 8,000, between about 7,200 and about 8,000, or between about 7,600 and about 8,000 plants per acre. In another aspect, a population of tobacco plants is in a soil type with low to medium fertility.

Also provided are containers of seeds from tobacco plants described. A container of tobacco seeds of the present disclosure may contain any number, weight, or volume of seeds. For example, a container can contain at least, or greater than, about 100, 200, 300, 400, 500, 600, 700, 800, 900, 1000, 1500, 2000, 2500, 3000, 3500, 4000 or more seeds. Alternatively, the container can contain at least, or greater than, about 50, 100, 200, 300, 400, 500, 600, 700, 800, 900, 1000, 1500, 2000, 2500, 3000, 3500, 4000 grams or more seeds. Containers of tobacco seeds may be any container available in the art. By way of non-limiting example, a container may be a box, a bag, a packet, a pouch, a tape roll, a tube, or a bottle.

### Curing/Products

Also provided is cured tobacco material made from a low-alkaloid or low-nicotine tobacco plant described. Further provided is cured tobacco material made from a tobacco plant described with higher levels of total alkaloid or nicotine.

“Curing” is the aging process that reduces moisture and brings about the destruction of chlorophyll giving tobacco leaves a golden color and by which starch is converted to sugar. Cured tobacco therefore has a higher reducing sugar content and a lower starch content compared to harvested green leaf. In an aspect, green leaf tobacco provided can be cured using conventional means, e.g., flue-cured, barn-cured, fire-cured, air-cured or sun-cured. See, for example, Tso (1999, Chapter 1 in Tobacco, Production, Chemistry and Technology, Davis & Nielsen, eds., Blackwell Publishing, Oxford) for a description of different types of curing methods. Cured tobacco is usually aged in a wooden drum (e.g., a hogshead) or cardboard cartons in compressed conditions for several years (e.g., two to five years), at a moisture content ranging from 10% to about 25%. See, U.S. Pat. Nos. 4,516,590 and 5,372,149. Cured and aged tobacco then can be further processed. Further processing includes conditioning the tobacco under vacuum with or without the introduction of steam at various temperatures, pasteurization, and fermentation. Fermentation typically is characterized by high initial moisture content, heat generation, and a 10 to 20% loss of dry weight. See, e.g., U.S. Pat. Nos. 4,528,993, 4,660,577, 4,848,373, 5,372,149; U.S. Publication No. 2005/0178398; and Tso (1999, Chapter 1 in Tobacco, Production, Chemistry and Technology, Davis & Nielsen, eds., Blackwell Publishing, Oxford). Cure, aged, and fermented tobacco can be further processed (e.g., cut, shredded, expanded, or blended). See, for example, U.S. Pat. Nos. 4,528,993; 4,660,577; and 4,987,907. In an aspect, the cured tobacco material of the present disclosure is sun-cured. In another aspect, the cured tobacco material of the present disclosure is flue-cured, air-cured, or fire-cured.

Tobacco material obtained from the tobacco lines, varieties or hybrids of the present disclosure can be used to make tobacco products. As used herein, “tobacco product” is defined as any product made or derived from tobacco that is intended for human use or consumption.

Tobacco products provided include, without limitation, cigarette products (e.g., cigarettes and bidi cigarettes), cigar products (e.g., cigar wrapping tobacco and cigarillos), pipe tobacco products, products derived from tobacco, tobacco-derived nicotine products, smokeless tobacco products (e.g., moist snuff, dry snuff, and chewing tobacco), films, chewables, tabs, shaped parts, gels, consumable units, insoluble matrices, hollow shapes, reconstituted tobacco, expanded tobacco, and the like. See, e.g., U.S. Patent Publication No. 2006/0191548.

As used herein, “cigarette” refers a tobacco product having a “rod” and “filler”. The cigarette “rod” includes the cigarette paper, filter, plug wrap (used to contain filtration materials), tipping paper that holds the cigarette paper (including the filler) to the filter, and all glues that hold these components together. The “filler” includes (1) all tobaccos, including but not limited to reconstituted and expanded tobacco, (2) non-tobacco substitutes (including but not limited to herbs, non-tobacco plant materials and other spices that may accompany tobaccos rolled within the cigarette paper), (3) casings, (4) flavorings, and (5) all other additives (that are mixed into tobaccos and substitutes and rolled into the cigarette).

As used herein, “reconstituted tobacco” refers to a part of tobacco filler made from tobacco dust and other tobacco scrap material, processed into sheet form and cut into strips to resemble tobacco. In addition to the cost savings, reconstituted tobacco is very important for its contribution to cigarette taste from processing flavor development using reactions between ammonia and sugars.

As used herein, “expanded tobacco” refers to a part of tobacco filler which is processed through expansion of suitable gases so that the tobacco is “puffed” resulting in reduced density and greater filling capacity. It reduces the weight of tobacco used in cigarettes.

Tobacco products derived from plants of the present disclosure also include cigarettes and other smoking articles, particularly those smoking articles including filter elements, where the rod of smokable material includes cured tobacco within a tobacco blend. In an aspect, a tobacco product of the present disclosure is selected from the group consisting of a cigarillo, a non-ventilated recess filter cigarette, a vented recess filter cigarette, a cigar, snuff, pipe tobacco, cigar tobacco, cigarette tobacco, chewing tobacco, leaf tobacco, hookah tobacco, shredded tobacco, and cut tobacco. In another aspect, a tobacco product of the present disclosure is a smokeless tobacco product. Smokeless tobacco products are not combusted and include, but not limited to, chewing tobacco, moist smokeless tobacco, snus, and dry snuff. Chewing tobacco is coarsely divided tobacco leaf that is typically packaged in a large pouch-like package and used in a plug or twist. Moist smokeless tobacco is a moist, more finely divided tobacco that is provided in loose form or in pouch form and is typically packaged in round cans and used as a pinch or in a pouch placed between an adult tobacco consumer's cheek and gum. Snus is a heat treated smokeless tobacco. Dry snuff is finely ground tobacco that is placed in the mouth or used nasally. In a further aspect, a tobacco product of the present disclosure is selected from the group consisting of loose leaf chewing tobacco, plug chewing tobacco, moist snuff, and nasal snuff. In yet another aspect, a tobacco product of the present disclosure is selected from the group consisting of an electronically heated cigarette, an e-cigarette, an electronic vaporing device.

In an aspect, a tobacco product of the present disclosure can be a blended tobacco product. In one aspect, a blended tobacco product comprises cured tobacco materials. In an aspect, a cured tobacco material constitutes about at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of cured tobacco in a tobacco blend by weight. In one aspect, a cured tobacco material constitutes about at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of cured tobacco in a tobacco blend by volume.

In an aspect, a tobacco product of the present disclosure can be a low nicotine tobacco product. In a further aspect, a tobacco product of the present disclosure may comprise nornicotine at a level of less than about 3 mg/g. For example, the nornicotine content in such a product can be about 3.0 mg/g, 2.5 mg/g, 2.0 mg/g, 1.5 mg/g, 1.0 mg/g, 750 μg/g, 500 pg/g, 250 pg/g, 100 pg/g, 75 pg/g, 50 pg/g, 25 pg/g, 10 pg/g, 7.0 pg/g, 5.0 pg/g, 4.0 pg/g, 2.0 pg/g, 1.0 pg/g, 0.5 pg/g, 0.4 pg/g, 0.2 pg/g, 0.1 pg/g, 0.05 pg/g, 0.01 pg/g, or undetectable.

In an aspect, cured tobacco material or tobacco products provided comprise an average nicotine or total alkaloid level selected from the group consisting of about 0.01%, 0.02%, 0.05%, 0.75%, 0.1%, 0.15%, 0.2%, 0.3%, 0.35%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 5%, 6%, 7%, 8%, and 9% on a dry weight basis. In another aspect, cured tobacco material or tobacco products provided comprise an average nicotine or total alkaloid level selected from the group consisting of about between 0.01% and 0.02%, between 0.02% and 0.05%, between 0.05% and 0.75%, between 0.75% and 0.1%, between 0.1% and 0.15%, between 0.15% and 0.2%, between 0.2% and 0.3%, between 0.3% and 0.35%, between 0.35% and 0.4%, between 0.4% and 0.5%, between 0.5% and 0.6%, between 0.6% and 0.7%, between 0.7% and 0.8%, between 0.8% and 0.9%, between 0.9% and 1%, between 1% and 1.1%, between 1.1% and 1.2%, between 1.2% and 1.3%, between 1.3% and 1.4%, between 1.4% and 1.5%, between 1.5% and 1.6%, between 1.6% and 1.7%, between 1.7% and 1.8%, between 1.8% and 1.9%, between 1.9% and 2%, between 2% and 2.1%, between 2.1% and 2.2%, between 2.2% and 2.3%, between 2.3% and 2.4%, between 2.4% and 2.5%, between 2.5% and 2.6%, between 2.6% and 2.7%, between 2.7% and 2.8%, between 2.8% and 2.9%, between 2.9% and 3%, between 3% and 3.1%, between 3.1% and 3.2%, between 3.2% and 3.3%, between 3.3% and 3.4%, between 3.4% and 3.5%, and between 3.5% and 3.6% on a dry weight basis. In a further aspect, cured tobacco material or tobacco products provided comprise an average nicotine or total alkaloid level selected from the group consisting of about between 0.01% and 0.1%, between 0.02% and 0.2%, between 0.03% and 0.3%, between 0.04% and 0.4%, between 0.05% and 0.5%, between 0.75% and 1%, between 0.1% and 1.5%, between 0.15% and 2%, between 0.2% and 3%, and between 0.3% and 3.5% on a dry weight basis.

The present disclosure further provides a method manufacturing a tobacco product comprising tobacco material from tobacco plants disclosed. In an aspect, methods comprise conditioning aged tobacco material made from tobacco plants to increase its moisture content from between about 12.5% and about 13.5% to about 21%, blending the conditioned tobacco material to produce a desirable blend. In an aspect, the method of manufacturing a tobacco product further comprises casing or flavoring the blend. Generally, during the casing process, casing or sauce materials are added to blends to enhance their quality by balancing the chemical composition and to develop certain desired flavor characteristics. Further details for the casing process can be found in Tobacco Production, Chemistry and Technology, Edited by L. Davis and M. Nielsen, Blackwell Science, 1999.

Tobacco material provided can be also processed using methods including, but not limited to, heat treatment (e.g., cooking, toasting), flavoring, enzyme treatment, expansion and/or curing. Both fermented and non-fermented tobaccos can be processed using these techniques. Examples of suitable processed tobaccos include dark air-cured, dark fire cured, burley, flue cured, and cigar filler or wrapper, as well as the products from the whole leaf stemming operation. In an aspect, tobacco fibers include up to 70% dark tobacco on a fresh weight basis. For example, tobacco can be conditioned by heating, sweating and/or pasteurizing steps as described in U.S. Publication Nos. 2004/0118422 or 2005/0178398.

Tobacco material provided can be subject to fermentation. Fermenting typically is characterized by high initial moisture content, heat generation, and a 10 to 20% loss of dry weight. See, e.g., U.S. Pat. Nos. 4,528,993; 4,660,577; 4,848,373; and 5,372,149. In addition to modifying the aroma of the leaf, fermentation can change either or both the color and texture of a leaf. Also during the fermentation process, evolution gases can be produced, oxygen can be taken up, the pH can change, and the amount of water retained can change. See, for example, U.S. Publication No. 2005/0178398 and Tso (1999, Chapter 1 in Tobacco, Production, Chemistry and Technology, Davis & Nielsen, eds., Blackwell Publishing, Oxford). Cured, or cured and fermented tobacco can be further processed (e.g., cut, expanded, blended, milled or comminuted) prior to incorporation into the oral product. The tobacco, in some cases, is long cut fermented cured moist tobacco having an oven volatiles content of between 48 and 50 weight percent prior to mixing with the copolymer and optionally flavorants and other additives.

In an aspect, tobacco material provided can be processed to a desired size. In an aspect, tobacco fibers can be processed to have an average fiber size of less than 200 micrometers. In an aspect, tobacco fibers are between 75 and 125 micrometers. In another aspect, tobacco fibers are processed to have a size of 75 micrometers or less. In an aspect, tobacco fibers include long cut tobacco, which can be cut or shredded into widths of about 10 cuts/inch up to about 110 cuts/inch and lengths of about 0.1 inches up to about 1 inch. Double cut tobacco fibers can have a range of particle sizes such that about 70% of the double cut tobacco fibers falls between the mesh sizes of −20 mesh and 80 mesh.

Tobacco material provided can be processed to have a total oven volatiles content of about 10% by weight or greater; about 20% by weight or greater; about 40% by weight or greater; about 15% by weight to about 25% by weight; about 20% by weight to about 30% by weight; about 30% by weight to about 50% by weight; about 45% by weight to about 65% by weight; or about 50% by weight to about 60% by weight. Those of skill in the art will appreciate that “moist” tobacco typically refers to tobacco that has an oven volatiles content of between about 40% by weight and about 60% by weight (e.g., about 45% by weight to about 55% by weight, or about 50% by weight). As used herein, “oven volatiles” are determined by calculating the percentage of weight loss for a sample after drying the sample in a pre-warmed forced draft oven at 110° C. for 3.25 hours. The oral product can have a different overall oven volatiles content than the oven volatiles content of the tobacco fibers used to make the oral product. The processing steps described can reduce or increase the oven volatiles content.

### Breeding

The present disclosure also provides methods for breeding tobacco lines, cultivars, or varieties comprising a desirable level of total alkaloid or nicotine, e.g., low nicotine or nicotine free, and desired leaf quality or grade level. Breeding can be carried out via any known procedures. DNA fingerprinting, SNP mapping, haplotype mapping or similar technologies may be used in a marker-assisted selection (MAS) breeding program to transfer or breed a desirable trait or allele into a tobacco plant. For example, a breeder can create segregating populations in a F₂ or backcross generation using F₁ hybrid plants or further crossing the F₁ hybrid plants with other donor plants with an agronomically desirable genotype. Plants in the F₂ or backcross generations can be screened for a desired agronomic trait or a desirable chemical profile using one of the techniques known in the art or listed herein. Depending on the expected inheritance pattern or the MAS technology used, self-pollination of selected plants before each cycle of backcrossing to aid identification of the desired individual plants can be performed. Backcrossing or other breeding procedure can be repeated until the desired phenotype of the recurrent parent is recovered. A recurrent parent in the present disclosure can be a flue-cured variety, a Burley variety, a dark air-cured variety, a dark fire-cured variety, or an Oriental variety. Other breeding techniques can be found, for example, in Wernsman, E. A., and Rufty, R. C. 1987. Chapter Seventeen. Tobacco. Pages 669-698 In: Cultivar Development. Crop Species. W. H. Fehr (ed.), MacMillan Publishing Go., Inc., New York, N. Y., incorporated herein by reference in their entirety.

Results of a plant breeding program using the tobacco plants described includes useful lines, cultivars, varieties, progeny, inbreds, and hybrids of the present disclosure. As used herein, the term “variety” refers to a population of plants that share constant characteristics which separate them from other plants of the same species. A variety is often, although not always, sold commercially. While possessing one or more distinctive traits, a variety is further characterized by a very small overall variation between individuals within that variety. A “pure line” variety may be created by several generations of self-pollination and selection, or vegetative propagation from a single parent using tissue or cell culture techniques. A variety can be essentially derived from another line or variety. As defined by the International Convention for the Protection of New Varieties of Plants (Dec. 2, 1961, as revised at Geneva on Nov. 10, 1972; on Oct. 23, 1978; and on Mar. 19, 1991), a variety is “essentially derived” from an initial variety if: a) it is predominantly derived from the initial variety, or from a variety that is predominantly derived from the initial variety, while retaining the expression of the essential characteristics that result from the genotype or combination of genotypes of the initial variety; b) it is clearly distinguishable from the initial variety; and c) except for the differences which result from the act of derivation, it conforms to the initial variety in the expression of the essential characteristics that result from the genotype or combination of genotypes of the initial variety. Essentially derived varieties can be obtained, for example, by the selection of a natural or induced mutant, a somaclonal variant, a variant individual from plants of the initial variety, backcrossing, or transformation. A first tobacco variety and a second tobacco variety from which the first variety is essentially derived, are considered as having essentially identical genetic background. A “line” as distinguished from a variety most often denotes a group of plants used non-commercially, for example in plant research. A line typically displays little overall variation between individuals for one or more traits of interest, although there may be some variation between individuals for other traits.

It is understood that any tobacco plant of the present disclosure can further comprise additional agronomically desirable traits, for example, by transformation with a genetic construct or transgene using a technique known in the art. Without limitation, an example of a desired trait is herbicide resistance, pest resistance, disease resistance; high yield; high grade index value; curability; curing quality; mechanical harvestability; holding ability; leaf quality; height, plant maturation (e.g., early maturing, early to medium maturing, medium maturing, medium to late maturing, or late maturing); stalk size (e.g., a small, medium, or a large stalk); or leaf number per plant (e.g., a small (e.g., 5-10 leaves), medium (e.g., 11-15 leaves), or large (e.g., 16-21) number of leaves), or any combination. In an aspect, low-nicotine or nicotine-free tobacco plants or seeds disclosed comprise one or more transgenes expressing one or more insecticidal proteins, such as, for example, a crystal protein of Bacillus thuringiensis or a vegetative insecticidal protein from Bacillus cereus, such as VIP3 (see, for example, Estruch et al. (1997) Nat. Biotechnol. 15:137). In another aspect, tobacco plants further comprise an introgressed trait conferring resistance to brown stem rot (U.S. Pat. No. 5,689,035) or resistance to cyst nematodes (U.S. Pat. No. 5,491,081).

The present disclosure also provides tobacco plants comprising an altered nicotine or total alkaloid level but having a yield comparable to the yield of corresponding initial tobacco plants without such a nicotine level alternation. In an aspect, a low-nicotine or nicotine-free tobacco variety provides a yield selected from the group consisting of about between 1200 and 3500, between 1300 and 3400, between 1400 and 3300, between 1500 and 3200, between 1600 and 3100, between 1700 and 3000, between 1800 and 2900, between 1900 and 2800, between 2000 and 2700, between 2100 and 2600, between 2200 and 2500, and between 2300 and 2400 pounds/acre. In another aspect, a low-nicotine or nicotine-free tobacco variety provides a yield selected from the group consisting of about between 1200 and 3500, between 1300 and 3500, between 1400 and 3500, between 1500 and 3500, between 1600 and 3500, between 1700 and 3500, between 1800 and 3500, between 1900 and 3500, between 2000 and 3500, between 2100 and 3500, between 2200 and 3500, between 2300 and 3500, between 2400 and 3500, between 2500 and 3500, between 2600 and 3500, between 2700 and 3500, between 2800 and 3500, between 2900 and 3500, between 3000 and 3500, and between 3100 and 3500 pounds/acre. In a further aspect, low-nicotine or nicotine-free tobacco plants provide a yield between 65% and 130%, between 70% and 130%, between 75% and 130%, between 80% and 130%, between 85% and 130%, between 90% and 130%, between 95% and 130%, between 100% and 130%, between 105% and 130%, between 110% and 130%, between 115% and 130%, or between 120% and 130% of the yield of a control plant having essentially identical genetic background except a genetic modification providing the low-nicotine or nicotine-free trait. In a further aspect, low-nicotine or nicotine-free tobacco plants provide a yield between 70% and 125%, between 75% and 120%, between 80% and 115%, between 85% and 110%, or between 90% and 100% of the yield of a control plant having essentially identical genetic background except a genetic modification providing the low-nicotine or nicotine-free trait.

In an aspect, an ADC, AO, or ODC mutant or transgenic tobacco plant exhibits one or more, two or more, three or more, or all of the traits selected from the group consisting of: increased yield as compared to the low-alkaloid background control, accelerated ripening and senescence as compared to the low-alkaloid background control, reduced susceptibility to insect herbivory as compared to the low-alkaloid background control, and reduced polyamine content after topping as compared to the low-alkaloid background control. In an aspect, an ADC, AO, or ODC mutant or transgenic tobacco plant in a low-alkaloid background exhibits one or more, two or more, three or more, or all of the traits selected from the group consisting of: increased yield as compared to LA BU21, accelerated ripening and senescence as compared to LA BU21, reduced susceptibility to insect herbivory as compared to LA BU21, and reduced polyamine content after topping as compared to LA BU21.

In an aspect, an ADC, AO, or ODC mutant or transgenic tobacco plant (e.g., a low-nicotine, nicotine-free, or low-alkaloid tobacco variety) does not exhibit one or more, two or more, three or more, or all of the LA BU21 traits selected from the group consisting of lower yield, delayed ripening and senescence of leaves, higher susceptibility to insect herbivory, increased polyamine content after topping, higher chlorophyll, more mesophyll cells per unit leaf area, and poor end-product quality after curing. In an aspect, a tobacco plant disclosed (e.g., a low-nicotine, nicotine-free, or low-alkaloid tobacco variety) does not exhibit two or more of the LA BU21 traits selected from the group consisting of lower yield, delayed ripening and senescence, higher susceptibility to insect herbivory, increased polyamine content after topping, higher chlorophyll, more mesophyll cells per unit leaf area, and poor end-product quality after curing. In an aspect, a tobacco plant disclosed (e.g., a low-nicotine, nicotine-free, or low-alkaloid tobacco variety) does not exhibit three or more of the LA BU21 traits selected from the group consisting of lower yield, delayed ripening and senescence, higher susceptibility to insect herbivory, increased polyamine content after topping, higher chlorophyll, more mesophyll cells per unit leaf area, and poor end-product quality after curing. In an aspect, a tobacco plant disclosed (e.g., a low-nicotine, nicotine-free, or low-alkaloid tobacco variety) exhibits at a lower level compared to LA BU21, LAFC53, or LN KY171, one or more, two or more, three or more, or all of the LA BU21 traits selected from the group consisting of lower yield, delayed ripening and senescence, higher susceptibility to insect herbivory, increased polyamine content after topping, higher chlorophyll, more mesophyll cells per unit leaf area, and poor end-product quality after curing. In an aspect, a tobacco plant disclosed (e.g., a low-nicotine, nicotine-free, or low-alkaloid tobacco variety) exhibits at a lower level compared to LA BU21, LAFC53, or LN KY171, two or more of the LA BU21 traits selected from the group consisting of lower yield, delayed ripening and senescence, higher susceptibility to insect herbivory, increased polyamine content after topping, higher chlorophyll, more mesophyll cells per unit leaf area, and poor end-product quality after curing. In an aspect, a tobacco plant disclosed (e.g., a low-nicotine, nicotine-free, or low-alkaloid tobacco variety) exhibits at a lower level compared to LA BU21, LAFC53, or LN KY171, three or more, or all of the LA BU21 traits selected from the group consisting of lower yield, delayed ripening and senescence, higher susceptibility to insect herbivory, increased polyamine content after topping, higher chlorophyll, more mesophyll cells per unit leaf area, and poor end-product quality after curing.

In an aspect, a modified tobacco plant (e.g., a low-nicotine, nicotine-free, or low-alkaloid tobacco variety) comprises an ADC, AO, or ODC genetic modification and a further genetic modification conferring a desired trait (e.g., low-nicotine, nicotine-free, or low-alkaloid) without substantially impacting a trait selected from the group consisting of yield, ripening and senescence, susceptibility to insect herbivory, polyamine content after topping, chlorophyll level, mesophyll cell number per unit leaf area, and end-product quality after curing.

In an aspect, an ADC, AO, or ODC mutant or transgenic tobacco plant comprises a modification conferring a desired trait (e.g., low-nicotine, nicotine-free, or low-alkaloid) and further comprises a trait substantially comparable to an unmodified control plant, where the trait is selected from the group consisting of yield, ripening and senescence, susceptibility to insect herbivory, polyamine content after topping, chlorophyll level, mesophyll cell number per unit leaf area, and end-product quality after curing.

In an aspect, tobacco plants provided are hybrid plants. Hybrids can be produced by preventing self-pollination of female parent plants (e.g., seed parents) of a first variety, permitting pollen from male parent plants of a second variety to fertilize the female parent plants, and allowing F₁ hybrid seeds to form on the female plants. Self-pollination of female plants can be prevented by emasculating the flowers at an early stage of flower development. Alternatively, pollen formation can be prevented on the female parent plants using a form of male sterility. For example, male sterility can be produced by male sterility (MS), or transgenic male sterility where a transgene inhibits microsporogenesis and/or pollen formation, or self-incompatibility. Female parent plants containing MS are particularly useful. In aspects in which the female parent plants are MS, pollen may be harvested from male fertile plants and applied manually to the stigmas of MS female parent plants, and the resulting F₁ seed is harvested.

Plants can be used to form single-cross tobacco F₁ hybrids. Pollen from a male parent plant is manually transferred to an emasculated female parent plant or a female parent plant that is male sterile to form F₁ seed. Alternatively, three-way crosses can be carried out where a single-cross F₁ hybrid is used as a female parent and is crossed with a different male parent. As another alternative, double-cross hybrids can be created where the F₁ progeny of two different single-crosses are themselves crossed. Self-incompatibility can be used to particular advantage to prevent self-pollination of female parents when forming a double-cross hybrid.

In an aspect, a low-nicotine or nicotine-free tobacco variety is male sterile. In another aspect, a low-nicotine or nicotine-free tobacco variety is cytoplasmic male sterile. Male sterile tobacco plants may be produced by any method known in the art. Methods of producing male sterile tobacco are described in Wernsman, E. A., and Rufty, R. C. 1987. Chapter Seventeen. Tobacco. Pages 669-698 In: Cultivar Development. Crop Species. W. H. Fehr (ed.), MacMillan Publishing Go., Inc., New York, N.Y. 761 pp.

In a further aspect, tobacco parts provided include, but are not limited to, a leaf, a stem, a root, a seed, a flower, pollen, an anther, an ovule, a pedicel, a fruit, a meristem, a cotyledon, a hypocotyl, a pod, an embryo, endosperm, an explant, a callus, a tissue culture, a shoot, a cell, and a protoplast. In an aspect, tobacco part provided does not include seed. In an aspect, this disclosure provides tobacco plant cells, tissues, and organs that are not reproductive material and do not mediate the natural reproduction of the plant. In another aspect, this disclosure also provides tobacco plant cells, tissues, and organs that are reproductive material and mediate the natural reproduction of the plant. In another aspect, this disclosure provides tobacco plant cells, tissues, and organs that cannot maintain themselves via photosynthesis. In another aspect, this disclosure provides somatic tobacco plant cells. Somatic cells, contrary to germline cells, do not mediate plant reproduction.

The provided cells, tissues and organs may be from seed, fruit, leaf, cotyledon, hypocotyl, meristem, embryos, endosperm, root, shoot, stem, pod, flower, infloresence, stalk, pedicel, style, stigma, receptacle, petal, sepal, pollen, anther, filament, ovary, ovule, pericarp, phloem, vascular tissue. In another aspect, this disclosure provides a tobacco plant chloroplast. In a further aspect, this disclosure provides epidermal cells, stomata cell, leaf or root hairs, a storage root, or a tuber. In another aspect, this disclosure provides a tobacco protoplast.

Skilled artisans understand that tobacco plants naturally reproduce via seeds, not via asexual reproduction or vegetative propagation. In an aspect, this disclosure provides tobacco endosperm. In another aspect, this disclosure provides tobacco endosperm cells. In a further aspect, this disclosure provides a male or female sterile tobacco plant, which cannot reproduce without human intervention. Skilled artisans further understand that cured tobacco does not constitute a living organism and is not capable of growth or reproduction.

### Nucleic Acid/Polypeptide

In an aspect, the present disclosure provides a nucleic acid molecule comprising at least about 40%, 45%, 50%, 55%, 60%, 65%, 70%, 75%, 80%, 85%, 90%, 91%, 92%, 93%, 94%, 95%, 96%, 97%, 98%, 99%, or 100% identity to a sequence selected from the group consisting of SEQ ID NOs: 1 to 36, and fragments thereof. In an aspect, the present disclosure provides a polypeptide or protein comprising at least about 40%, 45%, 50%, 55%, 60%, 65%, 70%, 75%, 80%, 85%, 90%, 91%, 92%, 93%, 94%, 95%, 96%, 97%, 98%, 99%, or 100% identity or similarity to an amino acid sequence selected from the group consisting of SEQ ID NOs: 37 to 54. In another aspect, the present disclosure provides a biologically active variant of a protein having an amino acid sequence selected from the group consisting of SEQ ID NOs: 37 to 54. A biologically active variant of a protein of the present disclosure may differ from that protein by as few as 1-15 amino acid residues, as few as 10, as few as 9, as few as 8, as few as 7, as few as 6, as few as 5, as few as 4, as few as 3, as few as 2, or as few as 1 amino acid residue. Also provided are orthologous genes or proteins of genes or proteins comprising a sequence selected from the group consisting of SEQ ID NOs: 1 to 54. “Orthologs” are genes derived from a common ancestral gene and which are found in different species as a result of speciation. Orthologs may share at least 60%, 70%, 75%, 80%, 85%, 90%, 91%, 92%, 93%, 94%, 95%, 96%, 97%, 98%, 99%, or greater sequence identity or similarity at the nucleotide sequence and/or the protein sequence level. Functions of orthologs are often highly conserved among species.

As used herein, the term “sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are deemed to have “sequence similarity” or “similarity.” For any protein sequences provided here, also contemplated are functional homolog proteins that differ in one or more amino acids as a result of one or more of well-known conservative amino acid substitutions, e.g., valine is a conservative substitute for alanine and threonine is a conservative substitute for serine. Conservative substitutions for an amino acid within the native sequence can be selected from other members of a class to which the naturally occurring amino acid belongs. Representative amino acids within these various classes include, but are not limited to: (1) acidic (negatively charged) amino acids such as aspartic acid and glutamic acid; (2) basic (positively charged) amino acids such as arginine, histidine, and lysine; (3) neutral polar amino acids such as glycine, serine, threonine, cysteine, tyrosine, asparagine, and glutamine; and (4) neutral nonpolar (hydrophobic) amino acids such as alanine, leucine, isoleucine, valine, proline, phenylalanine, tryptophan, and methionine. Conserved substitutes for an amino acid within a native amino acid sequence can be selected from other members of the group to which the naturally occurring amino acid belongs. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Naturally conservative amino acids substitution groups are: valine-leucine, valine-isoleucine, phenylalanine-tyrosine, lysine-arginine, alanine-valine, aspartic acid-glutamic acid, and asparagine-glutamine. A further aspect of the disclosure includes proteins that differ in one or more amino acids from those of a described protein sequence as the result of deletion or insertion of one or more amino acids in a native sequence.

Nucleic acid molecules, polypeptides, or proteins provided can be isolated or substantially purified. An “isolated” or “purified” nucleic acid molecule, polypeptide, protein, or biologically active portion thereof, is substantially or essentially free from components that normally accompany or interact with the polynucleotide or protein as found in its naturally occurring environment. For example, an isolated or purified polynucleotide or protein is substantially free of other cellular material, or culture medium when produced by recombinant techniques, or substantially free of chemical precursors or other chemicals when chemically synthesized.

Having now generally described the disclosure, the same will be more readily understood through reference to the following examples that are provided by way of illustration, and are not intended to be limiting of the present disclosure, unless specified.

### EXAMPLES

### Example 1: RNAi Approach

Plant alkaloids comprise a large group of nitrogen-containing metabolites that are widely distributed throughout the plant kingdom. Alkaloids of Nicotiana tabacum L. (tobacco), and especially so that of nicotine, are secondary metabolites that have, since long ago, attracted widespread attention in biology, commerce, society, and medicine (Tso and Jeffrey 1961; Leete 1977; Waller and Nowacki 1978; Baldwin 1989; Dewey and Xie 2013; Patra et al. 2013). Commercial tobacco cultivars typically produce alkaloids at levels between 2-6% of the total dry biomass weight. In typical commercial tobacco plants, nicotine accounts for about 90% of the total alkaloid pool (Tayoub et al. 2015; Moghbel et al. 2017), with the secondary alkaloids nornicotine (a demethylated derivative of nicotine), anatabine and anabasine making up most of the remainder (Saitoh et al. 1985; Sisson and Severson 1990). Recent advances in sequencing and molecular biology have led to the characterization of the majority of the genes coding for the enzymes that are responsible for the biosynthesis of these secondary metabolites (Dewey and Xie 2013).

Availability of the genome sequence of tobacco and the knowledge of many of the structural and regulatory genes involved in the nicotine biosynthetic pathway (Kajikawa et al. 2017) afforded the possibility of manipulating tobacco gene expression so as to perturb biosynthetic pathways and, thereby, alter the leaf alkaloid content. For example, Kajikawa et al. (2017) reported on phylogenetic and expression analyses, which revealed a series of structural genes of the nicotine biosynthetic pathway, forming a regulon and operating under the control of jasmonate-responsive ETHYLENE RESPONSE FACTOR (ERF) transcription factors.

Putrescine, an important polyamine precursor, is thought to be derived from ornithine via the activity of the enzyme ornithine decarboxylase (ODC) and possibly from arginine via the activity of the arginine decarboxylase (ADC). Putresine may serve as the reactant to generate the pyrollidine ring of nicotine in Nicotiana tabacum and related species. To test for the arginine biosynthetic route in N. tabacum, Chintapakorn and Hamill (2007) used an antisense approach with the hairy root culture system of tobacco, to down-regulate ADC activity in transformant plants. They found concentrations of nicotine to be comparable in antisense-ADC and control lines throughout most of their respective culture cycles, except at the later stages of growth, when the nicotine content of ADC-antisense lines is ˜20% lower than that in the controls (Chintapakorn and Hamill (2007). They found levels of anatabine, the second most abundant alkaloid typically in N. tabacum, to be slightly elevated in two ADC-antisense lines at the latter stages of their culture cycles compared to controls. Their work suggested that the ADC mediated route to putrescine plays a role, but is not of primary importance, in providing the pyrollidine ring for nicotine synthesis.

To test for the ornithine biosynthetic route in N. tabacum, DeBoer et al (2011) used RNAi methodology also with the hairy root culture system to down-regulate ODC transcript levels in N. tabacum. They observed a marked effect upon the alkaloid profile of transgenic tissues, with ODC transcript down-regulation leading to lower nicotine and increased anatabine levels in both cultured hairy roots and intact greenhouse-grown plants. They concluded that the ornithine metabolite and the ODC catalyzed biosynthetic pathway to putrescine is more important than the ADC mediated pathway in nicotine biosynthesis and in defining the nicotine:anatabine ratio in N. tabacum. These finding are further validated by DeBoer et al. (2013), who showed that RNAi-mediated down-regulation of the ornithine decarboxylase (ODC) expression in Nicotiana glauca hindered the well-known wound-stress stimulation of nicotine and anabasine biosynthesis and accumulation.

Here, a systematic effort is undertaken to apply RNAi technology and to down-regulate the expression of several alkaloid biosynthesis-related genes in Nicotiana tabacum, including the arginine decarboxylase, agmatine deiminase, aspartate oxidase, arginase, ornithine decarboxylase, and S′adenosyl-L:-methionine (SAM) synthase. This effort helps achieve a more integrated understanding of the interplay between these different genes and pathways in nicotine biosynthesis.

### Example 2: Plant Material

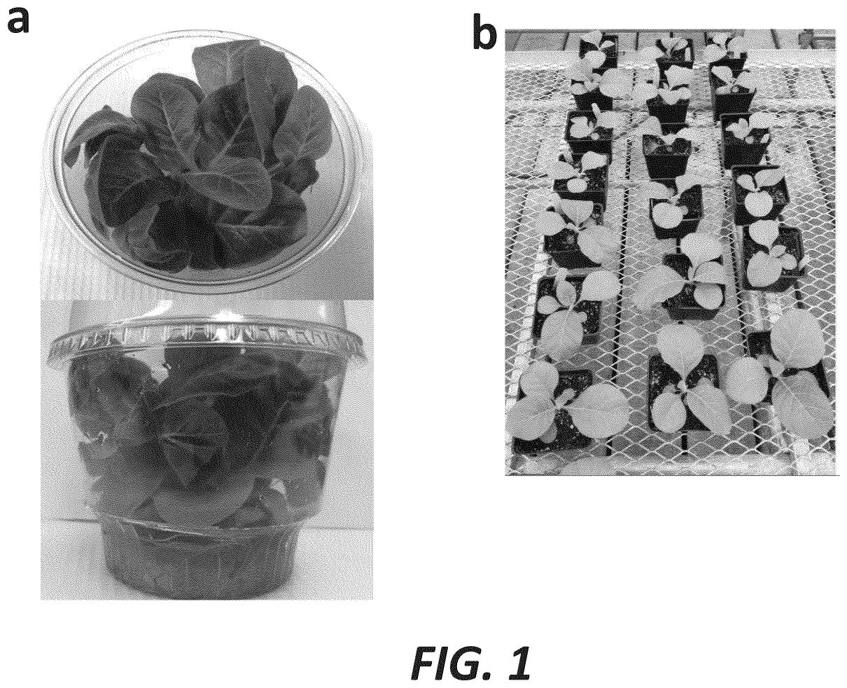

The plant material employed in this work is Nicotiana tabacum, cv K326-ALCS3 (wild type tobacco). In-vitro plantlets in Dixie cups are obtained after seed germination and initial growth on solid Murashige and Skoog (MS; 1962) agar media, supplemented with vitamins and 30 g L⁻¹ sucrose. Seedlings are maintained at 24° C. under a 16/8-h photoperiod (FIG. 1a).

For stable transformation, 1 cm×1 cm leaf pieces from these tobacco seedlings are infected with transgenic Agrobacterium tumefaciens upon immersing the leaves for 5 min in 25 mL YEB cell suspension containing 1 g L⁻¹ yeast extract, 5 g L⁻¹ meat extract, 5 g L⁻¹ bacto peptone, 5 g L⁻¹ sucrose, 492.8 mg L⁻¹ MgSO₄-7H₂O, plus 0.2 mM of acetosyringone, pH 6.8. The optical density (OD₆₀₀) of the A. tumefaciens cells in the YEB media is between 0.8 and 1. After immersing, the leaf pieces are dried upon gentle blotting with sterile Whatman paper, then transferred to Petri agar plates containing MS media and incubated in darkness for two days.

For selection and regeneration, the treated leaf pieces are transferred to agar plates containing MS media supplemented with 500 mg L⁻¹ cefotaxime, 150 mg L⁻¹ kanamycin, 1 mg L^{-1 6}-benzylaminopurine (BA), 1 mg L⁻¹ thiamine-HCl and 100 mg L⁻¹ myo inositol. Three rounds of transfer under selection are sufficient to enable rooting of the regenerants, upon which the latter are transferred to MS agar media in Dixie cups containing 500 mg L⁻¹ cefotaxime and 150 mg L⁻¹ kanamycin. The plantlets obtained after these steps of selection and regeneration in the presence of kanamycin are considered to be the TO transformant generation.

Rooted transformants after approximately 5-weeks of growth in the Dixie cups are transferred to soil in the greenhouse and cultivated under ambient sunlight conditions (FIG. 1b). After flowering, T1 seeds are collected, sterilized, cataloged, germinated under kanamycin selection, and individually cultivated in Dixie cups in-vitro in the presence of 150 mg L⁻¹ kanamycin. These T1 plantlets are later transferred to the greenhouse, as the T1 transformant generation.

Topping of the T1 tobacco plants in the greenhouse is applied when they started to flower. In this approach, the flower head and the shoot down to the first completely expanded leaf from the top are removed. After two weeks of further growth and leaf expansion, the 3^{rd} and 4^{th} leaves from the top are harvested for analysis.

### Example 3: Bacteria and Plasmids

To transform the tobacco plants, nine strains of Agrobacterium tumefaciens LBA4404 are generated and used: the wild type control and eight engineered strains carrying the binary plant expression vector p45-2-7-1 with the respective nucleotides encoding the RNAi sequences and the sequence for antibiotic selection (kanamycin) to serve as the selectable marker in both bacteria and plant transformants. These sequences are preceded by the constitutive Cassava Vein Mosaic Virus (CsVMV), serving as the promoter, and followed by the nopaline synthase gene (NOS) terminator.

The RNAi designs are based on the transcript sequences that encode the proteins listed in Table 8. The coding regions of the genes of interest (Ornithine decarboxylase—ODC, Arginine decarboxylase—ADC, Aspartate oxidase—AO, S-adenosylmethionine synthetase—SAMS, Agmatine deiminase—AIC, and Arginase—ARG) are retrieved from an internal NT3.1 database. The cDNA sequences of the above-mentioned genes, and the RNAi construct design along with the corresponding nucleotide sequences are given in Table 9. For each gene of interest, a unique region of about 230 bp to 350 bp is selected and inserted in the forward and reverse orientation across the 2^{nd} intron of Arabidopsis thaliana Actin-11 gene (GenBank accession #BT005593.1), respectively. Due to high sequence similarity in target genes, ODC-1a and ODC-1b can be targeted by one RNAi construct (ODC-RNAi). Similarly, ADC-1a and ADC-1b can be targeted by one RNAi construct (ADC-RNAi). The entire cassette is synthesized at Genscript (Piscataway, NJ) and cloned under the control of the Cassava vein mosaic virus promoter and nopaline synthase-terminator. The binary vector contains the kanamycin resistance gene, NPTII, for transgenic plant selection. The plasmid sequences are verified by PCR and Sanger sequencing using 2 sets of primers: CSVMV-F with Intron-R, and Intron-F with NosT-R (see Table 10). The plasmids are then transformed into Agrobacterium tumefaciens and positive clones are used for transformation of tobacco. The aspartate oxidase and SAM synthase entails the design, construction, and use of two and three RNAi constructs, respectively. Only a single RNAi construct is employed for the other RNAi constructs.

TABLE 8
Gene name, GenBank ID, and locator number for the alkaloid biosynthesis
genes manipulated here. RNAi constructs are designed (please see Supplementary
materials, page 12) and, through Agrobacterium tumefaciens transformation
are instilled in the nuclear genome of Nicotiana tabacum.

| | | | Agrobacterium |
|---|---|---|---|
| Gene name, GenBank ID, | Protein | RNAi | tumefaciens |
| and Locator Number | name | designation | construct |
| ADC-1a: NM_001325190.1 (g67073) | Arginine | ADC | At-ADC |
| ADC-1b: XM_016577301.1 (g1739) | decarboxylase | | |
| AIC-1a: XM_016610112.1 (g78883) | Agmatine | AIC | At-AIC |
| AIC-1b: XM_016615863.1 (g61368) | deiminase | | |
| AO-1: XR_001648132.1 (g34809) | Aspartate | AO1 | At-AO1 |
| AO-3: XM_016595053.1 (g34814) | oxidase | AO2 | At-AO2 |
| AO-2: XM_016633697.1 (g83551a) | | | |
| AO-4: (g83551b) | | | |
| Arginase-1a: XM_016589408.1 | Arginase | ARG | At-ARG |
| (g76552) | | | |
| Arginase-1b: XM_016589407.1 | | | |
| (g29644) | | | |
| ODC-1a: NM_001325697.1 (g49499) | Ornithine | ODC | At-ODC |
| ODC-1b: AF233849.1 (g65779) | decarboxylase | | |
| SAMS-1a: XM_016641833.1 (g95117) | SAM | S1 | At-S1 |
| SAMS-1b: XM_016625263.1 (g23092) | synthase | S2 | At-S2 |
| SAMS-2a: XM_016583083.1 (g22730) | | S3 | At-S3 |
| SAMS-2b: AF321140.1 (g94670) | | | |
| SAMS-3a: XM_016608142.1 (g10384) | | | |
| SAMS-3b: NM_001325403.1 (g41661) | | | |

TABLE 9
Gene name and sequences of the alkaloid biosynthesis genes
manipulated here. Genomic DNA Sequences include regions
such as promoter, 5′ UTR, introns, 3′ UTR, and
terminator. The RNAi sequence refers to a gene-specific
sequence used to generate an inverted repeat RNAi-encoding
cassette. As shown, certain RNAi sequences can target multiple
genes due to the high similarity of these gene sequences.

| | | Genomic | | | |
|---|---|---|---|---|---|
| | | DNA | cDNA | Protein | RNAi |
| | | Sequence | Sequence | Sequence | Sequence |
| Gene | Gene | (SEQ | (SEQ | (SEQ | (SEQ |
| Name | ID | ID NO.) | ID NO.) | ID NO.) | ID NO.) |
| ADC-1a | g67073 | 1 | 19 | 37 | 55 |
| ADC-1b | g1739 | 2 | 20 | 38 | 55 |
| AIC-1a | g78883 | 3 | 21 | 39 | 56 |
| AIC-1b | g61368 | 4 | 22 | 40 | 56 |
| AO-1 | g34809 | 5 | 23 | 41 | 57 |
| AO-3 | g34814 | 6 | 24 | 42 | 58 |
| AO-2 | g83551a | 7 | 25 | 43 | 59 |
| AO-4 | g83551b | 8 | 26 | 44 | 59 |
| Arginase-1a | g76552 | 9 | 27 | 45 | 60 |
| Arginase-1b | g29644 | 10 | 28 | 46 | 60 |
| ODC-1a | g49499 | 11 | 29 | 47 | 61 |
| ODC-1b | g65779 | 12 | 30 | 48 | 61 |
| SAMS-1a | g95117 | 13 | 31 | 49 | 62 |
| SAMS-1b | g23092 | 14 | 32 | 50 | 62 |
| SAMS-2a | g22730 | 15 | 33 | 51 | 63 |
| SAMS-2b | g94670 | 16 | 34 | 52 | 63 |
| SAMS-3a | g10384 | 17 | 35 | 53 | 64 |
| SAMS-3b | g41661 | 18 | 36 | 54 | 64 |

### Example 4: Agroinfiltration

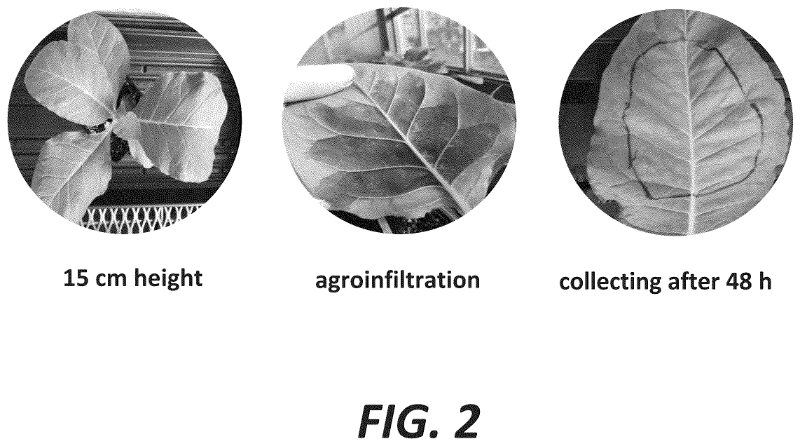

About 15 cm tall greenhouse-cultivated tobacco plants at about the same developmental stage are used for transient expression experiments with the various RNAi constructs. For infiltration, an overnight culture of Agrobacterium tumefaciens, with cells grown at 28° C., is centrifuged and the cell pellet is resuspended in agroinfiltration buffer (10 mM MES, pH=5.7, 10 mM MgCl₂, and 0.2 mM acetosyringone) to a final OD₆₀₀=0.5. Cells are incubated in this medium for 3-h without shaking. A minimum of three leaves per Agrobacterium type, including the wild type control, are infiltrated by pressing the tip of a 3 mL sterile syringe with the Agrobacterium mix into the abaxial side of the leaves (FIG. 2). The agroinfiltrated leaves are harvested after two days of incubation, frozen in liquid nitrogen and subsequently kept at −80° C. until ready to use.

### Example 5: Alkaloid Extractions

For all the exemplified data herein, total leaf alkaloid extractions are made using fresh or frozen leaf material. Fresh leaf pieces of 1 cm×1 cm or 50 mg of freeze-dried leaf material is macerated in 1 mL of 100% methanol and incubated in the presence of this solvent for 2 h. During this time, the samples are subjected to an ultrasonic ice-water bath. Following a brief centrifugation to pellet debris, the supernatant is collected and acidified with 500 μL of 2% (v:v) H₂SO₄, and the hydrophobic neutral compounds are removed with CHCl₃ (2×500 μL). The remaining polar fraction is subsequently basified upon addition of 200 μL NH₄OH (25%), and alkaloids extracted with CHCl₃ (3×500 μL). The organic solvent (CHCl₃) is evaporated, and the samples are dissolved in pure methanol prior to gas chromatography (GC), or in phosphate buffer solution (71.6 g L⁻¹ of Na₂HPO₃, pH 4.7) for colorimetric analysis.

### Example 6: Total Alkaloid Quantifications

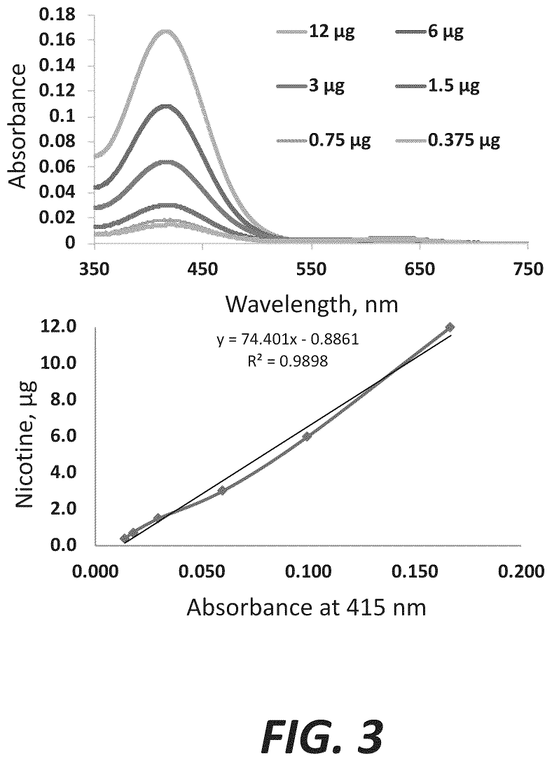

For all the exemplified data herein, a spectrophotometric method, developed by Patel et al. (2015), Int J Pharm Pharm Sci 7:249-251, is applied with some modifications. Briefly, alkaloids extracted from each single fresh leaf disc, or 50 mg of freeze-dried leaf material, are resuspended in 200 μL phosphate buffer solution (pH 4.7), mixed with 200 μL bromocresol green solution (BCG) (1 mM stock), then 400 μL of chloroform are added to extract the alkaloids. Finally, the absorbance spectrum of the solution is measured in the 350 to 550 nm region with a UV-VIS Shimadzu UV-1800 spectrophotometer. (FIG. 3, upper). A calibration curve is constructed of the absorbance maximum at 415 nm, as a function of the concentration of nicotine in the solution. Nicotine is selected as the molecule of choice for this calibration curve, as it is the most abundant alkaloid in tobacco leaves. The calibration curve served as a standard, comprising 0.375, 0.75, 1.50, 3, 6 and 12 μg nicotine in 400 μL volume (FIG. 3, lower).

### Example 7: Direct Nicotine Detection and Quantification

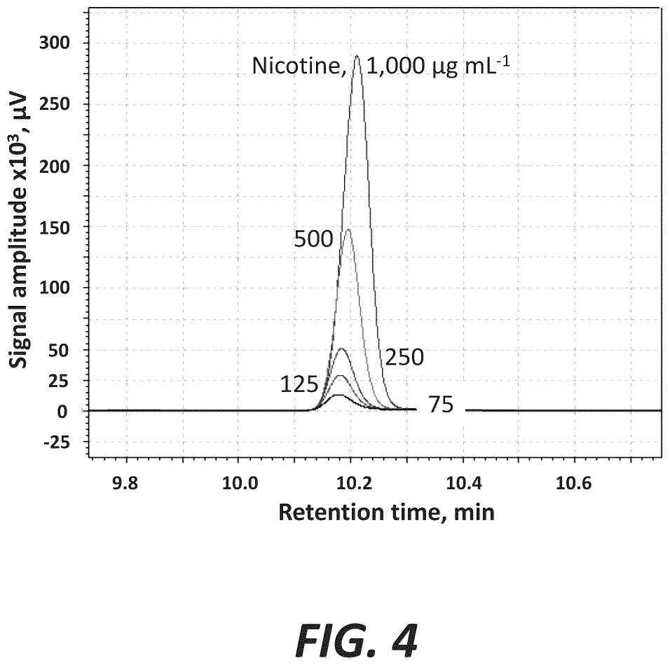

For all the exemplified data herein, to quantify the specific level of nicotine in the leaves, alkaloids extracted from 50 mg of freeze-dried material are subjected to a GC-FID analysis with a Shimadzu GC-2014 Gas Chromatography apparatus equipped with a flame-ionization detector (FID). A fused silica capillary column of 30 m, 0.32 mm ID and 0.25 μm of film thickness (Rtx-5 Resteck) is used with N₂ as the carrier gas at a flow rate of 1 mL min⁻¹. The temperature profile is 60° C., followed by a temperature rate increase of 12° C. min⁻¹ up to 290° C. The injector and detector temperatures are set to 290° C., the injection volume is 8 μL and the split is set at 20:1 (FIG. 4). This analysis shows a linear response of the apparatus to nicotine concentration and a detection limit of 12.5 μg nicotine mL⁻¹.

### Example 8: Genomic DNA PCR and RT-qPCR

To identify and verify positive RNAi transformants, presence of the kanamycin selectable marker is tested by genomic DNA PCR using the Phire Plant Direct PCR Master Mix (Thermo SCIENTIFIC). Experimental conditions for the PCR reaction were: 95° C. for 5 min, then 35 cycles comprising 95° C. for 60 s; 60° C. for 30 s; 72° C. for 60 s, and 72° C. for 5 min. Presence of the target genes in tobacco leaves after agroinfiltration is verified by resolving the genomic DNA PCR products in an agarose gel (1%). The elongation factor 1a (EF-1a) is used for expression normalization, as a nuclear-encoded reference gene. For RT-qPCR, 2 μL of cDNA is used and each sample is run in triplicate, under the following conditions: 95° C. for 2 min, then 35 cycles comprising 95° C. for 10 s; 60° C. for 20 s; 72° C. for 20 s, and 72° C. for 2 min.

Total RNA from the plant material is isolated using the TRI Reagent® (Sigma-Aldrich). cDNA is prepared from 1 μg total RNA treated with DNase I, RNase-free (Thermo SCIENTIFIC) and synthesized with M-MuL V Reverse Transcriptase (New England BioLabs® Inc.). Expression levels in the seedlings are tested with the CFX96 Touch Real-Time PCR Detection System (Bio-Rad). The reaction mix is prepared with Luna® Universal qPCR Master Mix (NEB) and each sample is run in triplicate, under the following conditions: 95° C. for 2 min, then 40 cycles comprising 95° C. for 10 s; 60° C. for 20 s; 72° C. for 20 s, followed by a melting curve. For these experiments, primers specific for the various genes of interest are designed with the assistance of Primer-BLAST (Table 10).

TABLE 10
Primers specific for the various genes of interest and other
primers. Primer ID are coded. For example, ODC_qPCR_FW represents
a forward primer used in qPCR for an ODC gene. Primers are
designed such that all homolgous genes are recognized by a
single pair of primers.

| Primer ID | Sequence | SEQ ID NO. |
|---|---|---|
| ODC_qPCR_FW | ACGTTTCCGACGACTGTGTT | 65 |
| ODC_qPCR_RV | GCAGCTCCGGTAACTGGTAA | 66 |
| ADC_qPCR_FW | GTGTCACAGAGCGATAGCCC | 67 |
| ADC_qPCR_RV | TGCTTGAGCGTCTCGAACAT | 68 |
| AO_qPCR_FW | TGTTGTGCCAACCTGGTAGT | 69 |
| AO_qPCR_RV | GTTGGCAACCTCTCGCTTTC | 70 |
| SAM_qPCR_FW | TGACTTCAGGCCTGGAATGAT | 71 |
| SAM_qPCR_RV | CCTTGACAGTCTCCCAGGTG | 72 |
| AIC_qPCR_FW | GGTACAAGGCTTGCTGCTTC | 73 |
| AIC_qPCR_RV | TCTGGAAACGCCAGTGAGAG | 74 |
| Arg_qPCR_FW | GCGGTCTCTCTTTCCGTGAT | 75 |
| Arg_qPCR_RV | GCAGTCATGCCATCAACAGT | 76 |
| ElongationF_FW | TGGTCAGGAGATTGCGAAAGAGC | 77 |
| ElongationF_RV | ACGCAAAACGCTCCAATGGTG | 78 |
| KanR_FW | CAAGATGGATTGCACGCAGG | 79 |
| KanR_RV | TGATATTCGGCAAGCAGGCA | 80 |
| CSVMV-F | CCAGAAGGTAATTATCCAAG | 81 |
| Intron-R | ACAAAGCCAAGAAAGGGTACT | 82 |
| Intron-F | AGATCTTCAACACCTACACCATT | 83 |
| NosT-R | AGTTGCTCGAGGAATTCCCG | 84 |

### Example 9: Polyamines Extraction and Quantification

For all the exemplified data herein, to extract free polyamines, 250 mg of fresh leaves are homogenized and incubated with 1 mL 5% perchloric acid on ice for 30 min. The mix is centrifuged at 12,000 g for 10 min at 4° C., and 0.8 mL of the supernatant are transferred to a new 2 mL tube. This supernatant is mixed with 0.8 mL of oversaturated sodium carbonate solution, 40 μL of 0.5 mM 1,7-Heptanediamine (HDT), and 10 μL of isobutylchloroformate. Following incubation for 30 min at 35° C., 200 μL of toluene are added, mixed well, and centrifuged at 10,000 g for 1 min. Aliquots of 100 μL of the organic layer are used for analysis by GC-FID.

The GC-FID analysis is performed with a Shimadzu GC-2014 Gas chromatography apparatus equipped with a flame-ionization detector (FID), as above. The temperature profile in this case is 110° C., followed by a temperature rate increase of 30° C. min⁻¹ up to 320° C. held for 13 min. The injector and detector temperatures are set to 250° C., the injection volume is 8 μL and the split is set at 15:1. Calibration curves are conducted using Putrescine (PUT), Cadaverine (CAD), HDT-like internal standard, Spermidine (Spd), and Spermine (Spm). Calibration curves are measured with the above standards at concentrations of 10, 5, 2.5, 1.25 and 0.625 mM for each of these compounds.

### Example 10: Evaluation of Transient Expression

In order to evaluate the effect of the RNAi constructs on the respective gene expression, leaves from tobacco plants in the same developmental stage are agroinfiltrated for transient expression measurements. Nine different Agrobacterium tumefaceins strains (At-ADC, At-AIC, At-AO1, At-AO2, At-ARG, At-ODC, At-SAMS 1, At-SAMS 2, and At-SAMS3) containing the respective RNAi constructs (Table 8) and the control strain are used to inoculate the tobacco plants. Following a 2-day incubation with the plasmids, the inoculated leaves are harvested, frozen in liquid nitrogen, and stored at −80° C. until ready to use. Total RNA is extracted from each leaf sample and RT-qPCR analysis is performed. Levels of gene expression at the mRNA level are reported as percentage of each gene transcript in the presence of the RNAi construct, compared to those of the control. The following levels are noted (±standard error of the mean): ADC=88% (±10%), AIC=33% (±28%), AO1=63% (±14%), AO2=67% (±30%), ARG=84% (±15%), ODC=62% (±15%), SAMS1=62% (±10%), SAMS2=70% (±16%), SAMS3=67% (±17%). These results provide an initial assessment on the effect of the RNAi constructs on the corresponding gene expression.

### Example 11: Screening of the Genomic Transformants

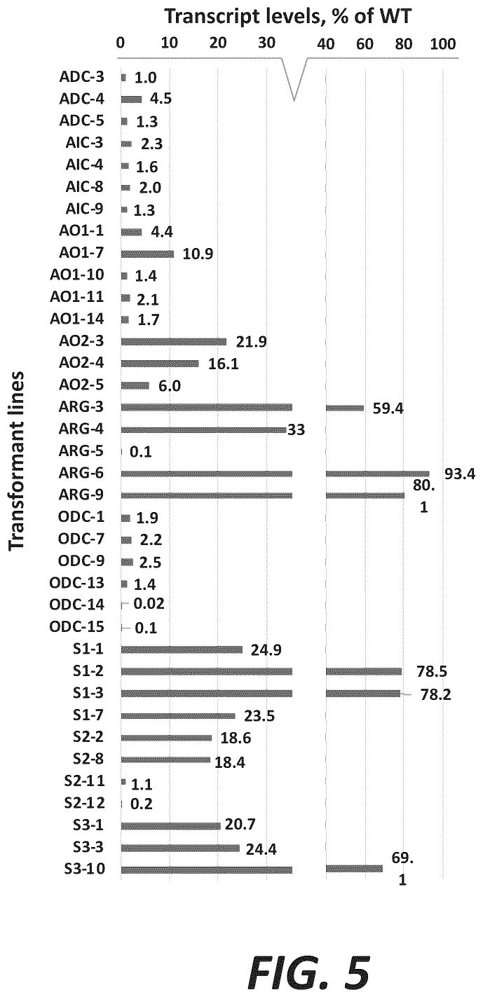

About 120 TO RNAi and control plants are cultivated, first in-vitro in the lab, until they reached a height of about 10 cm. These are selected for antibiotic resistance and further tested by PCR analysis for the presence of the RNAi transgenes. They are transferred to the greenhouse for growth in soil, and for T1 seed generation by self-fertilization. T1 seeds are harvested, sterilized, and germinated first in-vitro in the lab in the presence of kanamycin. Leaf samples from the emanating T1 seedlings are tested to evaluate the level of target gene expression by RT-qPCR (FIG. 5). Transcript levels are plotted as a fraction (%) of the corresponding one in the wild type, thus providing a measure of gene expression in T1 RNAi transformant tobacco plants, and also a measure of the efficacy of the RNAi approach to gene expression downregulation. The results show a substantially lower transcript level, reaching as low as 1% of those in the wild type, for all independent event lines derived from the ADC, AIC, AO, and ODC RNAi transformations (FIG. 5). The ARG and SAMS transformant lines show mixed and/or inconsistent results, with some lines having transcript levels comparable to the wild type, while others show substantially lower levels. A minimum of 3 plants from each transformation event with lower levels of expression of the target genes are selected for further growth in the greenhouse and subsequent analysis. In total, 36 lines comprising the 9 RNAi constructs plus the wild type are evaluated. The specific number of transformants by each RNAi construct is shown in Table 11 and FIG. 5 (RNAi transformant lines). Only three lines from independent events are examined for the ADC, AO2 and SAMS1, whereas six lines from independent events are examined for the ODC RNAi transformants. Intermediate number of lines from independent events are examined in the case of the other RNAi transformants (Table 11).

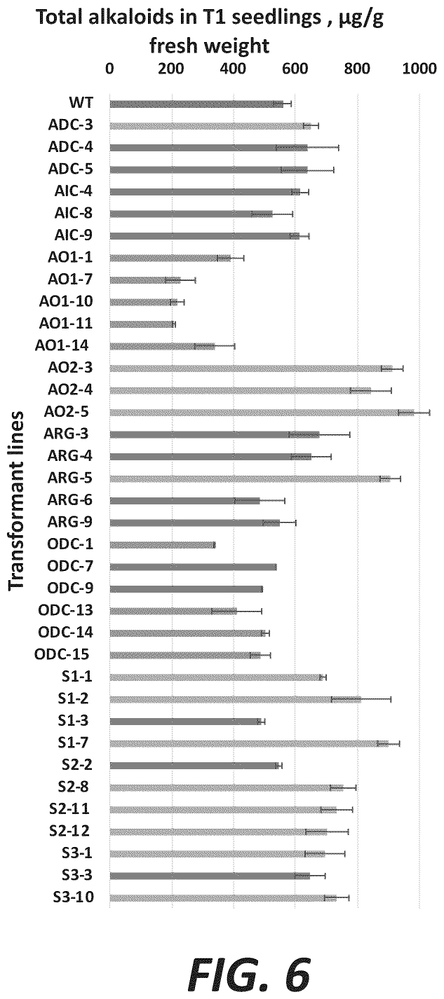

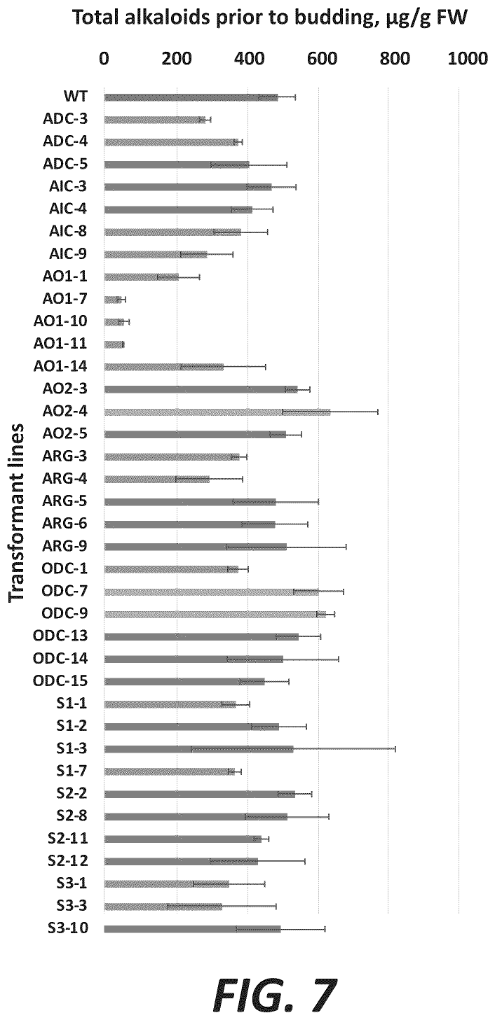

A preliminary measurement of total alkaloid content is performed in each of the T1 Dixie cup seedlings referenced in Table 11. FIG. 6 shows the values for these 36 lines selected to be transferred to the greenhouse. In this early vegetative stage, only the AO1 RNAi and some of the ODC RNAi lines show a significantly lower total alkaloids content compared to the wild type control (WT). In contrast, all AO2 and SAMS RNAi lines show significantly greater alkaloid content values than the wild type (WT). A subsequent measurement is undertaken at a later developmental stage with mature plants grown in the greenhouse following the early budding stage (FIG. 7). At this plant development stage, the content of total alkaloids in most of the transgenic lines is lower compared to the control (WT), except for some of the AO2 and ADC lines, which continued to show significantly greater alkaloid content values.

TABLE 11
RNAi constructs and RNAi transformant lines with
the lowest level of transcripts detected in fully
expanded leaves from mature tobacco plants.

| RNAi | Lines with lowest gene expression |
|---|---|
| construct | and low content of total alkaloids |
| ADC | 3, 4, 5 |
| AIC | 3, 4, 8, 9 |
| AO1 | 1, 7, 10, 11, 14 |
| AO2 | 3, 4, 5 |
| ARG | 3, 4, 5, 6, 9 |
| ODC | 1, 7, 9, 13, 14, 15 |
| SAMS 1 | 1, 2, 3, 7 |
| SAMS 2 | 2, 8, 11, 12 |
| SAMS3 | 1, 3, 10 |

### Example 12: Leaf Nicotine Analysis

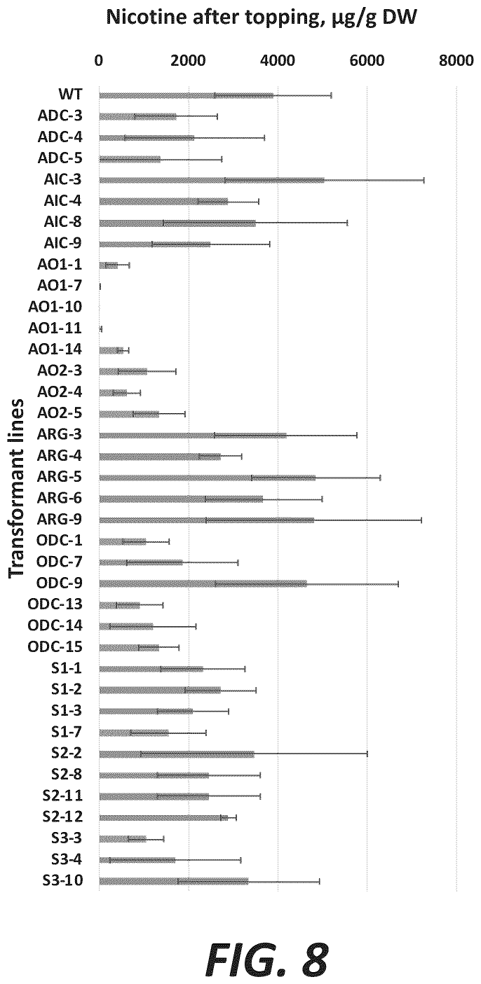

For the quantification of nicotine, the flower head and the shoot down to the first completely expanded leaf from the top are removed. Two weeks after this topping off, the 3^{rd} and 4^{th} leaves from the top are harvested, midrib removed, and the leaves are frozen in liquid nitrogen and lyophilized. The samples are ground and used for total alkaloid extraction. The average nicotine content of the leaves in three biological replicates from each T1 line is shown in FIG. 8. All transformants expressing the AO1-RNAi and AO2-RNAi have the lowest values of nicotine compared to the control (WT). The latter had 3.9±1.3 mg NCT per g DW. The nicotine content range of the AO1-RNAi lines vary between 0 and 0.26 mg NCT per g DW. The nicotine content range of the AO2-RNAi lines vary between 0.30 and 0.64 mg NCT per g DW. The next most significant suppression of nicotine content is observed in the ODC-RNAi lines 1, 13, 14 and 15. Also, ADC-RNAi has at least two lines with lower than wild type nicotine content. In contrast, lines AIC-RNAi, ARG-RNAi and SAMS2-RNAi do not display significant differences compared with the wild type control.

### Example 13: Polyamines Profile

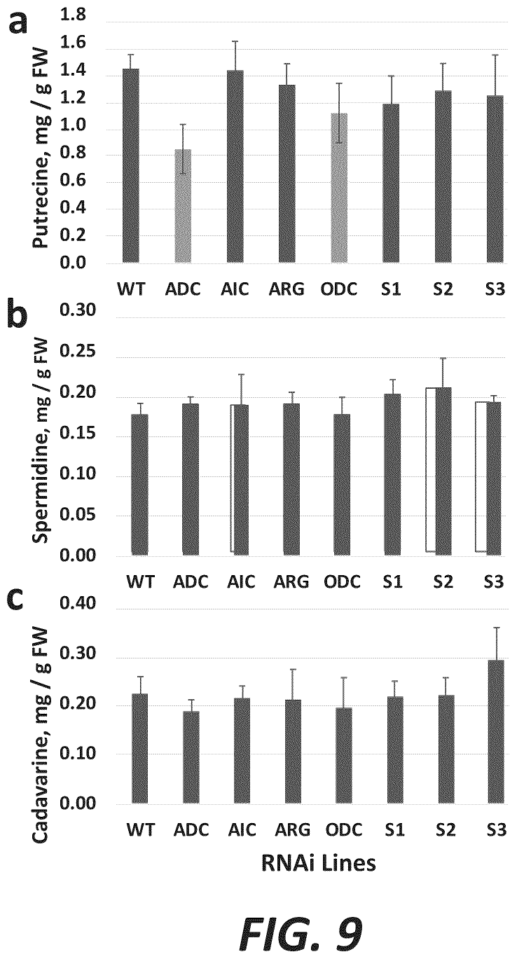

The samples for polyamines determination are taken from the topped-off plants, as is the case of the samples for nicotine analysis. FIG. 9a shows the mean putrescine content in the various RNAi lines. Compared to wild type ADC-RNAi and ODC-RNAi lines have significant lower content of PUT, while AIC-RNAi, ARG-RNAi and SAMS-RNAi lines have levels of putrescine equivalent to that measured in the control (FIG. 9A, WT). Putrescine levels seem to be lower in the ADC-RNAi than the ODC-RNAi lines, however, the uncertainty (error bars) in the two measurements would prevent drawing such an unequivocal conclusion. The spermidine (FIG. 9b) and cadaverine (FIG. 9C) content of the RNAi transformants is statistically invariable from that of the control.

### Example 14: Leaf Phenotype of the RNAi Transformants

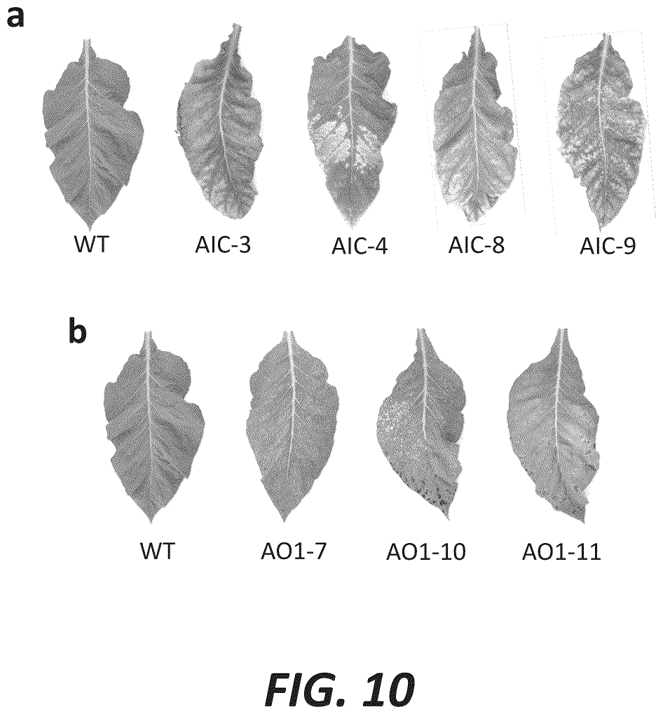

Differences in the phenotype of the leaves are noted between the various transformants and the wild type control. In all AIC-RNAi lines (FIG. 10a), the fully expanded and oldest (lower) leaves show a premature turning to yellow then reddish-brown coloration. They initially develop concentric ring spots, which expand and merge to form larger leaf areas of tissue reminiscent of senescence. In general, the photosynthetic viability of the mature and oldest (lower) leaves is negatively affected in the plants expressing the AIC-RNAi construct. This phenomenology is limited to the lower leaves and does not seem to affect the upper leaves of these plants, including those that are sampled for alkaloids, nicotine, and polyamines analysis.

In only some of the AO1-RNAi lines (FIG. 10b), the fully expanded and oldest (lower) leaves also turn yellow, develop white spots, which later become reddish brown. The lines that develop this phenotype (AO1 lines 7, 10, and 11) are the ones with the lowest level of nicotine content in the upper leaves. Interestingly, none of the AO-2-RNAi plants develop the early senescence phenotype.

It should be noted that these coloration changes described above affect the oldest leaves of these transformants, whereas higher up leaves, including the 3^{rd} and 4^{th} leaves from the top that are harvested for alkaloids and nicotine analyses, have a normally green pigmentation and otherwise healthy phenotype, very much like the wild type control.

### Example 15: Plant Data Conclusion

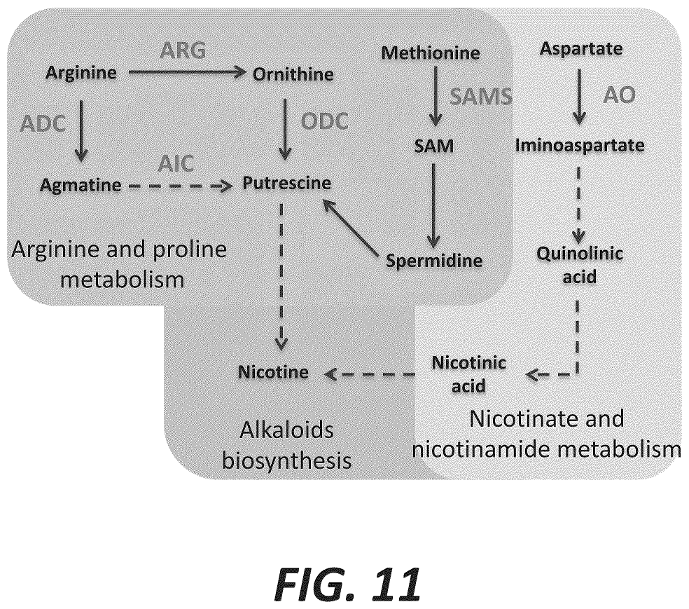

The exemplified results above show that nicotine content in tobacco leaves is attenuated the most in plants expressing the arginine decarboxylase (ADC), ornithine decarboxylase (ODC) and aspartate oxidase (AO) in an RNAi configuration. The ODC results support that an ornithine to putrescine reaction (FIG. 11), catalyzed by the enzyme ornithine decarboxylase (ODC), is an important step in alkaloid and nicotine biosynthesis and accumulation. The results also show that the nicotinate and nicotinamide metabolic pathway is also important in the synthesis and accumulation of nicotine, in a putative process schematically shown in FIG. 11. Conversely, the results strengthen the notion that steps of the arginine and proline metabolism and the associated enzymes, including the agmatine deiminase (AIC), and arginase (ARG) (FIG. 11) do not play a significant role in determining leaf nicotine levels. An intermediate nicotine attenuation effect is noted upon the RNAi-mediated down-regulation of the S′adenosyl-L:-methionine (SAM) synthase enzymes, suggesting that methionine to spermidine to putrescine may also contribute to nicotine synthesis and accumulation. In sum, results presented here suggest that multiple different biosynthetic pathways catalyzed by a variety of different enzymes may feed substrate toward nicotine biosynthesis in commercial cultivars of tobacco.

### Example 16: Random Mutagenesis

Random mutagenesis of tobacco plants are performed using Ethyl methanesulfonate (EMS) mutagenesis or fast neutron bombardment. EMS mutagenesis consists of chemically inducing random point mutations over the length of the genome. Fast neutron mutagenesis consists of exposing seeds to neutron bombardment which causes large deletions through double stranded DNA breakage.

For EMS mutagenesis, one gram (approximately 10,000 seeds) of Tennessee 90 tobacco (TN90) seeds are washed in 0.1% Tween for fifteen minutes and then soaked in 30 ml of ddH2O for two hours. One hundred fifty (150) μl of 0.5% EMS (Sigma, Catalogue No. M-0880) is then mixed into the seed/ddH2O solution and incubated for 8-12 hours (rotating at 30 rpm) under a hood at room temperature (RT; approximately 20° C.). The liquid then is removed from the seeds and mixed into 1 M NaOH overnight for decontamination and disposal. The seeds are then washed twice with 100 ml ddH2O for 2-4 hours. The washed seeds were then suspended in 0.1% agar solution.

The EMS-treated seeds in the agar solution are evenly spread onto water-soaked Carolina's Choice Tobacco Mix (Carolina Soil Company, Kinston, NC) in flats at ˜2000 seeds/flat. The flats are then covered with plastic wrap and placed in a growth chamber. Once the seedlings emerge from the soil, the plastic wrap is punctured to allow humidity to decline gradually. The plastic wrap is completely removed after two weeks. Flats are moved to a greenhouse and fertilized with NPK fertilizer. The seedlings re plugged into a float tray and grown until transplanting size. The plants are subsequently transplanted into a field. During growth, the plants self-pollinate to form M1 seeds. At the mature stage, five capsules are harvested from each plant and individual designations are given to the set of seeds from each plant. This forms the M1 population. A composite of M1 seed from each M0 plant are grown, and leaves from M1 plants are collected for DNA extraction. Target genes are amplified and sequenced for mutation identification. Mutations are identified in each of the genes listed in Tables 8 and 9, with a focus on all ADC, AO, and ODC genes. Higher-order mutation combinations are also produced to achieve, e.g., double mutants in both ADC genes, both ODC genes, or triple mutants in all three AO genes.

### Example 17: Targeted Mutagenesis

Tobacco lines with low nicotine are produced by introducing mutations into and around each of the target genes listed in Tables 8 and 9 via precise genome engineering technologies, for example, Transcription activator-like effector nucleases (TALENs), meganuclease, zinc finger nuclease, and CRISPR(Cas9 system, Cpf1 system, or Csm1 system). Genome modifications are made in commercial tobacco varieties such as TN90, K326 and Narrow Leaf Madole. All genes listed in Tables 8 and 9 are edited, with a focus on ADC, AO, and ODC genes.

For example, CRISPR guide RNAs are designed and synthesized to recognize specific target sequences. Guide RNA(s) and an accompanying nucleic acid encoding a Cas9, Cpf1, or Csm1 protein (either as a DNA plasmid form or as a mRNA form) are then used to transform tobacco protoplasts. CRISPR-Cas9/Cpf1/Csm1 ribonucleoprotein complexes recognize specific NCG target sequences and introduce a double strand break (DSB). The endogenous non-homologous end-joining (NHEJ) DNA repair system fix the DSB, which may introduce nucleotide deletion, insertion, or substitution and result in potential loss-of-function mutations. Alternatively, a donor nucleic acid molecule with a desired sequence is included in protoplast transformation to serve as a template molecule to introduce the desired sequence at or around the CRISPR target site.

Tobacco protoplasts are isolated from TN90 tobacco leaves growing in Magenta boxes in a growth chamber. Well-expanded leaves (5 cm) from 3-4-week-old plants are cut into 0.5 to 1-mm leaf strips from the middle part of a leaf. Leaf strips are transferred into the prepared enzyme solution (1% cellulase R10, 0.25% macerozyme R10, 0.4 M mannitol, 20 mM KCl, 20 mM MES (pH 5.7), 10 mM CaCl2, 0.1% BSA) by dipping both sides of the strips. Leaf strips are vacuum infiltrated for 30 min in the dark using a desiccator with continuing digestion in the dark for 4 hour to overnight at room temperature without shaking. Protoplasts are filtered in 100 μm nylon filter and purified with 3 ml Lymphoprep. Protoplasts are centrifuged and washed with W5n solution (154 mM NaCl, 125 mM CaCl₂), 5 mM KCl, 2 mM MES, 991 mg/l glucose pH 5.7) and suspended in W5n solution at the concentration of 5×105/ml. Protoplasts are kept on ice for 30 min to settle at the bottom of the tube by gravity. W5n solution was moved and protoplasts were re-suspended in P2 solution at room temperature. 50 μl DNA (10-20 μg of plasmid), 500 μl protoplasts (2×105 protoplasts) and 550 μl of PEG solution (40%, v/v 10 ml 4 g PEG4000, 0.2 M mannitol, 0.1 M CaCl2) are mixed gently in a 15-ml microfuge tube, and the mixture incubated at room temperature for 5 min.

Protoplasts are pelleted and re-suspended with 1 ml 2×8EN1 (8EN1: MS salt without NH₄NO₃, MS vitamin, 0.2% myo-Inositol, 4 mM MES, 1 mg/l NAA, 1 mg/l IAA, 0.5 M mannitol, 0.5 mg/l BAP, 1.5% sucrose). Transformed protoplasts are jellified with equal amount of low-meting agarose (LMA), and 0.2 ml of protoplast-LAM is dropped to form a bead. 10 ml 8EN1 is added to the bead, and in 7 days, 5 ml 8EN1 is taken out and 5 ml 8EN2 (8EN1 with 0.25 M mannitol) is added; after another 7 days (14 day), 10 ml 8EN2 is taken out and 10 ml 8EN2 is added; in another 7 days (21 day), 5 ml 8EN2 is taken out and 5 ml 8EN3 (8EN1 with 3% sucrose and without mannitol) is added; after another 7 days (28 day), 10 ml 8EN3 is taken out and 10 ml 8EN3 is added. Protoplasts are kept for two weeks until micro-callus growth. Callus is transferred to NCM solid media until it reaches about 5 mm (usually about two weeks). Callus was transferred to TOM-Kan solid media to grow shoots, and transformed tobacco plants were regenerated using the methods described herein. Callus or regenerated plants are tested and selected for gene editing events with desired mutations in target genes. Both loss-of-function alleles (e.g., early stop codon or frame shift) or other types of mutations (e.g., gain-of-function or neomorphic) are generated.

### Example 18: Related References

Baldwin I T (1989) Mechanism of damage-induced alkaloid production in wild tobacco. J Chem Ecol 15:1661-1680
Bai Y, Pattanaik S, Patra B, Werkman J R, Xie C H, Yuan L (2011) Flavonoid-related basic helix-loop-helix regulators, NtAn1a and NtAn1b, of tobacco have originated from two ancestors and are functionally active. Planta 234(2):363-75
Chintapakorn Y, Hamill J D (2007) Antisense-mediated reduction in ADC activity causes minor alterations in the alkaloid profile of cultured hairy roots and regenerated transgenic plants of Nicotiana tabacum. Phytochemistry 68:2465-2479
DeBoer K D, Dalton H L, Edward F J, Hamill, J D (2011) RNAi-mediated downregulation of ornithine decarboxylase (ODC) leads to reduced nicotine and increased anatabine levels in transgenic Nicotiana tabacum L. Phytochemistry 72:344-355
DeBoer K D, Dalton H L, Edward F J, Ryan S M, Hamill, J D (2013) RNAi-mediated down-regulation of ornithine decarboxylase (ODC) impedes wound-stress stimulation of anabasine synthesis in Nicotiana glauca. Phytochemistry 86:21-28
Dewey R E, Xie J (2013) Molecular genetics of alkaloid biosynthesis in Nicotiana tabacum. Phytochemistry 94: 10-27
Gray J C, Kung S D, Wildman S G, Sheen S J (1974) Origin of Nicotiana tabacum L. detected by polypeptide composition of fraction I protein. Nature 252, 226
Henry J B, Vann M C, Lewis R S (2019) Agronomic practices affecting nicotine concentration in flue-cured tobacco: a review. Agronomy Journal 111:1-9
Kajikawa M, Sierro N, Kawaguchi H, Bakaher N, Ivanov N V, Hashimoto T, Shoji T (2017) Genomic insights into the evolution of the nicotine biosynthesis pathway in tobacco. Plant Physiol 174(2):999-1011
Kirst H, Shen Y X, Vamvaka E, Betterle N, Xu D M, Warek U, Strickland J A, Melis A (2018) Downregulation of the CpSRP43 gene expression confers a truncated light-harvesting antenna (TLA) and enhances biomass and leaf-to-stem ratio in Nicotiana tabacum canopies. Planta 248:139-154
Kung S D, Sakano K, Gray J C, Wildman S G (1975) The evolution of fraction I protein during the origin of a new species of Nicotiana. J Mol Evol. 7(1):59-64
Leete E (1977) Biosynthesis and metabolism of the tobacco alkaloids. Proc. Am. Chem. Soc. Symp. 173:365-388
Moghbel N, Ryu B, Ratsch, A, Steadman K J (2017) Nicotine alkaloid levels, and nicotine to nornicotine conversion, in Australian Nicotiana species used as chewing tobacco. Heliyon 3(11), e00469
Murashige T, Skoog F (1962) A Revised medium for rapid growth and bio assays with tobacco tissue cultures. Physiol Plant 15:473-497
Patra B, Schluttenhofer C, Wu Y, Pattanaik S, Yuan L (2013) Transcriptional regulation of secondary metabolite biosynthesis in plants. Biochim Biophys Acta 1829(11): 1236-1247
Patel R K, Patel J B, Trivedi P D (2015) Spectrophotometric method for the estimation of total alkaloids in the Tinospora cordifolia M. and its herbal formulations. Int J Pharm Pharm Sci 7:249-251
Payyavula R S, Navarre D A, Kuhl J C, Pantoja A, Pillai S S (2012) Differential effects of environment on potato phenylpropanoid and carotenoid expression. BMC Plant Biol. 12:39. doi: 10.1186/1471-2229-12-39.
Payyavula R S, Navarre D A, Kuhl J, Pantoja A (2013) Developmental effects on phenolic, flavonol, anthocyanin, and carotenoid metabolites and gene expression in potatoes. J Agric Food Chem. 61(30):7357-7365
Saitoh F, Nona M, Kawashima N (1985) The alkaloid contents of sixty Nicotiana species. Phytochemistry 24:477-480f
Singh S K, Wu, Y Ghosh J S, Pattanaik S, Fisher C, Wang Y, Lawson D, Yuan L (2015) RNA-sequencing reveals global transcriptomic changes in Nicotiana tabacum responding to topping and treatment of axillary-shoot control chemicals. Scientific Reports 5:18148
Sisson V A, Severson R F (1990) Alkaloid composition of the Nicotiana species. Beitr. Tabakforsch 14:327-339
Tayoub G, Sulaiman H, Alorfi M (2015) Determination of nicotine levels in the leaves of some Nicotiana tabacum varieties cultivated in Syria Herba Pol 61(4): 23-30
Tso T C, Jeffrey R N (1961) Biochemical studies on tobacco alkaloids. IV. The dynamic state of nicotine supplied to N. rustica. Arch Biochem Biophys 92:253-256
Waller G R, Nowacki E K (1978) Alkaloid biology and metabolism in plants. Plenum Press, New York

1.-150. (canceled)

151. A modified tobacco plant, or part thereof, comprising a non-natural mutation in an endogenous polynucleotide having a nucleic acid sequence encoding a polypeptide having 100% identity or similarity to an amino acid sequence selected from the group consisting of SEQ ID NOs: 37 to 54, wherein the non-natural mutation reduces or eliminates expression or activity of the polypeptide as compared to a control tobacco plant lacking the non-natural mutation.

152. The modified tobacco plant, or part thereof, of claim 151, wherein the endogenous polynucleotide comprises a sequence selected from the group consisting of SEQ ID NOs: 1 to 36.

153. The modified tobacco plant, or part thereof, of claim 151, wherein the tobacco plant is a Nicotiana tabacum plant.

154. The modified tobacco plant, or part thereof, of claim 151, wherein the modified tobacco plant comprises a reduced level of nicotine relative to a control tobacco plant not having the non-natural mutation.

155. The modified tobacco plant, or part thereof, of claim 154, wherein the modified tobacco plant is a low-alkaloid tobacco plant.

156. The modified tobacco plant, or part thereof, of claim 155, wherein the modified tobacco plant produces a leaf, when cured, having a comparable or higher USDA grade index value than that of a comparable leaf of the control tobacco plant when grown and cured in similar conditions.

157. The modified tobacco plant, or part thereof, of claim 156, wherein the higher USDA grade index value is at least 5% higher than that of the comparable leaf of the control tobacco plant

158. The modified tobacco plant, or part thereof, of claim 151, wherein the modified tobacco plant produces a leaf comprising a level of polyamines within 20% of the level of polyamines produced in a control tobacco plant lacking the non-natural mutation when grown under comparable conditions.

159. The modified tobacco plant, or part thereof, of claim 151, wherein the modified tobacco plant produces a leaf comprising a level of chlorophyll within 20% of the level of chlorophyll produced in a control tobacco plant lacking the non-natural mutation when grown under comparable conditions.

160. The modified tobacco plant, or part thereof, of claim 151, wherein the modified tobacco plant is of a tobacco variety selected from the group consisting of a flue-cured variety, a bright variety, a Burley variety, a Virginia variety, a Maryland variety, a dark variety, an Oriental variety, and a Turkish variety.

161. The modified tobacco plant, or part thereof, of claim 151, wherein the modified tobacco plant is of a variety selected from the group consisting of the varieties listed in Tables 1-7.

162. Cured tobacco material comprising material from the tobacco plant or plant part of claim 151.

163. The cured tobacco material of claim 162, wherein the endogenous polynucleotide comprises a sequence selected from the group consisting of SEQ ID NOs: 1 to 36.

164. The cured tobacco material of claim 162, wherein the cured tobacco material comprises cured leaf material, cured stem material, or both cured leaf material and cured stem material.

165. The cured tobacco material of claim 162, wherein the cured tobacco material is selected from the group consisting of flue cured tobacco material, air cured tobacco material, fire cured tobacco material, and sun cured tobacco material.

166. A tobacco product comprising the cured tobacco material of claim 162.

167. The tobacco product of claim 166, wherein the tobacco product is selected from the group consisting of a cigarette, a cigarillo, a non-ventilated recess filter cigarette, a vented recess filter cigarette, a cigar, snuff, pipe tobacco, cigar tobacco, cigarette tobacco, chewing tobacco, leaf tobacco, shredded tobacco, and cut tobacco.

168. The tobacco product of claim 166, wherein said tobacco product is selected from the group consisting of reconstituted tobacco, loose leaf chewing tobacco, plug chewing tobacco, moist snuff, snus, and nasal snuff.

169. A tobacco blend comprising the cured tobacco material of claim 162.

170. Reconstituted tobacco comprising the cured tobacco material of claim 162.


SEQUENCE LISTING (84 sequences)

SEQ ID NO: 1 - DNA, 3061 bp, Artificial Sequence
  Description of Artificial Sequence Synthetic polynucleotide
ggatgatggtggtgataatcgctaagtatggtggtggtggtggtggcatggtgatagttgataatggtaggcggtggcgacaattaataatgaatatggatgatagcatcttaataaaattaagtctctgttatggatcttaatcatacagacctattcagacccattaagtgattgtgaagtaagaaaaaaaaatacttaatgattaagatcttaataattaagattcagactttaaaaataaacgcatttaatgtctaaaatttgaatgattaagattcaaatctccattaaagcaaacaaatgaggccttagaatctcacattaatcggtcctaagctaaacacaagaaattaaattaagcaacacaagagccacatatgttaatgtgaattgatattgataaatatcgtcatattaatattaccagaacacacttgtgataaaagacaaatcaattaccgattcaccgattttggggagagagatagagaatactttttaagttaatagagaagacaatctgacacgcacactaacgggaccaacatgctgcttccttatttacaatttctcacctctttcctcttaaagcaccaaaatgtctgcaatcgatcctttcctgtgtctgagctttatgtaaaaaaataatgaatggattagcaatccagtattgtaaaaccattgttaccactctaatctttgcctataattgcatcttaattatgtttgattctcaaaacttaagccatgattaggcaaagaattaatacgaatcgcttaaagattttccgcaaagcaaacttgtgtcacaatggtacaatgcatccatctctctctccatgaaacagagttttttcattaaacttgaaagatttttatttttaaataactttaaaatgtgtgccataaaattaaattttttgtatatttagttatgaatttattaataacgtattagctttatctaatatttaattattaaaaaatatatattttttgagattttaaatttgaataacttatttattttttatatgtaatattaaaaatattttttatttataacaaacaaacaatatttaaacttcaagtattattataggtgtataacaatcattcgatgtaacaacaaaataatatcaaaagagacgggcaataatatagaagtttgactgtagtttgtaatatgaaagtggttacttattaattaaaaagtgaataagataaaaacatctttctttgtcccaatatatgtgatgcatgtcaaatttcggaagtcaaataatattttttaaccatcattttatatattttttaaatattttaaattattaattattatgactcataatattttcaaatatataaattttattttaaaaaaactaaagaatctatgtcaaatttgcgataaaaattaagaaatttaagtctcgatatacaaaatgcatcacatgaattgaaacggaagaagtataaattagtttaaatattttctttgtgtatggagtaattttcttaacaataataaaattgctggagcaaagtgcaacttgagaagagagaacatgagcgtgtcggtggtttagtagagtagtaagtcccactatgtcatcagtgtcatatagcaaatcatcacttaccattttcgctctttagctttttaaatactctacgcaagtgcaagtgcaagtgcaaccctcttttatatatatatatatatatatatatatatatatatatatatatatatgcttccctttcatggggtttgatggctctaaggagacaaataacctttttaataacgcccctttcaaatcagcttctttaatttggttgcgtatagggaataagattcagatttaagacatgggtcatgttgtggatttaattagaacagattactgagtacgtaattaacccccaaaaaacaaagattaactggaggtgaggtccaaagatcaaagagtcatttcggagttatatatatatatatggaacagaaaggccaagaaaaggaaatgggcataccaaactccattggctataatccatcttcaagtttttcagatactagaagaaatgaaaaagcaggcagtggaaatgtcattctcagccctatccaaactctaaatcagcacaaccaacattccctacaaggacatagccaactaaattcacatcaattagaagaagaaagggatccacttgttgcagctaccaatagagcacaactacgacaacaatcgacaacctcaggaaatggcacgaccacgaccacgaccagctcatcatcagtggttagacgataccgagaatgtctcaagaatcatgttgcaagcatgggaggacatgtccttgacggatgtggcgaattcatgccaagcgaagaagaaggtgcgctaaagtgtgcagcttgtaattgtcatcgaaattttcatcggagagaaactttctccgaggctgagacgcagatagctcgcccacgccccccatattctacattcaatccaccaatacatagtagtagcaattaccaccacaattatagtaccgcgtcaccagtgatgatgacttttggaaggaatgctgaatcatcaagtgagcatctaaatttgttcaattctaatgccggaggaggacaaagttcgaagtcgaagaagagatttaggacaaagttcacagcggaacaaaaagaaagaatgcatgaatttgctgagaaggtgggatggagtatacagaagcaagacgaacaagaattgcaacacttctgtaaccaagtgggtgtcaagagacaagttttcagaatatggatgcacaacagcaaacaagcaaataagaagagacaaatgtaaggcgtcattactgcgcctaataaattaaaccttgtttcagctcaaattagatacttttattagggactaatttctttgtgcataaatcaaagagacaacaagctagaaacgatatattcatttttcgatacaatttctgaactctaattagctgcttacttcgataatttttccctgttttacccttatcattaactatt

SEQ ID NO: 2 - DNA, 4208 bp, Artificial Sequence
  Description of Artificial Sequence Synthetic polynucleotide
ccagagatagcaaggtagttgcatggcgtacgcaggtctgccttgctccttcctatccttagtattatatattttagtcttatcttgtattgagtagacaatattggactgtattttagtggctcatgactagtgacaccaggatcaggtagtgtttgatgtatttgcgcatttgttttccccttattttgacatattaaaaggaaagatttgtgatcatatttgattagaaaatgaatttacaaaagaccttatatttttagtgttgaatcagctggcctagtactgtgataggtaccattacgatcggggtggttggggtcgtgacaccctacttgtgggattttaatggattgttggtgttgttagtgttgtaactatttacccacgacaataactattttcctttattaagcatatgacaataattcctaaatatttgtactaccctgagatatccgaagactcatttttctttgcctttattaccatgttcttatgtttcttgtaaagtcacaatactaatataaaaaatatataggactcgtctacaaacctctagggataattgaactgtgtcatggtcaggacagggcctcgacctacccgtcaaacttgtctatataaaatgaactccaaggtatagatcttgtaactccgagaaaatggagcttacgaaccaagctaatgtttgactttgtctacaatgaaggtctatctagttgtctatcagaacccgcaggcatgaaatgcagcgtccccagcaaaaggtagtagatagttggaagttgaaatataccaacaagtcccataacataacacaaacctaattgaggaataacgcataacaacatctaaacaacgaatcacacctcaaatcaaatataaatgaactttgaacttcaacttccaaaactcacactgaaacatatcaaatcaactcggaatgaactcaactttcaacttttaccaattagcgttgataccttctagaaatatccaaatgcaaatccgggcatacgccggagtccaaaatcaccatccggacctaacggaaccatgttcttatgtggcaagaatgcagatgctacgcatcaaaatgcactctcaaggacaaccatatttagccgttggttgaaagactagaactagcatcatttggtgttgtgaacttgtcaacaggaaagatgatgagaaggccataaacaaggctttgacaatctcatccttagaattgaatgaattgttcctaaatcgtcatatatatcttttatttcttgcttgacgttaagttatttttgtcgtcgtgcattattcttctctatttttatttttggtattgatttaaaacacttaatgttttgctcacctcactacttaaggtttttcagacatgtacgtgtgtcaaatgcttctagtttttcttatgattcattggtttaagcctataaagtggaagaactagaagtgcggcttatgttagatccccttattataatgtaaaagaaagtgtatttgatagtttcaatcttaataataattttcaacatgcatggatcgaggtgttatttatctacttagtttctttctactttggttttcgtttactttgtctagatcaaattaagtttcgtcatagaagattaataatggattttgcatatactaccattcagcacaagaaagcataccaagaatttaatttctatcagaagtaaacaaccaacacctcaatattggctagtgaaggtggcgtgacagatttgccaggtatactatacagtcaaaattgcacaattacagttgggctgctatccaagaattttgaagagatgtccaggtggtcaacattggcaaagaaataggcgataccacttgcaacaaaagcaaaaggcaatatatataatatttgtaaaatatcactataaaggaagatgttgcctacatattaaaaaatcataaatttcatatacctttactactcttgaaattttcaaataatgtatcatcttctttctcccatagaagccattgtaggacttgtaacctttgcatttctactctacttcctatggacaaaaaaacaatcaaaaatcctaaacccactgcctccaaaaatcccaggtggatggccagtaatcggccatctcttttatttcaagaacaatggcgatgatgaccgccatttttctcaaaaactcggagacttagctgacaaatatggtcccgtcttcacattccggttagggtttcgtcgtttcttggcggtgagtagttatgaagctatgaaagaatgcttctctaccaatgatatccatttcgccgatcggccagctttactttacggagaatacctttgctataacaatgccatgcttgctgttgccaaatatggcccttactggaaaaaaaatcgaaagctagtcaatcaagaacttctctccgttagtcggctcgaaaaattcaaacatgttagattttctatagttcagaaaaatattaaagaactatatgatggtgattcaccaatggtgaagataaaccttagtgattggatagataaattgactttcgacatcattttgaaaatggttgttgggaagacctataataatggacatggagaaatactgaaagcagcttttcagaagttcatggttcaagctatggagattgagctctatgatgtttttcacattccatttttcaagtggttggatcttacagggaatattaaggctatgaaacaaactttcaaagacattgataatattatccaaggttggttagatgagcacattaagaagagagaaacaaaggatgttggaggtgaaaatgaacaagattttattgatgtggtgctttctaagaggagcaacgaacatcttggcgatggttactctcatgacaccaccatcaaagcaaccgtattcgtaagtaaattaatctctgcttgcattttaacatgttgtcggaggcaatttatcttatattttaggttactatattcgtatatatataatatcactttaatttttatagactgattttttaagtggcttagtggtgtatagattcttctacactttcagtatttcgaagtcaaactttaatttatggattcagttgataaactctatcaccgtaggtaatgaatttttatatttttggtatattttaacctattgtagcaatggatttcaattaaagaaaaaaagaggcttttgtacctttataacatatcttcaatttgactggagtattttcatttttttcttaaccaactcatgcatactaggatatgaacaatcatgcatcttcatctctttagtttcttctttgtaagtcatctttttaactgttctttgtccatatgtatagactttggtcttggatgcaacagacacacttgcacttcatataaagtgggtaatggcgttaatgataaacaataagaatgtcatgaagaaagcacaagaagagatggacaccattgttggtagagatagatgggtagaagagagtgatatcaagaatttggtgtatcttcaagcaattgttaaagaagtattacgattacatccacctgcacctttgtcagtacaacacctatctgtaaaagattgtgttgtcaatggataccatattcctaaggggactgcactacttacaaatattatgaaactgcaacgagatcctcaaatatgggcagatcctgataaattcgatccagagagattcttgacaactcatgctgcaattgactatcgagggcagcactatgagttgataccgtttggtacggggagacgagcttgtcccgcaatgaattactcattgcaagtggaacacctttcaattgctcatatgatccaaggtttcaattttgcaactacgacaaacgagcctttggatatgaaacaaggtgtgggtctaactttacctaagaagacagatgttgaagtgctaattacacctcgcctgcctcctacgctctatcaatattaatatgttttgttgtcgcgattcgttctgatttgtccctcaatgtttaaaaaaaatgttttattttggttaagattgtttcacagattgttattcatctattttactaataagttattgtaaaccataaataataagaagtttttgtctccgataagcacttctcgggcagaagtcttaagaatgagctcagacctctatgg

SEQ ID NO: 3 - DNA, 9146 bp, Artificial Sequence
  Description of Artificial Sequence Synthetic polynucleotide
gacgccttatggtggactgcaaacatttctttcgctgcccgctgttctccatatatggtcgtaatgccatctttcgtgggaaactttatcacctggtataaggttgacggtaataccctcatgttgtgtatccaaggcctgccgagtaatgcattgtacctcatgtcgccgtcgatgacttggaactcggtgttctaaatcacgccggacgtgttgatcgggagggtgatctctcctttccttacttcgccggtcatgttgaggccatggaggactcgagggatgggggcgactcgattgagtagccccagttgttctactacttctaatctaattatgttggccgagctgcctggatccatgagcacgtgctttattctagtattatttaaaagaaatgagattaccagcgcgtcgttgtgcggttctatcatggcctcgaggtcctcatcgatgaacgcgagggtgtcttcgggcacgcggtttcgggtcggtccctcttccgtggtggacattctcgtccgtttgaacatgggtccttggggaacataggttcctccaataatcatatggacgtcatgtcggagagcctccccttctgccccggtcgagtttggtggcgtaacgacatctggagtagccatgcccttctctttgctgttttcgaggttctgattccccattatattcaccgagctagacattttgtcttgaaattcgaagatcttggacaagaaaaagtgtgaaagataacctgcgttatgtagtgaaaccagcaagaaaataatcaatattatttttagccccacggtggacgccaaactatttaccgtgaaaatggtaataacaattaaatttgttgatgggactctaaaaatacatgatctatttttatgctagttgttagagcagttgatgctagatatgtgaagataaaagataaaacgcgagctggaaaagagttgtgatcaaaccgaggggttagctgtttgggcctcaggctgactggtgaaggacctcgaggtcgatgcctgggctcgggctcgagctatcgggggtaaccggggaaggactaacagttagaatataattaagggatgctttttacgccgatatcaagcaataaatgaagaacatgtacgaaggcaataaatgaaagtggtagtctcgagcgagtaagctagagagaaaagaaagagagagagttgttattgatcttgtgtagaatagttggagcaacctacttacaaagagccaatgatcccctttatataagaggggaaatccaaacacagtacaagatacattaaatgataaagaaagcgggatgggacaactaactagcttcatgatgtagacgtagacaagcgtttgactagcctggtagacttaaccggccctggatgcattcttcggaagcctcccatacttgtcggggtctcgaccaacattatctccagcccgagtgtcgatagattttgaggagtacccggcccaaggtctcaagcctccgaggcgatctcctgaagtaccttatcaacgagaaattggtccccccaatttcaccgtacacaaagttgtttctgaagttttgtgagtgatttgagggaaaattttgaaaaatagctttttggggttttttaaatttttgaaattttcaaatgttcaagtgaaaattggaatttttatgaacaaacgctcatttcggaaaaaagggaattttttttgggaaaaagggaattttttttatgtccaaacggctcttaattcaccaaagaggaaaagacaaaattccagggcacactttatttgacgaatcttttcttttggaaaaatactaaaaaaagaatcttaacagtgtcacaactcacgcacttaattttgttgtctttctgactcctatcttttttctgttaatttacacgattcaataaataaaagccaaaattggtgaaaatagcacatatttatagaggaagaagaagaggaggaggttagagatggatatgaaggggacgccagcggaaaatggataccgtatgcctgctgaatgggaatcccactctcattgttggatgggctggcctgttagtactctctatttttagctgtttatgttactattttgcttgccttaatctttagtaccatttttttaggggtaagtaagttagctatgtgtatgatgatggctaatcatggatatttgcctccgtttaggtggagaagttttacccgagcagtttccttttagctcaaattagtctattcatgtaggagaatttgatgatttttcaattgcaagttcatattcgacttgaaatctaatgaagttgcctttgagtgggacttcatccggattagttgggcaccaaagcgggtatcagggtgtgggaaacgaagaaaaaaagatttaatctagagtgtggatgcttcggcagatttagctctatgcttgggtttaacagaaaccactgctttcagcttagaccttctatccatatacaatataattaataataatactaataacaacgaaataaataaatgtataaaataatataaaacatgttcgtgtttaatatcatccactccaaacccattgattctaatcctggtttggcactatgtgagtgtgcgggtgtgggtgcggtggaggtttttcaaatcatctagaggataggttattaacaagtgagaatccttttatgcttttcaaatatagcagtgtaattcagcatcatgcctatatgtagctggcaaaaataagtactgtgccaattaggatcatcggacgataatgggatttggtgcaactcattatttcatgtgagtttgcatatcattcaagaacatgattcatttcttttcttgatcgttgtacacgtattttttaaagctgttctgttgcaagtgagaagatactttatgtggtttctagaagaaagtaggatcactgttcatgatcctctcatttctgattatctgctgaagttgtctttggttgcgtttagtctcagatgtctccttttgaaactttatattggagttccatcttagtagagtatggctggttgaatagtttaagcaatgtgagcaatctcacagtctgaactacagatttcacatttcttaatgacattctatgacttcatctgtatggttgacatcttgacatgactctttattgtttcttaaaaggcaacacatcttcttccctgttcgattagtacttttattttgtttccttttttggctcagttttccatggagtatgatattcaggtagtaatttcttctgaagacgcagagatttcggtttctgtcggctgatagcttatttttgagatgtaggagcgtccagataactggcgaaacaatgcactccacggtcaacgtgtatttgccaatgtagcatctgctatctcaagattcgaaccagtaacagtctgtgctagtgctgctcaggtttgcagtttttggaaggcaccaaaaaagttttctcagtacgtcatgatagtccctctttagttctgaaaagaatcatatctatgttgaacagtgggaaaatgcacgcagccagttaccaaaacatatcagggttgtggagatgagcatgaatgattcttggtttcgtgatactggtccaactgtaagcttttgtttcctttctgtggagtgcaacattgtatttacttttagctgtcacaccacacatggagttacaagaaaatcatatctctatccatgtagtttgtcatcagaaataccgcagcaaatgcagaacatgtagcgtcgagtgttgcaggaattgattggaccttcaatagctggggtggtaagagatttggatctcagtcttgatatctatatgaggaacatgctgttttacttttcttttttcacgtgaaagttatcaactggtatatcctgtctcaggagttttcatccgttagttacaaaatgcagcccaatttccattaattttaccgcaatatttttcttgggaggtttaacaaatattctcaataccccaatctgcaatttacgttggaaaattctgattgatcccactaaagacgtctttctttttttccccaaatgttaatcatcatatgatttagctaatggccaatctgacgtgatggtactttatgttgagtttttccttcccaaatgcttgaggctttcatagccttccagctccagcatatgacacgattattctacagcttttcatgtcatatggaatccagacaaatatagtttgatgatatgttgaagggctcaggatatttatcttctgcgttttgtagtctgctttaattgcctagaacttctagaaagagaaatatacctgtgtgtctttgttctataagcaaatagtgtttcgtatccattaaatataagtaattgacaggtgctttcatcaattttagaatgcttctagaagttgctgaacttttcagacatgtttgtgctgatgatcagtctgtagtgtgttgttttgttatttgaatctcatgtgtgctttggttctgtcttgccatagtaatattgtctcaaaatcatatccagtaatatagagttgctcttagattgatccaagattcttttggtttctttcttgcgggcaaaacgcattcatgatcgtgatatttgaaatatctaggtgttgatgatggatgttacgaagactggagtcatgaccttcttgttgcaagaaaggtctgcaagtttcttatgctgcagatgtgtcttgttaatgacacattactggttactaaatacccatgttctattgagttatccattatgttcttcttgtttagattttggcaattgaaaaggtcccaaggtttccacactctatgattcttgaaggtggaagcattcatgtcgatggagaaggtacatacattttattcattttatggactcgacttgatttccttataatttaaaagtgagtctctttctccatttcagagtaatcatgtttgagagttgatttcgatagctgggaattatttatgaagctccagtcatagacaaactgcttcatctgcctgtaagatattaagaaaacgaagtatggaagtgaaaaaacagactcttgaactaaatgacatcttctttaattttgtagctagcaatacaaagtactgaattctccaaatttaatttagaatgcttaaaaaagagttgaaaggttctgctggtaagtgattttcatctacttacccggttatccagttccatgggggtgtcttcatctgtttgcgttgaatcccatatcgtattctgaattcagatgctactcttatgattgttggttagatgcaaagagcatgctgtcttatcctctattatcttctcttcagggacttgtctcacgacagaagagtgcctcctgaacaaaaacaggaacccccatttgtctaaggaacaaattgagaatgaactaaaggcatatcttggagtcaggaagattatttggctgcctcgagggttgtttggtattaattctgcttgccatcttatatttttctttgcgcatagttgagataatggacttgtgtccttgacctgcaaaagttcgtgctttcatgtggtagtgttggatttggcaaagttctatccttggttgtctcattaggatgatgtgaatccattcctaagttgcgcggactcttcgattttggtgtcgtccccgtcccgatacgagacggggatgggagtgggagcgggatacgtcccggatacggtcaactgactttggacactttgatcggagtcatcgacaaattaggagaaaaattgagattttgatttctcaaaatcaaaaataaaatagatttaggagaatgaaagaataatgtccatcttggagaatgaaagattgaattctacaatattcatgtaagtttttttcacggaatatctctcaaaatttacaatatttttatatctattttttattagccgaatcctcgcacccgtatccatatccggatccgcacccctgaatcttaaaatttagattttgctgaatccgatactcggatccgtccccgtatcgaatacccgcacccgaatccgagcaacttagatccattctcatttttgcataagagaacaactgtgagaatcttgaaattttataggttaattagcacgactgtttccatcggtgcttttagcatgttccctcatgcaggtctccaaaacttcttttggtcaatccttttttcttcaaactggccttttttgagacaggaaaattaggaaaggagactgtaaactgcagtttgaggttaagccaagtgtgtaaggttttaagaactgaacctgtatggaaataaaaaagaattgattattgagccggttataacattgtggtaggatcaaaatactcttaaataatctaaaatacttgacaaaattttaattataatgcagagtcacgttcaaaacttatatttatctaacattgtggtccggcccttccccggaccccgggcatagcgggagcttagtgcaccgggctgccttagtcacgttcaaaacttttctctaattaacaatcccaagggatctacataaaaaagattactccctcttttccactttgtttggcatatttactatttggagaatcaaagaggttcttttttttaccatgtttatcttaaatatttttttcaatatttccaattatcaattattttgacttatagtactttttagattgtttccaaatatgctaattctatttccagaaaattgaaaaatttatgtccgaattcacatagaaaattaagtaatttgaccctcgtactccgaaccatgccaaacaaaatggaatggtggattattaattttggtaatcggaatgaagttggtattttgaaattttgttaattacttaagagaatacaaacctataaaacccttttttattagagtttaagtgttgttttcaaataacatattggggttaccggttcaaagaattcgtgttttagtatttatctttcaaactttgtgcagtggaccttgagatgatgtttcgtttttctttttcatttgtttacaaaataataatagagaagtgaacagctagactttagtttactgttgatgctgcaggtgatgatgatacaaatggtcatattgacaacatgtgctgttttgtgaagcctggtgttgttttattgtcatggaccgatgatcaatctgatccacagtatgagagatccattgaagccctttctattcttgaaactacttctgatgcgaatggtagaaaactacaagtgatcaaactccatgtacctgggccactctgtatgacagatgaggaggcagatggaatatttcaggtgatgtttctggtacctctttggtgacattattactgcccgatgagaaaggggttgaactcatacttgtcttaacgtaatctacttggatcctggattccattccattaaggcattcattccctgtcatacataaaattttgaaaagaaagagaaaattgcggagctgtctaaaaagaaatttcgatgcatctcaagtcaacgtttgccagactttgtgaatatgatataggcatgatgatttatttctctttctttttctatttatatgcaaaacattaatgggtaggatgatattcatcagttcgaattttggatcaaagataactatatacggattagatttgttttaaaatgttgctgtcttcctttctgtggaaacgtaattatttttactcacagaacggtgaaatatcataaatatgtgtatataattaactgaaacaactatccttttactttattgagttgactcaaaagtcagtcccagaggaattgcttggcccttaggttccgagcttctgccggatttcttggtcatgttctgtgaaatgacagaatgttaacttgacctttttgtaatggctgcttgaaatattcatgattcaggggatatatacctatagctttttgaaaaatatgatcttctgaagctgaattatatcttgaggaacatcatattagtgacattataggtattatgcaatactaatctttcgttctttccaaagttctccaaccacttttgtagctttacaattctacttgctttcttgaggatctacaaccgaggaaaaaatcaagcatcttttctctgttttttctttcaagtaaatcattctgatttgatcctactgttttcctacaggatggtgatgcaaaacctaggacttcaggtacaaggcttgctgcttcgtacgtaaacttctacattgccaatggcgctattattactccacaatttggggacaaaaaatgggatgaagaagctattagagtcctctcactggcgtttccagatcatgaggtaagcaaagaacttggtctggttactatttgcaatggtcattagtcattcggcataatgggtagagctgtatgcaaaattaccaggctttagtacattttaaataatgatgattgatatgcttatgcattcatgtgctgaggaatgtaattgttaaacctatcttacacacatgcacaaggttagctccattatcatttagcattgtagattaggcgttgtaaatgagagagtgttaatgtgtacataattatgtcaaactttcatcgcaaccactacatttgaaatatgcattagaaacgactaaactgattgtactctcattggaggtgggaggaaaattcatgtttcaactcaaactcttgcaccccatgtctcctcatgtttcagttattaatctgaacagtccctcccgaactcgtgtaatttgaaaatggttgttggtaggtggtaggaattgaaggtgcaagggagattgtattgggaggtggcaatatacattgtatcacgcagcagcagccaaccggtccataatattcacccaagattataaatggttccatatcttagcagaaggataactattacttggcatatgcaagttataaaatggaattgtctgtaatctggcgaatagcagagattccatgattgattccgcacttgacacatggaaaatgacttaatttttatgtattttctttttctttaagagtctgaattgcaaaaataat

SEQ ID NO: 4 - DNA, 9170 bp, Artificial Sequence
  Description of Artificial Sequence Synthetic polynucleotide
gaaaaatatgtcattgaatatgtcattaagcatagaaatatgtcattgaatgtcattaagcatttgagaatatcatagagaaatagtagacatacaccataatttaatttttcttataatacttttatgataatcaatacacgaaatgtaatttgatactcagtctacttgcatactcacttatacgactacaccgggcgtgttattgttgttgtctacttgacatatctagttcaaatgttcaccacgataatatttgtttgaaaacaaacctaaaaattttgaaccaaaaccacggtatctttggttggtgactgcctccccccatcccccaacccaaaacccccaaacaaaaagcaatgaatacagtatcaattataaaaccaaattaatagaaacaaaccgagacaattgaatatttaacctttttctcatttcatgtcaaatcatgaaccaactttcactttatgatgaataaataaataaaattaaagtgaacacgattatatttaatattatccgggagcacttgagaatttcaaagtaatgtgaaagcgatacaaaattgtcaagtttaactagtttattaaagtatatactatgtccacaagtgacattttggtgatctaaaataaatggccttttacataatcaagaaggaattaatttttttttttaaaatttatcattatttacatatcctaatatatcaatttaataatatgcaaattttaattaagggtagtttagtcaaatattaaaaaatcacttattatgaatcagagggagtattaatcaaaagatcatatgtgttgattgtagggatgtgcataattagaaaataccgaattaccataccgaaatcgaaaattttggtacttggtattcgataatttggtatttggtatgttatttgatttaatttttttttttttaaaatagttgatgttaggtatggtatttggtatttacaaaaaatgataccgaaatatataaattatacaattcacatattattgattataatatactaagttacataatacttttttttggtatgcatgttgaggctcatgttaagacaataaaagctcatgtacttctataaaaaaatggaggaacaaggtagcatgcagattttgattcttcttctttaggatgccaagcatcttaaagtaatacttttacgattttaggatttttttctcgtatattgagttgcatttgttggattagtccttgttgtattctctaactccagcaattagctctaaacaattaacaaaatattttcaataattatatttattcctctatatactttttctctctctatgggttgatacatactggttttggacaattttttgttgatagtcacatgagcttagaacatttttatttttgtatttgtgagcagtggcggacgcacatggtggaacacgggttcaactgaacccgcttcgtcaaaaaataatactgtgtatatgtataaattacgattaaagttgcataaattttgtataaatattttatttgaacccacttgacagctactattttacgactaaacttatgtatttctaagattgaacccgcttgcataaaattctgtatccgtcactatttgtgagtactttaattaagaatattacagtttatggctttatgcaccaattaatgttgaaaccgaacaaaccaaaattaccgtaccgaataaaccgaaatcgaaaggagaaaagctgaaccacaccgaatataattaatatattgatataatattttaactacattattcccttctttttaaatatatttaatttaattccccaaagaggaaaagataatattccacggtacacataatttgaagaatcttttcttttggaaaaatactaaaaaaggaatcttaacagtgtcatgattccatatatatgtgaaaagagcacatacattgtgaagcttgtgtttagaggaagaagaagaagaagaggttagagatggatctgaaggggacgccagttgaaaatggataccgtatgcctgctgaatgggaatcccactctcattgttggatgggctggcctgttagtactctctaattttagctgtttatgttactattttgcttgccttattattattatttatttattttcaaatttttagggggcaagtaagttagctgtgtgtatgatgatggctaatcatggatgtttgcctccgtttaggtggataagttttacctgaacagtttccttttagctcaaattagtctattcatgtaggagaatttgatgaattttcaattggaagttcatattcgacttgaaatctaatgaagttgcctttgcgctatatttcccaaagtgggacttactggcgcgattccggattagtcgagccccaaagcaggtaacaaggtgcgggaaacccagaaaacaagatttaatctagagtgtggatgctgaggcagatctagctctacgcctggggtttaacagaaaccactgctttcagcttagaccttctatgcacatgcaataataattaataataatactaataaaaatgaaataaataaatgcataaaataaaataaaacatatacgtgtataatatcacccactccaaacccattgactctaatcctggtttggcactatgtgagtgtgcgggtgcggtggaggtgtttcaaatcatctagagaataggttattaacaagtgagagtccttttatgcttatcaaatatagcagtgtaattcagcgtcatgcctatatgtagctgcaaaaataagtactgtgccaattaggatcattggacgatcatgggatttggtgcaactcattgtttcatgtgagtttgcatatcattcaagaacatgattcatttcctttcttgatcgttgtacaagtgttttttttaatctgttctgttgcaagtgagaagatactttatgtggtttctagaacaaagtaggatcactgttcatgatcctctcgtttctgattatctgctgaagttgtctttggctgcgtttagtctcagatgtctccttttgaaactttatattggagttccatcttagtagagtatggccggttgaatagtttaagcaatgtgagcaatctcgcagtctgaactacagatttcacatatcttaatgacattctatgacatcatctgtatggttgacatcttgacatgtctctttataatctcttaaaagacaacacatcttcttccctgttcgattagtactctctcccaattttattttgttttccctccttttttcttggctcaatttttcttggcttccctggaccccgcgcacagtgggagcttagtgcaccgggctgcccctttttttatgatattcaggtagtattttcttctgaagacgcagagatttaggtttctctgggctgatagctttatttttgatatgtaggaacgtccagataactggcgaaacaatgcattccatggtcaacatgtatttgccaatgtagcatctgctatctcaagattcgaaccagtaacagtctgtgctagtgctgctcaggtttgcagttactggaaggcccaaataaattttctcgttatgtcatgattgtttctcttttgtcctaataagaatcatatctatgctgaacagtgggaaaatgcacgcaacctcttacctaaacatatcagggttgtggagatgagcatgaatgattcttggtttcgtgatactggtccaactgtgagattctgtttcctttctgtggagttcaaattgaatttgcttttaactgccacaccacacaggagttacaagaaaattatatacctctgtctatgtagtttgtcatcagaaataccacatcagaagatactgaacatgtagcgccaagtgttgcaggaattgattggaccttcaatagctggggtggtaagggaatttgatctcactcttgatatccatgaggaactactgtgctgtttttcttttcttttttccttttttcacaccaaactggtgtatcctgtctcaggagttttcatatgtcagtaaatgcagcccgatttccattaattttacggcaatatttttcttgggaggtttaacaaatatattcaacaccctatctgcaatttacaggactaaaagttctaactcatgattatcttgtttgtagaaaactattactcctccatagggcatttttaaagctatatgctctggagtctgaaatgtgaatatttatttgtgaagggaaaattctcatcagataatttaactaatagccaatctgacatgatggtactttatgttgagtttttccttcccaaatgcttgaggctttcttagcattccagctccagcatgacaattatctacagctagtcatgtcatatggaatccagcgggatatagtttgacgatctgttgaagggctcaggatatttatcttctgtgtttagtggtcttctttgattgcctagaacttctagaaaggggaatatacctgtgtgtgtttgttctataagcaaatagtatttcgtatccgttaaatataagtaactgttgtagagctttcatcagttgtagaatgcttctggaagttgctgaatttttcagatttctttgtgctgatgatcacttgatcagtctgtagtgtctttgttttatttgaatctcatgtgtgctttggttctgtcttgccatagtaatattgtctcaaaaccatgtcggcgtatccagtaatatagagttgctcttagattgatccaagatcctttttgtttctttcccgcggacaaaacacattcatgattgagattttaaaatatctaggtgttgatgatggctgttacgaagactggagtcatgaccttcttgttgcaagaaaggtctgcaagtttcttatgctgcagatgtgtcttgttgatagtacattattggttactaaatacacacgtgctattgagttatccattatgttcttcttgtttagattttggcaattgaaaaggtcccaaggtttccacactctatgattcttgaaggtggaagcattcatgtcgatggagaaggtacatacattttatttattttatggacgagacttgatttctatatagtttaaaagagtctctttctccatttcagagtaatcatgtttgagagttgatttcagtagctgtgatttatttatgaagctccagtagataaactgccaatcatctgcctgtaagatattaagcaagcgaagtatggaagtgaataaaacagaaagcttctctgtacaaaacttaattattaactcttgaactaaatgacatcttctttaattttgtagctagcaatacaaagtactgaatttaccaaatttaatttagaatgctttaaaaaagagttgaaaagttctgctggtaaatgactttcatctacttacacggttatccagttccatggggtgtcttcagctgtttgcgttgaatctcatattgtattctgatttcagatactactattatggttgttgatcagacgcaaagagcatgctttcttatcctcttttatcttctcttcagggacttgtctcacgacagaagagtgcctcctgaacaaaaacaggaacctacatttgtctaaggaacagattgagaatgaactaaaagcatatcttggagtcaggaagattatttggctgcctcgagggttgcttggtattcattctgcttgtcatctaatatttttctgtgcgcatagttgatataattgaattgtgtccctgcaaaagttcgtgctttcatgtggtagtgttggacgtagcaaagttctatccttggttgtatcattaggatgatgtgtatcctttctcatttttgcataagagaacagccatgagaatcttgaaatttataggttaattagcacgactgtgcttttaagatatttgtgcatgttccgtcatggaggtctccaaacttcttttgtcaatccttttttcttcatgctggccctttttgagacagtaaactgcagtttggggttaaagcaagtgtgtatggttttaagacatgaacctgtatggaaataaaaaatacttgattattgattcagttacaacattggggtaggataaaaatacacttcaataatctaaaatacttgacagagttttaattataatgcagagtcacattaaaaaaaattctctaattaacaattcaaggtgatctacataaaaaagattactccctctttttcactttatttggcacatttactagttggagagttaaagaggttctaatttgaccacatttttcttaaatattttttcaatatttccaattatcaactattttgacttatagtactttttatgtagtttccaaatatgcaaattttatttccaaaaacttgaagaatttatgttcaaattcatatagaaaattaagtactttgaccctcgtgctccgaaccatgcaaacaaaatggaacgatggagtattaattttggtaatcggaacgaagttattattttgaaattttgttaattacttaagagattactaacctataaaacatttttttagaatatgttgttggattacattttatccggaccacatttgtgggattacactgggtatgttgttgttagtttaagtgtcattttcaaataacatattgggatgttatccgttgaaagaattcgttttttgttatttatcttcaaactttgtgcagtgggccttgagatgatgtttcctttttctttttcatttgtttacaaaataataatagagaagtgaacaactagacttcagtttactgttgatgctgcaggtgatgatgatacaaatggtcatattgacaacatgtgctgttttgtgaagcctggtgttgttttattgtcttggaccgatgatcaatctgatccacagtatgcgagatccattgaagccctttctattcttgaaactacttctgatgccaatggtagaaaactacaagtgatcaaactccatgtacctgggccactctgtatgacagatgaggaggcagatggaatatttcaggtgatgtttctggtacctctttggtgacattattactgcccgatgagaaaagggttgaactcatacttggatcctggattccattatgcattcattccctgtcatacataaaattttgaaaagaaaaagaaaattgcagagctgtctaaaaagaaatttcgatgcatctcaagtcgacttttgccagatttgtgaatatgatataggcatgaggatttagttctctttctttttctatttatatgcaaaacatcaatgcgtaggatgatattcatcagttcgaattttggatcaaagataactatatatggattagatttgttttaaaatgttgctgtcttcctttccatggaaacgtaattatttttactcacagaacagtaaaatatcataaatatgtgtatataattaactgaaacaactatccttttacgtcattgagttgactcaaaagtcagtcctttgcttggcccttaggttacgagcttctgctggatttctcggtcatgttctgtgaaatgacagaatgtaacttgaccttttgtaacggctgcttgaaatattagtgacatttgaaatcttctgaagctgaattatatcttgaagaacatcatattagtgacattataggtcttatgcaatactaatcttttcgttgtttccaaagttctccaagcacttttgtagctttacaattctacttgctttcttgaggaactacaatccgagtaaaaagtcaagcatcttttctctgatatgatcccactgtttacctaacaggatggtgatgctaaacctaggacttcaggtacaaggcttgctgcttcatacgttaacttctacattgccaatggcgcaattattactccgcaatttggggataaaaaatgggatgaagaagctattagagtcctctcactggcatttccagatcatgaggtaagcaaagaacttggtctggttactatttgcaatggtcattagtcattcaacataatgggtagagccgtatgcaaaattaccatgcaccttgcagtaacgctgcagttcatgacctagaatgatctcagttcatcaggaaataaaatgacctcctcatatttatcaataataataataatcataataacaataatataagccgagggtctttcggaaacagcctctctactccttcggggtaggggtaaggtctgcgtacacactaccctccccagaccccattaatgggatttgactgggttgttgttgttgttgttgttgttgttgttgttgttataataacaattaaaaggattccttgtcatgagaaagagtccttatagaaggatgtttaagagaaacgtataacaatgcagtgaaagaaaaaaggacaaagaccaggctttagtgcattttagataatgacgattgatatgcttagcattcatgtgcttaggagtgtaattgttaaacctactttacatacatgcacagagttagctccattatcatttagcactgtagactgtagactaggtgttgtaattgagagatgtgttaatgcgtacataattatgtcaaactttcatcgcaaccactacagtcgccactacatttgaaatatgcattagaaacgactaaactgattgtactctcatcggaggtgggtggaaaattcatgtttcaactcaaattcttgcaccccatatctcctcatgtttcagttataaatctgctgtccctccagaactcatgcaaattgaaaatggttggtggcaggtggtaggaattgaaggtgcaagggagattgtattgggaggtggaaatatacattgtatcacgcagcagcagccaagcggcccagaatattcacccaagattttaaatggttccacatcttagcagagggataactaatatttggcatgtgcaagtgataacatggagctgtccgtaatctggctaatatcagagattctatgaatgatatccttacttgacatctggtagatgaactttattttatgtattttgctttttctttcaagatatggtgttactcgcttattgaggacatgaccatggatagaaaggggtggagg

SEQ ID NO: 5 - DNA, 5276 bp, Artificial Sequence
  Description of Artificial Sequence Synthetic polynucleotide
attaaatttgactatgatgtaatatttatttaattatcttttatcaatgatttcacttttagttgatttatcctttaaaaaaattttgtaataaatgattatataagtggtgaatgttaattatatataatgaatatggaaaaaagaaaaagatagtattagtttgaaggaaataaaaaatgaaaagtaaatagaagataataatataatatagaagagagagaagatttggtgtaaaaaattgtgtaatggttgaattaaatatggtgttacatcattttgatgtgaaatttggtgttagggttgcagatggcctaagaggagtgacctattgtctagatagtaataatgcagttttgtttaactaaagtagtggttatatgttcaactttagaggtttttgcttttacttgtatatatttttttcctggtgaataaacttttagttacccaaaaataataataattgagttaaaagaattgttacaaagaaacacaaataaggtaatattgtaaaccacgaagaaggtcagtgtgtgagatacaaaatgtcaggagaggtgtctgaaattattgtttttggcattgctgagaacattaacaataagaaaaaagcattaacaatcaacatatattcaaattggtaaaaaaacatgttcaaattggtgggttagaataagttaggacccattaactatttaatcagtttaaatttaggtagactctattatttgattgtagatataaaagataatattaaagagagaaactaccaacccctgtaacattaaaaaaagttagaaacaatgttcattagaagatccaaaaggcaaaagataaaaataaagagaaataagatgaccgaaaccaacaattgatcgagcaaacaataaacaaagaggatagagaagaaaaactcacgaggaatcgagaaagacaaaggagaaggaaagagatatggaaaaatgaaaatcttagtttcagatggagtgctgaaggcaaaagggttaggaaaaattaaatcacgtgggaattggaagtgaataaatgtaaggccatagcacctcaaaatatacacgcagtcagaggctggacgggaccaggatttcaagtttatgggttcggaattccaatagtttgaagttactgtgttctagattaataatttatacatattcaatgatatttttaagacaaatatagtgttcgaaccaaagctactaggttcggccgaacccgctcccgccactatagctccacccttgcatgcagcaattaagtactccataatgattgaattacacataaacaaatgcataagcaacgtggtgtttgcaagtgaacagctgaaagtccatagcacctcaaaaaagactaaggtaagggtatcaatacaagcagcaattaagtaataaggaccaaatcacacagtatccctaaacaaatacttattaagtgatgaggtacgaatcggaatcgcacagtatcactaaacaaatgcatacaccacgtgggtgttatggaagtgaagtcataagcacctcaaaaatggaccaaattacacagttatccctaaacaaatgcataataccgaaactacccatgcacgaccataatacttcacttattatatagttatggcttctgcttccttggccaacaatcagttcatctctaacttcgaattccagttccattttcacttgacacacttcgaagcgccatctttgctactttaaggtaattgtatatttcaaaagtttgttttgcttatctgttttggaatgacagcggtaattgtgttgtttaacaaatttacatgctccaaatgaatggaagaagagttctaacttcaagaccatggccagaggccagttgttaattcctaagatcatcctgaacaaaatcttctctatatgtagcctctacgtactatattcgttcatgctttaatgtgtaatgcataacaagattttaccttaaatatccagatcgatcatatggcaactggtatcgcttcaggatgcggacagttacatttgaggaagcctgtctacttgaggaatagctatggaaataaagctcactctcattccaatgtgattctcaacggcacgcaaaaccagatcgcttggtacgttttaggggtaaaccagtaatatattttttcatgacgtgacacaattctagaagtagtcatttcctgattccttaggaaaagggaaggttatttcgtcccattttatatatcatagttacacattcttatatttaatagtaacaatttaactttaaaatacctcttttacccttaatgaaatgatttatagcgaaacacatatccattgctcagtttacaccataaatttcaaaaatattcttaaatattgtgttcagtcaaacatcatcacataaattgagacattgagacatagaggagtactatgttaattttctgatggtgatatgctttaccaggtctagttgggtttcaaatgtcttgcgagttaatagaagtagctatccacaatgtcaagtgataaaaacaaactggaagtctcggcgaggaacaatcaaatctagccagcagagagatggatcagttactaggtacttcgatttcattgtgattggtagtggaattgctggccttcgatatgcacttgaggttgccaagcatggaactgtggctgtgataaccaaggctgagccacatgagagtagcactaactatgctcaaggtggtgtaagtgctgtgctctgccctttggattcagtggagaaccacatacaagatacaattgtggcaggtgcttacctctgtgataaggagactgttaaagtaagttcacacgaatatatacagtttaatttggagactatttccccccttttgcttttgctataagtgtatctctcctttaatttctccaggtagtgtgtactgaaggacctgagagaattagagaactgatcgctataggtgcttcattcgatcatggggaggacggaaatctggatctagccagggaaggaggccactcccatcgtcgaattgtccatgctgctgatatgactggcagagagattgaaagagccttattagaggcagtgtttaagaatcctaatatacatgtgtttcaacaccattttgctatcgatttgttgaccactcaggttagtttattagtgtgctatagttgagtaaaggtcgcaattgaactattcagcaaggatttgatgttgtaatttgtatggcaaatgcaggatggttctgacgtagtatgtcatggcgttgatactatacacacggaaacgaaggaggtaagtaacagttgctttcactcatgttgacttttcaacattcttcaaaatgacataaattgattttcttacttgtgtattttaggttataagattcatttcaaaagtgactttgctagcatcaggtggagttggacatatctatccaagtactactaatccgacggtatggtttcaatatcttttcctccaaaatcttgttagttatgattaatgtcatgctccaaaaataaaagatctggacatggcactcgatcgatgctttgaaacttgtgacagagcgaaccaacatccattacatacttagtgcaattagaaatatgagctcgctgccctaaatgcctaaaaaaggattctgtttttatactttgaccatttgtgccaacagaaaagtttaaccctgttgaatgtgggcttgtttaactttctaattcatattgacattatgattataggttgcaactggtgatggaatggctatggctcatcgagctcaagctgtaatttccaacatggagtaagtaatcctcagtagcattatttttattgtacaatatctctgcctctgctttagtgagttttgtgctgtaaaatctgctgacctagatttcatccttttttttttctttcctgatttctgcatcttgttcagcatttcgttcagaatttgcactttcgactaaaaacacaagggtgtgttcaaaaatttgcattattaataacaattctcttcctcgtgttgccaggtttgtgcaattccacccaactgccttggctgatgaagggcttcccaacataccaagtgccagagagaatgcttttttgataactgaagctgtcagaggtgatggaggcatcctttataacttagatatggaaagatttatgccaatgtatgatgaaagagcagaacttgccccgagagatgtggtagcaagaagtatagatgaccagctcaaaaagcgtggcgaaaagtatgttcttcttgatatcagtcacaagcccagagagaaggttctgtcccattttcctaatatagctgctgagtgtctccgccatgggttagacataacacagcagccgattccggtggttcctgctgctcactacatgtgtggtggagtccgtgctggactcgagggtgagactaatgtgcaaggtctttatgtggcaggtgaagttgcatgtactggtttacatggtgcaaaccgacttgctagcaactcattgcttgaagcactagtgtttgcacgaagagctgtacagccttcaattgatcatgtgaacgtgtctagaattgataacggtgcatcaagttggtggccgcggcctgtagcccccctggcactaggagatacagtacttaacaaagtcatccgtcggacaagggaagtgaggaaagaactacagtcaatcatgtgggaatatgttggaattgttaggtctacctcaagactaaatactgctgagaagagaatcaaagagttggagttggaatgggaaacatacctgtttcagcatggctgggaaccaacaatggttggagtagaggcttgtgagatgaggaatctcttctgttgtgccaacctggtagttagcagtgctctttctcgacaagagagtcgtgggcttcactacaccactgattttcctcatgttgaggaaagcgagaggttgccaacggttatctttccttctcagcgaaatagcacatggagcacacggcaattacacgcgcagccgataagttagatatctcttacctatttgccattcttcctgccaaaacatctccagtgcaggacatgatttgtcgattttaccgggttgcaggggtgtatctatcggaaggggtatgggttcatatgaacacatactcctctccatagactatgtataataatgtatatttcttcaatgttatttaaatatgcaagtgtgtcagccaagct

SEQ ID NO: 6 - DNA, 21289 bp, Artificial Sequence
  Description of Artificial Sequence Synthetic polynucleotide
gatcggtacacctgtgtatgaacttccagcatattatgctgcaactccaacacgcagaaagttccagcataatatacgagagattggcgcacatgtgtatcagtgtatgaacttccagcatattatactggacgagtatattatactggaacttcaacataagtatactggaactccaatatattatactgaagttccaatataatatactggaattccagtatattatgctgaagtatttttcggattttgaacaatattttcgatcagatttatctttatatgaaaaatagttaaattttaattaattttaaaattgtagctatttttgaacgaacgtaaatctaattgaatttctccaaacaattttcttccttgttcgcgctcaactacaggattcggtgcgttaatgtggatctattattaaactgtactaatataagtaagataacaataaaggaggaaacaacctaaaaggtgcgggtttcccgatgccgacaggagagtcaaaaagaaagacttgtttatttcgaccaagaaaatataaaaaaagactggattcgtgtattctttttgtgaaagaacaaattggattccatttcctaaaagcgaaggctcaaactgaattgtccaaattggttgtggaggaaaagcgactggggtcctccgcaacttcctttttctttaacgtcacagattttacaccacggaaactgcatgcgattaagttaacgcattgattagctaaaaggccataatttgaaaaggaggtttttttatacagggtggattccaaggtgacgactgcagcttttcgttatatattccggctctctctcttcagttctctaattagctcgaattctgcttccttggccaacaatcagttaatttgtaacttcgaattcgagttcaattctacttcggagcgccatttttgccccgttaaggtaactgtattgttcaaaactttttttgttttgtcgatctatttttttttgttttgttttggaataataactatagttgtaattttttgggtgggggatgtggttgtaatttagtctagggttctgttaggttggggatgctagaatcttgagaagctttataaatatacgaacatgcagttgtatgataaactgatagccggtagatcctgttgttgcgaacatgctatgctagctcaattctgctctctgttccctttttctactgttttctttccctggtaaagcaacacaggctgacacaatcctagcccaaatgagagtaacaaagtttccagtctttagaaggtgaaatctataagcaaatagatgtgaatgtaagtgccttctggggcatacgattaacaaatagacgtgaatttaaatcactgcaggaattgagagaagaaatatagagtgtgttcggtataactgaaaatgttttcacttattctccagtatttggctggtgagggcaaaatatttccggagaatatttacaaatgtttgtttggtgagtggaaattgtttcctaatgttgttacaaaataatttactttttgatttcatttaggccccttaaagttcttgaaagattaactgcagaaaaaagtattcaacatgacatgtcattcttgtaagaacaagtcattaagaaaaagaaaaataatatattaaaattagaaagtttctaaatatatgaaaatgtcaatcttttgtaaataattcaagaaaatgaataatgacagtaatttaaaagtatcacagatttacataaagaaaggaaaaggaaaagagagaatggtacatgaaattttaaatgctttggctagattcaaattccatactcctattaaaagaaatgcctgacaggtaagaacatataggtactgttgtgtcaacatgcaatagcatttggtcactttggagagagtatagagaaaaagagaaaatggcagagggaagtgggagggaggcaagagccgattgtctagcgccaaaaagtttgatatgttctggatattggaaattgactttccttgggttcttaggtaagttagtttcatcaaaattaaaggaaaatgttttctttaggaaaatgtttccccaacaatttaggccaaccaaacatggtaaaattggaaaagattatcaagaaaatgttttctttcataccaaatacacccataagtaggagggattgtgataaattttgcaagttgtttatgtattttcagagatctatttacctgtcatacatgttctacaaaacacgagtccatgtatatatcattgcacttctcctttacaacttatttaatttctatttaaacagctttagcgcgtaagagctgttcatttccttgaacaccattgccaaagaacggtttaatgagggaaaatgaaaatttagctatgttggcaagtcctgagtcttagagcaaatgatcttcattgggtcttattgtttccttgcaatacttgttcagtcttgtaagttctgattttcgctaaaacaactctttttagtcgtcacttgttacttacaatttcctttctcctaatagaaacgtgtccggaaagctgacttttggccctgtatgtgtttctggtttttggagccttcagaatgatagtactagctgttcaggtgtctattgagggtcttcatcaaagcaacagtaaggaaatagttagaaaaactgaaaaggacttctccttaacgtgtttcagtctcttgattttaaaggaaatgcattttgtagatacattggcattcattgaaaatggccttgactcaatacctatgtcatgtgacatcaggaagggaaagcaatgatttctctttctgtttcagtaataagtgatgaagtcttgtggcaacttccatttgtaaattcattttgtacaagtggattatagaaaattagttgataagttcatacctttcttttctctactttttctcttgcaatatgagatatcacaggttagagacatgatgcacattcatttgtggttcacttgaatgagaaaaaagtatatgtttcttctttttatgtttcgtttccttttggttttcagtatctggtatcgaagttgtgtaataaacagtttgttccacgcatgtgctgctgcagcattaacatggactagccatcttgtcgtaggagttcaatttgtggaattccggtttaagcccatcaactgtggaatctctaaatattttcattgaattcaccatacgttagatgatatgttttctcatggtcctttcaagcacttctccttctgtttaaatgtctccacatgcttttctgccccttcaatatcagaatttgtctaaaagttcctttttctttaataaatctactgctccaaatgaatgatagaagagttgtaaagagttctgacttcaagaccaagcacagttttggttgggctttcaaattagcttataggaagttgttcctaagatcgtcctgaaacaaaatcttttctttttgtagcttctactgtattcattcatgctttaatgcaatttataacaaggtttcccctaaatatccagagtctttaattttgctttcgagtcccagctgcgcaaaacgtattaccgaagaatagttttctgggagatgttacaagagtttggttagaagggtctttcccttctaaagtgtttggttcaagttatctaaatgtcaagagccaatattttgttattaagctcaattccttgccctgtttgggtgtttttagaggtaatcaagatgatttttaaatgtttagtttcactaacattctaacctcagaattgggtgtgccaatctgatatcaattcctttgacttggattgtcagtcaggaagatcatatggcaactggtatcgcttcaggaagcggacagttacatttgaggaagcctgtctactggaggagtagctatggaaaagctcactgtcattccaatgcgatcctgaatggcatgcaaaaccagatcccttggtccgtattaggatgatgtttcttcgcctatctctcctctctatctttccctgctttattatcttcattctattgccccttctcctctcttttgggggaataattgtcaaaatgttttattttggagctttcaagatgttattacattccaatttgtacggacataggcattcacgtgttggctgttggactgtttattcctcccatgagaacttactggaaagaatctttgtatacgtgtttaggcataaaccagtaatatcttttttcatgagataccagaatcctaaaagttttttttttgagtcatttcctgattctttaggaaaaaaagaaaggttatcattttcattttctggtggtgatatgctacttaaattttccaggtcttattgggtttccaaattcttgcgagttcatagaagtaactactcacaatgtcaagtgaaaacaaactggaagtctcacaccggaacaatcaattcttgccagagagatggctcaactaggtattttgattttgctgtgattggtagtggaattgctggccttagatatgctctagaagttgcaaagcatggaactgtagctgtgataaccaaggctgagcctcatgagagtaacactaactatgctcaaggtggtgtaagtgctgtgttctgccctatggattcagtggagagccacatgcaagacacaattgtggcaggtgcttacctctgtgatgaggagactgttagagtaagttcacagaatattattgtctcttcagttccgtgaagcagtttgccctttttcagttttttctctttggaagctgtttcctttgcttttccaaatttttccaggactgcttggttggatgaataggaaagttctccatctttcaggttgaaacgaaaggaactttctgtttagacggcttttgccacaggatctagaactgccaaaaaatggaaaagggtaagagcaatcgggcagttaggcttgttttcaattagaagctgaagagccaagcaataatactaaagaaaggctctatgcctattccttccttctgatgtaagtgcttacaagttggaaccgaagaaagagttggagagttgagaaaccaatggttggtaatatggacgacttcctctggtttatgtagtgctcctattcttcatggttcagattgttgaatttgtctttgaacatcgtgagaaggcacgggagaaaatatgattgatattattctcatatgttaaacatgatacaatgagccctgtatatacataacatgtcctactcctaatacatatgggattagggttacataactattctaacactcccctcaagctggtgcatataaatcatatgtaccgagcttgttacatatgtagttaatacgaggaccagtgagagacttggtgaaaatatctgcaagctgatcattcgacttcacaaactttgtagcaatatctcccgagagtatcttttctctgacaaaatgacagtcaatctcaatgtgtttagttctctcatgaaacactggatttgatgcaatatgaagagcagcttgattatcacacacaagtcccatctgactaatctcaccaaatttcaactccttgagcaactgtttgatccaaattagctcacatgtcaccatagccattgctcgatattctgcttttgcactagaccgagcaaccacattctgtttcttgctcttccaagataccaaatttcctcctactaagacacaatatccagaaatagaacgtctatcagaaggtgatcctgcccaatcagcatctgagtatccaacgatctgctcatggcctcgatcctcaaacaataaacctttgcctggagctgattttatataccgaagaatacgaacaactgcatcccaatgactatcacacggagaatccataaactgactcacaacactcacaggaaaggaaatgtcaggtcttgtcactgtaaggtaatttaatttaccaaccaaccgcctatatattgtaggattgctaagaggctccccctgtcctggcagaagtttagaattcggatccatcggagtgtcaacaggtctacaacctgtcattcctgtctcctcaagaatatctaaggcatacttccgttgtgagatcacaatatctgagctagactgagcgatctcaatacctagaaaatactttagtttgcccagttccttagtctgaaagcgttgaaagagatgttgcttcaatttagtaataccatcctggtcattgccggtaataacaatattgtcaacatagaccaccggataaatatagagactcgaagcaaaatgccgatagaatacagagtgatcagcttcactccgagtcatgccaaattcctggataactatgctgaacttaccaaatcaggctcgaggagattgctttagaccataaagtaatcggcgcaagtgacatactaggccacgagactccccctgagcaacaaacctaggtggttgctccatataaacttcatcctcaaggtcaccgtgtaaaaaaacattcttaatgtccagctgatagagtggccaatggcgaacaacaaccatggatagaaaaaggcggactgatgctatcttagccacgggagagaaggtatcactgtaatcaagcccaaatatctgagtatatcccttggcaacaagacgagccttaagacgatcaaccttaccatccggatcaaccttgactgcatacacccaacgacaaccaacactagatttacctgaaggaagagggacaagctcccaagtaccactcgtatgtaaatcagacatctcgtcaatcctgtcgccatcctggatgagacaatgcttcacctgtagacttagggatggaaacagaggacaaagaagatagaaaagcataatagggtgaagacaaacgatgataacttaaaccgacataatggggattaggattaagggtggtccgtatacctttccgaagtgcaatcgatgtactaggaagagacaagtccgcagtaggaccagggtcaggtgcaggacgtgaatcagctgggcctgatgctgggtgcagatgacgatggtatgtcaagagtggtgttcctgtggcagggagtgtaagaggagccacactagactccccaacggttggaatgggtaagacttctgtggcggaaggtgaagaaggagatatagtagaatccttaaaggtcggtataggtaagacctcagatatatcaaggtggtcagaagaggtaaagaaaggtttagactcaaaaaatgtgacgtcggaggacataaagtacctacgaagatcaagtgaataacaacgatatcccttctgaacacgagaataaccaaggaagacacacttgagagcacgaggagctaacttatctttcccaggggctaagttatgaacgaaacaagtgctcccaaaaacacgagggggaacagagtataagggtgacgggggaaacaatactgcatacaaaatctgattctggatgggagatgaaggcatccaattaaccaaataacaagctgtgagaactgcatcgccccaaaaacgcaacggaacatgagattcaatgagaagtgtgtgagcagtctcaatgatgtgcctattctttctatctgcaaccctattttgctgaggggtataaggacaagaggtctgatgaataatttcttgagaagtcataaactggtaaaattgagaggataaatactctaaggcattatcactgcgaaaagtgcgaataaaaacacgaaattgatttttaatttcagcacaaaaattctggaatatagaaaacaactcagaacgatctttcattaagaaaatccaagtacatcttgaatgatcatcaataaaactaacaaaataacgaaatcccaaggttgaattgactctactaggaccccatatatcagaatgaactaaagaaaaaatagactctgcatgactctcaatactacggggaaaggaggctcaggtatgtttctcgagctgacatgactctcattctaatgtagataaactagacaaactaggcaccatcttctgaagcttggataaacttggatgtcctaaatgtctgcgaattaggtctggaggatctgtaactagacatgccttggaggaattgagtgagttaaggtagtaaaggccttctgattcaagtcttgttccaatcgtctgtcccgtactgcggtcctgcataataaaagaatcatcaataaaatatataccccaatggagggcacgagtcaaatgactaacagatgtaagattaaaaggacagccagggacataaagaacggaatctagagtgacagagggtaggggattcgcttgtccaacaccttttactttagtttgagacccattggctaaagtaacagtgggaagagactgtgaatacgcaatatttgacaaaagtgatttattaccaaaaatatgatcagaagcggctgagtccacaatccattgtccaagagtactagactgggaaacacaagcaaaagaattaccagcaacagaagtatcagtctgagcaatagaggctacttgtggagatgtttgcttatttgctcgatattgaaggaactcattatactccccttcagataaagaaaatccctggttacctgtagtctcggtctgagcaacataagcatttttgggtgggcgaccatgtaaagaatagcacacgttactagtgtgtccaagtttatgacaataagagcacttgagtctagatttccaaaacgacctcctcctcgtctattctccatagtttgagatgcccgattgtccactgactgggatacgaggacagatgagtcaagtgtctgtgatgagctcactgggtgacttggtgctgcagcaaggcgaagtaatcgagagaataattcatcaactgtggggacagtcggtctagccaaaatctggtcacgtactgaatcaaggtcattagggagtccagcgagtgtaagaactacaatatcctctgtcgttgctcttgttgcttttcaatactagcagaaactggcatcaacgtctcaaattcttccatgactgcctgtacttgttccaagtaagtagatatatccaattcctgtttcttcaagcttgtcattcgcaatattacatcatagagacgagatatgtcattagtgtataaattacgagccttttcccaaactaaataacatgtatggaatggacggaacaagagcatcaatttggaatcaatagatcgccacaagatactacataactgagcattgaccttctcccaaagtgttttggccttttcatcaccttcgctagccttttttgttaaatgatcttgaactcgttgacctttacaccacaactcgaaagacgaagcccaagctaagtagtttgaacctcccattaaaggttctgaggtaatcgtaacaccggaattgccagaacccgggtttttagacccgaaagcatcgactcccaaagacatcgttggattatcaccaaataacagaaagaatcaactgacaacttgctgaaacaggtgaaggaaacactgtttttgccggaaaattactgtagctgccggaaaaagtcggtgtagtcgccggaaaaactcaaagtggtcggaatcaaacgaaaaaggtatggagactcggaattactaggcgatccgactgtccaactggaagtttttcaaaaaaatggttggaacagtgctcacgcgccagtgcgtggggcgcttgggtcagatctcgccagaaagtttttggcgcgtgagggcgcgtgtacgtgagttttccggtggggtttactagggtttggtcgccggagcctgaggagtcttgtggtgttattggttttttgcacacaccaacaaaaattgatgtgattacgagcagccttctaagtcgccgtaaaattgcacggcgacgaggtctttcttcccggaagtcgctggaatgatgcacaacgataggtttctcactaaagctctgataccatgtgagaaggcacgggagaaaatatgattgatattaaacatgatacaatgagccctatatatacataatatgtcctactcctaatacatatgagattaggattacataactattctaacaaacattatgaaaagagagtgaaggtgacatgcaggaaggaagagataaggataagacaggaaattcacatgtgttgtcttttggttttattttgggatttcatttagtttgtgccacagtaccatgtttttttttcttaatgagaacacacttctgacttccctaaatcagcaccaattttctgagggtaactgctgataaggatagacactccaaggttatccaggattcctagcttaagttgtatcatataagtttctctcctatcccctcaagagtcctatgtacagaaaactctatcccttctctctcgagctttagctccggagtccatttacttttgttaatgctaacaactcttgcggagttgtgaagtgccttcttctgccgagagtagtaaggcccaatggacttcgcatctagtaggcccattatcaacatacaatgagggagaagggatagagttctctgtatataactcttgtggggataagagagaatcttatatgatacaacttaagctaggtgttagcggaaacgtaaaagaaagaacacaagatttaacgtggttcggatcaaaataatcctacgtccaccagagaacaattgcccttttaatattaacaaaggaaggggagatttcccaattacacttaagagaatttctctcttaactctctactcactacaatgtattgtattattttgggatgatttctacaagtgaaggagtgcatctatttatataggtaaagatgacatcaagaggaggtagtttgatgtcatgggtgacatcaaaggaggaagcttcctcctagcatccacaccaactctttccaccaactcttccaattggcatgccattgttgactaaacataaaccaacattttcagcatgccattgttaactaaacataaaccaacattttcactagggatcctggattgccttggagtgtctatccttatcaaccgcttagtttgctgtatactattttcatttgtctctgtaaatatatactggtttcaaggaagttttgtgttgtgcttggtagatgggatatcacaactaatatcttcttatcagttgataaaatcctggtccatgtaattctgctttttatgcaatctatgttctaaatgaaatttatgagatggctgatttattactccaaaattgtgtttagtctactcttttaaaatcttcgtttttataagtatatctcctttctccaggttgtgtgtaccgaaggacctgagagaattagagaactgattgctatgggtgcttcattcgatcatggggaggacgggaatctgcatctagccagggaagggggccactcccatcgtcgaattgtccatgctgctgatatgactggcagagagatagaaagggccctattagaggcagtttttaagcatcctaatatacatgtgtttcaacaccattttgctatagattttttgaccactcaggtaattcttttctttgctcttcttgtttattattgagaaaatacttctgctctagcatcaatttggtccaaaatgaagggttgttgctataatctaattagtcttggcattattcttttgcttatgttgtttgtgcagttgaactgtttttccatgtaaacatttgctaatgttaatttgtatgccaaatgcaggatggttctgacataatatgtcatggcgttgatgctataaacacggaaacgcaggaagtaagtacagttgctttcactcatgttgacctccatgtattgaacatttcctcacaatgacataaattgatttcttaggcttctgcgctcttactgtgagtttgtaggttataagattcatttcaaaagtgactttgctggcatcaggtggagctgggcatatctatccaagtactactaatccaccggtatgagtttcaatatcttttcctccataatattttcatttatgagggatgtcactccttataatcaataagataatcagagtcctaaacagtgatatggggggagaaggtgggaagggaatggtggctaatactgtaatagttaatttcatgactagcatacatttgcatcttattgttggcgataagcttcgtcacaaagtatctttcgtaactcataaggaccttaacaattgacattggtgcaaacaacattccctagagtatgaaacatggcctgattttaattatttaaaccctacagcacttgcatgttaagctaatgtgcaattagaaataagggctggctgccctaagccacaaaaagaggaaaaacccaaatatacccctgtacttcgtgaaaaggtctaagatttattctccctttgaagtttggctcgctggtgccctcgccgctaaacttttggactatatttacccctatttgctaatggaccttggtttgagacttaacaacaacaacaatatacccggtgtaatcccacaagtggagtctgaggaagcgtgtcaagataatagaaggaaaaagatatttaggagagaaacaataaattgcacaagacaagacaagatttaggtggttcgacaatttttgcctactccacggccacacaaagaatagctctttattaattgaagagagaaagaagaagttttgggatgatctacaaatgaaaatgtagacccctatttataagcatttgaatgccctgccgaggtaagcgcttacatcataagaatgccgatgtaagcgcttacatcataagaatgctgaggtaagcgcttacatcacaagaatgtcgatgtaagcgcttacatcatattttcatctcttttcgatttttctttcttttgttttgtctactttcacatacaacaacaagtttggtcctatcaatctccctctcaaacctatgttacatcaagaaagaaaataaagaaagatttgctattctctggtggcgccttcactctatcagtcttgaaggctaactgaggcagtgcacagcctcagtttgtcaacaccgacaactttggtcaacatgtctgacgggttctttaatcctggtatcttttgtagggaaagagttccttcatttatcaactctcggatatgatgatacctcaactggatatgcttcgatcttgcatgaaacactggattcttgtcaagatgaattgcactctggctatcgctgaaaagctcataattgtcctgttctttacctagctctttaagaaaattcttgagccaaatcatctcttttccagcttctgagattgccatgtactctgcttcagtggtagatagagcaacacttttctgaagtctggacatccaactaacagcagtaccaccaaaggtgaacatgtagcccgtggtactttttcgactatccagatcgccacctaaatttgcatcagtaaaaccttgtaagataatattgctctttttaaaacaaagtgccatacctgaagtgcctttgagatatcgcaatatccattttacaccttcccaatgctcctttcctggatctgacatgtatctactgacaactccaactgcatgggctatgtcaggtctagtgcaaaccatatcatacatcaaacttcctactgctgaagcatacagaactttggacatgtacttcctttcttcatctgtcttaggtgattggtcctttgacagatttagatgacttccaagtggagtgcttctggtctttgcatcatgaagactgaacctgcttagtaccttctgtatgtacttttcttgagacaactttaaggttccttccgacctgtttctgctaatcctcatcccaagcatttgcttagctggtcctaagtctttcatatcaaactcctccgccagttgttgcttaaccaagttgatctcatttatgctagatcctgcaattagcatatcatcaacgtacaacaataaaatgatataggattcatcaagatttttgatataacagcaatggtccatctcacatcgtgtgaaaccatttttatgcatgaatccatcaaatttcttgtaccattgtcggggagcttgtttcaaaccatacaaactcttcttcaacttacacacaaggttttctttaccagaaacttgaaaaccttcaggttgcttcatgtagatgtcttcttcaaggtcaccatgcaagaaagcagttttaacatctagctgctccaaatgcaaattttctgcagctacgatacttagcaccaacctgatagtagttaatttgactacaggagagaagatctcggtgtagtcaattcattccttctgctgaaagtcttttactactaatcgtgctttgtatctcttcttaccatcatgctcttccttgactctgtacacccacttgttctgcaatgccttctttccttttggtaactccgtaagtatccatgttttattcttttgaagagaattcatctcttctttcatggctagcttccacttatcagagtctatcacctgcattgcttcaacaaaatgctctggttctccagcatcagtaagaagtaaatagtgaagagagagttaatttatttggagcattcgtgactcttttagatctcctcaatgtaggttcatgagtaacagaatccgaatttgattcaggtcctgattccagatttggttctggatttgattctgtctctggttccacttcaggttcggcttctagttcttcttcaaattcggcttctagttcttcatcttcaatttcagaatcagttgtaatccctctagctacttcattttctgagatttcttctaactcaactgtttcagacattgtctgtttgctggtgttggttggttctacttcaagcttgtccttgtacatcacattttcattaaatgtgacattcctgtgtcttaggatctttctattcggatcatcccaaaaccgataaccaaaattatcatcaccatagccaataaagaaacatttctttgctttaggatcaagcttatctctatcattagagtttacatgcacataagcaacacaaccaaaaattttcagatgtgagagagttacctcctttcctgtccatacctcctcaggaatttcaaaattcagcggtacagagggtcccctgtttatgaggtaagctgccgtgttaacagcctcagcccaaaaatacttcggcaatccagaatgtattctcatacttctggctcgttcattcagggttctgttcatcctctcagcaacaccattttgttccggtgttccaggaactgtcttgatcattctgatcccattctccgagcaaaatgctttgaactcttggctatcatactctcctccattgtcagacttcagacattttaactttagacttgtctgattttcaacttcagctttccatcttttaaaggtaacaaatacatcagatttatttttcagaaaataaactcatacctttcttgtggaatcatcaatgaaggtgacataatagcgtgagcctcctagagaggttacaggagctggtccccacacatctgtatgcactagttccagcttctctttctttggcgtccttcccacctttgagaaactaactctcttttgtttcccgtaaatgcaatcttcgcacaaacctaattcaacatgttttaggtttgacaacttctttttggatgccaaaagcttcattcccttctcactcatatgcccgagcctccggtgccacaatgttgtatcacgaccatgatcaactgttgctatagtatctctttctattgcagttgcatgcagtgttccccttttgaagcctcgtgccacaaccaaattccctttggttatcttccacgatccgttgtcgaatgttgttgtatatccttcatcgtcaatctgacccatagatatcagatttttcttgaggccaggaacatgtcgtacattttgcaatttccatagcgtgccttgtgaagtctttatataaacttcaccttttccggcaatgtcgagaggttcgccgtctgctagataaactttcccaaattttccagcaatataattatgcaataattctttgcatgatgtagagtgaaaggacgcacctgagtccagaatccaagattcaactggactgtctgcacaacaaattagtgcatcaaagacttgttcagcaattacatttgctgaattttcttccttcttcttctttggttctctacattgactactgtagtgaccctttttatcgcaattccaacaagtaatgtccttgcgatttttggattgtcctcttctccttgactttgatctgccacgaccataaatctgtcctctttggttgattctccccctgctttcggtactaaaagcagatcctggggaatcacttgattcttttcggcgaatatcttcgcttagaaccaagtctctaatatcattcaatttgagtttggtacttcctgatgaactgctaactgcagttactgttgcagaccaactctccggtagagatgatagtagaatcaacgccctgatttcgtcatcaattgttatattaacagaactcaactgagttaatattgtattaaactcattgatatgttccgtgactgatccaccttctgtcatttttaagtggaacaatcgacgcatcaaatacactttatttgaagcagatggcttctcatacatatttgataacgccttcatcaggcctgcagtggtcttctcgttaatgatgttaaacgccacatttcgtgttagcgtcaaacgaatcacaccaagagcttgacgatctaatagatcccaatccgctttggacatagtctccggtttcacctcggtcagaggtaagtgtaatttttctggtacaaatagtcctctatttgcattttccagaatccaaaatctttgccattgaacttgtcaatcttaaccttcccttcttccgatgccatttttcacaaaaataatttatgtgaatagtgcttgtgaatagtaacgatgatcaatagtgtcgcactattcttgtgaatagtacctgcaccaatactgtacttttctaccaggattacactgtctgtgctctgataccaattgttgaggaagcgtgtcaagataatagaaggaaaaagatatttaggagagaaacaataaattgcacaagacaacacaagatttacgtggttcggcaatttttgcctactccacggccacacaaagaatagctctttattaattgaagagagaaagaagaagttttgggatgatctacaaatgaaaatgtagacccctatttataagcatttgaatgccctgctgaggtaagcgcttacatcataagaatgctgatgtaagcgcttacatcataagaatgctgatgtaagcgcttacatcataagaatgctgaggtaagcgcttacatcataagaatgctgacgcttacatcatattttcatctctttttgatttttctttcttttgttttgtctactttcacatacaacaacaagtgtaatacccgggtatattgttgttgttgttgttactcatactgctgctactcatctacgtgatcgagaagtcacaacaacaaattctgggcagaaccaaattggtacaaccatcccagatatttcagaggaggaaagaagactcttggatgcaatggtgagttctaatcccttaaatcctgtaaatcctacttgcttctagcactggaatgttgagtatgagcaggaacaaattgcaaaaaatagaacaacatcaaagcaaggcaatggtgacaaatacacttggtcaatctgcattgaagacaattgttatcgaccaaaatgttccacaacctgtgcacctggggagagacttgtatgaagacggagaggaagaagcaatgttacatcagtgtagagcagaggcagcaagaaaaggggatctatcaccaatgcatagcggcaaaggtaagaggactcatacaaggaaatttagttgggacgacaaagttagtgattttttaaatgttaggcgacctccaatgagagttgccaaacaaaagaaggccgccccaactacatctacaaagtccaatcattcaaaaaagaaatcatgaattttgaatacatcttgaagaattggattgtgatgaaaaagagttacaaactgaagaaattacaagttcggacaagaagggacaacattttttcggaacaagtcggtgaaggttctattacggccgatagaggggtggcggctcgttgatggggttgtaaagttgtaatggtggtgaccggtgaaggttctattttggtgagaccatggaattgagagggggaaggtgctagctgagaaagtgttgaagtagtgaagcggtgagggagtctctgtggggagctgttggtgggttggtgaggttgtttggcggtttcagatgcagaggtgtaacaagtcggacaagaattggtcagacagggcagtttttctggcaatttttttttttaaaattttttttgaaggttctgtggtgctcaaacttctatgaaattttcagggatggttaattatggttggggtggttgctgcctagaggattagataaaggtggaagtggttgtttagttatagagggagtactttgggcgacggtggagaatggtttggggtgagactttgggcagcaattgtttgcgaattggatggtgaaggttgactagatgaagtgggttttgggcaacaacatgttggggaatggaagcattactgtgttacaatggagaaggggaatagatggagatctttgcgggaaatgggggaaaatggcctaagggcagcgacggggtgcaggggttaatgggagagggttaaagggggagggaggtttattaattagccacgtgtgggacatgtcacatgtggcacattctggtgctaaactgagtttttgagattaactaaaaaagagatcaaatgatttttaatcatcagctgttagcaataagggtagatatagtccaaaagtttaagggcaccagtgagccaaactttaaaccaagggtaaatctagaccttttcacaaagtacaaggacaaatatggaccttccctcctacccctccccccaaaaaaaaaaaaaaaaaagaaacaataaagattctgggtattaacagatgatcctttccttggaagtgtggattcttatctcataagagatgaaggtaataaattcttattctatacaccaatatttttctgtacaataaaagcaagacacattgttcaaagttgtataacctgggttaggcatctactgtggcgctgttatgctcgttatcaattgtttttctcttacggtcatttctataagtttttggagctactaattggtggtggggcccgattctttttttatatttgaccatttgtgcctttattaaggaacagaaaagttttaatttgttgaatgtgggcatgctggctgtaggttgcaactggagatggaatggctatggctcatcgagctcaagctgtaatttccaacatggagtaagtgatcctcagttgcattgcattcttattgtccaatatcactgtgtctgctttagtgagttgtgctgtaaaatctgctaatctagatttcatccattttttgctttcttgatttctgcatcctgttctgcaattcattctgaatttgcacttttgactaaaaatacaaggtatgttcaaaatgttgtgcggggtgacattggatctgacttgaatttttcaggataaagggcatcaaattttcttcattgaggggatttaattgatatagaaaagatttctcgactagcagtccgctctctaattgatggtcctcctttgatcgtcttttggatggtagacaagtctttgtttgcgaggttgatgaactatccccaacggttgcattattaattacaattctcttcttcatgttgccaggtttgtgcaattccatccaactgccttggctgatgaaggccttcccaacagaccaagtgccagagagaatgcttttttgataactgaagctgtcagaggtgatggaggcatcctttacaacttagatatggagagatttatgccaatgtatgacaaaagagcagagcttgccccgagagatgtggtagcaagaagtatagatgaccagctcaaaaagcgtggcgaaaagtatgttcttcttgatatcagtcacaagcccagagagaaggttctgtctcattttcctaatatagctgctgagtgtctccgccatgggttagacataacacagcagccgattccggtggttcctgctgctcactacatgtgtggtggagttcgtgctggacttgagggtgagactaatgtgcaaggcctttatgtggcaggtgaagttgcatgtactggtttacatggtgctaaccgacttgctagcaactcgttgcttgaagcactagtgtttgcacgaagagctgtacaaccttcaattgatcacgtgaatgtgtctagaattgatcactgtgcttcaagttggtggccgcgacctgtagccccagtggtaataggagatacagtacttaacaaagtcattcgtcggacaagggaagtgaggaaagaactacagtcaatcatgtgggaatatgttggaattgttaggtctacctcaagactaaccgcagctgagaagagaatcaatgagttggagttggaatgggaaacatacctatttcagcatggctgggaaccaacaatggttggactagaggcttgtgagatgaggaatctcttctgttgtgccaacctggtagttagcagtgctctttctcgacacgagagtcgcgggcttcattacaccattgattttcctcatgttgtggaaagcaagaggttgccaacaatcatttttccttcacagcgaaatagctcgtggagctcacggcaattacacaggcagcagatatgttagatgctcttacctatttgccaatccttcctgctaaaacttgtcaagcgcagggcatgattagtttttaggtaaagaagatgcttatgggttattgtcattcttcctgccattacttttggggcagggcaacgtcattctttggtgaaaaaaatatatatctatacatacaacccatataattttgaatgtgtttccctatt

SEQ ID NO: 7 - DNA, 5238 bp, Artificial Sequence
  Description of Artificial Sequence Synthetic polynucleotide
tcttagtttattatatatataccatgacctgatctcgtcactactcgaccgaggttaggcttggcacttattgggtaccgttgtggtatactcatgctactcttctgcacatattttttgtgtgcagatccaggtgctctttatcagtgagccgggacagctttggagacttcaaggtatatcttccgcgtccgcagacctcggagtccccttctactcttccccacgtccattatcttctgtattttccttttgttagactctgaagtatagagacactagattttccttctgttgtttgtaacttacgatattccgggttttgggagagttatgtacatttcgagagtgaccgtggtatatgccgagcgacatcttaatttcttatttagaatctacctgtaaattgctagttttaatgttttcgtttcatttccgcaaaagtgttaggcttacctagtcgtagagactaggtgtcgtcacgacggttcaagaagggagaacttgggtcgtgacaccttaatattattgtgccatttcgaaccaaaatattaagtaatttttattataattcaaactattaatagtaaatttatgttttttttgtatatataacaagaaaagagtaatctgatcaggaaaaaacatagattcaaattagaactcatgatattactgttcaaactgtaataactttaccatttgaatttttatgactattttgaatgtcaagtaaacatttcaaggtacaataaaaagaaagagcagacaattggtatcttctttagaaatatttgtttctatattttcaggtccacttctaatgtaacacatattttttcttatgttaaaagaagacatctcatcttatttaatactattaacaaatgaactatgaaaaatgcaaactctttgattgttcacaacttgacgaggtttaaataaattataatctttgtcttaaatatacgcgctaagaaactaccaaaatgtgttgcctatgaattttgtgctaccaaaaatagaatatgaggaaataacaactcacaaacacaaagaaaattaagttgatgaaatattgtccaccttttatttcttcttcattaatgtttaatcccaacaacataagtttaaaaccaaatgattaactcaaaaatgcaatagtttttattgaataaagatgcatatgaaatatccgtatttaaacatattatttaaattaactaaaagatctttgcaaaacaattggagtaatacaattaaggaaattatttttgtaataaaatgttgggacatgctcagcagcatggccagccctcctgtagtatacatatatgcagcaattaagtagtccttaatgattgaattacattacacctaaacaaatgcataaaccacgtggtatttgcaagtgaacagctgaaagtccatagcacctcaaaaaagactaaggtaacgtgggtatcaatatatgcagcaattaagtaataaggaccgaatcacatagtatccctaaacaaatataagtgatacatagtatcccctaaacaaatataagtgataaggtacgaatcactaaacaaatgcataaaccacgtgggcgtttggaagtgaagtcataagcacctctaaaattgatcaaattacacagttatccctaaacaaatgcataataccgaaactacccctgcacgaccataatacttcacttatatagttatggcttctgcttccttggccagcaatcatttcatctgtaacttcgaattccagttccattttcacttgatcgtacttcgaagcgccatattttcccctttaagctaaggtaattgtattgtttaacaaatttacatgttccaaatgaatattaattgttaattcctattatcatcctgaaacaaaattttctctatatgtagcctttacgtactatattcgctcatgctttaatgtgtaatgcataacaagattttaccttaaatatccagatcgatcatatggcaactggtatcgcttcaggatgcggacagttacacttgaggaagcctgtctacttgaggaatagctatggaaataaagctcactgtcattccaatgtgattctcaacggcacgcaaaaccagatcgcttggtacgttttaggggtaaaccagtaatatctttttcatgaaatgattaatagtcaaactattatccatggctcactttacgcaataaattttaaaagtattcttaaacatcgcgctaataatctgatggtgatatgcttttccaggtctagttgggtttcaaatgtcttgcgagttaatagaagtagctatccagaatgtcaagtgatcaagacaaactggaagtctagccgaggaacaatcaaatcgtgccagcagagagatggatcagttactaggtacttcgatttcactgtgattggtagtggaattgctggccttcgatatgcacttgaggttgccaagcatggaactgtggctgtaataaccaaggctgagccacatgagagtagcactaactatgctcaaggtggtgtaagtgctgtgctctgccctttggattcagtggagagccatatgcaagatacaattgtggcaggtgcttatctctgtgataaggagactgttagagtaagttcacacagaatatattcagtttggagactataaatttcccccttttgcctttactataagtgtatctctcctttaatttctccaggtagtgtgtactgaaggacctgagagaattagagaactgatcgctatgggtgcttcattcgatcatggggaggacggaaatctggatctagccagggaaggaggccactcccatcgtcgaattgtccatgctgctgatatgactggcagagagatagaaagagctctattagaggcagtttttaagaatcctaatatacatgtgtttcaacaccattttgctatagatttgttgaccactcaggttagtttattagtgtgaaaaaagtttttctatagttgtgtaaaggtggcaattgaactgttcagtaaggatttaatgttgtaatttgtatgccaaatgcaggatggttctgacatagtatgtcatggcgttgatactatacacacggaaacgaaggaggtaagcaccagttgctttcactcatgttgacttttgaacatttcttcaaaaggacacaaattgatttttatggcttctgcgctcttacttgtgttttttaggttataagattcatttcaaaagtgactttgctggcatcaggtggagttggacatatctatccaagtactactaatccgacggtatgggtttcaatatcttttcctacaaaatcttgttagttataattaatgtcacgctccaaaaataaatgagctggacatggcactcgaacgatgccttgaaactagtgacagagcgaaccaacatccattatatactccctgccctaaatgcctaaaaaaggattctgggtagtcgaataacagatgattctctgcctggaagtgtgaattcttattctatacacccataatttatgtacagtaaaagcaagatacatggttcaaagttttactaattacccgggtcaggcatctgctgtggctctgtaattggtggttgggtcaattctgtttttatactttgaccatttgtgccaacagaaaagtttaaccctgttgaatgtgggcttgtttaaatttctaattcatattgacattatgatcataggttgcaactggtgatggaatggctatggctcatcgagctcaagctgtaatttccaacatggagtaagtgatcctcagttgcattatttttattgtacaatatctctgcctctgctttagtgagttttgtgctgtaaaatctgctgatctagatttcatcctttttttccccctgatttctgcatcttgttcagcatttcgttcagaatttgcactttcgactaaaaacacaagggtgttaaaaaatttgcattattaataacgattctcttcctcgtgttgccaggtttgtgcaattccacccaactgccttggctgatgaaggccttcccaacataccaagtgccagagagaatgcttttttgataactgaagctgtcagaggtgatggaggcatcctttacaacttagatatggaaagatttatgccaatgtatgatgaaagagcagaacttgccccgagagatgtggtagcaagaagtatagatgaccagctcaaaaagcgtggcgaaaagtatgttcttcttgatatcagtcacaagcccagagagaaggttctttctcattttcctaatatagctgctgagtgtctccgccatgggttagacataacacagcagccgattccggtggttcctgctgctcactacatgtgtggtggagtccgtgctggactcgagggtgagactaatgtgcaaggtctttatgtggcaggtgaagttgcatgtactggtttacatggtgctaaccgacttgctagcaactcattgcttgaagcactagtgtttgcacgaagagctgtacagccttcaattgatcatgtgaacgtgtctagaattgatcacggtgcttcaagttggtggccgcggcctgtagcccccatggtactaggagatacagtacttaacaaagtcatctgtcggacaagggaagtgaggaaagaactacagtcaatcatgtgggaatatgttggaattgttaggtctaactcaagactaaacactgctgagaagagaatcagagagttggagttggaatgggaaacatacctatttcagcatggctgggaaccaacaatggttggagtagaggcttgtgagatgaggaatctcttctgttgtgccaacttggtagttagcagtgctctttctcgacatgagagtcgtgggcttcactacaccactgattttcctcatgttgaggaaagcgagaggttgccaacggtcatttttccttctcagcgaaataactcatggagctcacgccaattacacgcgcagccgataagttagatatctctttcctatttgccattcttcctgccaaaacatctccagtgcaggacatgatttgtcgattttaccgggttgcaggggtgtatctattggaaggggtatgtgttcacatgaacatatactcttttccctaaattatgtataataatatatatttcttcaatgttttttaagtatgtaagtgtgtcagtcaagct

SEQ ID NO: 8 - DNA, 24404 bp, Artificial Sequence
  Description of Artificial Sequence Synthetic polynucleotide
cttattcctatcccgaaggagtagagaggctgtttccgaaagaccctcggctcaagaaaacaaaaagacaaaaggacaaaaggagacaatattagtatcaccacagcaatcataggaaaaataagaacaccatgaaatccagaagaaagatgcaaagcaaaagcgatacctagtaaataggtcctgcactgaaaagcgaaatagtaagacacaacattgccactagctatcttagacaaaaatcctacctggctagtcccacaatggtacaaagtaaggcaagactcaactacctcctaacctacaaccctaatactcgacctccattgaacaccattgacaaagaaagatttaataagggaaaatgaaaatttagataggttggcaagtccttagtcttagagtaaatgatcttcattgggtcttattgtttccttgcaatacttgttcagccttgtaggttctgctttttgctaaaacaactctttttagtcgtcacttgtcacttacaatttcctttctcctaatagaaacgtgtccggaaagctgacttttggccctgtatgtgtttctgttttttggagccttcagaatgatagtactagctgttcaggtgtctattgaaggtcttcattgaagcaacagtaaggacatagttagaaaaactgttgctgaaacaaaaagagaaatcattgctttcgcttcctgatgttacatgacataagtattgagtcaaggccattttaaatgaatgccaatgtatctatgttcaggtgtctattgagggtcttcattgaagcaacagtaaggacatagttagaaaaactgaaaaggacttcttcttaacgtgtttcagtctcatgattttaaaggaaatgcattttgtagatacattggcattcatttaaaatggccttgactcaatacttacgtcatgtaacatcaggaagggaaagcaatgatttctctttttgtttcagcaataagtgatgaagtcttgtggcaacttccatttgtaaattcattctgtacaagtggattatagcaattagttgataagttcatacctttcttttctctactttttctcttgcaatatgagatatcacaggattagagacatgatgcacatacatttgtggttcacttgaatgaaaaaaaagtatacgtttcttctttttatgtttcatttccttttggtttttcagtatctggtatggaagttgtgtaatgaacagttttttacacgcatgttctgctgcagcattaacatggactagccatcttgtcgtgtgagtccaatttgtggaattccagtttaagcccatcaattgttgaatctctaaatattttcattgaattgaccatatgttagatgatatgttttctcattgtcctttcaagcatttctccttttgtttaaatgtctccacatgcttttctaccccttcaatttctgatattttctaaaagttcctttttctttaataaatctactgctccaaatgaatgaaagaagagttgtgaagagttctgacttcaagaccaagcacagttttggttgggctttcaattttagcttttaggaagttgttcctaagattgtcctgaaaccaaatcttttctttttgtagcttctactatattcgttcatgctttaatgcaattcataacaaggtttcccttaaatatccagagtctttaattttgctttcgagtcccagctgtgcaaaacgtattactgaagaatagttttctgggagatgttacaagattttgttagaagggtctttcccttctaaagtgtttggttcaagttatctaaatgtcaagagccgatattttgttattaagctcaattctttgccttgtttgggtgtttttagaagtaatcaagatgatctttaaatgtttagtttcacaaacattctaacctcggaattgggagtgctaatctgatatgaattcctttgacctggattgtcagtcaggaagatcatatggcaactggtatcgcttcagtaagcggacagttacatttgaggaagcctgtctactggaggagtagctatggaaaagctcagtgtcattccaatgtgatcctgaatggcatgcaaaaccagatcccttggtacgtattaggatgatgtttcttcacctatctctccttctctctctatctttccctgctgtattatcttctttctattgccccatctcctctcttctgggggaatgattgccaaaatattttattctggagcttttaagatgttattacatttcaaattgtacggagataggcattcacgcgttggctgttggactgtttattcctccaatgagaacgtactggaaagaatctttgtatacgtgtttaggcataaaccagtaatatctttttttcatgagataccagaatcctaaaagtttttcttttttagtcatttcctgattctttagaaaaaaaagaaaggttatcattttcattttctggtggtgatatgctacttaaattttccaggtcttattgggtttccaaattcttgcgagttcatagaagtaactattcacaatgtcaagtgaaaacaaactggaagtctcacagcggaacaatcaattcttgccagagagatggctcaactaggtatttcgatttcgcggtgattggtagtggaattgctggccttcgatatgctcttgaagttgccaaacatggaactgtagctgtgataaccaaagctgagccacatgagagtaacactaactatgctcaaggtggtgtaagtgctgtgctctgccctatggattcagtggagagccacatgcaagatacaattgttgcaggtgcttacctctgtgatgaggagactgttagagtaagttcacagaatgttattgtctcttcagttccatgaagcagtttgccctttttcagttttttctcattggagagtgtttccttttacttttccaaatttttcccggaatacttggttggatgaataggaaagttctccatctttcagtttgaaaccaaaggacctttctgtttagacggcttttgccacaggatctagagctgccaaaaattggaaaagagtaagagcaatggggcagttaggcttgttttcaatttgaagctgaagtgccaagcaacaatactacaagaaaggctctatgtctattccttgcttctgatataagtgcttaaaagttggaactgaagaaagagttggagagttgggaaaccaatggttggtaatatggacgatttccactggtttatgtagtactcctattctttatggttcagattgttgacttttgtctttgaacattagggaaagagagtgaaggtgacatgcaggaaagaagagataagaataagacaggaaattcacatgtgttgtatcttggtttgattttgggatttcatttagacagtaccatgttttcttcttttaaggagaacacacaaaagtacctttaacacttctgacttccctaaatcagcaccaattttttgagggtaactgctgataaggatagacactccaaggcgatccaggattcctagtcatataagattctctcctatccccacaagggtcctatgtgcagaaaactctatcccttctccctcgagctttaacctaggaatccatttgattatgtgaaaaagcacaagagaaaaatatattattgatttattaactatgttacaatgagccctatttataagtatacataatacatacaacaacaacaaccacccagtatagtcccactagtggggtctggggagggtagtgtgtacgtagaccttacacctaccctggggtagagaggctgtttccgataagaccctcggctctctccttccaagaactctccaccttgctcttggggtgactcgaactcacaaccttttggttggaagtggtgggtgctaaccactagagcaacccactcttgtcataatacataacctactcctattacatacgggactaggactaattatatatatatacacacataactatctaacactcctcctcaagctggtgcatataaattatatgtaccgaacttgttacatatgtaactaatacgaggaccagtgagaaacttggtgaaaatgtcagcaagttgatcattcgacttcaaaaactttatagcaatatctcatgcgagtattttttttctgacaaagtgacagtcaatctcaatgtgtttagttctctcatggaacactggatttgatgcaagatgaatagcagcttgattatcacacacaagtttcatctggcttatctcaccaaattttaactccttgagcaactgtttgatccaaattagctcacatattgccatagccattgctcgatattctgcttctgcactagaccgagcaaccacattctgtttcttgctcttccaagacaccaaattacctcctactaagacacaatatccagatatagaacgtctatcagaaggtgattttgcccaatcaacatctgagtatctaacgatctgctcatggcctcgatcctcaaacaataatcctttgtctcgggctgattttatataccgaaaaatgcggacaactgcatcccaatgactatcacacggagaatccataaactgactcacaacactcataggaaaggaaatatgaggtctaatcactgtgaggtaatttaaattaccgaccaaccgtctatatcttgcgggatcgctgaacggctccctcgtcctcgcagaagtttagaattcggatccatcggtgtgtcaataggtctacaacctgtcattcctgtcttctcaagaatgtctaaggcatacttccgttgtgagatcacaatatctgagctagactgagcgacctcaatacccaaaaaatactttaatctgcccagatccttaccctgaaagtgctcaaagagatgttgcttcaacttagtaataccatcctgatcattgccggtaataacaagatcgtcaacataatggaccagataaatacagagatttgaagcagaatgctgataaaacacggagtgatcagcttcactacgagccatgccaaactcctggataactgcgctgaacttaccaaactaggctcgatgagattgctttagaccataaactgaccggcgcaagcgacatacaaagccactagactccccctgagtaacaaactagatggttgctccatataaacttcatcctcaaggtcaccatgaagaaaaacattcttaatgtccaattgatagagaggccaatgacgaacaacatccatggataagaaaaggcggactaatgctattttagccacgggagagaaagtatcactgtaatcaagcctgaataattgagtatatcctttggcaactagacgggccttaagtttttcagcctggctatccggaccaactttgactgcatacacccaacgacaaccaacagtagattttcctgaatgaagaggaacaagctcccaagtactactcgtatgtaaagcaaacatctcgtcaaccatagcccgtcgccatcctagatgagatagtgcttcacttgtagacttagggatgaaaacaaaggacaaaaaagatacaaaagcataatgagggtgatgacgggcgatgataacttaaaccgacataatggggattaggattaagtgtggttcatatacctttccgaagtgcaatcggctgactaggaggagcaagtccgcagtaggagcagggtaaggtgcaggacgtgaatcagctgggcttgatgctgggcgcggacgacgacgataagtcaagagtggtgttcctgtggctggggttctaggaggagcaacactagattccttaacggtttgtgtgggtaagacttttgtgattgagaatgtaggaggagctatagttggtggggctagagctagaatggggctgtaggtgatggagacgtggaggaaggtgaaggggaaatagtgaatgaatctccaaaaagatggaactggtaatacctcaaaaatgtctaagtgaccacctaggcatgtgaagtatgactgagtttcaaagaaggtaacatcagcagacataaggcactgctggaggtcaggcgagtagcatcgataccccttacctaaaaatacacacttaagagcacggggagctaacttatcttttcttggagtaaggttatgaacaaaacatgtgctcccaaagacacgaggtggaagagagaagaagggtgagtgaggaaacaacaaagagaatggaacttgattctggatagctgagtatgtcatacaattaataagataacaagatgtgagaactgcatcccccaaaaatcgcaacggaacatgatattgtatgagtagggtacgggcagtctcaatgagatggctatttttcttttccgctaccccattttgttgagatgtatatggacaagatgtttgatgaatgatcttatgagatttcataaactgctgaaatataggagacaaataatctggggcattttcactatgaaatgtgcggatagaaatcccgaattgattttgaatttcagcacggaaaacaactcagatcgatttttcatcaagaatatccaagtgcacctggaataatcatcaatgaaactatcaaagtagcggaatcccaaggtagaactgacctgactaggatccctaacatctgaatggactaaagtgaaaggtgactctgctcgattatcagagcgtcaagggaaacgagagtgagtatgcttaccgagctgacatggctcacacactagagtgtacaagtgagacaaatcaggtaccattttctgaagttttgataaactgtgatgtcccaactgtttatgtaataaatctggtgaatcagtaataaaacaagttgttgatatgggttcatgtgatttagcaaggataaggtaataaagtccatgtgattcatgtccggtaccaataatccgccccgtactgcattcctgcataaaaacaaggtcatcaagaaataaaacagaacatttaagtgatttggctaagcgactaacaactatgagattaaaaggactaccaggaacataaagaactgaattcaaaggtaaagaaggaagtggacttgcttgacctattccagttgccgcagtttgagacccattggccattgtgaccattggaagagattgatgataagaaatagtagtgaaagcagatttattaccatatgatgagacgctcctaaatcaataacccaattactgggagaagaagattgtgagacacaagtaacgctagtacctgtttgaacaacattagcaatctcataagatgtcttttacatgctttgtacagaaggaactctatatattccgataaagaaaccatctggattggattcaatgcattggatccgacggatacaaatgacattataatgttcacgctggaaaaatcagaaaatattctggaaactactgttcacgccagaaaaatatgaaagtggtaggaatttggtttaaattggatgggtaggctcggaattgcaaggtaaacaagttgtcctgaagagtcgtcgccaaaaaagggccgaaaatagccccacgcgcgtcgcgtgacatctgaacttagccggtaatttttttttctgtcggcgtgtgagggcgcgtggagggtggtctgtcggagatatttttacgggttggtcgtcaaaggctgatctacttcgtggtggtgttggtttttgcacaacactgatgaaaagtggttagttcttgcggtagacctgtgtttgctggaaagacttccggttgactgatttctcttcccggccgctggaatttatgcacaacgataaatctctcacgatttctctaatagcatgtgagaaagcacgggagaaaaatatattattgatttattaactacttataagtatacataatacataacctactcatattacatacgggactaggactgattatatatattcacattctaacaaattttcttaatgctaacaactcatgcagagttgtgacatgcccaattctgctgagagtagtaaggcccagcggatttcgcatctactaggcccattaacaacatacaagagggagaggggatagagttctctgtacataggactcttgtggggataggagagaatcttattatatgatacaacttaagctaggaatcctggatagcctgggagtgctgtatattattttcatttgtctctgtaaatatatactggcttcaaggaagttttgcgttcttgggtgacgggacatcacaactggtatcttcttatcagttgagataatcgtcgtccatgcaattgtactttttatgcagtctctgtgctaaatgaatttacaagatgactgatttattactccaacattgtgtttagtatactcttttgaagtcttcatttttataagtgtatctcctttctccaggttgtgtgtaccgaaggacctgagagaattaaagaactgattgctatgggtgcttcattcgatcatggggaggatggaaatctgcatctagccagggaagggggccactcccatcgtcgaattgtccatgctgctgatatgactggcagagagatagaaagggccctattagaggcagtttttaagcatcctaatatacatgtgtttcaacaccattttgctatagatttgttgaccactcaggtaagtttattagtgtgaaaattttgctatagttgtgtaaaggtggcaattgaactgttcagtaaggatttaatgttaatttgtatgccaaatgcaggatggttctgacatagtatgtcatggcgttgatactataaacacggaaacacaggaggtaagtacagttgctttcactcatgttaacctccatgttttgaacatttcctcaaaatgacataaattgatttctttggcttctgcgctcttattttgagtttgcaggttataagattcatttcaaaagtgactttgctggcatcaggtggagctgggcatacctatcctagtactactaatccgccggtatgggtttcaatatcttttccttcataatgttgttagttatgagggcactccttgtaatcagtaagattattcagtcctaaacagtgatatggggggagaaggtggggaagggaatccctggtgctgggtggctaatagttacttttatgactagcatacatatgcatttagttgttggcgataagcttcgtcacaaagtatcattcgtaattcataaggaccttaacaattgacattggtgcaaacaacattccatagagtatgaaacatggcctgatttaaaaccctttcatgttaagctaacgtgctaattagaaataacagctgactgccctaagtcacaaaaaagggacaagtccaaatttacccttgtactttgcgaaaaggtttagatttatcatccggttgaagttagtttggctcactagtgcctcgctgttaaacttttggacgatatttacccttatttgctaaaggacctttgtttgaggtttaattgtctctaagtatactgaataacaccagagactgccacgtgtcccacacgtggcctgaaattaatccctcccctttaacctgaccccgtttgaccctgaaaagtaacccccctctcactaaaattaaccacccctcttacccccacccttttaccacaaacaaggtggccaacggaaaaacaactcctacactgtccgtcttcattacacttctgcaaccaccaaacaacctcaccaacccaccaatagcaccccagagactcactgctcctccacttcaaccctttctcggcgaacaccttcccctcagttccacggatctcaccaaaatagaaccttcatcagtcaccaccattacaactgtttacccgaaaaacggatagagttgaatttattcgtagttctaaggatatgtgatatagctcaatacaaattgtaaggataaaaggaaaatatcaaatatggaccgcaaagaagactaaataaacaaagttgtaacaaaagcgatttttgggaccaagcaagatgaatcagtttatgaggcttaaaagaataactctcgaatatcagagaatatgttgcttgaattacaatctaaactccaaaaaaactgcccttccagaaatgataaaacccctctttatagtagagggaccttactttaaatataattaaaaaatactcagtgggagatccatgataaatcaacttttccacaattcctgccaagattctctcatctagtgggattgcaacggcttatgtctgtgagtccgatctgactcgggcttttgatctcggcttgagcttgattctaactcgagctctcgatctttggcttaagctcgattctaactcggacccttgaattgattcggggtcaatgttggttggtctctagctcataagcttgatagctttactctgcatcatagttagatttggatttgagctcaataacaatatcgaactcgacattgatcggcccctcgggttcgaggcttgtttgttccatcttcggaaactatctcgaagtctcacttcgatcgatcatcattcgaactcgatcagacgtgcgaaggccgaaatctatttcgaccgtatacagatagtcccctcgcttcttagaaagaatgtggtgagaaacgatatgatcttttaacggctcgatcggattataacctgacgtttccatcgggctcgatcatgacgcatatgatagctgttccgtcggtttagtctttcaaggcatttaatgcatgtcagatggtggtcggccactgctgatactgaaccgccatcgcttaaacctataaataacccttccttttatcatttaccacttttacatcttcaatcttccaaatttcccaagtcccttttcgcattacccgtaaatctgtgatttctttttacaaaagcccctcttcaattctaccaaatctttgtcactttcttctattcctcacttttgaattcgaaaatggcgaaaacatcacaaaccattcctcagaaagagaaggcttcatcttcacaatgtgctgccgataagacaccagcaaaaccacgatctgaggagtgcgttctcggggcgtgtgttcttacttctgattttaaggtcgataaaggctcatcggtccctggccgatgtgagccagtatcgaggtacatatgttcgataaccgagaagcacctcaaacagctaaagaaagattgcaattgggataaaaaagaaataatgattccttcttctgacgaagatatcacttcttacgtgaaagggtttttgaatgtgtatacttaccctttcacgttaggtcccctcgattccgttattattgacttctgtcgtcaataccaaataaccataggctaggtccatccttctttctggcggatcgttatcctgatccgatactttgtgagcaaaatcgaggggatgccgttcaccctcgaccatcttatccgattgtaccgtcctcgactttttcaaggagggttaataaaacttcagcgtcgggctaccaaggctctgttctcgagcattgacgaggacagggatcggggctggatgggcaggttcgttcgagtgaggacttcggacctgattccgaccgaaaagatgccctttcccgaggagtggaatatgaaacgtaagtgtgatcctgctgttacttctattttgttctttcatttcttttctcaacgacactcttctttttgtgatgtagcggttccctggatgcccggagcagttctcgatctcaagaactgggtacgagctcttgtttcgacctttacatacgccgagcgctcatgccgtgatttgtcaaagggtggtgggaggccaagaatcatggtaagcccctttctagtactctttgataattcgaacgaggtggtttccaaatattttaataaattttttcatatccaggtttgggcaaagatgcagttttgaggcccttgtccaacgaggaagaaacttcggcctctgttccaaagccggtgaagggaaataaaaagaaaagagccaccgttcccgaagatccaaacccgaagaagaggacggctcgtaagccgaacaagaatgccattcctttgaccgtggaatcagttctgcgtctaagggatgaagatgaagaagaagaagaagaagaaaatgatgggtccgcgctggcggcccgaacgaagagaaccaccgatgccccatcggcagctggatcgatgatgctttatgaggctccgcctcgaactgaggatataccggagaaagattcgggtggaatccctgaattatcggagatcgaagacgcatctcatcggagccaacgggtgggtgatatgtctgaaagggccctcgaatcccttcgaaccgaagagaatgctccaagtaattcattttggggcagcagcaatcgaagattcgcccacctttcccgcttttttcgcaggggtgattcgggaagcccaagctctgggggccctcgatctagataggcctcacgatgaagaggatccctttcgtgacctgtttaccggtatcgaggacgttgccggcactggtgatgaatcagatctttttcacggggtacggcaggctttgaatcaggtaagccttaaaattattttgtcggtactatctttatgtttacttttcattaacttcgtttcttgtttctttgtaggcaacgacagtttatcgagaagcatgttctcggtcccaaaaagcgctgcgtcgatacgaggccgaccttcaacgggttactgatgagagaaactccctaacacttcttttagggcagagagaagaggaaataaaagacctccgagctgagttggccaaggcttatcgagatcaggccgatttgtctgagcaggtaatgatacttttaaaagcctatgggcttgataccggaacgatggctaatttttcggtctcacagttgcagcaaaaaattgagatgatcgggaagcttcgtgaggaggtcgacgtgataaaaacagagtctttgcagtggaaagaaggtatgaaccgctttgatgcagagaaagaaactgctcgagcctagttatcatctgccgaaacccaacttcagagaatgaaagaaaaaggcctggttcaagcaagaagaatagaggagctcgaggctcggttggcctctgtacttgccaaggccgaatctgatgccgaaaaggcaaaagccgatgcggatgcactcgtggccgtctatcgggccgatgctgaagctgcccaggtctaagcaagagaggcagccgagactgccgatactcgagcacattgggtcgccgaacttgctaagtgccgatctcggagggaaaccctcgaggagatccatgctcgtggtttcgatctcgctgaagagataaaaagggccaaagaactcgaagccgatgctgaagccctggtttccgatgacgatgatgatgatgacaatgatgatgatgatgggagtaagagcggatccgagaacgggggggagcccgatagagaagagaccgctcctgaggatgaccaggaagcttagtccttaatttttacgttgtaatcaatcatgtagacaattttgtatatagacaatattgccgacttgcttctgttttgtgaagactttattcatgccttatgaatgttttcacaaggatttcaacaacttaatcaaatttggacttcgtagcctctataatcgagcgagtgcttattcaaacttgaagtgatgtagcccttaggcttattagttgagtcaatgattcgagctcgaagaaatatagcccgtaggcgtaatggtcgagtgagtgcttgctcgaactcgaaataaaagtagcctgtaggcttagtagtcgagtgaatgattcgaactcgaagtaatgtagtagcccgtaggcgtaatggtcgagtgagtgcttgctcgaactcgaaataaaagtagcccgtaggcttagtagtcgagtgaatgattcgaactcgaagtaatgtagcccgtaggcgtaatggtcgagtgagtgcttgctcgaactcgaaataaagtagcccgtaggcttagtagtcgagtgaatgattcgaactcgaagtaatgtagcccgtaggcgtaatggtcgagtgagtgcttgctcgaactcgaaataaaagtagcccgtaggcttagtagtcgagtgaatgattcgaactcgaagtaatgtagcccgtaggcgtaatggtcgagtgagtgcttgctcgaactcgaaataaaagaatagtagcccgtaggcttagtagtcgagtgaatgattcgaactcgaagtaatgtagcccgtaggcgtaatggtcgagtgagtgcttctcgaactcgaagtaatgtagcccgtaggcgtaatggtcgagtgagtgcttgctcgaactcgaaataaaagtagcccgtaggcttagtcttgaactcgaaataaaagtagcccgtaggcttagtagtcgagtgaatgattcgaactcgaagtaatgtagcccgtaggcgtaatggtcgagtgagtgcttctcgaactcgaaataaaagtagcccgtaggcttagtagtcgagtgaatgattcgaagtcgaagtattgtagcccgtatgcgtaatggtcgagtgagtgcttgctcgaactcgaaataaaagtagcccgtaggcttagtggtcgagtttatcttaattctgtttgcataataaatctccaaatagaggaatttttcttggatataagatgttggcaaagaggagaactttctttgcaagtcactatacatgtgtttatgtttcgtgtctgggttcgggccaactacatgagcatggatcgttttgaccatttggcttttacaacttttcctattggaaacccgttgttgcgaaataactttcttgcatcagaatttgatatattcgagggctaatgccccccagtattcgaggtcgattgtaaagaggcctcggatactgttgtaagcgcagcacgatcattggttgcctcattaaaaaccttgccgaaaaacccatttgggataaaaccggtctaagggaaaaagagtgcaacacgtgctttcagatcttaaggcttcgtattgaaggatccatcttagctcctgatcgaacttctgcaggggtcagttttagaatgtaaatgaatatgggagggtcgtaccttagcagtagtatcgttttagatgtgacacattccaattgcttgataattgtttgctgtttataatgccgagcttgtatgatccctttccgacgttctcgagaacctgatatggtccttcccagttcggtcctagttttccttgttcggatttcgggtattgagggtgactttccttagcactaagtcctcgggcttgaaatggcggagcttggttcttcgattataatatctttcgattcgttgcttttgggcggccaattggacgagagcagcttctcgttttttgtccaataattcgaggctagtgttcatagcctcgttatttgattcttctgtcgtgtatcgaaatctggtactgggttcgccgacttcgactggtatcaatgcttcggacccatatactaaggagaacggggttgcccccgtactggattttgacgtcgttcgatatgccctaaggactttgggtaggatttctctccactttcttttagcgtcgttcagcctcttctttaggttttgaatgatagttttgttcgttgattcgacctgtccgttcccactggggtgatacggtgttgataatatccttcttattttgtgatcttcgaagaatttcgtcactttgctgcccacaaattgtttcccattgtcacatactatttcggcgggtatcccgaatcggcatacgatatgatcccagattaagtctataacttctctctctcttactttctcgaacgcctgtgcttcaacccatttagagaaatagtcagtcataaataaaatgaatttagctttacctggggtcgatggcagagggccgacgatatccattccccatttcatgaatggccatggggataggaccgagtgaagttgttctccgggttgatgtatcattggcgcaaacctttgacatttgtcacattttcgaacaaatgccttcgcatctttgcccatatcgatccaataataccctgccctgattattttttcggactaatggatcgacaccggagtgattcccacaagtgccctcgtgcacctcacgtaagatgtaatcggtatctcctggacccaagcatactgccaatggtccatcgaatgtccttcggtatagcgttccatctgaagccaacgtgaatcgagcagctttagtccgtagggcccttgaatctttaggatccgatgggagctttccgttctttaagtattcaatatacttatttctccaatcccaggttaagctcgtagaatttatctgggcgtgtcctttttcgatcacggatctcgagagttgaacgacagtccccgagctcaaatcaccttcctcgaccgatgatcccaaattcgcaagtgcatcatcctcactgttttgctctcgtggaacatgttgtaaagtccattgtttgaaatggtgcaaagtgacatgtagtttgtccaaatacctttgcattctatcttctcgaacttcgaaggttttgtttacttgacttaccaccaacaagagtcacatttggcttcaatgacttctgctcccaagtttttaactagctcgagacctgcaatcatgacctcatactcggcctcgttgttagtcaacctggtagttttaatagattgcctaatagtgttacccgtgggtggctttaaaactatgcctagcccggaccccttcacgttcgaagccctatccgtaaaaagggtccataccctcgatgacgtacccgatttcaacaggagttctttttcgacttcgggtacgagggttggcgtgaaatcggccacgaagtctgctaaaatttgagacttgatggccgtatggggttgatattcgatatcgtacccactgagttcgacgatccatttggccaatcggcctgatagttcgggcttgtgcaaaatattacgaagtgggtaagtcattaatacgcatatggggtaacattgaaagtacggtcgtaactttctagaggcgcttatcagtgcaagtgccaatttttctaggtgcggatatctagtttctgcttctcctaaggttcgacttatataataaatgggaaattgcgtaccttgctcttcccgaactaggacaccgcttactgcgatttccgatactgccaagtataagcaaagtttttcgtctgtttttggtgtgtgaagtagtggtgggctcgacagatattgttttagttcctttaatgcctgttggcattccggggtccaagcgaaatcgttcttctttttgagtagggagaaaaatttgtgacttcgatctgatgatctcgaaatgaatcggcctaaggccgcaatccgtcccgttagcctttgtacaactttcacgctgtccacgatggtgatgtcttcgatggctttgattttatcagggttaatctcgattccccgatttgacaccatgaagccgaggaacttgcccgaaccgaccccggaagcacatttttcggggttgagcttcatgttgtatttcctcaaaatctcgaacgtttcctgcaaatgggtcctctgcgcgcagggatttaactagcatgtcatcaatataaacttccattgatttacctatttgttattcgaacattttatttactaagcattggtaagtagcccctgcattttttagcccgaagagcatcacattataacaatatgttccatatttggtgacaaacgaagtcttttcctggtcctccgagttcatctggatttgattatacccggagtaggtatcgagaaaagtgaggatctcgtgaccggccgtggcatcgatcatgcgatcgatgttgagtagcggaaaagaatctttggggcatgctttgttcaaatccttatagtccacacacattctaagtttattcccttttttaggcactacaactatattggctaaccatttgggatatttcacctcccgaatggaccctatcttgagaagtttggttacctcgtcttttatgaatgcgtgttttacctcggactgaggtcttctcttttgcttcaccggtctgaacttagggtccaagcttagccgatgcgtcgttatgtccggtgggatccctgttatatctaaatgggaccaggcaaagcaatcaatgttatcgataagaaattgaataagtttcttcctgagttcggggctcaaccccgttcccaggtatacctttcgttcgggccagtgctagattagtgtgacttgctccggctcctcaattgttgatttggtggcgtcggatcgagggatcctctgatcatcatcttcttcgatcttccggttatctggctgggtcgaagccgatgtctgtgattgctatttggtatctcgttctccttctgagcccgatccctttattggtgaaggcgaggatattggtttcgcttcctcgacagcgaacatttcttttgcggctggttgttctccgtacactgttttgactcctctcgatgttgggaatttgaggacctggtgtagggtcgaaggtacaactctcatgttgtggatccatggccttccgaaaagggcgttgtatctcatgtcgccttcgatcacgtgaaacttcgtttcctggatggttccggccacgtttattggtagaattatctcgcctttggtggtttcacatgccatattgaatccgtttagaaccagagttgcgggtacgatctggtcctgcaggccgagctcttctacgaccttcaatctgatgatgttggccgagctacctggatcaatcaacacacgcttaactttagttttattcatgagtacggatattaccggtgcatcgttatgaggttgtatgactccttctgcatcttcgtcattgaaggacaaagttcctatgggtgtgtaatcttgagttcgagatcgattttctctcacaatcgatgttttagtgcgtttaagcactagtccctgaggggtatcggcgccgccaatgatcatgtgaatgacgtgctgtggttcttcctgttcgttttgcttgccgaaatccctgtttttaaaatggttcttcgccctatcgctcaaaaattcccgaaggtgccctttgttgaataaccgaactacttcctctcttagttgcctggaatcttctgttctgtggccatgggtgccatggtattcgcatatttgattgggattcctccgggcaggatcggtttgcatgggtcgaggccatttagtgtctttgatgcgtccgatagctgatacgatggcgtatgcatcaacgttgaagttatattccgataaccgaggtgcttccttagatccgacaggcctatcgaaactacttctgtttatcagcccccgggacccttgacctcgatcactttttttttattttgcttgaggttatgtctcgattcaccgactctgtggcttccgttgtacggttggtatcgatccccaatcagacctcgttctcgattagtatcccttcaattagcgggttcagaccccaattgatcatcttcgaccctaattttcgattggtaccgattgtgcacgtcggcacatgtgatggctgggtattcgatcaggttatgctttaattgccgtgaagccgtcgaacttcgctcgttcaaaccttgagtgaaagcctgaacagtccaatcgtctgtgactggtggcaagttcattcgttccatttgaaaacgagatacgaactctctcagcatctcgttatccttttgtcttaatcttgaagaggtccgatttccttgttgcgacttttatggcgccgacatgtgttttcacaaaagaatctgctaacatggcgaacgagtcgatgaaatttggcggtaggttgtaataccaaatcattgcccccttcgagagggtttctccgaatgtttttagcaacacagattcgatttcatcgtcttctaaatcattgcccttgatggcatatgtgtaagaagtgatgtgctcgttgggatcggtcgtcctgttatatttgggaatttcgggcatacagaattttttggggattgctttcagggctgcgctcgaggaaaaaggtttttgcacgaattttttcgaatacaacccttttatcattggtggagctcccgggatctgatcgaccctagagttataagtttctacttttttatcgttggtttcgactcgctttgtgagttcctcgagcaatttagcaatttcgggagtagtccccgattcttgctcatttgactttactatggctggttccgttctgtgagtgatttctcggggtggattgggctccggcctgctttgtagctgggtttggctctgcaactgagctattgctgcctgttgggcttgcaacatttcgaaaatcatacgcaagttaacgccgttttcctcgatgttttgggtatctcgagccgcagaccgagtgccaccatggatgctattctcgggttcagcatgtaggttcgcctcaatggctacatgcgaattgatatcaattggcgctccgattcgagctccaacggtgttcacaagtggtctttcagtactgggtgtcaagttgttttcgtcttgaagaccggccccgttatcgatcggtgaagccatttgattgatggttagtcgtagctaatccgaaattcaagatattttcggaaacaagcgtaaaacacaatggcgtgttttttcagattcgtatcaaataaccactgttatccttagccccacagtgggcgccaaactgtttactcgaaaaacggatagagttgaatttattcgtagttctaaggatatgtgatataactcaatacaaattgtaaggataaaaggaaaatatcaaatatggaccgcagagaagactaaataaacaaagttgtaacaaaagcgatttttgggaccaagcaagatgaatcagtttatgaggcttaaaagaataactctcgaatatcagaaaatatggtgcttgaattacaatctaaactccaaaaaaactgcccttccagaaatgataaacccctctttatagtagagggaccttactttaaatataattaaaaaatactcagtgggagatccatgataaatcagcttttccacaatttctgccaagattctctcatctagtgagattgcaacggcttatgtctgtgagtccgatctgactcgggcttttgatcttggcttgagcccgattctaactcgagctctcgatctttggcttaagctcgattctgactcggacccttgaattgattcggggtcaatgttggttggtctctggctcataagcttgatagctttactctgcatcatagttcgatttggattcgaactcaataacaatatcgaactcgacattgatcggcccctcgggttcgaagcttgtttgttccatcttcggaaaccatctcgaagtctcactttgatcgattatcattcgaactcgatcacacgtgcgaaagccggaatccatttcgaccgtatacaacagctttacaaccccatcaacgagccgcccctctaccatagcaaaacccatcgtctttcatttttaatggttgatggtgtcgcatagggaaatttttccggcgacatttttttttattttttaaatttttgaaggttctgtggtctcaaacttttatgaaaatttcagggatggttaattatggttggggtggttgctacctagagataacggtggaagtgattgtttaggtatagagggagtactttgggtgacggtggagaatggtttggggtgggattttgggaagctattgcttgagtattggatggtgaaagttaactagatgaagtgggtttggggcagcaacatgttgggcaatagaagcattactgtgttgcaatggagaaggggaatagatggagctcttcgctggaagttgggcaaaatggtctaagagcagtgccggggtgcaggggttaaagggagagggttaaaggggcagggaggtttattaattagccacttgtgggacatgtgacacattttggtgctaaacagagtaattagatattaattaaaaaaagagattaatcaactgttagcaaataagggtagatatagtccaaaagtttaacggtgagggcatcagtgagccaaacttcaaaccgagggtaaatctagacctttttgcaaagtacaaggggaaatatggaacacccccacccccccacccccaaaaaaaaaaaaaaaaaaaaaacttaaagttctgggtataatagatgatcctttccttggatgagtggattcttgtcaagacacattgttcaacgttgtaataacctgggttaggcatctgctgtggcgttgttatgctcgttatcaaatttgcctttcttttgaaatcatttctgtaagtttttggagcaactaattggcggtggggccaattctgtttttatatttgacaacttgtgcctttattaatgaaaaaaaaagtttttttaatgtgggcatgatggttgtaggttgcaactggagatggaatggctatggctcatcgagctcaagctgtaatttctaacatggagtaagtgatcctcagttgcattgcattctcattgtacaatatctctgcccctgctttagtgagttgtgctgtaaaatctgctaatcttgatttcatcctttttttgccttcttgatttctgcatcttgttctgctattcattcagaatttgcacttttgactaaaaatacaagggtgtgttcaaaatgttgtgcgggatgatattggatatgcaacttgaatttttcaggattaagggcatcaaattttcttcattgagggatttagttgatgtagaaaagatttctagactagcagtctgctctctaattgatggtcctcctttgatcatcttttggatggtagacaagtctttatttgcgaggttgatgaaggttgcattagtaattacaattctcttcctcgtgttgccaggtttgtgcaattccacccaaccgctttggctgatgaaggccttcccatcagaccaacaagaaccagagagaacgcttttttgataactgaagctgtcagaggtgatggaggcatcctttacaacttagatatggagagatttatgccaatgtatgatgaaagagcagagcttgccccgagagatgtggtagcaagaagtatagatgaccagctcaaaaagcgtggtgaaaagtatgttcttcttgatatcagttacaagccccgagagaaggttctttctcattttcctaatatagctgctgagtgtctccgccatgggttagacataacacagcagccgattccagtggttcctgctgctcactacatgtgtggtggagtccgtgctggactcgagggtgagactaacgtgcatggtctttatgtggcaggtgaagttgcatgtactggtttacatggtgcgaaccgacttgctagcaactcgttgcttgaagcactagtgtttgcacgaagagctgtacagccttcaattgatcacatgaatgtgtctagaattgatcacggtgcttcaagttggtggtcgcggcctgtagctcccatgttactaggagatacagtacatggcaaagtcattcaccggacaagggaagtaaggaaagaactgcagtcaatcatgtgggaatatgttggaattgttaggtctacctcaagactaaccgcagctgagaagagaatcagagagttggagttggaatgggaaacatacctatttcagcatggctgggaaccaacaatggttggagtagaggcttgtgagatgaggaatctcttctgttgtgccaacctggtagttaacagtgctctttctcgacacgagagtcgcggtcttcattacaccattgattttcctcatgttgaggaaagcaaaaggttgccaacgatcatttttccgtcacagcgaaatagctcatggagctcgcggcaattacacaggcagcagatatgttagatgctcttacctatttgccaatccttcctgctaaaacttgtcaagcgcagtgcatgattagtttttaggtaaagaagatgctcatgggttattgtcattcttcccgccattacatttggggcagggcaatgtcattctttggtgaaatatatatatatatatatatatatatatatatatactgtagacaataaatttgc

SEQ ID NO: 9 - DNA, 7407 bp, Artificial Sequence
  Description of Artificial Sequence Synthetic polynucleotide
atataaaagcaagcacgaggaattatcaagaatatttaattaggttggtccggttcaggagagggggatggattgggtttagcagttggtgattacgtgcaatgtgcgctaatctagttggataactacaggagtaatatttttaacattactggcagtctccacttttcacactccatttgctgcaataagcaaatatcactgactacaaaaaggactgcatgttcctcttcctctttcgctcttcggtgctcccacaaccctttcttccttaggtctattcaactcacctcagctctcggttctttgatccagtgtttgcctctgtttcttgcaggtatgataactttcttgctttgttccttttcataccctatttagaaccttattcttttccttcttactactatgttgatcttttacccttcaacaattaagctttttctgtcttgaattatgtgtttatttaatcatgttatctgcaaatttgatacctttgccgttttgttactatatcaaccattcaagaagtaagcttttgttcttgaatttggtgcttctttaaacattgttcatctataagtatgtacgtttgttgttgggttgtactatttccgtttacagagaaagtttagctcttcccagaaagataaccttggaaaaaataatgggagtaaaagaaaattgctttatttttggttgattaacaaaacaataggtaagaactggaaattgtacgggaatatgagcatgggtttggctatttagcttgagtaaaaaaattcctattttggaggatctctggtcaagaaaaaagtttgatttttttttgctcagacatacttatagcctgttttgccaagattttgtttttggaccaaaagtgcttttttttagtcaaaaagtgtttttttttggtcaaaattaaagtatttggccaagctttgagaagaaggagaagcattttcggagaagcagaaaaattagcttcttttcaaaaatacttttttgaaaaacacttttgagaaaaatacacttagaagcactttttaaaagcttggccaaacactaattgttgctcaaaagtgattttcaaattatttagccaaacacaaactacttctcacttgaaaagcactaaagcgcttttcaaataatcagattttagaaatttggccaaacatgctattataagtttactgcaaaaaatcttccttaattgttttttcttcccttttattttttctctatctgatgaaagaatatatatctactagtggggaatagttggaaataggttttagtcttgttagtaagttgattttgaacctatttcttttctaggagtattttgacctcatcatagatttatatattattatgcccttaaaaatttagagaacttttaatattatttatgtatattaaatgcttttatttttattcaatatcatgatgacatgagttgagcgtagctggagacatatagtcagtaaggattcatgtagccgaaccctgcttttttgagactgaggggtgagatgtagttgttgttggtgaggacatggcttaactatactactgcttctgtatgtttacaatttggttgacgacagactcttttaccagaaacaattgcaaaattgaaaagctcttcttgaaaaagcagacttattcctgtagatagaccatggttggcttgaaatcttaacagacttgtggagggtctttttaagtgccctctcttcttttggggatttaaataggagaaacatagcaatttggtggttgatgtttgttcataaacatgaccaaccgttgaagataggtcatataatatatctggtgaattttgttgcgtcacttcataccttgttagttcctctctatgttgccttgttcttgtctgtttagcagctctagatttttgtattaagttgtatgtccttttgttttttttatgttgaccagactttgcaggataaccagaattagcaaatatgaagagtgcaggaaggatgggaatccactatatgcagaaattgaacgcggcgaatgttccaaaagaattagtagaaaaaggacagaatcgtgttatagaagcatctcttacacttattcgtgaaagagcaaagcttaaggtaatgtaagacgtggaagaatgtaatttatagtggggcaaatatgatgaaatgatatgcattagtattgcttatttttgtatttggctacagcaagctaattgtgcaagtactaattatgagtagaaccttttgtaaaagggttttgaactaatggcctaacagcacccatgcaaaagctttctttggtttatcttggtggttcttaatggtgtgggtgattgtagattcagtattgagttctcatgtaaagtagttttacatatgataatcatcattagaaaaaagttttgtatgactggaattgaaatgcatgactaaacatttcgccagcagcagagatgtttctaagttttgtattttttgctttattacaggcaattgtttgccattttaaaaacagtgttaacgttctgcttgatctgtatccctacatcatcacttaatctagtcatacaaatgtaatgggaaagtggtaggtgtatcctaatatgataatggtcaatttctttatcatggttaatagtatagaacatgggggtaccaggggagaaggagagaaagaagctagcagctacttctgttgcattgtcttacaagctctcccattcgcactgacttacaagttggttctgctttgacgaatgagttatttttctttaagaccattgacgtgaatgagtgaccttattcccttcctgatctttttaagttattcttatagggagagcttattcgtgctctcggaggtgctgtggcttcaacgtctcttcttggagttccgttgggacataactcttcatttctccaagggccagcattcgctcctcctcgtatacgagaggctatttggtgtggcagcactaactccacgaccgaggaaggtaaatcaagttatctacctgttaaaacgattattcttcacatcatttacttcgtgcgagatgaggttcaaacgacaataatttctttggggagtatcaatcacaatcctatattcaataaatattgggttaagctttatactcattctattccatatcagatctagttccagttgagattttgatatacatggtgaaagccatctttcttaaccccccccccccccccccggattcttctcacgtcattcttgaaatctatttgtagtgactaaagagtgcaatgcgtattttttgcagaaccatctgcatttccatgtaaattaccgtacagaagtctggaaagtcaataatcactttcatcatgttgtgtaaggaagaattcattactctggaattgtttaatttaatagtgaggggaaagaagggggggggggggggggttcgaaaattgtaaagaagatgtaggtgtttgaacctaaaaccttagttcagtagtggttacaccagccaaactagcccctttatccaagattttatcttgacgcaaagcgcagctttcaattatattattagataagtggtcagaatcattcacttcgtagacctttttctctgcatttactagtagctgttctatctcccgttcgagctgctgcacatgtccatttggttcatttgccagatacctcggtgttttggcctcctctgttgtaggtcttgttgaagtgtgtgaactggctcatcgactgatttagagcccgtttggattggctgattgtaaatagctgataagtttgaagtgctgaaactgattttttaaaaaagtatttacgcgtttggatagacgtgctaaaactgataataagcagttgatgtgtttggtagaaaagtgctgataagctgttcttttaataaaatgacaaaaatatccttaaaaactttacaaaagattatgaattaaaaagtttcttgtaatgaaaaggatgaacaacaaatatggaatgaagaggaagttagaaaatttattttgggaaaaatattttgtgaattaaaaaatattactaaggataaactagtaaaagcctaggtcaaactaaaagtgcttataagctgaaaaatcataagttgggggtgaccaacttatgacttatggctgcttttagctcataagcactttgagtgtttaccaaacgcgtagataagccaaaagatgattataagccagtttgaccagcttataagcttagccaaacacctcttaaactgttagaaatggacattttctatttatttattttagattgcagtctcatcatgggcgggcttaattttcattgtgccacgcacgtaagtatttttgtgatggtcaatagtgagactcaaattcaagacccatgtatgctgataaatattaaagtgtgctaatcgtctcatcgaaaaccttaagctgctaggtagaagttacgtatttagttcattgttgtagaaattcgatgtcatggtgctaactttaattttggatatctgacgctttgctgctggtgctttgatgttctatgattaagagaagtgttgttttgctggttacaatactatattacatttttgtaaagcttagtagcctagattgagctattttttcggcaaatgcaccgtaatgaagtttcaaagattattggttgacaccaagagagaaaaataaaaataaattaattgaatcttaatgaagaaacttgatgttctaaattgttttaaccatgtgtggcttgaagcttatggtcgctcttccggattttgctcataatctgaagggttaatcaatcactatttcatgttccctttttctaggatgaccgcttgatctagagtaattcggtcttccttgttctcctattcatctcttgcatctactgatcagctttttagcaactgatcaatgttgtttggaagattttctctatttgacggatccttctagataacatgatattcaattatcactaggaagtctcactataaagtagcaccaaaaaatgctgtctgattgcttgaacagccaggcactcagtctaatttgctaaacctcctgaatgttattatcggcaattttcaccatacatgttgacattccttttagctaactgatattcgaagtttgaaatctaaacaagttatttctattacgcaggaaaagaattaaatgatccacgcatcttgaccgatgttggtgatgtgccagttcaagagttacgagatagtggcgttgatgatgataggttgatgagtatcataagtgaatctgtcaagctagtgatggaggaggtaaaccatcttttatgtaaataacttctgagaaagtatttggagtcctttttctgcaagggatggatactaaaatcataggccttgctaggacagtggcatccattcaagttagagaaattactaaatctgttttataacgtcatatcaaacttccgaaaccccctccctcccaaaaccaggaaaaaaaaaggatatataattttttatttctttttcttatgtctgagtcctacctgctccattctaaaattttagctttaactttatcttttctatgcttatattatttaaagttaccaatgtgagctctcaccttcattcatgtctaggagaagttaaaaagggaaaaggagagttctcttcatgacgtcatatgccgttctcatggatcttttatccataatatgatttttagagctggaaggacctgcttgagacagtttactgatgactatatgggcatcaaaatattgttgctttctctgcactgccacaatagtccttgccaaaattttcacctcaaagtgaacaaaagaaaaagatcttcagttgaatgaaagaaatcagtttttcccagctgttatccaatgaagcatcaaaactcacttttgttgtatgatttccttgcttctgaatggaatgttaatatactttgatgtcgggataaacaattctctacaattatgaactctactcaaagtagttcttattaggcaacggtttatatgcttcagtttctttctcttcttttccccgtacttaataatttgttaattcaattctcgtattgacttttcaggatccattgcgccccttggtgttagggggtgatcactccatatcctatcctgttgtaagagctgtgtctgaaaagcttggaggacctattgatatccttcaccttgatgctcatcctgatatttaccattcctttgaaggaaacaaatattcacatgcatcaagctttgcacgaattatggagggtggttacgctcgacggcttttgcaagtaatctaagctttccctatagaccaactaagcatgtatgactttgtactacatttcttgcattcctatgtaaagcgatctctcctgcaataggattagctgttcaatgatgtactctttgggtttacaagttgcctactctcgctagcataaacattagaaattgtttaacaaaacccttgatcttgtttgtaggtgggaattagatcaattaccaaagaaggtcgtgaacaagggaaaaggtttggcgtggagcaatatgaaatgcggactttttccagagaccgccagtttttggagaatctggtctgtctttctttaccacctctttcctttcccttcccctcttgttttttctccctgaattatatcttgcaaacgtgttcatcacttcaaaagtctcaaatgtgctgagtctatttcagatgtatattatcatatacttctctctgattctctgtatttcctactataagtcattatctcctttgatttcatgtcaaatgtagcaatactaaacttccaatgtctgtacactggcagtgcatcatttctacatttataatttttttttgtacattcagaaacttggtgaaggtgtgaagggcgtgtatatctcagtggatattgactgtatggatccagcatttgctcctggagtatctcatatagaaccaggcggtctctctttccgtgatgttctaaacatactgcataaccttcaagcggatgttgttggtgctgatgttgttgagttcaacccacagcgtgatactgttgatggcatgactgcgatggttgctgcgaagctggtaagagaacttactgccaagatatccaagtgatctgcacaggctattctaaatttatgaaggacgcagtactgtgcatcctatatagaccgctggcactagattgatactcatttttactgggaagtttaatagaggataagcaccaaagcaagtgtttatccaccttatcgtagcactgaaagcatattagaacttaaaatggaagtatcaggtaggcctagtagatgctc

SEQ ID NO: 10 - DNA, 7853 bp, Artificial Sequence
  Description of Artificial Sequence Synthetic polynucleotide
tttccttataaaagtggaccaaatactttaacttaaaaaaaagattttaaaaaatatatttttgactttgaagaagcagactataaaatgaaaaatccattcgtctcgtgctatctaaaatcaagcaggagccaggaggggtatcaagaatatttaattaggttggtcggacaaatccgtttagcgataggggatggattgggtttagcagtcagtgattacgtgcgaagtgcgctaatctagttggatcactaatattattggcaagactccatttttcacactccatttgctgcaagcagcaaatattaccaactacaaaaaggactgcatgccccttctccaccttcgctcttctgtccttccacaaccctttctcccttagatctattcaactctcggttctcttgacctgttgtttgcctctgcttcttgcaggtatgatcactttctcgctttgttcctcttcataccctatttagaaccttattcttttccttcttactactatgttaatcttttacccttcaacaattaagctttttctgtcttgaattatgtgcttatttaaatatgttatctgcaaatttgatacctttgctgttttgttactatatcaccttccaagccttcttcgtccttcttcccaataaatattgctttttcttgccattcaagaagtaagctttggttcttgaatttggtgcttctttaaacattgttcatctataagtatgtacgtctgttgttgggttgtactatttccgtttacagaggaagtttagctcttccaagaaagataaaaaataatgggagttaaagaaaattgctatacttttggttgattaaccaaagaatagctaggaactggaaattgtacgggaatatgaacatgggtttggctgtttagcttgagtaaaaaattcctattttggaggatcttcggtcaaggaaaaagtttgattttttcttttttccttgcttgtttttccttcccttctattttttcactatctgatggaagaaaaagatatctactagtggagaatagttggaaataggttttagtcatgttagtaagttgattttgaacctattgcttttctaatagtattttgaccttatcgtagatttatatatttttatgctattaaaaatataaacaacaacaacaacccagtataattccacttagtggggtctggggagggtagtatgtacgcagaccttacccctaccctagggtagagagactgtttccaaatagacccccgacatccttccctccaagaacttcccaccttgctcttggggagactcgaactcacaacctctcggttggaagtgggggttgcttaccagcagagcaacccctcttggggagattcgaactcacaacctctttaatattatttatgtatatttaattgcttttatttttattcaatatgacgatgacatgagttgagctcagctggagacatatggtcagtgaggattcatgtagccgaccccaactcttttgggactgaggggtgagatgtagttgtttttggtggtgacatgacttaactatactatgcctttctgtatgttcacaatttgattgacgacaaacccttttaccagaaacaattgcaaaattgaaaagctcttcttgaaaaagcagacttattcctgtagatatgccatggttggcttgaaatcttaacagacttgtggagggtcttttctaagtgtcctctcttcttttggggatttaagtaggaaaaacatagcaatttggtggttgatgtttgttcataaacatgaccaaccgttgaagataggtcatataatagatctggtgaattttgttgcgtcacttcatgctttgttagtttctctctatgttgtcttgttcttgtctgtttagcaactttagatttttgtattaagttgtatgtctttttgtttttctatgttgaccagactttgcaggataaccagaattagcaaatatgaagtgtgcaggaaggatgggaatccactatatgcagaaattgaacgcggcgaatgttccaaaagaattagtagaaaaaggacagaatcgtgttatagaagcatctcttacacttattcgtgaaagagcaaagcttaaggtaatgtaggatgtggaagaatgtaattttgagtgggcaactatggtgatatgatatgcattagtatttttgtatttggctacagcaagctaattgtgtaagtactagttgtgagtagaaccttttgtacaagggttttgaactaatggcctaacagcacccatgcaaaagctttctttggtttatcttggtggttcttaatggcgtgggtgtttgtagattcagcattgagttattatgtaaagtagttttacatatgataataatcattagaaaaaagttttatgtatgactggaaatgatatgcatgacaacatttcgccagcagcagagatatttttaggttttgtattgtttgccattttaaaagcagtgttaactttctgcttggtctgtatccctacatcatcacttaatctagtcatacaaatgcaatgggaaagtggaaggtgtatcctaatatgataatggtcaatttccttatcatggttaaggatagtatagaacatagggtaccaggagagaaggagagaaagaagctagcagctacttctgttgcattgtcttacaagctctccctttagggcttgttgtttctcgttcgcactgacctacaagttggttctgctttgacgattgagttatttttctgtaagaccgttgacgtgaatgaatgaccttattcccttcctgatctttttagtttattcttacagggagagcttattcgtgctctcggaggtgctgtggcctcaacgtctcttcttggagttcctttgggacataactcttcatttctccaggggccagcatttgctcctcctcgtatacgagaggccatttggtgtggcagcactaactccacgactgaggaaggtaaatcaagtatctacctgttaaaacgattattcttcacatcatttacttcgtgcgagatgagtttcaaaagactataatttccttggggaacatcaaatcacaatcccatattcaataaatattgggttaagctttatactcattctatccaatatcagatctagttgcagttgagattttgatatatatggtgaaagacatcccatcccccaccccctacccccggggggaggggggggggttctcctcatgtcatttttgaaaatctatgtgtagtgactaaagagtgcaatacatattatttgcagaaccatctgcatttccatgtaattactatacagaagtctggaaagtgaataatcactttcatcatgttgtgtaaggaagaatgcactactgtgaaattgtttaatttaatcatgagagggaagggggtggcggggtcggaaattgtaaagaagatgtaggtgtttgaacccaaaaccttagttcagcagtggttacaccagccaacctatagcctgtttggccaagcttatttttggccaaaagtgcttttttttggccaaaagcacttttggccaaaaattgaggtgtttgccaagcttctggaaggaaaaaaagtgcttttgaggagaagcagaagcaattttggagaagcagaaaaaagtagcttctctccaaaaagcacttttttgagaagcacttttgagaaaaatacacttagaagcagttttttaaagcttggccaaacactaattgctgctcagaagtgcttttcaaactaattagccaaacacaaactgcttctcaccaaaagtacttttgagaaaaacacttttgaaaaaaagcacttctcaaaataaactgatttttgcagcttggccaaacgggctagcccctttatccaaaattttatcttgacgcgaagtgtagctttcaattatattagtagataagtggtcagaatcattcacttcgtaaatctttttctctgcatttattagtagccgttctatctccagctcgagctgctgcgcatgtctatttggttcatttgccagatacctcggtgttttggcctcctctgttgtcggttttgttgaagtgtgtgaactggctcatcgacagatttaaactgttaaaaatggatattttctatttatttattttagattgcagctccatcatggtcgggcctaattttcattgtgccaagcacgtaagtatttttgtgatggtcaatagtgacactcgaattcgagacccatgtctgctgtaccctgataaatattcatctcatcgaaaaccttaagctgctaggtagaggttatgtatttagttcattgctgtagaaattccgttgtcttggtgctaactttcattttggatatctgacgcttttctgctgttgcttggatgttctatgattaagagaaatgttgcattgctggttagcatactatattacatttttgtaaagcttagtagcctagattgagcaatttttcggctaatgcactacaatgccattaaaactcggaaatgatcttaccttttagtagagcagtttaatttccttttatgtgtcaatagaacatgatactaaaggaatagtgaattgtaatgaagtttcaaagattattggttggacccaagagagaaaaagaaaaataaacaattgaatcttaacgaagaaacttgatgttctaaatcgttttaaccatgtgtggcttgaagcttatggtcgctcttccggttttgctcaaaatctgaagggaatcaatcactatttcatgttcccttttctgctatgaccccttgatctagagtaattcggtcttccttgttcctcctattcatctcttgcatctactgatcagcttttcagcaactgatcaatgttgtttggaagattttctctatttgacggatccttctggacaagtaaaagttcaagtaccaccaggaagtcacaccataaagtagcaccaaaaagtgctatctgattgcttgaacagccagacactcagtctaatttgctgaacctcttgaatgttattatcggcaattttcactatacatgttgacattccttttagctcaccgatattcgaaatctgaaatctaaacaagttatttctattatgcaggaaaagaattaaatgattcacgcatcttaaccgatgttggtgatgtgccagttcaagagttgcgagatagtggcgttgatgatgataggttgatgagtatcataagtgaatctgtcaagctagtgatggaggaggtaaaccatcttttatgtaaataacttctgagaaagtatttggtgtcctttttctgcaagggatggatactaaaatcataggccttgctagaacggtgggagggccgccgcccaccccacccacccaccccggcgcacccattcaagttagagaatttactaaatccattttataacgtcatatcaaacttccgaaaccccctcccacccaaaaccaggaaacaaaaaagatataatttagagtatttctttttcttacatttgactcctacctgctctattctagaattttagctttaactttatcttttctatgtatattatttgaagttaccaatgtgaaaaattagctctcaccttcatccatatctaagagaagttaaaaagggtaaaggagagttctcttcatgacctcatatgctgttctcatggatcttttatccataacatgatttttggagctggaaggacctgcttgagacagtttactgatgactatatgggcatcaaaatattgttgcctttctgtactgccacaatagtcctagccaattttttcacctcaaagtgaacaactaaacagaaaaagatcttccgttgaatgaaagaaatcagcttttcccagctgttatccaatgaaacatcaaaactcacttttgttgtatgatttccttgcttctgaatggaatgttaatatactttgatgtcgggataaacaattctctacaattatgaactctactcaaagtagttcttattaggcaatggtttatatgcttcggtttcttttctttttcccgtacttaataatttgttaattcaattctcatattgacttttcaggatccattgcgccccttggtgttagggggtgatcactccatatcctatcctgttgtaagagctgtgtctgaaaagcttggaggacctattgatatccttcaccttgatgctcatcctgatatttaccattcctttgaaggaaacaaatattcacatgcatcaagctttgcacgaattatggagggtggttacgctcgacggcttttgcaagtaatctaaattttccctatagaccaactaagcatgtataactttgtactacatttcttgcattcctatgtaaaacgatctctcctgcgataggataagctgttcaatgatgtactctttgggtttacaagtagcttactctcgctagcataaaccttagaaattgtttaacaaaacccttgatcttgtttgtaggtgggaattagatcaattacaaaagaaggtcgtgaacaaggaaaaaggtttggcgtggagcaatacgaaatgcgaactttttccagagaccgccaatttttggagaatctggtctgtctttctttaccatctctttcctttcccttccactcttctttcttctccccaaattatatacttgcaaacgtgttcatcagttcaggagtttcaaatgtgctgagtctattccagatgcatactactctcatatttctctctgattctctgtatatcctactataagttatcccctttggtttcatattttgcgttatgttatcaaatgtaaacactgccagacctcgaatgcctatacactggcatttcatcatttatttgctacattgataatctcaattggtaaatacttttgtacatttcagaaacttggtgaaggtgcgaagggcgtgtatatttctgtggatatcgactgtatggatccagcatttgctcccggagtatctcatatagaaccaggcggtctctctttccgtgatgttctaaacatactgcataaccttcaagcggatgttgttggtgctgatgttgttgagttcaacccacagcgtgatactgttgatggcatgactgcaatggttgctgcgaagctggtaagagaacttactgccaagatatccaagtgatctgcacaggttattctaaatttatgaaggacgcaatactgtgcatcctatatagaccgcttgcgttagatttatactcacttttattgggaagtttaatagaggataagcaccaaagaaagtgtttattcaccttattgtagcactgaaaccgtattagaacttaaaatggaagtatcaggtaggcctagtagatgctcagttttccattgcaagatcaaattagacaatgagaattatttcatgttattgattgggatatctgtaagctcacctcccaaaataagctttcttcgctgctcctacttatatctggtaagtaatgcaaatcttgttgtgtacggatgttatttaatcttttgtggtgtagaacaaagcgtttaccaatggggcgaggcatagcccggaaccatgactttttagactgataattctaaaaataaaaattgaaataaaaacagattaatttttttttttaaaaaaatagagaaaatgcattaaacattatataattcaaaagcaatatactgtacaagaattgattttatccgggcataaattgtatatatgaaggggagccttggcgtaactggtaaagttgtttccatatgacca

SEQ ID NO: 11 - DNA, 3499 bp, Artificial Sequence
  Description of Artificial Sequence Synthetic polynucleotide
gagttcgagtcaccccaagagcaaggtggggagttcttggagggagggagctgagggtctatcggacctttctaccaagggtagggggaatttatatatttcttttcaaatctttcttgcataaacccaaaaaataaagcagactcagttgatgctaacatttgttgttgacggtaaagttgtctgtgtgacttataaattacgggttcgaaccgtaaaatcagtcactgatgtaggttgtctacattacatccatttgaagtacggctcttcgctaaaccctagtccctatataaacggaattgatgcttcgtgcaccggattgcttttattcaattgttgcaaacattttttttgcatcaacgtcaagctttaagatatgaactaacttgtctaatttttattttgcgtcgtacgaacatgtgctgcacaatatcttttttatatgtttgtttgttttttcctgacgcagtatgggtgtgtatattttttggtgggggcggagggctacgagactataaaattattaagatctgacaaattcgcttacgcggccataaatatgttagcatgccatgcatatataataacagaagatcaaatctaggattctatatatgcacatgtcgaaactttaaatatttaattataaatttaattattattatatactaacttgagtttattgtaaaacgacgaatacgaagagctaaagaacccaattaggccgctaaagaacccaattaggtaggccaaaactcaaagccaaaggggttgttggtgcctttggtaggggttggtgagacttttaaaaaggaaacttgactgttggagcttttgaaaggggtatatgctttgttctgagatgctaaattagttcattaacgataacaatttgtgctgagaccaccaaataggacaaaccatagttggctgtcctttccctaagatccacatttttcatgtgctcttttctttctttatcccgagatttgaaaataaaataaataaaattagagaaagaggctggctaagcaaaacaaatcatgcacttgcatctctattttagtatgagtaaattggaatagtgaaagataatacgaattggtgtgactgaatataaaatgttctcgcctttaatcagagatttctacttcaaaatttaaaaattaggttatttttaaggagcgttaaaatctttcatatttgactgtttacatataaatttggatatgttaatttcgaagaatgaacggttaaatcaaaaaggaaaaaattaaagaatagtggaagaggaaatgatggaatgaaagaggacttttaatatgattagaattatgtaataaatgaaattacaagtactagattacaactttaattatgtaatttacaaactaatacatatcctatttaaactaaactaagcaatttaattttaaaagtcacaagttaaaataaaaaaatacattaattaatattaatatgattaatgatagacattcattttgaaaattacgttccggtgcaaacacaatatactagtaattatttgctagtatactgttaatggaatctcccatagtatatgtggattaacttccactggtcttttcttgatcctctaatatgtcatagaaaaaatcattcaccgttggataatatatatttaattatcatttacctaacaaaaagtacttcccttcatcagatttacataaaggacttgttattaaattcgataccgttgaccacctaccccttcaacagctatttctcttaaaaaaacaaaaaagaagaaaatactgcgtagattacacaatattatcagtagtagtatctcttttcgtccctctatataatgataaacattttttgaggtttccccgtctcaaagggaacaagagaaacacaatattcatatattataattgaatccctagttttttttcccctttgattccttcctctcaattacctctctgttttcttcctttgtttggatggccggccaaacaataatcgtttccgggttgaacccggcggccattcttcagtccacaataggcggcggagcttcacctacagcggcggcggcggaaaacggtaccagaaaagtcatccctctctcaagagatgccctccaagatttcatgttatcaatcataacccaaaaattacaagatgagaaacaacctttttacgtgttagatttgggtgaagttgtttctcttatggaccaatggaaatctgctctcccaaatatccgtccattttacgctgttaaatgtaaccctgaaccgtcgttcctttcaattttatctgctatgggctcaaattttgattgtgctagccgagctgaaattgagtacgttttgtcccttggcatttcacctgaccgtattgtttttgcaaatccatgcaaaccggaatccgatattatttttgcagcaaaagttggggtgaatttaacaacgtacgattcagaagacgaggtttacaagatccgaaagcatcacccgaaatccgaactcttgctccgaatcaagccaatgttcgacggcaacgcgaggtgcccaatgggtccaaaatacggcgcgcttccagaagaagtcgagccgctgctccgggcagctcaggccgcccggctcaccgtctccggtgtctccttccacatcggcagcggagatgccgattcaaacgcttatctcggcgccatagccgcggctaaggaagtgtttgaaacagctgctaaactcggtatgtcgaaaatgactgttctagacgtcggcggcgggtttacatccggccaccagttcacaaccgccgccgtcgctgttagatcagctttaaaacaacacttcgatgatcaaccggagttgacaatcatagctgaaccgggccggttttttgcggagactgcgtttactttagcgacgacgattatagggaaaagagtgaggggagaattgagggagtattggattaacgacgggttgtacggttcgatgaactgtgtactttacgaccatgcgacggtgaatgcaacgccgttagctgttttgtcgaatcgtactaacgttacctgcggcgggtcgaaaacgtttccgacgactgtgtttgggcccacttgtgatgctcttgatactgttttaagggattaccagttaccggagctgcaggttaatgattggctagtttttcctaatatgggtgcttatactaaagctgctgggtccaattttaatggatttaatacgtccgccattgttactcacctcgcttatgcttatccaagctgatgaataacctctattaggaattaccacctgtggttttaatggttttttcttttttgggggattttttttcttaattttgttgtttttggtagtaatttatattccaaatcagcttgtaattctcttgtatgccataagaatgcaaggatttgctaattgtgatttgctctaatatggaagtttttaaaattagttattag

SEQ ID NO: 12 - DNA, 3502 bp, Artificial Sequence
  Description of Artificial Sequence Synthetic polynucleotide
atcagaccagacaaattagcttatgcggcaataattatgatagcatgcatattacataggatcaaatctaggattctatatggtcggatccagattataatttacggatttatggaattcaataacttttgttcaaatcttatttaataataaatttatcaaatactttaacatttaaggcgttgatatttaactataaacttaattattattgtatattaatttaaggttacttgagaaactcataaacttcgaatcgtaaatccaccaatttcgatgattcagacatgttaaatggataagcatagaagtcccagttaggacttgtgagaggttagccttgatgggtatggggaggggtagaggtaggtcctaagaagtcctgggagaggtgattagacgggacatgacacaactagagcttattgaggacatgacccttgataggagaccgtgaagttcgagaattagggtagaaggctagtaggtagtcgagcgtttctctttgtctttctttgtttgataacattagtgttagtttgatatacctttattcttagattgctattattacctaccgtttaattgttatcttggcttcggtcttctttttattttgttgttgtaactacttattgttattactttttttttattttttctttagctgagggtttatcggaaacatcttctctgtccttccaagatagaggtaaagctacatatattttatccttcccagatcccgcttatgaaaatttagtggatatgttgttgtttgttcgtaacgaacccactcatgccaaaattcaaagccaaaggggttgttggtgcctttggtaggggttggtgagacttttataaagggaacttggactgttggagcccttgaaaggggtatatgctttgttctgggatgctaaattagttcattaacgataacaatttgtgttgacaccaccaaataggacaagccacaattggctgtcctttccctaagatccacatttttcatgtgctcttttctttctttatcccgagatttgaaaataaaataaattaaattagagaaagaggctggctaagctaaaccaatcatgcacatgcatctccattttactattattaaaggtatgaatagattagaatagtgaaagataccaccaatcggtgtgattgaatgtaaaaaatttcacctttaatcagagattttcacttcaaattttaaaaattgggtcatctttaaggagctcaaaatccttcgtaattgaccttttactatataaatctgaatatttaaatttcgaacaatgaaccgaaaaaaaaaagaatagtgaaagaggagaagatgtaatgaaagaggacttttaatatgattagaattatgtaacaagtgaaattacaagtacaagattacaactttaattatgtaatttacaaatataatacatatcctatttaaactaaactaagcaatttaattttaaaagtcacaagttaaaataaaaacacacattaattaatatgattaatgatagacatttatttttaaaaatactccggtgcaaacacaatatacgagtaattatatgtggattgacttccactgattttttcttgatcctctaatatgtcgtagaaaaaatcatgcaccgttggataatatagatttaattatcatttacccaacaaaaagtacatcacttcatcagatttacataaatgacttattgtaattaaattcaataccattgaccacctaccccttcaacagctatttctctaaaaaaaaaaaaaaagaagaaaatactacgtagattacacaatattatcagtagtagtatcacttttcgtccctctatataatgataaacattttttgaggtttccccgtctcaaagggaacaagagaaacattcatattattgaatccctagtttcttttctttccctttgattccttcctctcatttacctctctcttttcttcctttgtttggatggccggccaaacaatcatcgtttccgggttgaacccggcggccattcttcagtccacaattggcggcggagcttctcctacagcggcggcggcggcggaaaacggcaccagaaaagtcatccctctctcaagagatgccttacaagatttcatgttatcaatcataacccaaaaattacaagatgagaaacaacctttttacgtgctagacttgggtgaggttgtttctcttatggaccaatggaaatctgctctcccaaatatccgtccattttacgctgttaaatgtaaccctgaaccgtcgttcctttcaattttatctgctatgggctcaaattttgattgtgctagccgagctgaaattgagtatgttttatctcttggcatttcacctgaccgtattgttttcgcaaatccatgcaaaccggaatccgatattatttttgcagcaaaagttggggtgaatcttacaacctatgattctgaagacgaggtttacaagatccgaaagcatcacccgaaatccgaactcttgctccgcatcaagcccatgctcgacggcaacgcgagatgcccaatgggcccgaaatacggcgcgcttccagaagaagtcgacccgctgctccgggcagctcaagccgcccgtctcaccgtatccggcgtctcattccacatcggtagcggagatgccgattcaaacgcttatctcggcgccatagccgcggctaaggaagtgtttgaaacagctgctaaactcgggatgtcgaaaatgactgttctagacgtcggcggcgggtttacatccggccaccagttcacaaccgccgccgtcgccgttaaatcagctttaaaacaacacttcgatgacgaaccggagttgacaatcatagctgaaccgggtcggttttttgcagagacggcgtttactttggcaacgacgattatagggaaaagagtgaggggtgaattgagggagtattggattaacgacgggctgtacggttcgatgaactgtgtactttacgaccatgcgacggtgaatgcaacgccgttagctgttctgtcgaatcgtagtaacgttacctgcggcgggtcgaaaacgtttccgacgactgtgtttgggcccacttgtgatgctcttgatactgttttaagggattaccagttaccggagctgcaggttaatgattggctggtttttcctaatatgggtgcttatactaaagctgctgggtccaattttaatggatttaatacttccgccattgttactcacctcgcttattcttatccaagctgatgaaccacctgtattaggaattactaccgtggttttgatggttttttccttttttgggtatcttttttttaattttgttgtttttggtagtaatttatattccaaatcagcttgtaattctcttgtatgccataagaatgcaaggatttgctaattgtgattttctctaatatggaagtttttaaaattagtttaagaaa

SEQ ID NO: 13 - DNA, 3373 bp, Artificial Sequence
  Description of Artificial Sequence Synthetic polynucleotide
gatacattaaagttgtctctgtgtgacctatacatcatgagttcgagctgtgaaagcaactactaatgcatatattagggcagactgtctatattatactttttggagtgcggtcctttgccgaatcttacatgaatacggaatactttgtgtaccgagctgcactttatctttatatataacttaaatcattttatgaatacaacaaaaacctacatcttatttaaaactcaaaaaactacctttttggcctactatttttggtaaactttgaactaatgcaagtcgtggtagtacctaatataagaaaaaagagagcgagtagctggctccgttctcccttcaattccacttcctataaatatacacacaaatcactcccactacacacacaatcaaatctccttctctctgctcttacttcttctttgtcttctcagatctaagagtttgtttcttcttcaaggtatgtacttaaatctaccacttaattcactctttaagctctgttttcctctccactagattcagatctagagtatatctgcatttttccttctatgatctcatcatttctgctgttaattttttgttttgttttgttttactcttaaatttctaaacttgaatgtactaagtttacggattctagagattaaggtgacaacatttttttgttgttatataattgggaattatttgttgtgttacgcatgggtcagggacggatacagcctagagttacctggaactcagtacttttgatgcatagtatattttcattaaaattgaaacatcactctacaaatattatggattcaatagaatttgaatcctatattcgcctccggcgtgagtactgacaagttggctatgatcagatctctttgtttttttaatgattttatctatattttttgcttatgttttactatcgtgtaattgtaaatcaatttagtttatccaattgataacctgcttttgtagaacgagggtaggatctgttaccaaagttgcaagtttttcgacatgaatttggtaattttacgtgctaactagaggagtaattggatctcattcttaagataagagttacattacgacagtcaaatctagtagactggtaccaaattagtgctaacagtggtgaaatacatgatatcatatgtgttaatgtaatgtgtttggatttggtaccaaattccttaaattgtagatgtatgtctgtttggttttagctgtcgcttgtgaatacaagtttggagttgattttggaatgaaaaggttttcacctttgatttcaccttattctctatccacacacatatttgtctagttttacacaatagaagatttgttcaaaccaaatgattgatggatcttgacttgtctcgggtctcccttggacatagtttatgtatagcaaataatgcagaattttttcggtaatgcagtgtaaattttgttactttacgggaacgtgtggtgtttgaaattcttataatcaaattttctgggcttaaatgagatccttatactttttaaaagaaataaatgagtccttttatgttaaatcatttaacgaaaccctgattgacttctccctgttccctctgaatgtttgtcgccaatagttgttggtccagaactgactgatgattgtgaactgtgacaaatattgcagtgacctctttgtttgatgaaacaagatacattgtgttttttaaataatagaaagcattgaggagtctgctcgaattttctgaattagaaaaaatcaactactcgctgttttatcttttcatggtattctgctactgagtgcagaaatctcactctctgtcagtgtgattgaattcttggttgtccttgtttttaaattcttgctgctttctcttgcctcatcatcatcggggatacgatcaacttttacctcaatttcttattaattgaacatttcaggattgcagtcttaacacattctctgtttcagggcagacatggaaacttttttgttcacctcagaatcagtcaatgaaggccatcccgacaagctctgtgaccaagtctcagatgccattcttgatgcttgccttgcacaagatccagagagcaaagttgcatgtgaaacctgcacaaagacaaacatggtcatggtctttggtgagatcacaaccaaagccaatgtagactatgaggagatagtacgtgatacttgtagaggcattgggttcacatcacctgatgtcggccttgatgctgacaactgcaaggtccttgtcaacatcgagcagcagagccctgacattgctcaaggagttcatggtcatcttacgaagaaaccagaggaaattggagctggtgaccaaggtcacatgtttggctatgccaccgatgaaactcctgagctcatgccccttactcatgttttggctactcagcttggtgccaagcttactgaagtgaggaagaacaagacttgtccatggctcagaccagatggcaagacacaagttacagttgagtacaagaatgagaatggtgccatggttcctattagagttcacactgttctcatctcaactcaacatgacgaaactgtcacaaatgagcagattgccaaggacttgaaagagcatgtaatcaaacccgtgatcccagctaagtaccttgatgagaaaaccatcttccacctcaacccatcaggtcgctttgtgattggtggtccacatggagatgctggactcactggcaggaaaatcatcattgatacctacggaggatggggcgctcatggtggaggtgccttctcagggaaagatcctactaaggtggacagaagtggtgcttatgttgttagacaggcagcaaagagtgtggttgctgcaggacttgctcgccgctgtattgtccaggtttcttatgcaattggtgtggcagaaccactctccgtgtttgttgacacttacaaaactggaaccattccggacaaggatattttggctttgatcaaggagaactttgacttcaggcctggaatgattgcaattaatcttgacttgaagagaggaggtaacttcaggtaccagaagactgctgcttatggtcacttcggccgtgatgaccctgatttcacctgggagactgtcaagcccctcaagcctaaggcttgaacaagtttactcgcaattttccagccgattaaatttcttatggtcattatttttgtggaaaaaaacaataaacaagcttcatagcatacattcgttatccaaaatctactttcaggctccatcgagcagtgggagaaagggacttcaagcattctgagatttcttagtagttggtccttttgaattattttttggtttta

SEQ ID NO: 14 - DNA, 3373 bp, Artificial Sequence
  Description of Artificial Sequence Synthetic polynucleotide
atttttatttaatggtatttgattggcatttttagattaaaataataatctaacacacaaaattaaacccgctatagagttggtgggtttgtaagctggtgggtcctactacgatggttggccattcggtgtcgtgctggaaactaagcacgcacttgtttgaggcattataataattatttagcaatcaaatcaattaggtaattatactaagtaatttaataaaaatagtaaaaatgtattactatacaagttaaaattcacattaataatataaattttcttttaattgttaatgtatataacttagaaggaagcctaggaacgatgataaaattatctttatatgacctacataatacgggttccagccgtgaaaataaacactaatagtatgtctatatcatatgctttgtgcagtggagcgtaccgtgctttgtcatatgtgctttgtcattgaatataattttgttaaaactcaaaaactgcctttttgggaaactttgaactgatgcaagtagtagtacctaataagaaaaagagagcgagtcgttggctcctttcccctttcaattccgcttcctatatatacacacacaaatctctcccattacacacccaatcaaatctcctcctctctgctctcacttcttctttgtcttctcagatctaagagtttgtttcttcttcaaggtatgtacttaattctaccacttaattcacttttcaagctctgtttttctctcgactagatttagatctagagtatatctgcatttattcttttatgatctcaacatttctgctgttaatgttttgtttcgttgttttactcttaaatttctaaacttgaatgtactaagtttaaggattctagagattaagggggaattatttgttttgttacgcatgatgcagcctaagttaccgggttcatatgaactcagtattttcgatgcagaatataaatttgtgtgtaaaaattcattaaaattgcaacattacgttaaaaatataatggattcaatgcaacaagccttaaaatattgaagcaatagaatttgaatcctagattcgcctctggcatgagtaactgacaagtttgtatgatcaaatctctttgtttattaatgattttatctatatcttttggttatgttttactatcatgttaaatcgtaaatagatttaatttatctcatttataacctgtttttgttgcacgagggtaggatctgtaaccagcgttgtaatttttgcacatgaatttggtagttttacgtgctaactagaggagtaattggatctcattatttaaataagagttacaatacgacagtcacacttggtggattggtaccaaattagtgctaacagtggtgaaatacataatatcatgtgtgtaaatgtaatgtaatatgtttggatttggtaccaaactccttaaattgtagatgcatgtctgtttggttttagctgtcgcttctgattacgagcttggagttgattttggaatgaaaaggttttcatgtttgatttcaccttattctatatccaaattccaaacacatatttgttctagtttttcaaaatggaagatttgttcaaaccaaatgtttgttgccaatagttgtcggtccagcattgacggtttgttgtgaaatgtgacaagtattgcatatatctctttgtttgatgtaacaagataccttgtgtttttaaatgagaaggcattgaagtgtctactaacattttctgaatttgaaaaaaccaaccacacttgtcaccatgttatcttgtcatggaatttcgctaccaagtggcagaagtatttttctctattggtatgattgaattcttgtttcatttttttaaagttctttatgctttctattgtctcatcatcaccggagatatgatcaacttttccctcaattttcgtgctagttgaatattttaggattgcagtcttaacacattctctctgtttcagggcagacatggaaactttcttgtttacctcagaatcagtcaacgaaggccatcccgacaagctctgtgaccaagtctcagatgccattcttgatgcttgccttgctcaagatcctgagagcaaggttgcatgtgaaacctgcacaaagacaaacatggtcatggtctttggtgagatcacaaccaaagccaatgtagactatgagaagatagtccgtgatacttgcagaggcattgggttcacctcaccagatgtcggccttgatgctgacaactgcaaggtccttgtcaacatcgagcagcagagccctgacattgcccaaggagttcatggtcatcttacaaagaaaccggaggaaattggagctggtgaccaaggtcacatgtttggctatgccaccgatgaaactcctgagctcatgccccttactcacgttttggctactcagcttggtgccaagcttactgaagtgaggaagaacaagacttgcccatggctcagaccagatggcaagacacaagttacagttgagtacaagaatgagaatggtgcaatggttcctattagagttcacaccgttctcatctcaactcaacatgatgaaactgtcacaaatgagcagattgcccgggacttgaaagagcatgtgatcaagccggtgatcccagctaagtaccttgatgagaaaaccatcttccacctcaacccatcaggtcgctttgtgattggtggtccccatggagatgctggactcactggcaggaaaatcatcattgacacctatggaggatggggcgctcatggtggaggcgcgttctcaggaaaggatcctactaaggtggatagaagtggtgcttatattgttagacaggcagcaaagagtgtggttgctgcaggacttgctcgccgctgtattgtccaagtttcttatgcaattggtgtggcagaaccactctccgtgtttgttgacagttacaaaaccggaaccattccagataaggatattttggctttgatcaaggagaactttgacttcaggcctggaatgattgcaattaatcttgacttgaagagaggaggtaacttcaggtaccagaagactgctgcttatggtcacttcggccgtgatgaccctgatttcacatgggagactgtcaaggccctcaagcctaaagcttgaacaagtttactcacaatttttcagccaattaaatttcttatggtcattatttttgtggaaaaaccaataaacaagcttcatagcatacattcaatatcgaaaatctactttcaggctccatcgagcagtgggagaaagggacttcaagcattctgagatttcttagtagttggtccttttgaattaatttttgggtgtaa

SEQ ID NO: 15 - DNA, 3373 bp, Artificial Sequence
  Description of Artificial Sequence Synthetic polynucleotide
atctcgactaattccacataatacatgctgcctccccccagcaaatcatctagtgtttttgtctccgttgggaattgaacatgagacctcatggtctaaccattcagggcgggacaccatcaaaataccctgaggctcacgtgtgaggcttagttctgtgaggcttacgccccaagcgtccgacagtacgcctgaaatattcctaatactcaatgctcaaggctcacctaacagttccttatgcagtcgtgatttttacctattgttaatttttcttccaataatattaactaaaacttaaataaaaatatcctcttaaatttgtgtttaatcaaatacattcgtataaaattgaagtcgttgtatatatatatcgataacgtagttaagcatgtatattacatattatttaatttagtctagaaatactattgaccgtaatttcataagaacttagctattctttttaagcggactgattgttacataatgtaaatttattttaaattattattgcaccacacttaaattaattctatatgaggaaatgtatatttatttgcggctcccagaaataatagtcgaaatatatatgtgtgtgtgcgcgcgcgctcgcgttatatacatataatatacattttttatacacctttggttagtttcgggcctttcggtcgatcgctcaattttgtattttgtccatttttatattccatatttagtaaattacaatcgtgaaggggagctttggcgtaactgacaaaattattgtcatgagagcaggaggtcacggggtcgagccgtaaaaataacctcttgcaaaaatatagtctaagactgcatacaatataccgtgtggtgcgatccgggctgcccttttacaatcgtgaatcagctccgaggtcccacttgagttggtaagccatgggttggttggtaaaaactcttattggaaagtgagaagaaatagagcgagtcattggtggcacgcctcttttttctcctacaattcagcttctatatatacttccccttaaacttcactactctgcccatacacacaaatacgcatctctcttttctctcatttcttcttcaatctttccacagatctaagagttttttcattttaaggtcagtatttttcaaatttattacgctgaatttactatttcattctttattttttgtttcattagatttagatctggtatatattctgcatgcatctatgacttgtcatagatttcagttaaatcatcatttcttctgttgatgtttttgtttgatttattgcaaaacgatgttttgttttcttatttttgatgttatatatatgttacgttattttctgctatcgctggacgtagtacttttttttccgacgtagtacttagatcctgtttataaagcaaagaacttttgttaattttcatgatattggacttttagctgctaaaattaactgtaatttgtcatattacgtcactcagatctcattgctaactgctaaactggttcgtacagtacattgagttagatcctgtcacaaatatgactttttggccaatggatttggttattacggttcaaatgattgcaaataaagaattattaatgaaatactttatatcatgtgcttaaattgaaatcactcatttttttcttggatgtgggaacctagatcaaataaagaaccggtctttgaaaaaaattaaagtagtgaaaacggcccaaaagacatgttgttgatctgaactttattattcatcattttattccacatagtgttagttctctatatattaccggagaggattcatatagtcgaacccaacttgtttgggattgaggtatagtgattgtttagatatgtccccttaagttgatggaattctctgttttcacatgtttattagcagctggactcattttcttgtgaatatagaacattttgggatagaaatgatgacatattttcttgatcttatcagctagacatggagactttcttgttcacctcagaatcagttaacgaagggcatcccgacaagctttgcgaccaggtctcagatgccattcttgacgcttgcctagaacaagatccagagagcaaggttgcatgtgaaacctgcacaaagacaaacatggtcatggtctttggagagataacaaccaaggccaaggtggactatgaaaagatagtacgcgacacttgcagaggcatcgggttcacctcagctgatgttggccttgatgctgaccactgcaaagtccttgtcaacattgagcagcagagccctgacattgcccaaggagtccatggtcatcttaccaagaaaccagaagaaatcggagctggtgaccaaggtcacatgtttggctatgccacagatgaaactcctgaactaatgccccttactcatgttttggctactcagcttggtgccaagcttactgaagtgaggaagaacaagacttgcccatggctcagaccggatggcaagacacaagttacagttgagtacaagaatgacaatggcgccatggttcctattagagttcacaccgttctcatctcaactcaacatgatgaaggtgtcacaaatgagcagattgcccaggacttgaaagagcatgtgataaagcccgtgatcccagcgaagtaccttgatgagaacaccatcttccacctcaacccatcaggtcgctttgtgattggtggtccacatggagatgctggactcactggcaggaaaattatcattgacacctatggaggttggggtgctcatggtggaggtgccttctcagggaaagatcctaccaaggtggacagaagtggtgcttatattgtgagacaagcagcaaagagtgtggtagcagcaggacttgctcgccgctgcattgtccaggtttcttatgcaattggtgttgcagaaccactctccgtgtttgttgacacttacaaaaccggaaccattccagacaaggatattctggctctgatcaaggagaactttgacttcaggcctggaatgatggcaattaaccttgacttactcagaggaggtaacttcaggtaccagaagactgctgcttatggtcactttggccgtgatgaccctgatttcacctgggagactgtcaaggccctcaagcctaaagcttgagagagtaccaactttcttgtgcctattattcttgttgcggaccaataaacaagctacattgagtatattgataccctattcttacggttaaggccctattgagtggagggagaccaaggaagtgtcttcatactttcgaggattctaagagctccgatagttgatcctgttgatttcttttcttgtttttgccgtactta

SEQ ID NO: 16 - DNA, 3373 bp, Artificial Sequence
  Description of Artificial Sequence Synthetic polynucleotide
agaagtagaagcagccaatgcactgcgcacatccaaaaatagaaaagcaaatgtactttcatgattggttacacagtatcaaagaggatcatcctaaggtaggaaagttggtgagagatctcattcatcaactataatttcatttcatttatttgaatttatatggacattttttcctgcttaaatcgtattaaagtcgttttatcagtaatagaatatataggcgtttggataattcgagttaacattaaaaaaaattatttgaaattaaagttactaagattttgaagctgaagtagtaaagtgaaagttttgtgaatggacaaagcaatatttaatttaaatacacacctcaactttaatttaaaagttaaactccctcttcaactttgaataatattattgtattctgaaaattctgaaagttgtgtaggtaagttaagcatgtacattacatacgtattatttgatttattgtaaacataagaataaccttgatttcataagaatttagttatatgcatggaaatgtaatgaatttcttactctatcaatataattaagattagcacttaatgttattaaatagagtttcttattagtccatccgtctcaaattatctgtcgtgtttttcttttacacgtcccttaagaaaatattaaatagaaaatgattttgattattttacccttatttatgtcttaagatataatctctcttcatttaatacttaatcataattaaggtatttgtagtctacaagaacaattaatattaagggtagaaaagaataaaaataattaatttactcttgaatttataaaatgagagataagagttttcgaaatcacggagtactttcttaattccttttaaatgattcaattttatattccttatttagtaaattaatacaatcgctaatcagatccgaggtcccacttgggttggttggtaaaaactcctatgggaaagtgagaagaaatagagcgagtcattggtggcacgccacttttttctcctataattcagcttctatatatacttcccctaaaacttcacaactctgcccatacacaaataagcatctctctctttcacttttctctcatttcttcttcaatctttccacagatctagagttttttttccatttaaaggtcagtatttttcaaatatattacgctgaattactattgcattctttatttttttgtttcattagatttaaatctggtatatattctgcatgcatctatgacttttcatagatttcagttaaatcatcatttctgctgttgatgtttttgtttgatttatttcaaagcgatgttttgatgttattcatatgtttgttacgttattttctgctattgcttgacgtagtacttagatctcgttagtaaagcggaaaacttttgttaatttttatgatttttgactttttagctgctaaaattaactgtaattgtcatactatgttactcggatctcattgctaaactggtttgtacagtacattgagttggatcctgttacaaatatgactttttggccaatggatttggttaattcggtgcaaatgatggcaaatgaagaattgttaatgaaatacgttatatcatgagcttaaaatgaaatcattccaatttttcttggatattgggaacttagatcaagtaaatcaacctgtctttgaaaaaatttaaagcaatgaaaacggggcagaagacatgttgttgatctgaacttcaatattcatcattttattccacaaagtgtagttctcttttccttaattagacattgtcaatgagaattcatatagccgaccctaacttgttttgaattgaggtgtaatagtagttgtttagatatgtccccttaagttgatggaattctaatgttttgacatgtttatgagcagcttgcctcattttctcgtgaatttcttttgagatagaaatgatgacatattttttaattttatcagctagacatggagactttcttgttcacctcagaatcagttaacgaagggcatcccgacaagctttgcgaccaggtgtcagatgccattcttgacgcttgcctagaacaagatccagagagcaaggttgcatgtgaaacctgcacaaagacaaacatggtcatggtctttggagagataacaaccaaggccaaggtggactatgaaaagatagtacgcgacacttgcagaggcattgggttcacctcagctgatgttggccttgatgctgaccactgcaaagtccttgtcaacattgagcagcagagccctgatattgcccaaggagtccatggtcatcttaccaagaaaccggaagaaattggagctggtgaccaaggtcacatgtttggctatgccacagatgaaactcctgaactcatgcctcttacccatgttttggctactcagcttggtgccaagcttaccgaagtgaggaagaacaagacttgcccatggctcagaccggatggcaagacacaagttacagttgagtacaagaatgacaatggtgccatggttcctattagagttcacaccgttctcatctcaactcaacatgatgaaggtgtcacaaatgagcagatcgcccaggacttgaaagaacatgtgatcaagcccgtgatcccagcgaagtaccttgatgagaacaccatcttccacctcaacccatcaggtcgctttgtgattggtggtccacatggagatgctggactcactggcaggaaaattatcatcgacacctatggaggttggggtgctcacggtggaggtgccttctcagggaaagatccgaccaaggtggacaggagtggtgcttatattgtgagacaagcagcaaagagtgtggtcgcagcaggacttgctcgccgctgcattgtccaggtttcttatgcaattggtgttgctgaaccactctccgtttttgttgacacttacaaaaccggaaccattccagacaaggatattttggctctgatcaaggagaactttgacttcaggcctggaatgattgcaattaaccttgacttgcttagaggaggtaacttcaggtaccagaagactgctgcttatggtcactttggccgtgatgaagctgatttcacctgggagactgtcaaggccctcaagcctaaagcttgagagacttccccactcacaacataaactaccaattttcttgtacctattattcttgttgcggaccaataaacaagcttcatttagtatattgatacccctactcttacatttaaggcgctattgagtggagggagaccgagtaagtgtcttcacactttcgaggattctaagagctctgctggttgatcctgttgatttct

SEQ ID NO: 17 - DNA, 3373 bp, Artificial Sequence
  Description of Artificial Sequence Synthetic polynucleotide
taagaacatgtgagtcatgactcaatgtgacgacgcacaaacccccgaaaatagaatggaaacgtacgttcaagattggctccactgaattttagggatcatctgaaccgttaaaataataaacagtgggtcccgaaaagagagggtaaagttggggtgctaaaccgtgtttttaaaaacaaaatggtgtggtgctaaatgggtcccacagaaggtcattgtgggccataggttggttggtgtacgaaatgaaagtgagaagaaagagagattgagaaagcgagtgaatcatacgcgccatttcccccttcaatttcgcttgtgtataaatactggaccacaacttccccaaactcgtgacttcacacagatcagatccaattctcttctgtctttcccttctctgctctcaaattcttcagatctacaaagttttcttcattttcagaggtatgtttttcaaatccatgccgcaaatttatagttgttactttcgtttttctgtatcaaattcgtaatttgtgtttactcctattagatctgttatgaatctcatgcatattttttgttgatttacgatctagatctcagcatttctaaggtgaatattctcgttatattgagatatatttatgaatatgagtatcatacatgttaggattcgtttggtaatagttgaatgtatgtaaatttagcatttgtctttcgacatttttcgatatggagtcttattcatgcattgcttaggttaatagttagatctcagcatttttgcagtgaatattctcgttatattgagttatatttgtgaacatgagtaccctatatgttgattcgtttggtaattgttgaatttctgtaaattttctcaatggaggcttatgcatgcattacttaggttaatggttagatctctttgtttactggtgatattatctttgattgttacgttatttgttgatattggtaaaaccagtagtcagatctcattgataaactagttttaccgtaacacagttgtatttcgaaaaaagtttgatattttttcttaaatttgggtaatttaagtgttaatcgtgagtataacggcttaagcctgtttgatcttgattaattattgaaaatgaaaaaggaaaatagttatttttgattgttgcgttgtctcatctgaagtaatactcaaatctcatttgtaaactccgtacaaggtgttgcatctgcataaaaaatgtaaattttcgtgaatttgggtgatttaagtgctaacaatgtaactcttcattgttaattttagaaaggaaaagccaaaaaagcaacaaataaaaacgttgactctcattattatattgtcttctaaggttatttgaagtgtgttaatctcattgctaaaaactggtgtttacagtaaaaaagttgcatcctgtacttgtatggtctacaagtgctatcagcaaaataattatggaagtgtgttatattatcatatgaataaaagctaaaatagtgttcaggttgatgattcaatcttgtattacttacattaagttagggaaatgttttttggagttctcagcaagaataccaaggatttttgaaaatgtcagttgagttttggggtaaaatctgtgatgaatcttcatgcatatttgatccttaaaatgttctggtccactagatctttttaataacaacgtttccactacaattatgacctagttatattgccaaataagtgttaatataataatggaggtcaactctggttattccatcagctttaatacttctggtcttctgcctctaggtggagaattcttgtcattgagcacctcaaatggttcctcgagatgattaaatttcttttctgttattgagttctgagttctatctactttttattgtctcatgtgtaatgcaagcagcttttccttgattttctggtaaattgaatattttaagataggttttaacacttgctcttaaatttgatcagggcagacatggaaactttcttgttcacctcagagtcagtcaatgaaggccaccccgacaagctctgtgaccaggtctcagatgcaattcttgatgcttgcttagaacaggatccagaaagcaaggttgcatgtgaaacctgcacaaagacaaacatggttatggtctttggagagatcacaaccaaggccactgtcgactatgagaagatagtacgtgacacatgcagaggcatcgggttcacctcagcagatgtcggccttgacgctgacaactgcaaggtccttgtcaacattgagcagcagagccctgacattgcccaaggtgttcacgggcatcttaccaagaaaccagaagagattggagctggtgaccaaggtcacatgtttggctatgccactgatgaaactccagagctcatgccccttacccatgttttggccactaagcttggcgccaagcttactgaagtgagaaagaacaagacttgcccatggctcagaccagatggcaagacccaagttactgttgagtacaagaacgacaatggtgccatggtccccattagagttcacactgttctcatctctactcaacatgacgaaactgtcacaaacgaccagattgcccaggacttgaaagagcatgtgattaaacctgtgatcccagctcagtaccttgatgagaataccatcttccacctcaacccatcaggtcgcttcgtcatcggtggaccacacggagatgctggacttaccggcaggaaaattatcattgacacctacggaggctggggtgcccatggtggaggtgctttctcaggaaaggatcctactaaggtggacaggagtggtgcttatattgttagacaggcagcaaagagtgtggtcgcctcaggacttgctcgccgctgtattgtgcaggtttcttatgctatcggtgtggctgaaccactttccgtgtttgttgacacttacaagactggaacaattccagacaaggatattttggctctgatcaaggagaactttgacttcaggcctggaatgatgtcaatcaatcttgacttgttaagaggaggcaacttcaggtaccagaagactgcagcttatggtcactttggccgtgatgaccccgacttcacatgggagactgtcaagagcctcaagcctaaagcttaagtgaggtcttgcattttggccattatttttcttgcagaccaataaacaagcttcatcatatcatgcattgttggcaggagaagggaatttgtgtctccattgaggattctacgagctcttgagtcattgaacatttgttatttttctttcttttttcacccttttctgcagtaccttatttttatgttgttactgttaag

SEQ ID NO: 18 - DNA, 4397 bp, Artificial Sequence
  Description of Artificial Sequence Synthetic polynucleotide
aaccaacttattattattagtagtagtattgttattgttatgctattattattattattattctttttttagattctcaccaactacaactaatagtaaaaacggaaaagggtcaaatatgttcttatattattgaaaaaaggtttaatattaccctccgttatactatcactctaaatatactcgtaacgtcatactatcggttcaaatatatttctgacatcatgaaagtggtacaaacctacctttcctccgttaactgcctcccctataaacacattttattgctacctcactatttcaccctatttgcagctaccaagttcaattactttgagattctatcattattttgaagactttagtgcctctacttcctcttgcttttcaaaatacttgttgcttctccgaccgatcctagaattgtacgtacctttcacttgaccttacaatttatgttttattttgcatttccttgtgttgcagctgaatttgtaagatctatttacggaaaatttagtagcacttttattttaacacaaaacgtaaagagagaaaaaatggtagccttccagaaatgtggttgaagagaaagaaacaaaaaaaattaactctcgtgtctaggttgcttttctttctcaccatggtcaattttgaaaccttaaactagtttacctgtatctttgcttagtttttttatatataaaataaaatgtattttcgtttgtatacatgaattttgaatagcaaaatcaaaattttttcggttcacattgtttcatattatctttagttgtcagcatttcttttgatgtagtttgtttgtatatttatgatctgtgatttttcttcaaatgaacgggaaagaaagttgtggggaagggatgaaaatgagagtataatggtgaggtggtaatgaaatgttgtcatagggaggcaattaatagaggaaggataagtttgtaccgcttttataatgtcaggagtatatgtggaccgatagtatgatgttaggggtatatttggatagtataacggaggacaatattagacctttccaatatgataggggcatatttgacccttttctgtagaaaaaattataaaaccatcttaatgggaacatgtgagtcataactaacgtaacgatacaaaaaaccccgaaaatagaatggaaatgtacgttcatgattggctacacgcaattttagggatcatctggcccgttaaaataaacagtgggtcccgcaaagagagggtaaagttggggtgctaaatcgtgttttaaacaaaaaaacggtgtggtgctaaatgggtcccacagaaggtcattgtgggccataggttggttggtgtatgaaatgaaagtgagaagaaagagagatcgagaaagcgagtgaatcatacgcgccattgtccccttcaatttcgcttgtgtataaatactggaccacaacttccccaaactcgtgaccatacacagatcagatccaattctcttctgtgcttcccttctctgctctcaaattcttcagatctacaaagttttcttcattttcagaggtatgtttttcaaatccatgccgcaaatttacagttgttacttcttttttctgtatcaaattggtaatgtgtttactcgtattagatctgttatgaatctcatgcatatttttttttgttgatttacgatctagatctcagcatttctgcggtgaatattctcgttatattgaattatatttatgaacatgagtatcatacatgttagattcgtttggtaataactgaatgtctgtaaattagtgtttttctatcgacatttttcgatatcgagtcgtattcatgaattacttaggttaatagttagatctcagcatttttgcggtgaatattctcgttatattcagttatatttgtgaacatgagtagttgagtaccatacatgttgattcgcttggtaattgttgaatgtctttaaattttcgatatggagacctatgcatgcattacttaggtgaatggttagatctctttgtttactagtgatgttatctttgattgttacgttttttgttaatgttggtaaaaactagttagccggatctcattgataaactagttttactgtaccaaggagttgtatttggacaaaagtttggtacttttcataaatttgggtagtttaagtgttaatcgcgagtgtaacggcataaacctgtttgatcttggttaattattgaaaaggaaaaaggaaaatagtaatttttaattgttgcgttgtctcatatgaagtactactcagatctcatttataatttataaataccttacaaggagttgcatcttcatatgatttaagggctaacaatgtaattcttcattgttaagtttagaaaggcaaaagccaataaagcaaccaaaaaagaagttgactctggttgttatattgtcttctaaggttatctgaggtgtgttgatctcattgctaaaaaccggtttttacagtaaaacaattgcatctttgtacttgtgcggttactgcaagtgctatcagcaaatattattatggaaaaacgttatattatcatattatcatatgagtaaaaggtaaaatagtgttagattgatgattcaatcttgtattgcttacatttagttagggaaatgttttttggagttctcagcaagaataccaaggatcttataaaaatctctgaagttgagttttggggtaaaatctgtgattagtcttcatgcatatttgatcctcaaaatgttctggtccactagatcttcttaataacagtgtttccattacaattatgacctagttatattgctaaatagtaagtgttaatataatggatggaggtcaactctggttattccatcatcactcttgagtctgactactttcattgtctcatgtgtaatgcaatcagcttttccctgattttcctggtaaattgaatattttaagataggttttaacactcgctcttaaatttgatcagggcagacatggaaactttcttgttcacctcagagtcagtcaatgaaggccaccccgacaagctctgcgaccaggtctcggatgcaattcttgatgcttgcttagaacaggatccagaaagcaaggttgcatgtgaaacctgcacaaagacaaacatggttatggtctttggagagatcacaaccaaggccactgttgactatgagaagatagtgcgtgacacatgcagaggcattgggttcacctcagcagatgttggccttgacgctgacaactgcaaggttcttgtcaacatcgagcagcagagccctgacattgcccaaggtgttcacggtcatcttaccaagaaaccagaagagattggagctggtgaccaaggtcacatgtttggctatgccactgatgaaaccccagagctcatgccccttacccatgttttggccactaagcttggtgccaagcttaccgaagtgaggaagaacaagacttgcccatggctcagaccagatggcaagacccaagttactgttgagtacaagaacgacaatggtgccatggtcccaattagagttcacactgttctcatctcaactcaacatgacgaaactgtcacaaacgaccagattgcccaggacttgaaagagcatgtgatcaaacctgtgatcccatctcagtaccttgatgagaataccatcttccacctcaacccatcaggtcgcttcgtcatcggtggaccacacggagatgctggacttaccggcaggaaaattatcattgacacctacggaggctggggtgcccatggtggaggtgctttctcaggaaaggaccctactaaggtggacaggagtggtgcttatattgttagacaggcagcaaagagtgtggtcgcctcaggacttgctcgccgctgtattgtgcaggtttcttatgctatcggtgtggctgaaccactttccgtgtttgttgacacttacaagactggaacaattccagacaaggatattttggctctgatcaaggagaactttgacttcaggcctggaatgatgtcaatcaaccttgacttgttaagaggaggcaacttcaggtaccagaagactgcagcttatggtcactttggccgtgatgaccccgacttcacatgggagactgtcaaggtcctcaagccaaaagcttaagtgaggtgtagccttttggccattatttttcttgcagaccaataaacaagcttcatcatatcatgcattggtggcaggagaagagaatttgtgtctccattggaggattctatgagctctgagtcattgaacatttgttatttttctttcttttttttcacccttttctgcagtaccttatttttattttgttactgtta

SEQ ID NO: 19 - DNA, 861 bp, Artificial Sequence
  Description of Artificial Sequence Synthetic polynucleotide
atggaacagaaaggccaagaaaaggaaatgggcataccaaactccattggctataatccatcttcaagtttttcagatactagaagaaatgaaaaagcaggcagtggaaatgtcattctcagccctatccaaactctaaatcagcacaaccaacattccctacaaggacatagccaactaaattcacatcaattagaagaagaaagggatccacttgttgcagctaccaatagagcacaactacgacaacaatcgacaacctcaggaaatggcacgaccacgaccacgaccagctcatcatcagtggttagacgataccgagaatgtctcaagaatcatgttgcaagcatgggaggacatgtccttgacggatgtggcgaattcatgccaagcgaagaagaaggtgcgctaaagtgtgcagcttgtaattgtcatcgaaattttcatcggagagaaactttctccgaggctgagacgcagatagctcgcccacgccccccatattctacattcaatccaccaatacatagtagtagcaattaccaccacaattatagtaccgcgtcaccagtgatgatgacttttggaaggaatgctgaatcatcaagtgagcatctaaatttgttcaattctaatgccggaggaggacaaagttcgaagtcgaagaagagatttaggacaaagttcacagcggaacaaaaagaaagaatgcatgaatttgctgagaaggtgggatggagtatacagaagcaagacgaacaagaattgcaacacttctgtaaccaagtgggtgtcaagagacaagttttcagaatatggatgcacaacagcaaacaagcaaataagaagagacaaatgtaa

SEQ ID NO: 20 - DNA, 1560 bp, Artificial Sequence
  Description of Artificial Sequence Synthetic polynucleotide
atgtatcatcttctttctcccatagaagccattgtaggacttgtaacctttgcatttctactctacttcctatggacaaaaaaacaatcaaaaatcctaaacccactgcctccaaaaatcccaggtggatggccagtaatcggccatctcttttatttcaagaacaatggcgatgatgaccgccatttttctcaaaaactcggagacttagctgacaaatatggtcccgtcttcacattccggttagggtttcgtcgtttcttggcggtgagtagttatgaagctatgaaagaatgcttctctaccaatgatatccatttcgccgatcggccagctttactttacggagaatacctttgctataacaatgccatgcttgctgttgccaaatatggcccttactggaaaaaaaatcgaaagctagtcaatcaagaacttctctccgttagtcggctcgaaaaattcaaacatgttagattttctatagttcagaaaaatattaaagaactatatgatggtgattcaccaatggtgaagataaaccttagtgattggatagataaattgactttcgacatcattttgaaaatggttgttgggaagacctataataatggacatggagaaatactgaaagcagcttttcagaagttcatggttcaagctatggagattgagctctatgatgtttttcacattccatttttcaagtggttggatcttacagggaatattaaggctatgaaacaaactttcaaagacattgataatattatccaaggttggttagatgagcacattaagaagagagaaacaaaggatgttggaggtgaaaatgaacaagattttattgatgtggtgctttctaagaggagcaacgaacatcttggcgatggttactctcatgacaccaccatcaaagcaaccgtattcactttggtcttggatgcaacagacacacttgcacttcatataaagtgggtaatggcgttaatgataaacaataagaatgtcatgaagaaagcacaagaagagatggacaccattgttggtagagatagatgggtagaagagagtgatatcaagaatttggtgtatcttcaagcaattgttaaagaagtattacgattacatccacctgcacctttgtcagtacaacacctatctgtaaaagattgtgttgtcaatggataccatattcctaaggggactgcactacttacaaatattatgaaactgcaacgagatcctcaaatatgggcagatcctgataaattcgatccagagagattcttgacaactcatgctgcaattgactatcgagggcagcactatgagttgataccgtttggtacggggagacgagcttgtcccgcaatgaattactcattgcaagtggaacacctttcaattgctcatatgatccaaggtttcaattttgcaactacgacaaacgagcctttggatatgaaacaaggtgtgggtctaactttacctaagaagacagatgttgaagtgctaattacacctcgcctgcctcctacgctctatcaatattaa

SEQ ID NO: 21 - DNA, 1125 bp, Artificial Sequence
  Description of Artificial Sequence Synthetic polynucleotide
atggatatgaaggggacgccagcggaaaatggataccgtatgcctgctgaatgggaatcccactctcattgttggatgggctggcctgagcgtccagataactggcgaaacaatgcactccacggtcaacgtgtatttgccaatgtagcatctgctatctcaagattcgaaccagtaacagtctgtgctagtgctgctcagtgggaaaatgcacgcagccagttaccaaaacatatcagggttgtggagatgagcatgaatgattcttggtttcgtgatactggtccaacttttgtcatcagaaataccgcagcaaatgcagaacatgtagcgtcgagtgttgcaggaattgattggaccttcaatagctggggtggtgttgatgatggatgttacgaagactggagtcatgaccttcttgttgcaagaaagattttggcaattgaaaaggtcccaaggtttccacactctatgattcttgaaggtggaagcattcatgtcgatggagaagggacttgtctcacgacagaagagtgcctcctgaacaaaaacaggaacccccatttgtctaaggaacaaattgagaatgaactaaaggcatatcttggagtcaggaagattatttggctgcctcgagggttgtttggtgatgatgatacaaatggtcatattgacaacatgtgctgttttgtgaagcctggtgttgttttattgtcatggaccgatgatcaatctgatccacagtatgagagatccattgaagccctttctattcttgaaactacttctgatgcgaatggtagaaaactacaagtgatcaaactccatgtacctgggccactctgtatgacagatgaggaggcagatggaatatttcaggatggtgatgcaaaacctaggacttcaggtacaaggcttgctgcttcgtacgtaaacttctacattgccaatggcgctattattactccacaatttggggacaaaaaatgggatgaagaagctattagagtcctctcactggcgtttccagatcatgaggtggtaggaattgaaggtgcaagggagattgtattgggaggtggcaatatacattgtatcacgcagcagcagccaaccggtccataa

SEQ ID NO: 22 - DNA, 1164 bp, Artificial Sequence
  Description of Artificial Sequence Synthetic polynucleotide
atggatctgaaggggacgccagttgaaaatggataccgtatgcctgctgaatgggaatcccactctcattgttggatgggctggcctgaacgtccagataactggcgaaacaatgcattccatggtcaacatgtatttgccaatgtagcatctgctatctcaagattcgaaccagtaacagtctgtgctagtgctgctcagtgggaaaatgcacgcaacctcttacctaaacatatcagggttgtggagatgagcatgaatgattcttggtttcgtgatactggtccaacttttgtcatcagaaataccacatcagaagatactgaacatgtagcgccaagtgttgcaggaattgattggaccttcaatagctggggtggtgttgatgatggctgttacgaagactggagtcatgaccttcttgttgcaagaaagattttggcaattgaaaaggtcccaaggtttccacactctatgattcttgaaggtggaagcattcatgtcgatggagaagggacttgtctcacgacagaagagtgcctcctgaacaaaaacaggaacctacatttgtctaaggaacagattgagaatgaactaaaagcatatcttggagtcaggaagattatttggctgcctcgagggttgcttggtgatgatgatacaaatggtcatattgacaacatgtgctgttttgtgaagcctggtgttgttttattgtcttggaccgatgatcaatctgatccacagtatgcgagatccattgaagccctttctattcttgaaactacttctgatgccaatggtagaaaactacaagtgatcaaactccatgtacctgggccactctgtatgacagatgaggaggcagatggaatatttcaggatggtgatgctaaacctaggacttcaggtacaaggcttgctgcttcatacgttaacttctacattgccaatggcgcaattattactccgcaatttggggataaaaaatgggatgaagaagctattagagtcctctcactggcatttccagatcatgaggtggtaggaattgaaggtgcaagggagattgtattgggaggtggaaatatacattgtatcacgcagcagcagccaagcggcccagaatattcacccaagattttaaatggttccacatcttag

SEQ ID NO: 23 - DNA, 1947 bp, Artificial Sequence
  Description of Artificial Sequence Synthetic polynucleotide
atggcaactggtatcgcttcaggatgcggacagttacatttgaggaagcctgtctacttgaggaatagctatggaaataaagctcactctcattccaatgtgattctcaacggcacgcaaaaccagatcgcttggtctagttgggtttcaaatgtcttgcgagttaatagaagtagctatccacaatgtcaagtgataaaaacaaactggaagtctcggcgaggaacaatcaaatctagccagcagagagatggatcagttactaggtacttcgatttcattgtgattggtagtggaattgctggccttcgatatgcacttgaggttgccaagcatggaactgtggctgtgataaccaaggctgagccacatgagagtagcactaactatgctcaaggtggtgtaagtgctgtgctctgccctttggattcagtggagaaccacatacaagatacaattgtggcaggtgcttacctctgtgataaggagactgttaaagtagtgtgtactgaaggacctgagagaattagagaactgatcgctataggtgcttcattcgatcatggggaggacggaaatctggatctagccagggaaggaggccactcccatcgtcgaattgtccatgctgctgatatgactggcagagagattgaaagagccttattagaggcagtgtttaagaatcctaatatacatgtgtttcaacaccattttgctatcgatttgttgaccactcaggatggttctgacgtagtatgtcatggcgttgatactatacacacggaaacgaaggaggttataagattcatttcaaaagtgactttgctagcatcaggtggagttggacatatctatccaagtactactaatccgacggttgcaactggtgatggaatggctatggctcatcgagctcaagctgtaatttccaacatggagtttgtgcaattccacccaactgccttggctgatgaagggcttcccaacataccaagtgccagagagaatgcttttttgataactgaagctgtcagaggtgatggaggcatcctttataacttagatatggaaagatttatgccaatgtatgatgaaagagcagaacttgccccgagagatgtggtagcaagaagtatagatgaccagctcaaaaagcgtggcgaaaagtatgttcttcttgatatcagtcacaagcccagagagaaggttctgtcccattttcctaatatagctgctgagtgtctccgccatgggttagacataacacagcagccgattccggtggttcctgctgctcactacatgtgtggtggagtccgtgctggactcgagggtgagactaatgtgcaaggtctttatgtggcaggtgaagttgcatgtactggtttacatggtgcaaaccgacttgctagcaactcattgcttgaagcactagtgtttgcacgaagagctgtacagccttcaattgatcatgtgaacgtgtctagaattgataacggtgcatcaagttggtggccgcggcctgtagcccccctggcactaggagatacagtacttaacaaagtcatccgtcggacaagggaagtgaggaaagaactacagtcaatcatgtgggaatatgttggaattgttaggtctacctcaagactaaatactgctgagaagagaatcaaagagttggagttggaatgggaaacatacctgtttcagcatggctgggaaccaacaatggttggagtagaggcttgtgagatgaggaatctcttctgttgtgccaacctggtagttagcagtgctctttctcgacaagagagtcgtgggcttcactacaccactgattttcctcatgttgaggaaagcgagaggttgccaacggttatctttccttctcagcgaaatagcacatggagcacacggcaattacacgcgcagccgataagttag

SEQ ID NO: 24 - DNA, 1989 bp, Artificial Sequence
  Description of Artificial Sequence Synthetic polynucleotide
atgttctggatattggaaattgactttccttgggttcttagtcaggaagatcatatggcaactggtatcgcttcaggaagcggacagttacatttgaggaagcctgtctactggaggagtagctatggaaaagctcactgtcattccaatgcgatcctgaatggcatgcaaaaccagatcccttggtcttattgggtttccaaattcttgcgagttcatagaagtaactactcacaatgtcaagtgaaaacaaactggaagtctcacaccggaacaatcaattcttgccagagagatggctcaactaggtattttgattttgctgtgattggtagtggaattgctggccttagatatgctctagaagttgcaaagcatggaactgtagctgtgataaccaaggctgagcctcatgagagtaacactaactatgctcaaggtggtgtaagtgctgtgttctgccctatggattcagtggagagccacatgcaagacacaattgtggcaggtgcttacctctgtgatgaggagactgttagagttgtgtgtaccgaaggacctgagagaattagagaactgattgctatgggtgcttcattcgatcatggggaggacgggaatctgcatctagccagggaagggggccactcccatcgtcgaattgtccatgctgctgatatgactggcagagagatagaaagggccctattagaggcagtttttaagcatcctaatatacatgtgtttcaacaccattttgctatagattttttgaccactcaggatggttctgacataatatgtcatggcgttgatgctataaacacggaaacgcaggaagttataagattcatttcaaaagtgactttgctggcatcaggtggagctgggcatatctatccaagtactactaatccaccggttgcaactggagatggaatggctatggctcatcgagctcaagctgtaatttccaacatggagtttgtgcaattccatccaactgccttggctgatgaaggccttcccaacagaccaagtgccagagagaatgcttttttgataactgaagctgtcagaggtgatggaggcatcctttacaacttagatatggagagatttatgccaatgtatgacaaaagagcagagcttgccccgagagatgtggtagcaagaagtatagatgaccagctcaaaaagcgtggcgaaaagtatgttcttcttgatatcagtcacaagcccagagagaaggttctgtctcattttcctaatatagctgctgagtgtctccgccatgggttagacataacacagcagccgattccggtggttcctgctgctcactacatgtgtggtggagttcgtgctggacttgagggtgagactaatgtgcaaggcctttatgtggcaggtgaagttgcatgtactggtttacatggtgctaaccgacttgctagcaactcgttgcttgaagcactagtgtttgcacgaagagctgtacaaccttcaattgatcacgtgaatgtgtctagaattgatcactgtgcttcaagttggtggccgcgacctgtagccccagtggtaataggagatacagtacttaacaaagtcattcgtcggacaagggaagtgaggaaagaactacagtcaatcatgtgggaatatgttggaattgttaggtctacctcaagactaaccgcagctgagaagagaatcaatgagttggagttggaatgggaaacatacctatttcagcatggctgggaaccaacaatggttggactagaggcttgtgagatgaggaatctcttctgttgtgccaacctggtagttagcagtgctctttctcgacacgagagtcgcgggcttcattacaccattgattttcctcatgttgtggaaagcaagaggttgccaacaatcatttttccttcacagcgaaatagctcgtggagctcacggcaattacacaggcagcagatatgttag

SEQ ID NO: 25 - DNA, 1947 bp, Artificial Sequence
  Description of Artificial Sequence Synthetic polynucleotide
atggcaactggtatcgcttcaggatgcggacagttacacttgaggaagcctgtctacttgaggaatagctatggaaataaagctcactgtcattccaatgtgattctcaacggcacgcaaaaccagatcgcttggtctagttgggtttcaaatgtcttgcgagttaatagaagtagctatccagaatgtcaagtgatcaagacaaactggaagtctagccgaggaacaatcaaatcgtgccagcagagagatggatcagttactaggtacttcgatttcactgtgattggtagtggaattgctggccttcgatatgcacttgaggttgccaagcatggaactgtggctgtaataaccaaggctgagccacatgagagtagcactaactatgctcaaggtggtgtaagtgctgtgctctgccctttggattcagtggagagccatatgcaagatacaattgtggcaggtgcttatctctgtgataaggagactgttagagtagtgtgtactgaaggacctgagagaattagagaactgatcgctatgggtgcttcattcgatcatggggaggacggaaatctggatctagccagggaaggaggccactcccatcgtcgaattgtccatgctgctgatatgactggcagagagatagaaagagctctattagaggcagtttttaagaatcctaatatacatgtgtttcaacaccattttgctatagatttgttgaccactcaggatggttctgacatagtatgtcatggcgttgatactatacacacggaaacgaaggaggttataagattcatttcaaaagtgactttgctggcatcaggtggagttggacatatctatccaagtactactaatccgacggttgcaactggtgatggaatggctatggctcatcgagctcaagctgtaatttccaacatggagtttgtgcaattccacccaactgccttggctgatgaaggccttcccaacataccaagtgccagagagaatgcttttttgataactgaagctgtcagaggtgatggaggcatcctttacaacttagatatggaaagatttatgccaatgtatgatgaaagagcagaacttgccccgagagatgtggtagcaagaagtatagatgaccagctcaaaaagcgtggcgaaaagtatgttcttcttgatatcagtcacaagcccagagagaaggttctttctcattttcctaatatagctgctgagtgtctccgccatgggttagacataacacagcagccgattccggtggttcctgctgctcactacatgtgtggtggagtccgtgctggactcgagggtgagactaatgtgcaaggtctttatgtggcaggtgaagttgcatgtactggtttacatggtgctaaccgacttgctagcaactcattgcttgaagcactagtgtttgcacgaagagctgtacagccttcaattgatcatgtgaacgtgtctagaattgatcacggtgcttcaagttggtggccgcggcctgtagcccccatggtactaggagatacagtacttaacaaagtcatctgtcggacaagggaagtgaggaaagaactacagtcaatcatgtgggaatatgttggaattgttaggtctaactcaagactaaacactgctgagaagagaatcagagagttggagttggaatgggaaacatacctatttcagcatggctgggaaccaacaatggttggagtagaggcttgtgagatgaggaatctcttctgttgtgccaacttggtagttagcagtgctctttctcgacatgagagtcgtgggcttcactacaccactgattttcctcatgttgaggaaagcgagaggttgccaacggtcatttttccttctcagcgaaataactcatggagctcacgccaattacacgcgcagccgataagttag

SEQ ID NO: 26 - DNA, 1938 bp, Artificial Sequence
  Description of Artificial Sequence Synthetic polynucleotide
atggcaactggtatcgcttcagtaagcggacagttacatttgaggaagcctgtctactggaggagtagctatggaaaagctcagtgtcattccaatgtgatcctgaatggcatgcaaaaccagatcccttggtcttattgggtttccaaattcttgcgagttcatagaagtaactattcacaatgtcaagtgaaaacaaactggaagtctcacagcggaacaatcaattcttgccagagagatggctcaactaggtatttcgatttcgcggtgattggtagtggaattgctggccttcgatatgctcttgaagttgccaaacatggaactgtagctgtgataaccaaagctgagccacatgagagtaacactaactatgctcaaggtggtgtaagtgctgtgctctgccctatggattcagtggagagccacatgcaagatacaattgttgcaggtgcttacctctgtgatgaggagactgttagagttgtgtgtaccgaaggacctgagagaattaaagaactgattgctatgggtgcttcattcgatcatggggaggatggaaatctgcatctagccagggaagggggccactcccatcgtcgaattgtccatgctgctgatatgactggcagagagatagaaagggccctattagaggcagtttttaagcatcctaatatacatgtgtttcaacaccattttgctatagatttgttgaccactcaggatggttctgacatagtatgtcatggcgttgatactataaacacggaaacacaggaggttataagattcatttcaaaagtgactttgctggcatcaggtggagctgggcatacctatcctagtactactaatccgccggttgcaactggagatggaatggctatggctcatcgagctcaagctgtaatttctaacatggagtttgtgcaattccacccaaccgctttggctgatgaaggccttcccatcagaccaacaagaaccagagagaacgcttttttgataactgaagctgtcagaggtgatggaggcatcctttacaacttagatatggagagatttatgccaatgtatgatgaaagagcagagcttgccccgagagatgtggtagcaagaagtatagatgaccagctcaaaaagcgtggtgaaaagtatgttcttcttgatatcagttacaagccccgagagaaggttctttctcattttcctaatatagctgctgagtgtctccgccatgggttagacataacacagcagccgattccagtggttcctgctgctcactacatgtgtggtggagtccgtgctggactcgagggtgagactaacgtgcatggtctttatgtggcaggtgaagttgcatgtactggtttacatggtgcgaaccgacttgctagcaactcgttgcttgaagcactagtgtttgcacgaagagctgtacagccttcaattgatcacatgaatgtgtctagaattgatcacggtgcttcaagttggtggtcgcggcctgtagctcccatgttactaggagatacagtacatggcaaagtcattcaccggacaagggaagtaaggaaagaactgcagtcaatcatgtgggaatatgttggaattgttaggtctacctcaagactaaccgcagctgagaagagaatcagagagttggagttggaatgggaaacatacctatttcagcatggctgggaaccaacaatggttggagtagaggcttgtgagatgaggaatctcttctgttgtgccaacctggtagttaacagtgctctttctcgacacgagagtcgcggtcttcattacaccattgattttcctcatgttgaggaaagcaaaaggttgccaacgatcatttttccgtcacagcgaaatagctcatggagctcgcggcaattacacaggcagcagatatgttag

SEQ ID NO: 27 - DNA, 1017 bp, Artificial Sequence
  Description of Artificial Sequence Synthetic polynucleotide
atgaagagtgcaggaaggatgggaatccactatatgcagaaattgaacgcggcgaatgttccaaaagaattagtagaaaaaggacagaatcgtgttatagaagcatctcttacacttattcgtgaaagagcaaagcttaagggagagcttattcgtgctctcggaggtgctgtggcttcaacgtctcttcttggagttccgttgggacataactcttcatttctccaagggccagcattcgctcctcctcgtatacgagaggctatttggtgtggcagcactaactccacgaccgaggaaggaaaagaattaaatgatccacgcatcttgaccgatgttggtgatgtgccagttcaagagttacgagatagtggcgttgatgatgataggttgatgagtatcataagtgaatctgtcaagctagtgatggaggaggatccattgcgccccttggtgttagggggtgatcactccatatcctatcctgttgtaagagctgtgtctgaaaagcttggaggacctattgatatccttcaccttgatgctcatcctgatatttaccattcctttgaaggaaacaaatattcacatgcatcaagctttgcacgaattatggagggtggttacgctcgacggcttttgcaagtgggaattagatcaattaccaaagaaggtcgtgaacaagggaaaaggtttggcgtggagcaatatgaaatgcggactttttccagagaccgccagtttttggagaatctgaaacttggtgaaggtgtgaagggcgtgtatatctcagtggatattgactgtatggatccagcatttgctcctggagtatctcatatagaaccaggcggtctctctttccgtgatgttctaaacatactgcataaccttcaagcggatgttgttggtgctgatgttgttgagttcaacccacagcgtgatactgttgatggcatgactgcgatggttgctgcgaagctggtaagagaacttactgccaagatatccaagtga

SEQ ID NO: 28 - DNA, 1029 bp, Artificial Sequence
  Description of Artificial Sequence Synthetic polynucleotide
atgaagtgtgcaggaaggatgggaatccactatatgcagaaattgaacgcggcgaatgttccaaaagaattagtagaaaaaggacagaatcgtgttatagaagcatctcttacacttattcgtgaaagagcaaagcttaagggagagcttattcgtgctctcggaggtgctgtggcctcaacgtctcttcttggagttcctttgggacataactcttcatttctccaggggccagcatttgctcctcctcgtatacgagaggccatttggtgtggcagcactaactccacgactgaggaaggaaaagaattaaatgattcacgcatcttaaccgatgttggtgatgtgccagttcaagagttgcgagatagtggcgttgatgatgataggttgatgagtatcataagtgaatctgtcaagctagtgatggaggaggatccattgcgccccttggtgttagggggtgatcactccatatcctatcctgttgtaagagctgtgtctgaaaagcttggaggacctattgatatccttcaccttgatgctcatcctgatatttaccattcctttgaaggaaacaaatattcacatgcatcaagctttgcacgaattatggagggtggttacgctcgacggcttttgcaagtgggaattagatcaattacaaaagaaggtcgtgaacaaggaaaaaggtttggcgtggagcaatacgaaatgcgaactttttccagagaccgccaatttttggagaatctgaaacttggtgaaggtgcgaagggcgtgtatatttctgtggatatcgactgtatggatccagcatttgctcccggagtatctcatatagaaccaggcggtctctctttccgtgatgttctaaacatactgcataaccttcaagcggatgttgttggtgctgatgttgttgagttcaacccacagcgtgatactgttgatggcatgactgcaatggttgctgcgaagctgaacaaagcgtttaccaatggggcgaggcatagcccggaaccatga

SEQ ID NO: 29 - DNA, 1299 bp, Artificial Sequence
  Description of Artificial Sequence Synthetic polynucleotide
atggccggccaaacaataatcgtttccgggttgaacccggcggccattcttcagtccacaataggcggcggagcttcacctacagcggcggcggcggaaaacggtaccagaaaagtcatccctctctcaagagatgccctccaagatttcatgttatcaatcataacccaaaaattacaagatgagaaacaacctttttacgtgttagatttgggtgaagttgtttctcttatggaccaatggaaatctgctctcccaaatatccgtccattttacgctgttaaatgtaaccctgaaccgtcgttcctttcaattttatctgctatgggctcaaattttgattgtgctagccgagctgaaattgagtacgttttgtcccttggcatttcacctgaccgtattgtttttgcaaatccatgcaaaccggaatccgatattatttttgcagcaaaagttggggtgaatttaacaacgtacgattcagaagacgaggtttacaagatccgaaagcatcacccgaaatccgaactcttgctccgaatcaagccaatgttcgacggcaacgcgaggtgcccaatgggtccaaaatacggcgcgcttccagaagaagtcgagccgctgctccgggcagctcaggccgcccggctcaccgtctccggtgtctccttccacatcggcagcggagatgccgattcaaacgcttatctcggcgccatagccgcggctaaggaagtgtttgaaacagctgctaaactcggtatgtcgaaaatgactgttctagacgtcggcggcgggtttacatccggccaccagttcacaaccgccgccgtcgctgttagatcagctttaaaacaacacttcgatgatcaaccggagttgacaatcatagctgaaccgggccggttttttgcggagactgcgtttactttagcgacgacgattatagggaaaagagtgaggggagaattgagggagtattggattaacgacgggttgtacggttcgatgaactgtgtactttacgaccatgcgacggtgaatgcaacgccgttagctgttttgtcgaatcgtactaacgttacctgcggcgggtcgaaaacgtttccgacgactgtgtttgggcccacttgtgatgctcttgatactgttttaagggattaccagttaccggagctgcaggttaatgattggctagtttttcctaatatgggtgcttatactaaagctgctgggtccaattttaatggatttaatacgtccgccattgttactcacctcgcttatgcttatccaagctga

SEQ ID NO: 30 - DNA, 1302 bp, Artificial Sequence
  Description of Artificial Sequence Synthetic polynucleotide
atggccggccaaacaatcatcgtttccgggttgaacccggcggccattcttcagtccacaattggcggcggagcttctcctacagcggcggcggcggcggaaaacggcaccagaaaagtcatccctctctcaagagatgccttacaagatttcatgttatcaatcataacccaaaaattacaagatgagaaacaacctttttacgtgctagacttgggtgaggttgtttctcttatggaccaatggaaatctgctctcccaaatatccgtccattttacgctgttaaatgtaaccctgaaccgtcgttcctttcaattttatctgctatgggctcaaattttgattgtgctagccgagctgaaattgagtatgttttatctcttggcatttcacctgaccgtattgttttcgcaaatccatgcaaaccggaatccgatattatttttgcagcaaaagttggggtgaatcttacaacctatgattctgaagacgaggtttacaagatccgaaagcatcacccgaaatccgaactcttgctccgcatcaagcccatgctcgacggcaacgcgagatgcccaatgggcccgaaatacggcgcgcttccagaagaagtcgacccgctgctccgggcagctcaagccgcccgtctcaccgtatccggcgtctcattccacatcggtagcggagatgccgattcaaacgcttatctcggcgccatagccgcggctaaggaagtgtttgaaacagctgctaaactcgggatgtcgaaaatgactgttctagacgtcggcggcgggtttacatccggccaccagttcacaaccgccgccgtcgccgttaaatcagctttaaaacaacacttcgatgacgaaccggagttgacaatcatagctgaaccgggtcggttttttgcagagacggcgtttactttggcaacgacgattatagggaaaagagtgaggggtgaattgagggagtattggattaacgacgggctgtacggttcgatgaactgtgtactttacgaccatgcgacggtgaatgcaacgccgttagctgttctgtcgaatcgtagtaacgttacctgcggcgggtcgaaaacgtttccgacgactgtgtttgggcccacttgtgatgctcttgatactgttttaagggattaccagttaccggagctgcaggttaatgattggctggtttttcctaatatgggtgcttatactaaagctgctgggtccaattttaatggatttaatacttccgccattgttactcacctcgcttattcttatccaagctga

SEQ ID NO: 31 - DNA, 1173 bp, Artificial Sequence
  Description of Artificial Sequence Synthetic polynucleotide
atggaaacttttttgttcacctcagaatcagtcaatgaaggccatcccgacaagctctgtgaccaagtctcagatgccattcttgatgcttgccttgcacaagatccagagagcaaagttgcatgtgaaacctgcacaaagacaaacatggtcatggtctttggtgagatcacaaccaaagccaatgtagactatgaggagatagtacgtgatacttgtagaggcattgggttcacatcacctgatgtcggccttgatgctgacaactgcaaggtccttgtcaacatcgagcagcagagccctgacattgctcaaggagttcatggtcatcttacgaagaaaccagaggaaattggagctggtgaccaaggtcacatgtttggctatgccaccgatgaaactcctgagctcatgccccttactcatgttttggctactcagcttggtgccaagcttactgaagtgaggaagaacaagacttgtccatggctcagaccagatggcaagacacaagttacagttgagtacaagaatgagaatggtgccatggttcctattagagttcacactgttctcatctcaactcaacatgacgaaactgtcacaaatgagcagattgccaaggacttgaaagagcatgtaatcaaacccgtgatcccagctaagtaccttgatgagaaaaccatcttccacctcaacccatcaggtcgctttgtgattggtggtccacatggagatgctggactcactggcaggaaaatcatcattgatacctacggaggatggggcgctcatggtggaggtgccttctcagggaaagatcctactaaggtggacagaagtggtgcttatgttgttagacaggcagcaaagagtgtggttgctgcaggacttgctcgccgctgtattgtccaggtttcttatgcaattggtgtggcagaaccactctccgtgtttgttgacacttacaaaactggaaccattccggacaaggatattttggctttgatcaaggagaactttgacttcaggcctggaatgattgcaattaatcttgacttgaagagaggaggtaacttcaggtaccagaagactgctgcttatggtcacttcggccgtgatgaccctgatttcacctgggagactgtcaagcccctcaagcctaaggcttga

SEQ ID NO: 32 - DNA, 1173 bp, Artificial Sequence
  Description of Artificial Sequence Synthetic polynucleotide
atggaaactttcttgtttacctcagaatcagtcaacgaaggccatcccgacaagctctgtgaccaagtctcagatgccattcttgatgcttgccttgctcaagatcctgagagcaaggttgcatgtgaaacctgcacaaagacaaacatggtcatggtctttggtgagatcacaaccaaagccaatgtagactatgagaagatagtccgtgatacttgcagaggcattgggttcacctcaccagatgtcggccttgatgctgacaactgcaaggtccttgtcaacatcgagcagcagagccctgacattgcccaaggagttcatggtcatcttacaaagaaaccggaggaaattggagctggtgaccaaggtcacatgtttggctatgccaccgatgaaactcctgagctcatgccccttactcacgttttggctactcagcttggtgccaagcttactgaagtgaggaagaacaagacttgcccatggctcagaccagatggcaagacacaagttacagttgagtacaagaatgagaatggtgcaatggttcctattagagttcacaccgttctcatctcaactcaacatgatgaaactgtcacaaatgagcagattgcccgggacttgaaagagcatgtgatcaagccggtgatcccagctaagtaccttgatgagaaaaccatcttccacctcaacccatcaggtcgctttgtgattggtggtccccatggagatgctggactcactggcaggaaaatcatcattgacacctatggaggatggggcgctcatggtggaggcgcgttctcaggaaaggatcctactaaggtggatagaagtggtgcttatattgttagacaggcagcaaagagtgtggttgctgcaggacttgctcgccgctgtattgtccaagtttcttatgcaattggtgtggcagaaccactctccgtgtttgttgacagttacaaaaccggaaccattccagataaggatattttggctttgatcaaggagaactttgacttcaggcctggaatgattgcaattaatcttgacttgaagagaggaggtaacttcaggtaccagaagactgctgcttatggtcacttcggccgtgatgaccctgatttcacatgggagactgtcaaggccctcaagcctaaagcttga

SEQ ID NO: 33 - DNA, 1173 bp, Artificial Sequence
  Description of Artificial Sequence Synthetic polynucleotide
atggagactttcttgttcacctcagaatcagttaacgaagggcatcccgacaagctttgcgaccaggtctcagatgccattcttgacgcttgcctagaacaagatccagagagcaaggttgcatgtgaaacctgcacaaagacaaacatggtcatggtctttggagagataacaaccaaggccaaggtggactatgaaaagatagtacgcgacacttgcagaggcatcgggttcacctcagctgatgttggccttgatgctgaccactgcaaagtccttgtcaacattgagcagcagagccctgacattgcccaaggagtccatggtcatcttaccaagaaaccagaagaaatcggagctggtgaccaaggtcacatgtttggctatgccacagatgaaactcctgaactaatgccccttactcatgttttggctactcagcttggtgccaagcttactgaagtgaggaagaacaagacttgcccatggctcagaccggatggcaagacacaagttacagttgagtacaagaatgacaatggcgccatggttcctattagagttcacaccgttctcatctcaactcaacatgatgaaggtgtcacaaatgagcagattgcccaggacttgaaagagcatgtgataaagcccgtgatcccagcgaagtaccttgatgagaacaccatcttccacctcaacccatcaggtcgctttgtgattggtggtccacatggagatgctggactcactggcaggaaaattatcattgacacctatggaggttggggtgctcatggtggaggtgccttctcagggaaagatcctaccaaggtggacagaagtggtgcttatattgtgagacaagcagcaaagagtgtggtagcagcaggacttgctcgccgctgcattgtccaggtttcttatgcaattggtgttgcagaaccactctccgtgtttgttgacacttacaaaaccggaaccattccagacaaggatattctggctctgatcaaggagaactttgacttcaggcctggaatgatggcaattaaccttgacttactcagaggaggtaacttcaggtaccagaagactgctgcttatggtcactttggccgtgatgaccctgatttcacctgggagactgtcaaggccctcaagcctaaagcttga

SEQ ID NO: 34 - DNA, 1173 bp, Artificial Sequence
  Description of Artificial Sequence Synthetic polynucleotide
atggagactttcttgttcacctcagaatcagttaacgaagggcatcccgacaagctttgcgaccaggtgtcagatgccattcttgacgcttgcctagaacaagatccagagagcaaggttgcatgtgaaacctgcacaaagacaaacatggtcatggtctttggagagataacaaccaaggccaaggtggactatgaaaagatagtacgcgacacttgcagaggcattgggttcacctcagctgatgttggccttgatgctgaccactgcaaagtccttgtcaacattgagcagcagagccctgatattgcccaaggagtccatggtcatcttaccaagaaaccggaagaaattggagctggtgaccaaggtcacatgtttggctatgccacagatgaaactcctgaactcatgcctcttacccatgttttggctactcagcttggtgccaagcttaccgaagtgaggaagaacaagacttgcccatggctcagaccggatggcaagacacaagttacagttgagtacaagaatgacaatggtgccatggttcctattagagttcacaccgttctcatctcaactcaacatgatgaaggtgtcacaaatgagcagatcgcccaggacttgaaagaacatgtgatcaagcccgtgatcccagcgaagtaccttgatgagaacaccatcttccacctcaacccatcaggtcgctttgtgattggtggtccacatggagatgctggactcactggcaggaaaattatcatcgacacctatggaggttggggtgctcacggtggaggtgccttctcagggaaagatccgaccaaggtggacaggagtggtgcttatattgtgagacaagcagcaaagagtgtggtcgcagcaggacttgctcgccgctgcattgtccaggtttcttatgcaattggtgttgctgaaccactctccgtttttgttgacacttacaaaaccggaaccattccagacaaggatattttggctctgatcaaggagaactttgacttcaggcctggaatgattgcaattaaccttgacttgcttagaggaggtaacttcaggtaccagaagactgctgcttatggtcactttggccgtgatgaagctgatttcacctgggagactgtcaaggccctcaagcctaaagcttga

SEQ ID NO: 35 - DNA, 1173 bp, Artificial Sequence
  Description of Artificial Sequence Synthetic polynucleotide
atggaaactttcttgttcacctcagagtcagtcaatgaaggccaccccgacaagctctgtgaccaggtctcagatgcaattcttgatgcttgcttagaacaggatccagaaagcaaggttgcatgtgaaacctgcacaaagacaaacatggttatggtctttggagagatcacaaccaaggccactgtcgactatgagaagatagtacgtgacacatgcagaggcatcgggttcacctcagcagatgtcggccttgacgctgacaactgcaaggtccttgtcaacattgagcagcagagccctgacattgcccaaggtgttcacgggcatcttaccaagaaaccagaagagattggagctggtgaccaaggtcacatgtttggctatgccactgatgaaactccagagctcatgccccttacccatgttttggccactaagcttggcgccaagcttactgaagtgagaaagaacaagacttgcccatggctcagaccagatggcaagacccaagttactgttgagtacaagaacgacaatggtgccatggtccccattagagttcacactgttctcatctctactcaacatgacgaaactgtcacaaacgaccagattgcccaggacttgaaagagcatgtgattaaacctgtgatcccagctcagtaccttgatgagaataccatcttccacctcaacccatcaggtcgcttcgtcatcggtggaccacacggagatgctggacttaccggcaggaaaattatcattgacacctacggaggctggggtgcccatggtggaggtgctttctcaggaaaggatcctactaaggtggacaggagtggtgcttatattgttagacaggcagcaaagagtgtggtcgcctcaggacttgctcgccgctgtattgtgcaggtttcttatgctatcggtgtggctgaaccactttccgtgtttgttgacacttacaagactggaacaattccagacaaggatattttggctctgatcaaggagaactttgacttcaggcctggaatgatgtcaatcaatcttgacttgttaagaggaggcaacttcaggtaccagaagactgcagcttatggtcactttggccgtgatgaccccgacttcacatgggagactgtcaagagcctcaagcctaaagcttaa

SEQ ID NO: 36 - DNA, 1143 bp, Artificial Sequence
  Description of Artificial Sequence Synthetic polynucleotide
atgcatgcattacttagggcagacatggaaactttcttgttcacctcagagtcagtcaatgaaggccaccccgacaagctctgcgaccaggtctcggatgcaattcttgatgcttgcttagaacaggatccagaaagcaaggttgcatgtgaaacctgcacaaagacaaacatggttatggtctttggagagatcacaaccaaggccactgttgactatgagaagatagtgcgtgacacatgcagaggcattgggttcacctcagcagatgttggccttgacgctgacaactgcaaggttcttgtcaacatcgagcagcagagccctgacattgcccaaggtcacatgtttggctatgccactgatgaaaccccagagctcatgccccttacccatgttttggccactaagcttggtgccaagcttaccgaagtgaggaagaacaagacttgcccatggctcagaccagatggcaagacccaagttactgttgagtacaagaacgacaatggtgccatggtcccaattagagttcacactgttctcatctcaactcaacatgacgaaactgtcacaaacgaccagattgcccaggacttgaaagagcatgtgatcaaacctgtgatcccatctcagtaccttgatgagaataccatcttccacctcaacccatcaggtcgcttcgtcatcggtggaccacacggagatgctggacttaccggcaggaaaattatcattgacacctacggaggctggggtgcccatggtggaggtgctttctcaggaaaggaccctactaaggtggacaggagtggtgcttatattgttagacaggcagcaaagagtgtggtcgcctcaggacttgctcgccgctgtattgtgcaggtttcttatgctatcggtgtggctgaaccactttccgtgtttgttgacacttacaagactggaacaattccagacaaggatattttggctctgatcaaggagaactttgacttcaggcctggaatgatgtcaatcaaccttgacttgttaagaggaggcaacttcaggtaccagaagactgcagcttatggtcactttggccgtgatgaccccgacttcacatgggagactgtcaaggtcctcaagccaaaagcttaa

SEQ ID NO: 37 - protein, 286 aa, Artificial Sequence
  Description of Artificial Sequence Synthetic polypeptide
MEQKGQEKEMGIPNSIGYNPSSSFSDTRRNEKAGSGNVILSPIQTLNQHNQHSLQGHSQLNSHQLEEERDPLVAATNRAQLRQQSTTSGNGTTTTTTSSSSVVRRYRECLKNHVASMGGHVLDGCGEFMPSEEEGALKCAACNCHRNFHRRETFSEAETQIARPRPPYSTFNPPIHSSSNYHHNYSTASPVMMTFGRNAESSSEHLNLFNSNAGGGQSSKSKKRFRTKFTAEQKERMHEFAEKVGWSIQKQDEQELQHFCNQVGVKRQVFRIWMHNSKQANKKRQM

SEQ ID NO: 38 - protein, 519 aa, Artificial Sequence
  Description of Artificial Sequence Synthetic polypeptide
MYHLLSPIEAIVGLVTFAFLLYFLWTKKQSKILNPLPPKIPGGWPVIGHLFYFKNNGDDDRHFSQKLGDLADKYGPVFTFRLGFRRFLAVSSYEAMKECFSTNDIHFADRPALLYGEYLCYNNAMLAVAKYGPYWKKNRKLVNQELLSVSRLEKFKHVRFSIVQKNIKELYDGDSPMVKINLSDWIDKLTFDIILKMVVGKTYNNGHGEILKAAFQKFMVQAMEIELYDVFHIPFFKWLDLTGNIKAMKQTFKDIDNIIQGWLDEHIKKRETKDVGGENEQDFIDVVLSKRSNEHLGDGYSHDTTIKATVFTLVLDATDTLALHIKWVMALMINNKNVMKKAQEEMDTIVGRDRWVEESDIKNLVYLQAIVKEVLRLHPPAPLSVQHLSVKDCVVNGYHIPKGTALLTNIMKLQRDPQIWADPDKFDPERFLTTHAAIDYRGQHYELIPFGTGRRACPAMNYSLQVEHLSIAHMIQGFNFATTTNEPLDMKQGVGLTLPKKTDVEVLITPRLPPTLYQY

SEQ ID NO: 39 - protein, 374 aa, Artificial Sequence
  Description of Artificial Sequence Synthetic polypeptide
MDMKGTPAENGYRMPAEWESHSHCWMGWPERPDNWRNNALHGQRVFANVASAISRFEPVTVCASAAQWENARSQLPKHIRVVEMSMNDSWFRDTGPTFVIRNTAANAEHVASSVAGIDWTFNSWGGVDDGCYEDWSHDLLVARKILAIEKVPRFPHSMILEGGSIHVDGEGTCLTTEECLLNKNRNPHLSKEQIENELKAYLGVRKIIWLPRGLFGDDDTNGHIDNMCCFVKPGVVLLSWTDDQSDPQYERSIEALSILETTSDANGRKLQVIKLHVPGPLCMTDEEADGIFQDGDAKPRTSGTRLAASYVNFYIANGAIITPQFGDKKWDEEAIRVLSLAFPDHEVVGIEGAREIVLGGGNIHCITQQQPTGP

SEQ ID NO: 40 - protein, 387 aa, Artificial Sequence
  Description of Artificial Sequence Synthetic polypeptide
MDLKGTPVENGYRMPAEWESHSHCWMGWPERPDNWRNNAFHGQHVFANVASAISRFEPVTVCASAAQWENARNLLPKHIRVVEMSMNDSWFRDTGPTFVIRNTTSEDTEHVAPSVAGIDWTFNSWGGVDDGCYEDWSHDLLVARKILAIEKVPRFPHSMILEGGSIHVDGEGTCLTTEECLLNKNRNLHLSKEQIENELKAYLGVRKIIWLPRGLLGDDDTNGHIDNMCCFVKPGVVLLSWTDDQSDPQYARSIEALSILETTSDANGRKLQVIKLHVPGPLCMTDEEADGIFQDGDAKPRTSGTRLAASYVNFYIANGAIITPQFGDKKWDEEAIRVLSLAFPDHEVVGIEGAREIVLGGGNIHCITQQQPSGPEYSPKILNGSTS

SEQ ID NO: 41 - protein, 648 aa, Artificial Sequence
  Description of Artificial Sequence Synthetic polypeptide
MATGIASGCGQLHLRKPVYLRNSYGNKAHSHSNVILNGTQNQIAWSSWVSNVLRVNRSSYPQCQVIKTNWKSRRGTIKSSQQRDGSVTRYFDFIVIGSGIAGLRYALEVAKHGTVAVITKAEPHESSTNYAQGGVSAVLCPLDSVENHIQDTIVAGAYLCDKETVKVVCTEGPERIRELIAIGASFDHGEDGNLDLAREGGHSHRRIVHAADMTGREIERALLEAVFKNPNIHVFQHHFAIDLLTTQDGSDVVCHGVDTIHTETKEVIRFISKVTLLASGGVGHIYPSTTNPTVATGDGMAMAHRAQAVISNMEFVQFHPTALADEGLPNIPSARENAFLITEAVRGDGGILYNLDMERFMPMYDERAELAPRDVVARSIDDQLKKRGEKYVLLDISHKPREKVLSHFPNIAAECLRHGLDITQQPIPVVPAAHYMCGGVRAGLEGETNVQGLYVAGEVACTGLHGANRLASNSLLEALVFARRAVQPSIDHVNVSRIDNGASSWWPRPVAPLALGDTVLNKVIRRTREVRKELQSIMWEYVGIVRSTSRLNTAEKRIKELELEWETYLFQHGWEPTMVGVEACEMRNLFCCANLVVSSALSRQESRGLHYTTDFPHVEESERLPTVIFPSQRNSTWSTRQLHAQPIS

SEQ ID NO: 42 - protein, 662 aa, Artificial Sequence
  Description of Artificial Sequence Synthetic polypeptide
MFWILEIDFPWVLSQEDHMATGIASGSGQLHLRKPVYWRSSYGKAHCHSNAILNGMQNQIPWSYWVSKFLRVHRSNYSQCQVKTNWKSHTGTINSCQRDGSTRYFDFAVIGSGIAGLRYALEVAKHGTVAVITKAEPHESNTNYAQGGVSAVFCPMDSVESHMQDTIVAGAYLCDEETVRVVCTEGPERIRELIAMGASFDHGEDGNLHLAREGGHSHRRIVHAADMTGREIERALLEAVFKHPNIHVFQHHFAIDFLTTQDGSDIICHGVDAINTETQEVIRFISKVTLLASGGAGHIYPSTTNPPVATGDGMAMAHRAQAVISNMEFVQFHPTALADEGLPNRPSARENAFLITEAVRGDGGILYNLDMERFMPMYDKRAELAPRDVVARSIDDQLKKRGEKYVLLDISHKPREKVLSHFPNIAAECLRHGLDITQQPIPVVPAAHYMCGGVRAGLEGETNVQGLYVAGEVACTGLHGANRLASNSLLEALVFARRAVQPSIDHVNVSRIDHCASSWWPRPVAPVVIGDTVLNKVIRRTREVRKELQSIMWEYVGIVRSTSRLTAAEKRINELELEWETYLFQHGWEPTMVGLEACEMRNLFCCANLVVSSALSRHESRGLHYTIDFPHVVESKRLPTIIFPSQRNSSWSSRQLHRQQIC

SEQ ID NO: 43 - protein, 648 aa, Artificial Sequence
  Description of Artificial Sequence Synthetic polypeptide
MATGIASGCGQLHLRKPVYLRNSYGNKAHCHSNVILNGTQNQIAWSSWVSNVLRVNRSSYPECQVIKTNWKSSRGTIKSCQQRDGSVTRYFDFTVIGSGIAGLRYALEVAKHGTVAVITKAEPHESSTNYAQGGVSAVLCPLDSVESHMQDTIVAGAYLCDKETVRVVCTEGPERIRELIAMGASFDHGEDGNLDLAREGGHSHRRIVHAADMTGREIERALLEAVFKNPNIHVFQHHFAIDLLTTQDGSDIVCHGVDTIHTETKEVIRFISKVTLLASGGVGHIYPSTTNPTVATGDGMAMAHRAQAVISNMEFVQFHPTALADEGLPNIPSARENAFLITEAVRGDGGILYNLDMERFMPMYDERAELAPRDVVARSIDDQLKKRGEKYVLLDISHKPREKVLSHFPNIAAECLRHGLDITQQPIPVVPAAHYMCGGVRAGLEGETNVQGLYVAGEVACTGLHGANRLASNSLLEALVFARRAVQPSIDHVNVSRIDHGASSWWPRPVAPMVLGDTVLNKVICRTREVRKELQSIMWEYVGIVRSNSRLNTAEKRIRELELEWETYLFQHGWEPTMVGVEACEMRNLFCCANLVVSSALSRHESRGLHYTTDFPHVEESERLPTVIFPSQRNNSWSSRQLHAQPIS

SEQ ID NO: 44 - protein, 645 aa, Artificial Sequence
  Description of Artificial Sequence Synthetic polypeptide
MATGIASVSGQLHLRKPVYWRSSYGKAQCHSNVILNGMQNQIPWSYWVSKFLRVHRSNYSQCQVKTNWKSHSGTINSCQRDGSTRYFDFAVIGSGIAGLRYALEVAKHGTVAVITKAEPHESNTNYAQGGVSAVLCPMDSVESHMQDTIVAGAYLCDEETVRVVCTEGPERIKELIAMGASFDHGEDGNLHLAREGGHSHRRIVHAADMTGREIERALLEAVFKHPNIHVFQHHFAIDLLTTQDGSDIVCHGVDTINTETQEVIRFISKVTLLASGGAGHTYPSTTNPPVATGDGMAMAHRAQAVISNMEFVQFHPTALADEGLPIRPTRTRENAFLITEAVRGDGGILYNLDMERFMPMYDERAELAPRDVVARSIDDQLKKRGEKYVLLDISYKPREKVLSHFPNIAAECLRHGLDITQQPIPVVPAAHYMCGGVRAGLEGETNVHGLYVAGEVACTGLHGANRLASNSLLEALVFARRAVQPSIDHMNVSRIDHGASSWWSRPVAPMLLGDTVHGKVIHRTREVRKELQSIMWEYVGIVRSTSRLTAAEKRIRELELEWETYLFQHGWEPTMVGVEACEMRNLFCCANLVVNSALSRHESRGLHYTIDFPHVEESKRLPTIIFPSQRNSSWSSRQLHRQQIC

SEQ ID NO: 45 - protein, 338 aa, Artificial Sequence
  Description of Artificial Sequence Synthetic polypeptide
MKSAGRMGIHYMQKLNAANVPKELVEKGQNRVIEASLTLIRERAKLKGELIRALGGAVASTSLLGVPLGHNSSFLQGPAFAPPRIREAIWCGSTNSTTEEGKELNDPRILTDVGDVPVQELRDSGVDDDRLMSIISESVKLVMEEDPLRPLVLGGDHSISYPVVRAVSEKLGGPIDILHLDAHPDIYHSFEGNKYSHASSFARIMEGGYARRLLQVGIRSITKEGREQGKRFGVEQYEMRTFSRDRQFLENLKLGEGVKGVYISVDIDCMDPAFAPGVSHIEPGGLSFRDVLNILHNLQADVVGADVVEFNPQRDTVDGMTAMVAAKLVRELTAKISK

SEQ ID NO: 46 - protein, 342 aa, Artificial Sequence
  Description of Artificial Sequence Synthetic polypeptide
MKCAGRMGIHYMQKLNAANVPKELVEKGQNRVIEASLTLIRERAKLKGELIRALGGAVASTSLLGVPLGHNSSFLQGPAFAPPRIREAIWCGSTNSTTEEGKELNDSRILTDVGDVPVQELRDSGVDDDRLMSIISESVKLVMEEDPLRPLVLGGDHSISYPVVRAVSEKLGGPIDILHLDAHPDIYHSFEGNKYSHASSFARIMEGGYARRLLQVGIRSITKEGREQGKRFGVEQYEMRTFSRDRQFLENLKLGEGAKGVYISVDIDCMDPAFAPGVSHIEPGGLSFRDVLNILHNLQADVVGADVVEFNPQRDTVDGMTAMVAAKLNKAFTNGARHSPEP

SEQ ID NO: 47 - protein, 432 aa, Artificial Sequence
  Description of Artificial Sequence Synthetic polypeptide
MAGQTIIVSGLNPAAILQSTIGGGASPTAAAAENGTRKVIPLSRDALQDFMLSIITQKLQDEKQPFYVLDLGEVVSLMDQWKSALPNIRPFYAVKCNPEPSFLSILSAMGSNFDCASRAEIEYVLSLGISPDRIVFANPCKPESDIIFAAKVGVNLTTYDSEDEVYKIRKHHPKSELLLRIKPMFDGNARCPMGPKYGALPEEVEPLLRAAQAARLTVSGVSFHIGSGDADSNAYLGAIAAAKEVFETAAKLGMSKMTVLDVGGGFTSGHQFTTAAVAVRSALKQHFDDQPELTIIAEPGRFFAETAFTLATTIIGKRVRGELREYWINDGLYGSMNCVLYDHATVNATPLAVLSNRTNVTCGGSKTFPTTVFGPTCDALDTVLRDYQLPELQVNDWLVFPNMGAYTKAAGSNFNGFNTSAIVTHLAYAYPS

SEQ ID NO: 48 - protein, 433 aa, Artificial Sequence
  Description of Artificial Sequence Synthetic polypeptide
MAGQTIIVSGLNPAAILQSTIGGGASPTAAAAAENGTRKVIPLSRDALQDFMLSIITQKLQDEKQPFYVLDLGEVVSLMDQWKSALPNIRPFYAVKCNPEPSFLSILSAMGSNFDCASRAEIEYVLSLGISPDRIVFANPCKPESDIIFAAKVGVNLTTYDSEDEVYKIRKHHPKSELLLRIKPMLDGNARCPMGPKYGALPEEVDPLLRAAQAARLTVSGVSFHIGSGDADSNAYLGAIAAAKEVFETAAKLGMSKMTVLDVGGGFTSGHQFTTAAVAVKSALKQHFDDEPELTIIAEPGRFFAETAFTLATTIIGKRVRGELREYWINDGLYGSMNCVLYDHATVNATPLAVLSNRSNVTCGGSKTFPTTVFGPTCDALDTVLRDYQLPELQVNDWLVFPNMGAYTKAAGSNFNGFNTSAIVTHLAYSYPS

SEQ ID NO: 49 - protein, 390 aa, Artificial Sequence
  Description of Artificial Sequence Synthetic polypeptide
METFLFTSESVNEGHPDKLCDQVSDAILDACLAQDPESKVACETCTKTNMVMVFGEITTKANVDYEEIVRDTCRGIGFTSPDVGLDADNCKVLVNIEQQSPDIAQGVHGHLTKKPEEIGAGDQGHMFGYATDETPELMPLTHVLATQLGAKLTEVRKNKTCPWLRPDGKTQVTVEYKNENGAMVPIRVHTVLISTQHDETVTNEQIAKDLKEHVIKPVIPAKYLDEKTIFHLNPSGRFVIGGPHGDAGLTGRKIIIDTYGGWGAHGGGAFSGKDPTKVDRSGAYVVRQAAKSVVAAGLARRCIVQVSYAIGVAEPLSVFVDTYKTGTIPDKDILALIKENFDFRPGMIAINLDLKRGGNFRYQKTAAYGHFGRDDPDFTWETVKPLKPKA

SEQ ID NO: 50 - protein, 390 aa, Artificial Sequence
  Description of Artificial Sequence Synthetic polypeptide
METFLFTSESVNEGHPDKLCDQVSDAILDACLAQDPESKVACETCTKTNMVMVFGEITTKANVDYEKIVRDTCRGIGFTSPDVGLDADNCKVLVNIEQQSPDIAQGVHGHLTKKPEEIGAGDQGHMFGYATDETPELMPLTHVLATQLGAKLTEVRKNKTCPWLRPDGKTQVTVEYKNENGAMVPIRVHTVLISTQHDETVTNEQIARDLKEHVIKPVIPAKYLDEKTIFHLNPSGRFVIGGPHGDAGLTGRKIIIDTYGGWGAHGGGAFSGKDPTKVDRSGAYIVRQAAKSVVAAGLARRCIVQVSYAIGVAEPLSVFVDSYKTGTIPDKDILALIKENFDFRPGMIAINLDLKRGGNFRYQKTAAYGHFGRDDPDFTWETVKALKPKA

SEQ ID NO: 51 - protein, 390 aa, Artificial Sequence
  Description of Artificial Sequence Synthetic polypeptide
METFLFTSESVNEGHPDKLCDQVSDAILDACLEQDPESKVACETCTKTNMVMVFGEITTKAKVDYEKIVRDTCRGIGFTSADVGLDADHCKVLVNIEQQSPDIAQGVHGHLTKKPEEIGAGDQGHMFGYATDETPELMPLTHVLATQLGAKLTEVRKNKTCPWLRPDGKTQVTVEYKNDNGAMVPIRVHTVLISTQHDEGVTNEQIAQDLKEHVIKPVIPAKYLDENTIFHLNPSGRFVIGGPHGDAGLTGRKIIIDTYGGWGAHGGGAFSGKDPTKVDRSGAYIVRQAAKSVVAAGLARRCIVQVSYAIGVAEPLSVFVDTYKTGTIPDKDILALIKENFDFRPGMMAINLDLLRGGNFRYQKTAAYGHFGRDDPDFTWETVKALKPKA

SEQ ID NO: 52 - protein, 390 aa, Artificial Sequence
  Description of Artificial Sequence Synthetic polypeptide
METFLFTSESVNEGHPDKLCDQVSDAILDACLEQDPESKVACETCTKTNMVMVFGEITTKAKVDYEKIVRDTCRGIGFTSADVGLDADHCKVLVNIEQQSPDIAQGVHGHLTKKPEEIGAGDQGHMFGYATDETPELMPLTHVLATQLGAKLTEVRKNKTCPWLRPDGKTQVTVEYKNDNGAMVPIRVHTVLISTQHDEGVTNEQIAQDLKEHVIKPVIPAKYLDENTIFHLNPSGRFVIGGPHGDAGLTGRKIIIDTYGGWGAHGGGAFSGKDPTKVDRSGAYIVRQAAKSVVAAGLARRCIVQVSYAIGVAEPLSVFVDTYKTGTIPDKDILALIKENFDFRPGMIAINLDLLRGGNFRYQKTAAYGHFGRDEADFTWETVKALKPKA

SEQ ID NO: 53 - protein, 390 aa, Artificial Sequence
  Description of Artificial Sequence Synthetic polypeptide
METFLFTSESVNEGHPDKLCDQVSDAILDACLEQDPESKVACETCTKTNMVMVFGEITTKATVDYEKIVRDTCRGIGFTSADVGLDADNCKVLVNIEQQSPDIAQGVHGHLTKKPEEIGAGDQGHMFGYATDETPELMPLTHVLATKLGAKLTEVRKNKTCPWLRPDGKTQVTVEYKNDNGAMVPIRVHTVLISTQHDETVTNDQIAQDLKEHVIKPVIPAQYLDENTIFHLNPSGRFVIGGPHGDAGLTGRKIIIDTYGGWGAHGGGAFSGKDPTKVDRSGAYIVRQAAKSVVASGLARRCIVQVSYAIGVAEPLSVFVDTYKTGTIPDKDILALIKENFDFRPGMMSINLDLLRGGNFRYQKTAAYGHFGRDDPDFTWETVKSLKPKA

SEQ ID NO: 54 - protein, 380 aa, Artificial Sequence
  Description of Artificial Sequence Synthetic polypeptide
MHALLRADMETFLFTSESVNEGHPDKLCDQVSDAILDACLEQDPESKVACETCTKTNMVMVFGEITTKATVDYEKIVRDTCRGIGFTSADVGLDADNCKVLVNIEQQSPDIAQGHMFGYATDETPELMPLTHVLATKLGAKLTEVRKNKTCPWLRPDGKTQVTVEYKNDNGAMVPIRVHTVLISTQHDETVTNDQIAQDLKEHVIKPVIPSQYLDENTIFHLNPSGRFVIGGPHGDAGLTGRKIIIDTYGGWGAHGGGAFSGKDPTKVDRSGAYIVRQAAKSVVASGLARRCIVQVSYAIGVAEPLSVFVDTYKTGTIPDKDILALIKENFDFRPGMMSINLDLLRGGNFRYQKTAAYGHFGRDDPDFTWETVKVLKPKA

SEQ ID NO: 55 - DNA, 300 bp, Artificial Sequence
  Description of Artificial Sequence Synthetic polynucleotide
ctccgggcgatgcagcacgagcctgaactcatgttcgagacgctcaagcaccgcgctgaagaattcgtgcacaatgacgacgaacaagaagaagataaagggcttgcatttgcatctttggccagcagcttagctcagtcattcaacaatatgccttaccttgttactaattcatcttgctgccttactgctgctgccaataacggtggctattactattgcaatgatgagaacattgttggagttggcgcagaatctgctgcagctgaagaagagctttggccttactgtgttgcttga

SEQ ID NO: 56 - DNA, 378 bp, Artificial Sequence
  Description of Artificial Sequence Synthetic polynucleotide
agtgttgcaggaattgattggaccttcaatagctggggtggtgttgatgatggatgttacgaagactggagtcatgaccttcttgttgcaagaaagattttggcaattgaaaaggtcccaaggtttccacactctatgattcttgaaggtggaagcattcatgtcgatggagaagggacttgtctcacgacagaagagtgcctcctgaacaaaaacaggaacccccatttgtctaaggaacaaattgagaatgaactaaaggcatatcttggagtcaggaagattatttggctgcctcgagggttgtttggtgatgatgatacaaatggtcatattgacaacatgtgctgttttgtgaagcctggtgttgttttattg

SEQ ID NO: 57 - DNA, 229 bp, Artificial Sequence
  Description of Artificial Sequence Synthetic polynucleotide
tgaggaatagctatggaaataaagctcactctcattccaatgtgattctcaacggcacgcaaaaccagatcgcttggtctagttgggtttcaaatgtcttgcgagttaatagaagtagctatccacaatgtcaagtgataaaaacaaactggaagtctcggcgaggaacaatcaaatctagccagcagagagatggatcagttactaggtacttcgatttcattgtgat

SEQ ID NO: 58 - DNA, 230 bp, Artificial Sequence
  Description of Artificial Sequence Synthetic polynucleotide
gaaaagctcactgtcattccaatgcgatcctgaatggcatgcaaaaccagatcccttggtcttattgggtttccaaattcttgcgagttcatagaagtaactactcacaatgtcaagtgaaaacaaactggaagtctcacaccggaacaatcaattcttgccagagagatggctcaactaggtattttgattttgctgtgattggtagtggaattgctggccttagatat

SEQ ID NO: 59 - DNA, 224 bp, Artificial Sequence
  Description of Artificial Sequence Synthetic polynucleotide
gaaaagctcagtgtcattccaatgtgatcctgaatggcatgcaaaaccagatcccttggtcttattgggtttccaaattcttgcgagttcatagaagtaactattcacaatgtcaagtgaaaacaaactggaagtctcacagcggaacaatcaattcttgccagagagatggctcaactaggtatttcgatttcgcggtgattggtagtggaattgctggcctt

SEQ ID NO: 60 - DNA, 302 bp, Artificial Sequence
  Description of Artificial Sequence Synthetic polynucleotide
cgagatagtggcgttgatgatgataggttgatgagtatcataagtgaatctgtcaagctagtgatggaggaggatccattgcgccccttggtgttagggggtgatcactccatatcctatcctgttgtaagagctgtgtctgaaaagcttggaggacctattgatatccttcaccttgatgctcatcctgatatttaccattcctttgaaggaaacaaatattcacatgcatcaagctttgcacgaattatggagggtggttacgctcgacggcttttgcaagtgggaattagatcaattac

SEQ ID NO: 61 - DNA, 347 bp, Artificial Sequence
  Description of Artificial Sequence Synthetic polynucleotide
gccattcttcagtccacaataggcggcggagcttcacctacagcggcggcggcggaaaacggtaccagaaaagtcatccctctctcaagagatgccctccaagatttcatgttatcaatcataacccaaaaattacaagatgagaaacaacctttttacgtgttagatttgggtgaagttgtttctcttatggaccaatggaaatctgctctcccaaatatccgtccattttacgctgttaaatgtaaccctgaaccgtcgttcctttcaattttatctgctatgggctcaaattttgattgtgctagccgagctgaaattgagtacgttttgtcccttggcatttc

SEQ ID NO: 62 - DNA, 281 bp, Artificial Sequence
  Description of Artificial Sequence Synthetic polynucleotide
gctttgtgattggtggtccacatggagatgctggactcactggcaggaaaatcatcattgatacctacggaggatggggcgctcatggtggaggtgccttctcagggaaagatcctactaaggtggacagaagtggtgcttatgttgttagacaggcagcaaagagtgtggttgctgcaggacttgctcgccgctgtattgtccaggtttcttatgcaattggtgtggcagaaccactctccgtgtttgttgacacttacaaaactggaaccattccggac

SEQ ID NO: 63 - DNA, 281 bp, Artificial Sequence
  Description of Artificial Sequence Synthetic polynucleotide
gctttgtgattggtggtccacatggagatgctggactcactggcaggaaaattatcattgacacctatggaggttggggtgctcatggtggaggtgccttctcagggaaagatcctaccaaggtggacagaagtggtgcttatattgtgagacaagcagcaaagagtgtggtagcagcaggacttgctcgccgctgcattgtccaggtttcttatgcaattggtgttgcagaaccactctccgtgtttgttgacacttacaaaaccggaaccattccagac

SEQ ID NO: 64 - DNA, 281 bp, Artificial Sequence
  Description of Artificial Sequence Synthetic polynucleotide
gcttcgtcatcggtggaccacacggagatgctggacttaccggcaggaaaattatcattgacacctacggaggctggggtgcccatggtggaggtgctttctcaggaaaggatcctactaaggtggacaggagtggtgcttatattgttagacaggcagcaaagagtgtggtcgcctcaggacttgctcgccgctgtattgtgcaggtttcttatgctatcggtgtggctgaaccactttccgtgtttgttgacacttacaagactggaacaattccagac

SEQ ID NO: 65 - DNA, 20 bp, Artificial Sequence
  Description of Artificial Sequence Synthetic primer
acgtttccgacgactgtgtt

SEQ ID NO: 66 - DNA, 20 bp, Artificial Sequence
  Description of Artificial Sequence Synthetic primer
gcagctccggtaactggtaa

SEQ ID NO: 67 - DNA, 20 bp, Artificial Sequence
  Description of Artificial Sequence Synthetic primer
gtgtcacagagcgatagccc

SEQ ID NO: 68 - DNA, 20 bp, Artificial Sequence
  Description of Artificial Sequence Synthetic primer
tgcttgagcgtctcgaacat

SEQ ID NO: 69 - DNA, 20 bp, Artificial Sequence
  Description of Artificial Sequence Synthetic primer
tgttgtgccaacctggtagt

SEQ ID NO: 70 - DNA, 20 bp, Artificial Sequence
  Description of Artificial Sequence Synthetic primer
gttggcaacctctcgctttc

SEQ ID NO: 71 - DNA, 21 bp, Artificial Sequence
  Description of Artificial Sequence Synthetic primer
tgacttcaggcctggaatgat

SEQ ID NO: 72 - DNA, 20 bp, Artificial Sequence
  Description of Artificial Sequence Synthetic primer
ccttgacagtctcccaggtg

SEQ ID NO: 73 - DNA, 20 bp, Artificial Sequence
  Description of Artificial Sequence Synthetic primer
ggtacaaggcttgctgcttc

SEQ ID NO: 74 - DNA, 20 bp, Artificial Sequence
  Description of Artificial Sequence Synthetic primer
tctggaaacgccagtgagag

SEQ ID NO: 75 - DNA, 20 bp, Artificial Sequence
  Description of Artificial Sequence Synthetic primer
gcggtctctctttccgtgat

SEQ ID NO: 76 - DNA, 20 bp, Artificial Sequence
  Description of Artificial Sequence Synthetic primer
gcagtcatgccatcaacagt

SEQ ID NO: 77 - DNA, 23 bp, Artificial Sequence
  Description of Artificial Sequence Synthetic primer
tggtcaggagattgcgaaagagc

SEQ ID NO: 78 - DNA, 21 bp, Artificial Sequence
  Description of Artificial Sequence Synthetic primer
acgcaaaacgctccaatggtg

SEQ ID NO: 79 - DNA, 20 bp, Artificial Sequence
  Description of Artificial Sequence Synthetic primer
caagatggattgcacgcagg

SEQ ID NO: 80 - DNA, 20 bp, Artificial Sequence
  Description of Artificial Sequence Synthetic primer
tgatattcggcaagcaggca

SEQ ID NO: 81 - DNA, 20 bp, Artificial Sequence
  Description of Artificial Sequence Synthetic primer
ccagaaggtaattatccaag

SEQ ID NO: 82 - DNA, 21 bp, Artificial Sequence
  Description of Artificial Sequence Synthetic primer
acaaagccaagaaagggtact

SEQ ID NO: 83 - DNA, 23 bp, Artificial Sequence
  Description of Artificial Sequence Synthetic primer
agatcttcaacacctacaccatt

SEQ ID NO: 84 - DNA, 20 bp, Artificial Sequence
  Description of Artificial Sequence Synthetic primer
agttgctcgaggaattcccg